(19) United States
(12) Patent Application Publication
(10) Pub. No.: US 2025/0081843 A1
(43) Pub. Date: Mar. 6, 2025

(54) ORGANIC COMPOUNDS AND ORGANIC LIGHT EMITTING DIODE COMPRISING THE SAME

(71) Applicant: MATERIAL SCIENCE CO.,LTD., Seoul (KR)

(72) Inventors: Hani JEON, Seoul (KR); Tae Wan LEE, Seoul (KR); Jie SONG, Seoul (KR); Sangmee KIM, Seoul (KR); Seunguk CHO, Seoul (KR); Seung Hye JEONG, Seoul (KR)

(21) Appl. No.: 18/796,562

(22) Filed: Aug. 7, 2024

(30) Foreign Application Priority Data
Aug. 7, 2023 (KR) 10-2023-0102825
Jul. 30, 2024 (KR) 10-2024-0101267

(51) Int. Cl. H10K 85/60; C07D 307/91; C07D 405/12; C07D 409/12; C09K 11/06; H10K 50/15

(52) U.S. Cl. CPC H10K 85/636; C07D 307/91; C07D 405/12; C07D 409/12; C09K 11/06; H10K 85/633; C09K 2211/1018; H10K 50/156; H10K 85/615; H10K 85/626; H10K 85/6572; H10K 85/6574; H10K 85/6576

(57) ABSTRACT

An organic compound represented by a Chemical Formula 1 in accordance with the present invention exhibits excellent hole injection and hole transport characteristics. In addition, an hole transport auxiliary layer of the organic light-emitting diode in accordance with the present invention contains the organic compound represented by the Chemical Formula 1 to lower an operation voltage, and improve efficiency, and lifetime characteristics of the organic light-emitting diode.

### CROSS-REFERENCE TO RELATED APPLICATION

This application claims priority from Korean Patent Applications No. 10-2023-0102825 filed on Aug. 7, 2023, and No. 10-2024-0101267 filed on Jul. 30, 2024 in the Korean Intellectual Property Office, and all the benefits accruing therefrom under 35 U.S.C. 119, the contents of which in its entirety are herein incorporated by reference.

### BACKGROUND

### Field

The present disclosure relates to an organic compound and an organic light-emitting diode including the same.

### Description of Related Art

An organic light-emitting diode (OLED) has a simpler structure compared to other flat panel display devices such as a liquid crystal display (LCD), a plasma display panel (PDP), and a field emission display (FED), and has various advantages in terms of a manufacturing process, and has excellent high luminance and wide viewing angle, fast response speed, and low operation voltage, and thus is being actively developed and commercialized as a flat display such as a wall-mounted TV, a backlight for a display, lighting, and billboards.

The organic light-emitting diode includes two electrodes, and an organic material layer therebetween. Electrons and holes from two electrodes are injected into a light-emitting layer in which excitons are generated via recombination of electrons and holes. When the generated excitons change from an excited state to a ground state, the light is generated.

The organic light-emitting diode may include at least one light-emitting layer. In general, the organic light-emitting diode having a plurality of light-emitting layers includes light-emitting layers that emit light beams with different peak wavelengths. Thus, a specific color may be rendered via a combination of the light beams with the different peak wavelengths.

The organic light-emitting diode may be classified into a top emission type light-emitting diode and a bottom emission type light-emitting diode. The top emission type light-emitting diode emits light generated in the light-emitting layer toward a translucent anode using a reflective cathode. On the other hand, in the bottom emission type light-emitting diode, light generated in the light-emitting layer is reflected from a reflective anode to be directed toward a transparent cathode, that is, toward a driving thin film transistor.

### Prior Patent Literature

[Prior Patent Document 1] KR 2019-0020514 A
[Prior Patent Document 2] KR 2083707 B1

### SUMMARY

A purpose of the present disclosure is to provide a novel organic compound and an organic light-emitting diode including the same.

Purposes of the present disclosure are not limited to the above-mentioned purpose. Other purposes and advantages of the present disclosure that are not mentioned may be understood based on following descriptions, and may be more clearly understood based on embodiments of the present disclosure. Further, it will be easily understood that the purposes and advantages of the present disclosure may be realized using means shown in the claims and combinations thereof.

According to one aspect of the present disclosure, an organic compound represented by a following Chemical Formula 1 is provided:

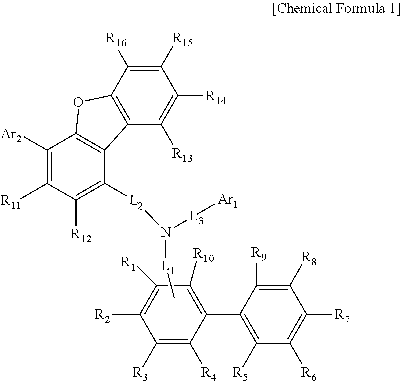

wherein in the Chemical Formula 1,
each of L₁ to L₃ independently represents one selected from a group consisting of a single bond, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, and a substituted or unsubstituted heteroarylalkyl group having 6 to 60 carbon atoms,
R₁ to R₁₆ and Ar are identical with or different from each other, wherein each of R₁ to R₁₆ and Ar independently represents one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6 to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms,
Ar₂ is a substituted or unsubstituted aryl group having 6 to 30 carbon atoms,
optionally, each of L₁ to L₃, R₁ to R₁₆, and Ar₁ and Ar₂ may be substituted with at least one substituent selected from a group consisting of deuterium, a cyano group, a trifluoromethyl group, a nitro group, a halogen group, a hydroxy group, a trimethylsilylethynyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an aralkyl group having 7 to 30 carbon atoms, a heteroaryl group having 5 to 60 carbon atoms, a heteroaralkyl group having 6 to 60 carbon atoms, an amine group, an alkylamino group having 1 to 30 carbon atoms, an aralkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms,
when the at least one substituent includes a plurality of substituents, the plurality of substituents are identical with or different from each other.

According to another aspect of the present disclosure, an organic light-emitting diode includes a positive electrode; a negative electrode facing the positive electrode; and at least one organic material layer disposed between the positive electrode and the negative electrode, wherein at least one of the at least one organic material layer contains the organic compound represented by the Chemical Formula 1.

The organic compound represented by the Chemical Formula 1 in accordance with the present disclosure exhibits excellent hole injection and hole transport characteristics.

In addition, an hole transport auxiliary layer of the organic light-emitting diode in accordance with the present disclosure contains the organic compound represented by the Chemical Formula 1 to lower an operation voltage, and improve efficiency, and lifetime characteristics of the organic light-emitting diode.

In addition, when the organic compound represented by the Chemical Formula 1 in accordance with the present disclosure is used as a material of the hole transport auxiliary layer, the hole transport auxiliary layer may have a suitable energy level at which the hole transport auxiliary layer may transfer holes from the hole transport layer to the light-emitting layer and may block electrons coming from the light-emitting layer.

Additionally, in the organic light-emitting diode in accordance with the present disclosure, even when the hole transport auxiliary layer containing the organic compound represented by the Chemical Formula 1 in accordance with the present disclosure may be combined with a light-emitting layer emitting light of any color, the light-emitting layer may excellently realize a color of target color coordinates.

The effect of the present disclosure is not limited to the effects mentioned above, and other effects not mentioned may be clearly understood by those skilled in the art from the entire description of the present disclosure.

### DETAILED DESCRIPTIONS

The above-mentioned purposes, features, and advantages are described in detail below, and accordingly, those skilled in the art in the technical field to which the present disclosure belongs will be able to easily implement the technical ideas of the present disclosure.

Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, “including”, “contain”, “containing”, etc. when used in this specification, specify the presence of the stated features, integers, operations, elements, and/or components, but do not preclude the presence or addition of one or more other features, integers, operations, elements, components, and/or portions thereof.

In descriptions of temporal relationships, for example, temporal precedent relationships between two events such as “after”, “subsequent to”, “before”, etc., another event may occur therebetween unless “directly after”, “directly subsequent” or “directly before” is not indicated.

In interpreting a numerical value, the value is interpreted as including an error range unless there is no separate explicit description thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the term “halogen group” includes fluorine, chlorine, bromine and iodine.

As used herein, the term “alkyl group” refers to both straight-chain alkyl radicals and branched-chain alkyl radicals. Unless otherwise specified, an alkyl group contains 1 to 30 carbon atoms. In this case, the alkyl group may include methyl, ethyl, propyl, isopropyl, butyl, secondary butyl, isobutyl, tert-butyl, pentyl, isoamyl, hexyl, etc., but is not limited thereto. Additionally, the alkyl group may be optionally substituted.

As used herein, the term “cycloalkyl group” refers to a cyclic alkyl radical. Unless otherwise specified, a cycloalkyl group contains 3 to 20 carbon atoms. In this case, the cycloalkyl group may include cyclopropyl, cyclobutyl, cyclopentyl, cyclohexyl, norbornyl, adamantyl, etc., but is not limited thereto. Additionally, the cycloalkyl group may be optionally substituted.

As used herein, the term “alkenyl group” refers to both straight-chain alkenyl radicals and branched-chain alkenyl radicals having one or more carbon-carbon double bonds. Unless otherwise specified, an alkenyl group contains 2 to 30 carbon atoms. In this case, the alkenyl group may include vinyl, allyl, isopropenyl, 2-butenyl, etc., but is not limited thereto. Additionally, the alkenyl group may be optionally substituted.

As used herein, the term “cycloalkenyl group” refers to a cyclic alkenyl radical. Unless otherwise specified, a cycloalkenyl group contains 3 to 20 carbon atoms. Additionally, the cycloalkenyl group may be optionally substituted.

As used herein, the term “alkynyl group” refers to both straight-chain and branched-chain alkynyl radicals having one or more carbon-carbon triple bonds. Unless otherwise specified, an alkynyl group contains 2 to 30 carbon atoms. In this case, an alkynyl group may include, but is not limited to, ethynyl, 2-propynyl, etc. Additionally, the alkynyl group may be optionally substituted.

As used herein, the term “cycloalkynyl group” refers to a cyclic alkynyl radical. Unless otherwise specified, a cycloalkynyl group contains 3 to 20 carbon atoms. Additionally, cycloalkynyl groups may be optionally substituted.

The terms “aralkyl group” and “arylalkyl group” as used herein are used interchangeably with each other and refer to an alkyl group having an aromatic group as a substituent. Additionally, the aralkyl group (arylalkyl group) may be optionally substituted.

The terms “aryl group” and “aromatic group” as used herein are used as having the same meaning, and the aryl group includes both a monocyclic group and a polycyclic group. The polycyclic group may include a “fused ring” in which two or more rings are fused with each other such that two carbons are common to two adjacent rings. Moreover, in the polycyclic group, two or more rings may be simply attached or fused to each other. Unless otherwise specified, the aryl group contains 6 to 30 carbon atoms. In this case, the aryl group may include phenyl, naphthyl, anthracenyl, phenanthrenyl, fluorenyl, dimethylfluorenyl, diphenylfluorenyl, spirofluorenyl, etc. but is not limited thereto. Additionally, the aryl group may be optionally substituted.

The terms “heteroaryl group” and “heteroaromatic group” as used herein are used as having the same meaning, and the heteroaryl group includes both a monocyclic group and a polycyclic group. The polycyclic group may include a “fused ring” in which two or more rings are fused with each other such that two carbons or heteroatoms are common to two adjacent rings. Moreover, in the polycyclic group, two or more rings may be simply attached or fused to each other. Unless otherwise specified, the heteroaryl group contains 1 to 60 carbon atoms. When the heteroaryl group has 1 or 2 carbon atoms, the heteroaryl group includes an additional hetero atom to form a ring. In addition, the heteroaryl group contains 1 to 30 carbon atoms. In this regard, one or more carbons of a ring are replaced with heteroatoms such as oxygen (O), nitrogen (N), sulfur (S), or selenium (Se). In this case, the heteroaryl group may include a 6-membered monocyclic ring such as pyridyl, pyrazinyl, pyrimidinyl, pyridazinyl, and triazinyl, a polycyclic ring such as phenoxathiinyl, indolizinyl, indolyl, purinyl, quinolyl, isoquinolyl, benzooxyzolyl, benzothiazolyl, dibenzooxyzolyl, dibenzothiazolyl, benzoimidazolyl, benzofuranyl, dibenzofuranyl, benzothiophenyl, dibenzothiophenyl, phenylcarbazolyl, 9-phenylcarbazolyl, and carbazolyl, and 2-furanyl, N-imidazolyl, 2-isoxazolyl, 2-pyridinyl, 2-pyrimidinyl etc. but is not limited thereto. Additionally, the heteroaryl group may be optionally substituted.

The term “heterocyclic group” as used herein means that at least one of the carbon atoms constituting an aryl group, a cycloalkyl group, a cycloalkenyl group, a cycloalkynyl group, an arylalkyl group, an arylamino group, etc. is substituted with a heteroatom such as oxygen (O), nitrogen (N), sulfur (S), etc. Referring to the above definition, the heterocyclic group may include a heteroaryl group, a heterocycloalkyl group, a heterocycloalkenyl group, a heterocycloalkynyl group, a heteroarylalkyl group, a heteroarylamino group, etc. Additionally, the heterocyclic group may be optionally substituted.

Unless otherwise specified, the term “carbon ring” as used herein may be used as including all of a “cycloalkyl group”, “cycloalkenyl group”, “cycloalkynyl group” as an alicyclic group and “aryl group (aromatic group)” as an aromatic ring group.

Each of the terms “heteroalkyl group”, “heteroalkenyl group”, “heteroalkynyl group”, and “heteroarylalkyl group” as used herein means that one or more of the carbon atoms constituting the group is substituted with a heteroatom such as oxygen (O), nitrogen (N), sulfur (S). Additionally, each of the heteroalkyl group, heteroalkenyl group, heteroalkynyl group, and heteroarylalkyl group may be optionally substituted.

As used herein, the term “alkylamino group,” “aralkylamino group,” “arylamino group,” or “heteroarylamino group” refers to an amino group (an amine group) into which an alkyl group, an aralkyl group, an aryl group, or a heteroaryl group is substituted. In this regard, the amino group (amine group) may include all of primary, secondary, and tertiary amino groups (amine groups). Further, the alkylamino group, the aralkylamino group, the arylamino group, and the heteroarylamino group may be optionally substituted.

As used herein, the term “alkylsilyl group”, “arylsilyl group”, “alkoxy group”, “aryloxy group”, “alkylthio group”, or “arylthio group” refers to each of a silyl group, an oxy group, and a thio group into which each of an alkyl group and an aryl group is substituted. Additionally, the alkylsilyl group, the arylsilyl group, the alkoxy group, the aryloxy group, the alkylthio group, and the arylthio group may be optionally substituted.

The terms “arylene group”, “arylalkylene group”, “heteroarylene group”, or “heteroarylalkylene group” as used herein means a group having two-substitutions in which the aryl group, arylalkyl group, heteroaryl group, or heteroarylalkyl group further includes one substitution. Additionally, the arylene group, arylalkylene group, heteroarylene group, and heteroarylalkylene group may be optionally substituted.

As used herein, the term “substituted” means that a hydrogen atom (H) binding to a carbon atom of a compound of the present disclosure is replaced with a substituent other than hydrogen. When there are a plurality of substituents, the substituents may be the same as or different from each other.

The substituent may independently include one selected from a group consisting of deuterium, a cyano group, a trifluoromethyl group, a nitro group, a halogen group, a hydroxy group, a trimethylsilylethynyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an aralkyl group having 7 to 30 carbon atoms, a heteroaryl group having 5 to 60 carbon atoms, a heteroaralkyl group having 6 to 60 carbon atoms, an amine group, an alkylamino group having 1 to 30 carbon atoms, an aralkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms.

Unless otherwise specified, a position at which the substitution occurs is not particularly limited as long as a hydrogen atom can be substituted with a substituent at the position. When two or more substituents, that is, the plurality of substituents are present, the substituents may be identical to or different from each other.

Subjects and substituents as defined in the present disclosure may be the same as or different from each other unless otherwise specified.

As used herein, a unit is based on weight (wt), unless specifically stated. For example, when “%” is written, this is interpreted as weight % (wt %).

Hereinafter, an organic compound and an organic light-emitting diode including the same according to the present disclosure will be described in detail.

The organic compound in accordance with the present disclosure may be represented by a following Chemical Formula 1:

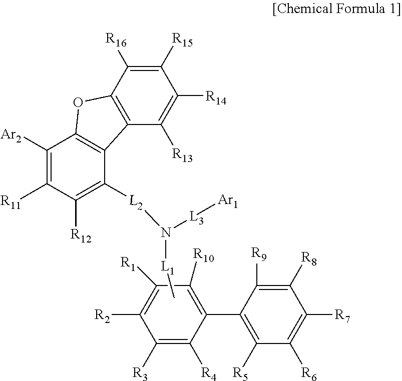

wherein in the Chemical Formula 1,
each of L₁ to L₃ independently represents one selected from a group consisting of a single bond, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, and a substituted or unsubstituted heteroarylalkyl group having 6 to 60 carbon atoms,
R₁ to R₁₆ and Ar are identical with or different from each other, wherein each of R₁ to R₁₆ and Ar independently represents one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6 to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms,
Ar2 is a substituted or unsubstituted aryl group having 6 to 30 carbon atoms,
optionally, each of L₁ to L₃, R₁ to R₁₆, and Ar₁ and Ar₂ may be substituted with at least one substituent selected from a group consisting of deuterium, a cyano group, a trifluoromethyl group, a nitro group, a halogen group, a hydroxy group, a trimethylsilylethynyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an aralkyl group having 7 to 30 carbon atoms, a heteroaryl group having 5 to 60 carbon atoms, a heteroaralkyl group having 6 to 60 carbon atoms, an amine group, an alkylamino group having 1 to 30 carbon atoms, an aralkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms, when the at least one substituent includes a plurality of substituents, the plurality of substituents are identical with or different from each other.

The compound represented by the Chemical Formula 1 is of a type of an amine structure NRR′R″, where R is a dibenzofuran group connected to N (nitrogen) at a position #1 via a linker L₂, R′ is a terphenyl group connected to N (nitrogen) via a linker L₁, and R″ is Ar₁ connected to N (nitrogen) via a linker L₃, wherein Ar₁ may be selected to be various. In addition, the substituent Ar₂ binds to a position #4 of the dibenzofuran group, thereby increasing conjugation and expanding an electron cloud of HOMO (Highest Occupied Molecular Orbital), thereby improving hole injection and hole transport characteristics. Furthermore, when the compound represented by the Chemical Formula 1 is used as a material of an hole transport auxiliary layer of an organic light-emitting diode, the hole transport auxiliary layer may have a suitable energy level at which the hole transport auxiliary layer transfers holes from the hole transport layer to the light-emitting layer and blocks electrons coming from the light-emitting layer. Thus, the compound represented by the Chemical Formula 1 may exhibit the characteristics suitable for use as the hole transport auxiliary layer of the organic light-emitting diode.

According to one embodiment of the present disclosure, R₇ may be a substituent represented by a following Chemical Formula 2:

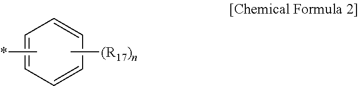

wherein in the Chemical Formula 2, * denotes a binding site, n is an integer of 0 to 5, and R₁₇ may independently represent one selected from a group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 5 to 60 carbon atoms.

According to one embodiment of the present disclosure, each of Ar₁ may independently represent a substituted or unsubstituted aryl group having 6 to 30 carbon atoms. When Ar₁ is an aryl group, hole mobility may be improved compared to when Ar₁ is a heteroaryl group. For example, Ar₁ may be a substituted or unsubstituted aryl group having 6 to 30 carbon atoms, a substituted or unsubstituted aryl group having 6 to 18 carbon atoms, or a substituted or unsubstituted aryl group having 6 to 15 carbon atoms. When the number of carbon atoms exceeds 18, the above characteristics of the compound as the material of the hole transport auxiliary layer may deteriorate. For example, Ar may be a substituted or unsubstituted phenyl group, a substituted or unsubstituted naphthyl group, a substituted or unsubstituted biphenyl group, a substituted or unsubstituted phenanthrene group, a substituted or unsubstituted dimethyl fluorene group, or a substituted or unsubstituted triphenylene group.

According to one embodiment of the present disclosure, Ar₂ may be one of substituents respectively represented by following Chemical Formula 3 to Chemical Formula 5:

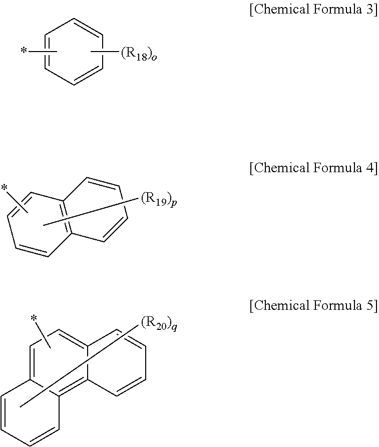

wherein in each of the Chemical Formulas 3 to 5, * denotes a binding site, n is an integer of 0 to 5, p is an integer from 0 to 7, and q is an integer from 0 to 9, and each of R₁₈ to R₂₀ may independently represent one selected from a group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 5 to 60 carbon atoms.

According to one embodiment of the present disclosure, L₁ may be a single bond.

According to one embodiment of the present disclosure, the Chemical Formula 1 may be selected from following Chemical Formula 6 to Chemical Formula 13, depending on a binding position relationship of three phenyls of the terphenyl group binding to the nitrogen (N) of an arylamine group:

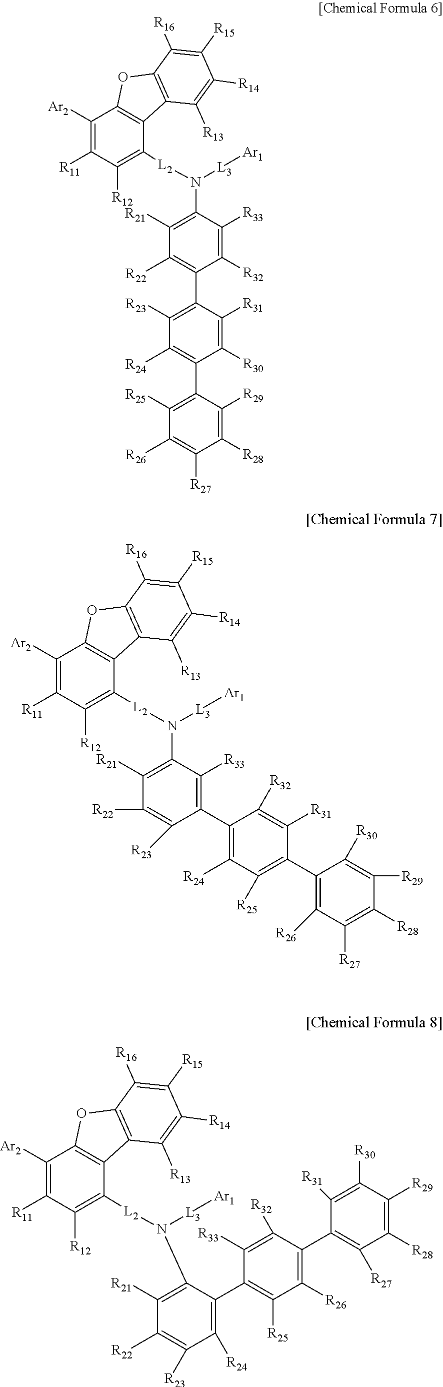

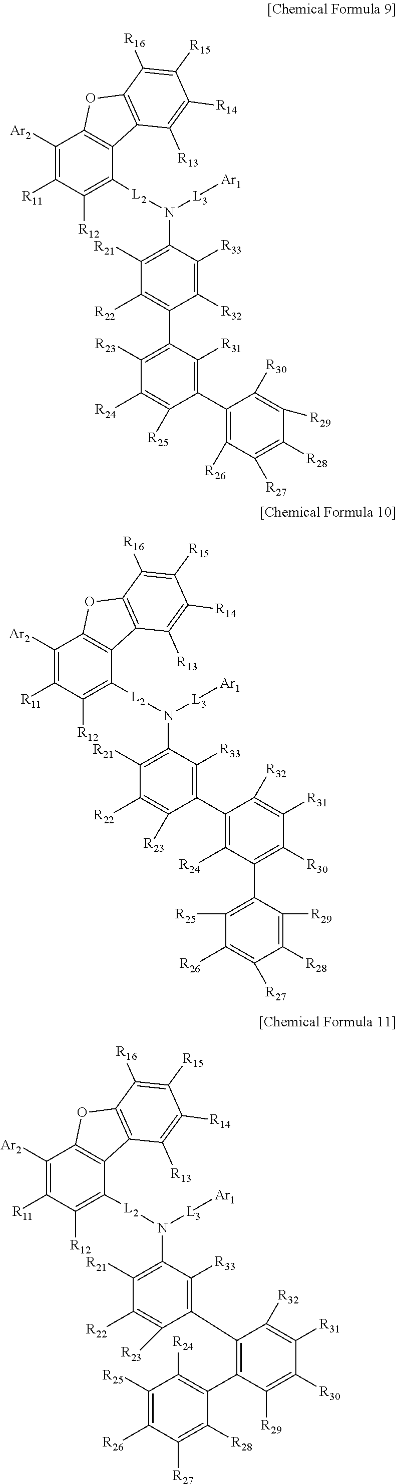

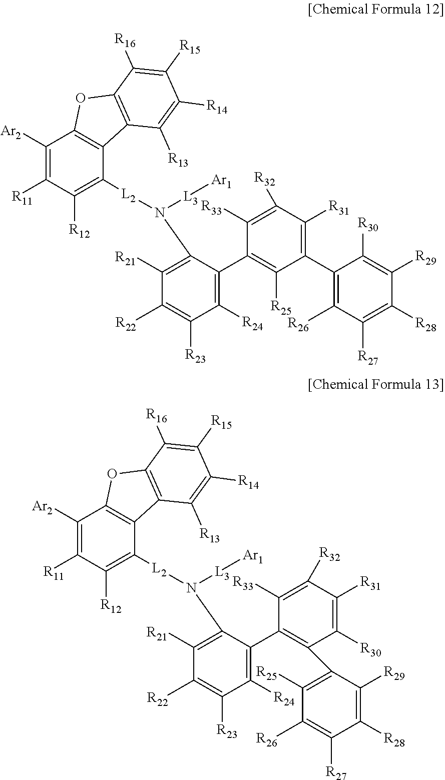

wherein in each of the Chemical Formulas 6 to 13, each of L₂, L₃, R₁₁ to R₁₆, and Ar₁ may be the same as defined in the Chemical Formula 1,
wherein R₂₁ to R₃₃ may be identical with or different from each other, wherein each of R₂₁ to R₃₃ may independently represent one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6
to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms.

According to one embodiment of the present disclosure, L₂ may be a single bond.

According to one embodiment of the present disclosure, L₂ may be selected as a single bond in the structures of Chemical Formulas 6 to 13, wherein the Chemical Formula 1 may be selected from a group consisting of following Chemical Formulas 14 to 45, based on a type of a structure binding to the Ar₂ position of the dibenzofuran group:

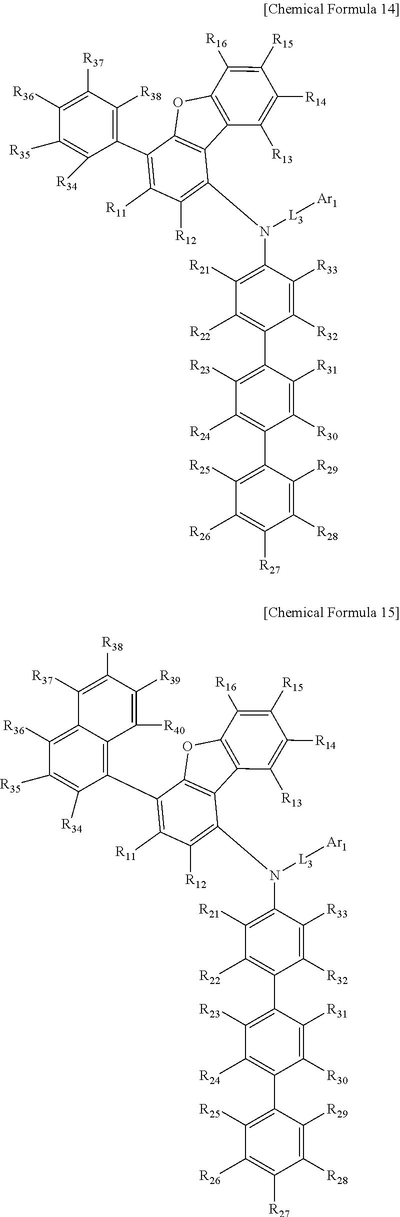

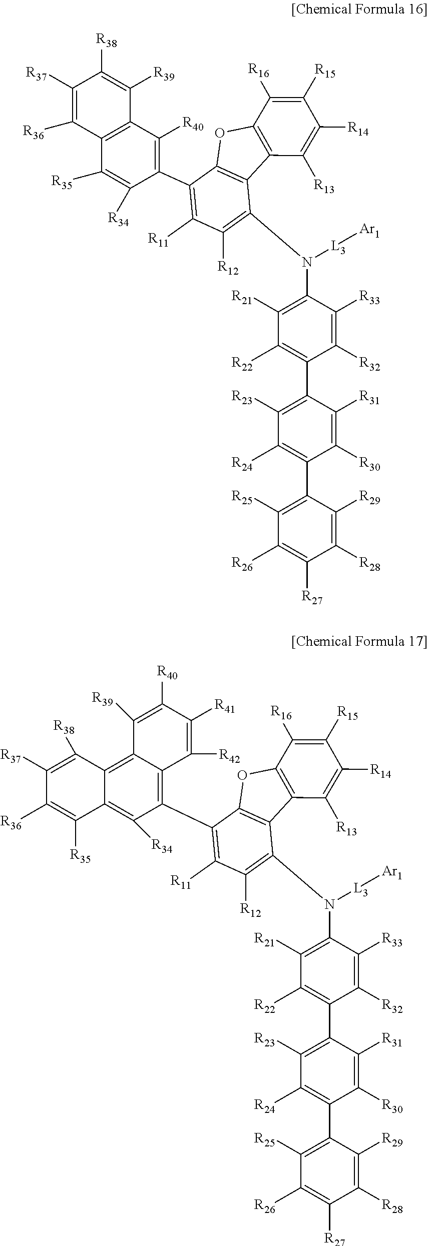

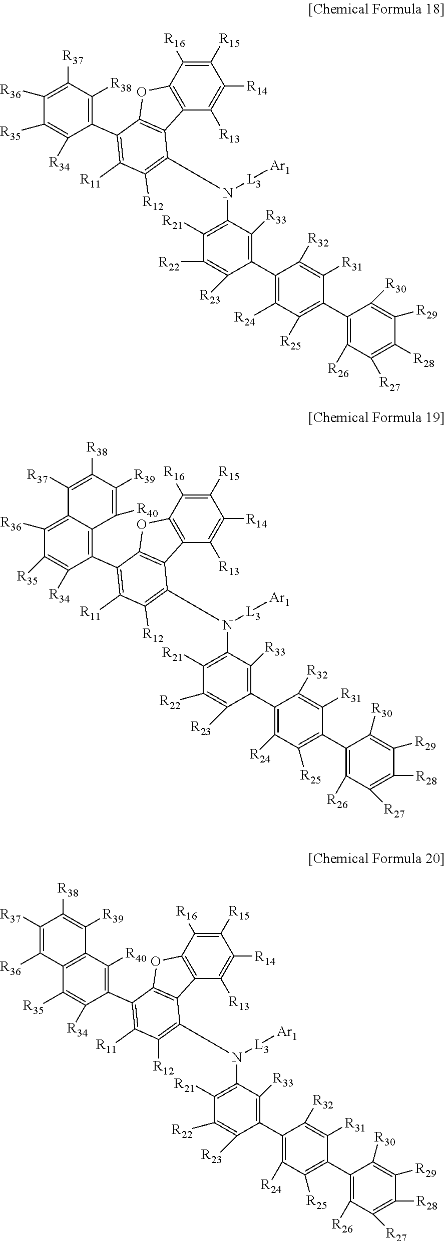

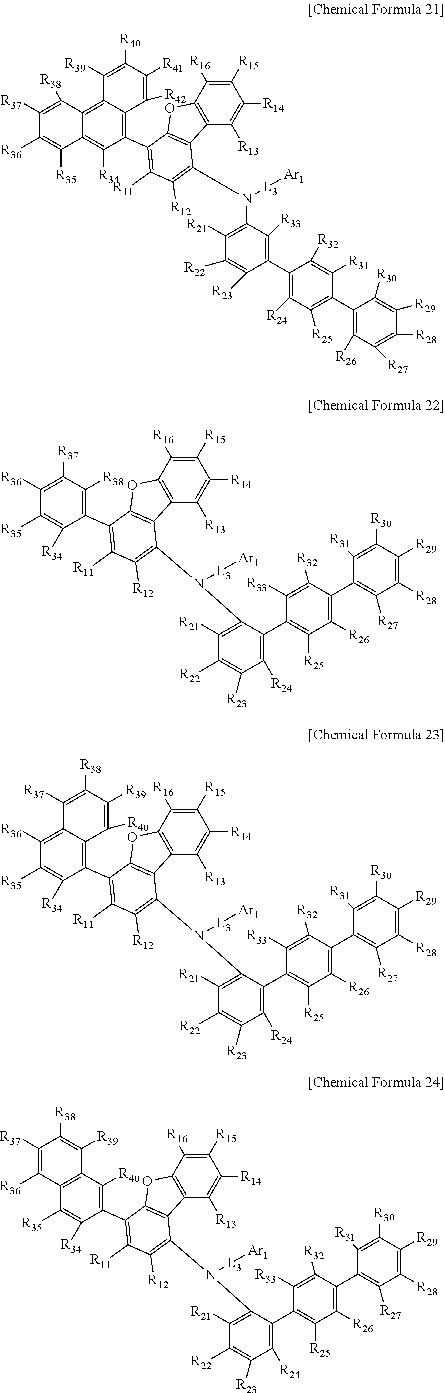

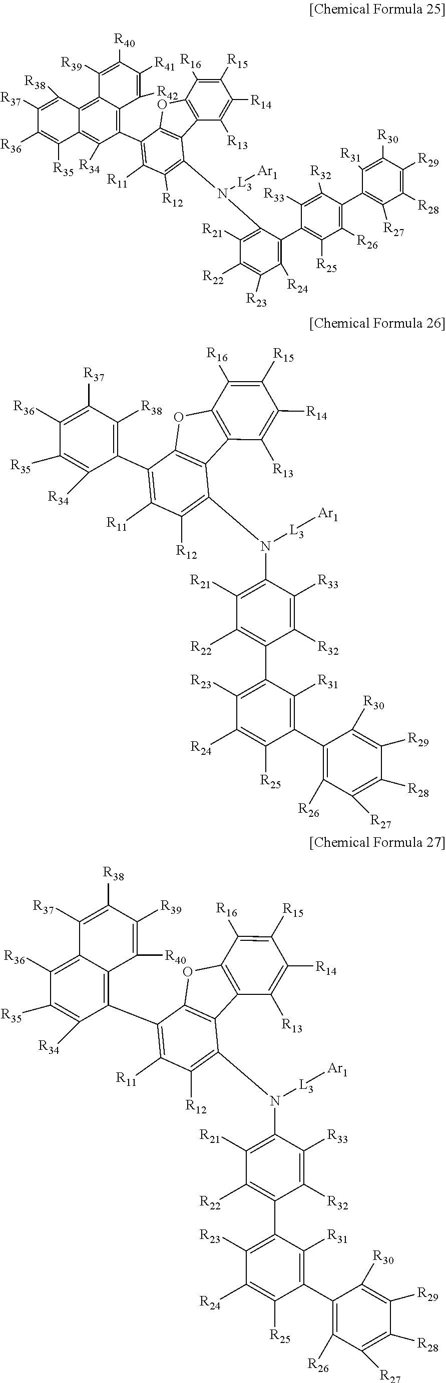

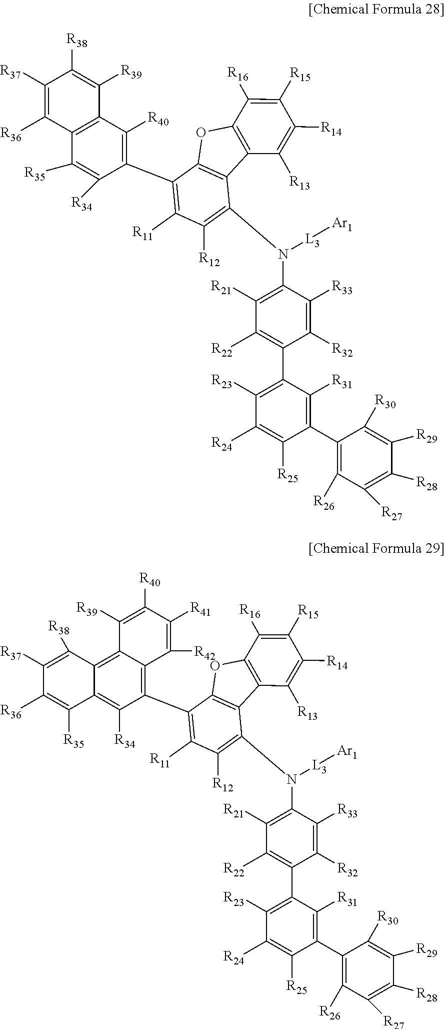

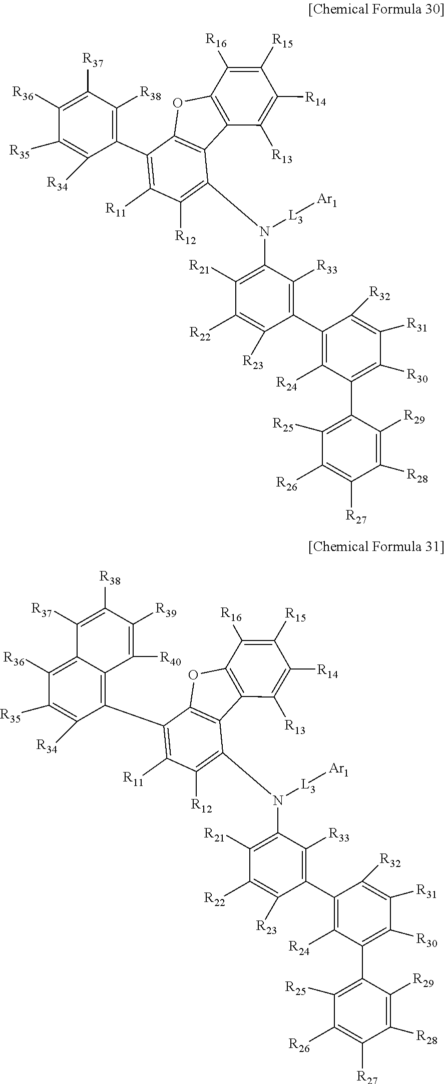

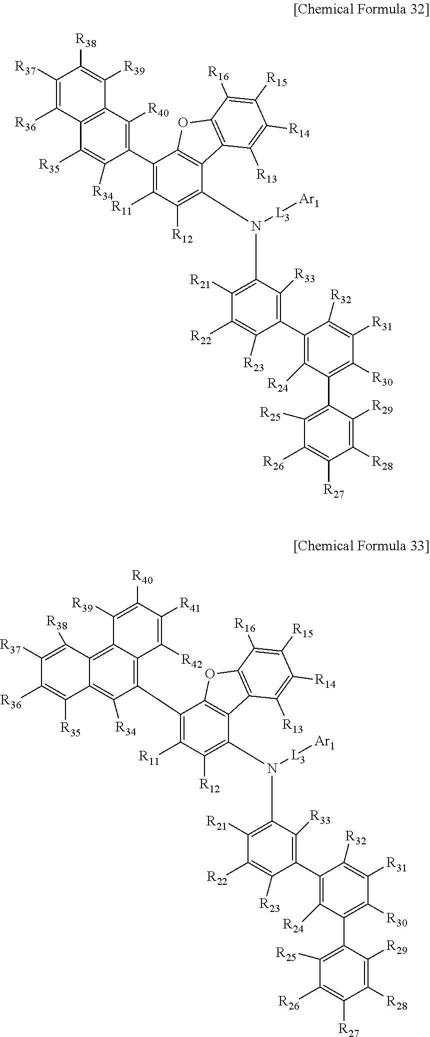

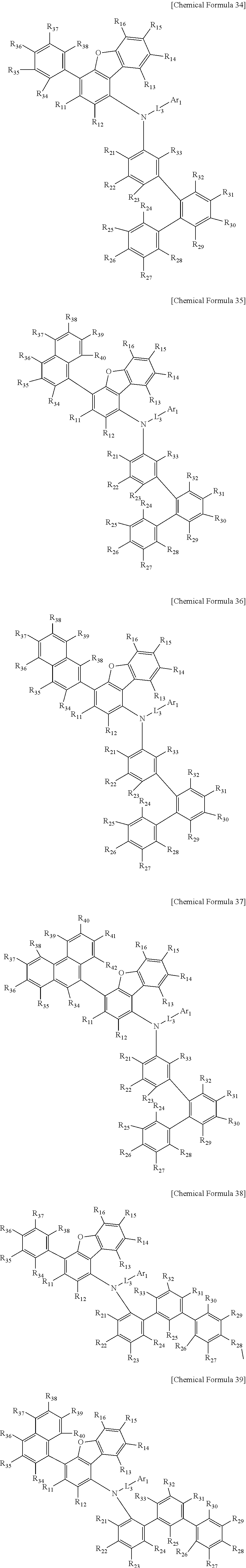

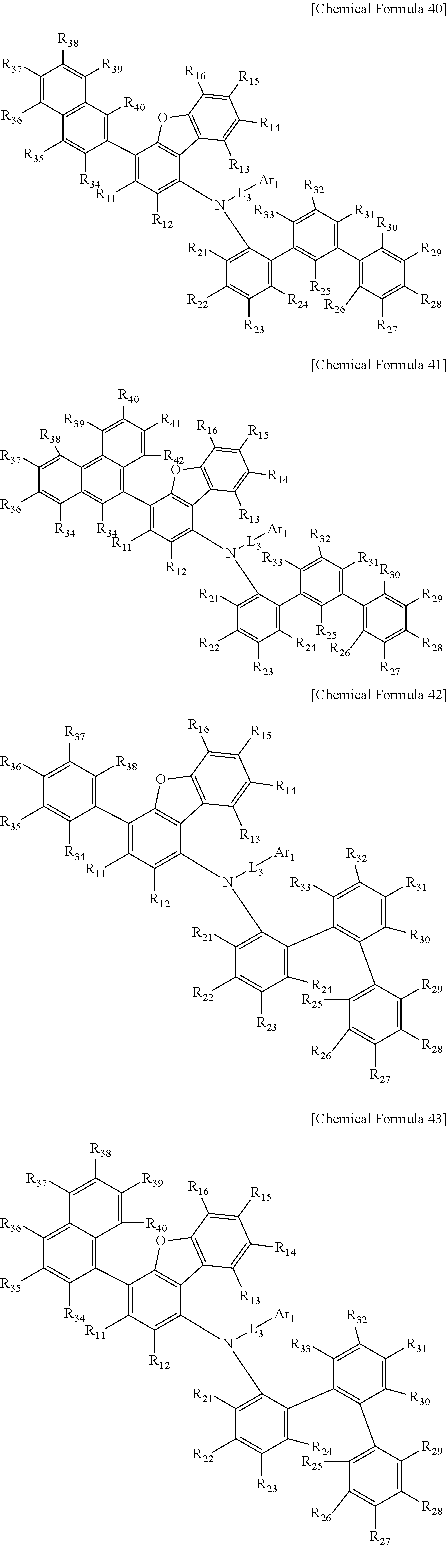

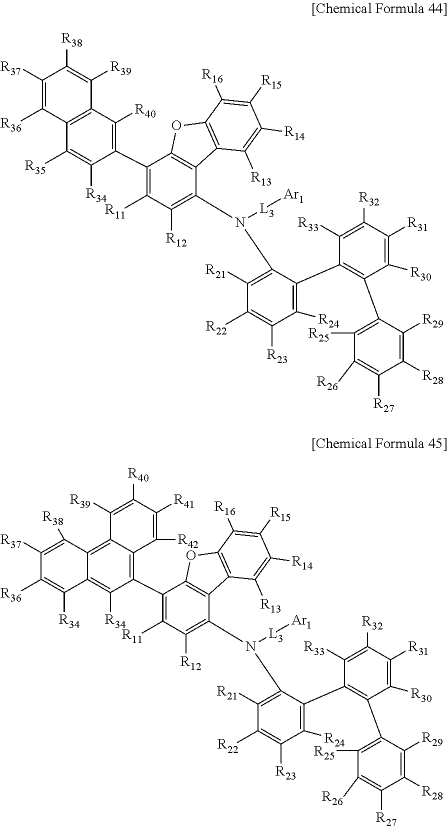

wherein in each of the Chemical Formulas 14 to 45, each of L₃, R₁₁ to R₁₆, Ar₁ and may be the same as defined in the Chemical Formula 1, R₂₁ to R₄₂ may be identical with or different from each other, wherein each of R₂₁ to R₄₂ independently represents one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6 to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms.

According to one embodiment of the present disclosure, the organic compound represented by the Chemical Formula 1 may be one selected from following compounds, and each of the following compounds may be further subjected to substitution.

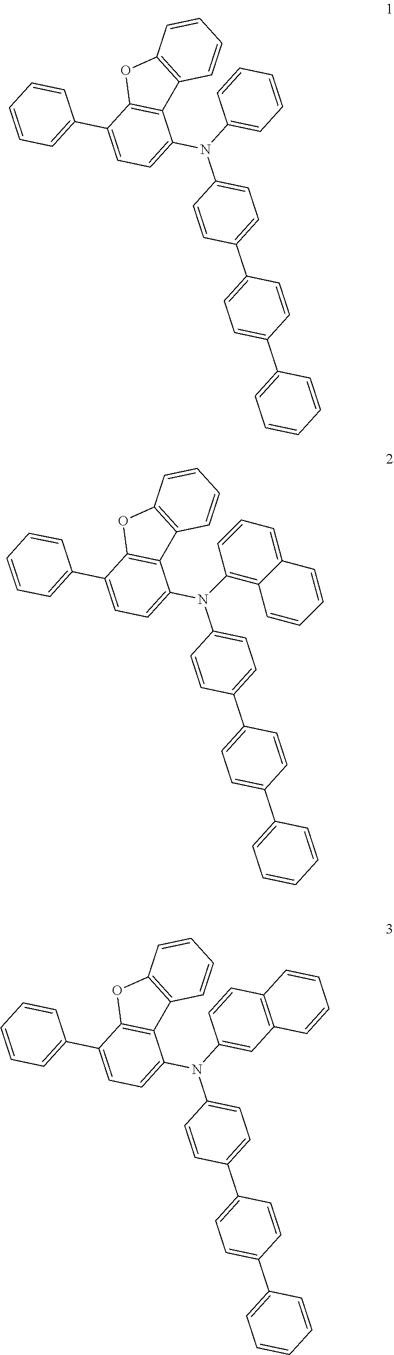 [SMILES: c1ccc(-c2ccc(-c3ccc(N(c4ccccc4)c4ccc(-c5ccccc5)c5oc6ccccc6c45)cc3)cc2)cc1 ; c1ccc(-c2ccc(-c3ccc(N(c4cccc5ccccc45)c4ccc(-c5ccccc5)c5oc6ccccc6c45)cc3)cc2)cc1 ; c1ccc(-c2ccc(-c3ccc(N(c4ccc5ccccc5c4)c4ccc(-c5ccccc5)c5oc6ccccc6c45)cc3)cc2)cc1]

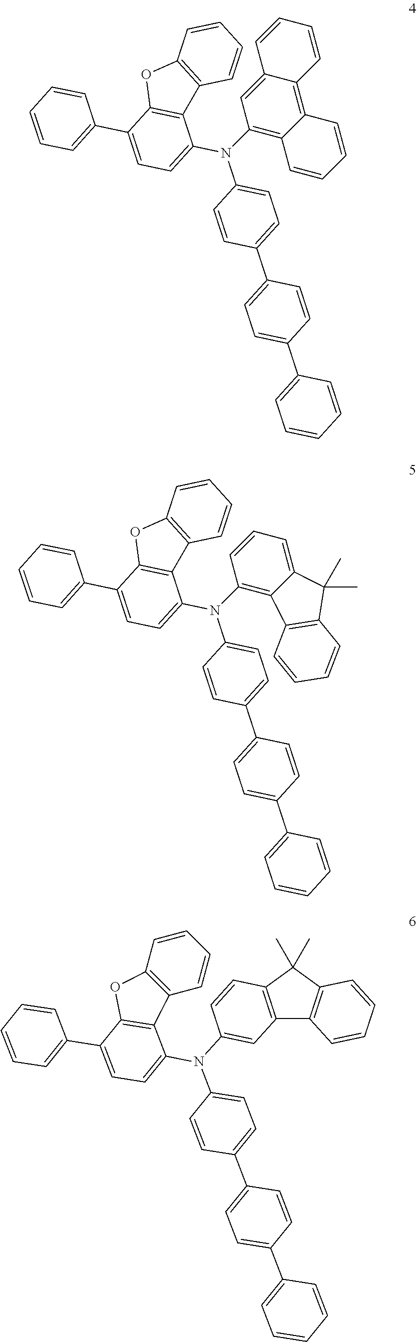

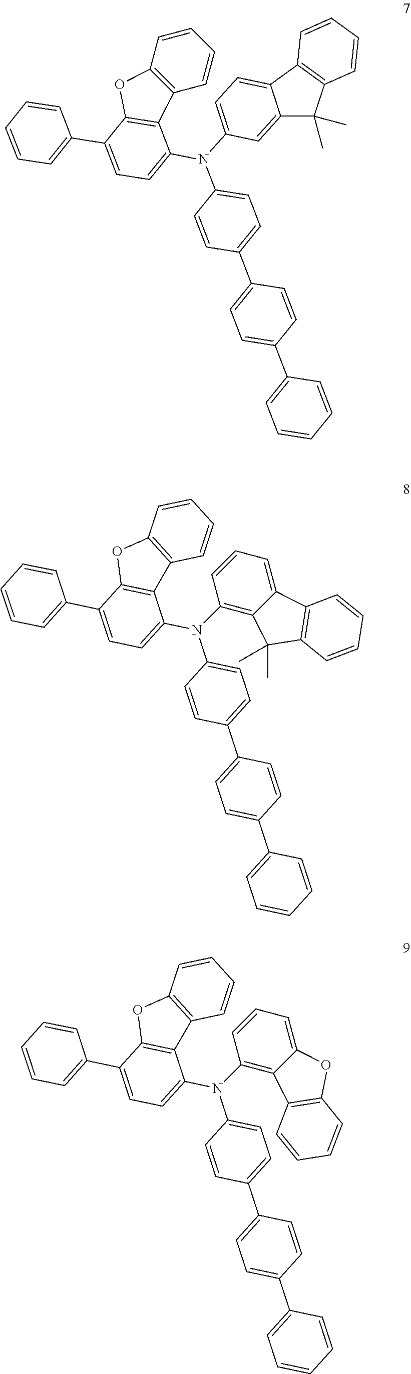 [SMILES: CC1(C)c2ccccc2-c2ccc(N(c3ccc(-c4ccc(-c5ccccc5)cc4)cc3)c3ccc(-c4ccccc4)c4oc5ccccc5c34)cc21 ; CC1(C)c2ccccc2-c2cccc(N(c3ccc(-c4ccc(-c5ccccc5)cc4)cc3)c3ccc(-c4ccccc4)c4oc5ccccc5c34)c21 ; c1ccc(-c2ccc(-c3ccc(N(c4cccc5oc6ccccc6c45)c4ccc(-c5ccccc5)c5oc6ccccc6c45)cc3)cc2)cc1]

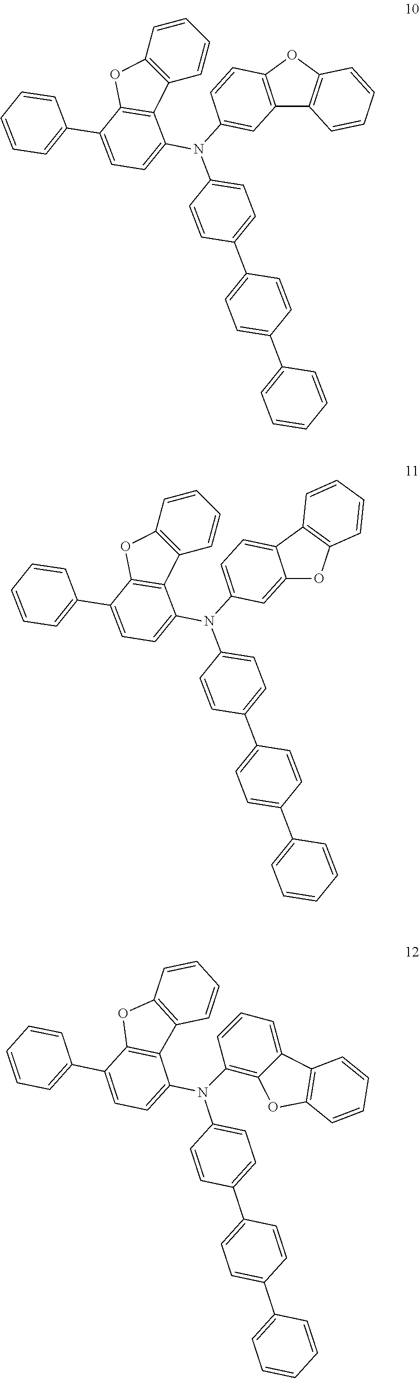 [SMILES: c1ccc(-c2ccc(-c3ccc(N(c4ccc5oc6ccccc6c5c4)c4ccc(-c5ccccc5)c5oc6ccccc6c45)cc3)cc2)cc1 ; c1ccc(-c2ccc(-c3ccc(N(c4ccc5c(c4)oc4ccccc45)c4ccc(-c5ccccc5)c5oc6ccccc6c45)cc3)cc2)cc1 ; c1ccc(-c2ccc(-c3ccc(N(c4cccc5c4oc4ccccc45)c4ccc(-c5ccccc5)c5oc6ccccc6c45)cc3)cc2)cc1]

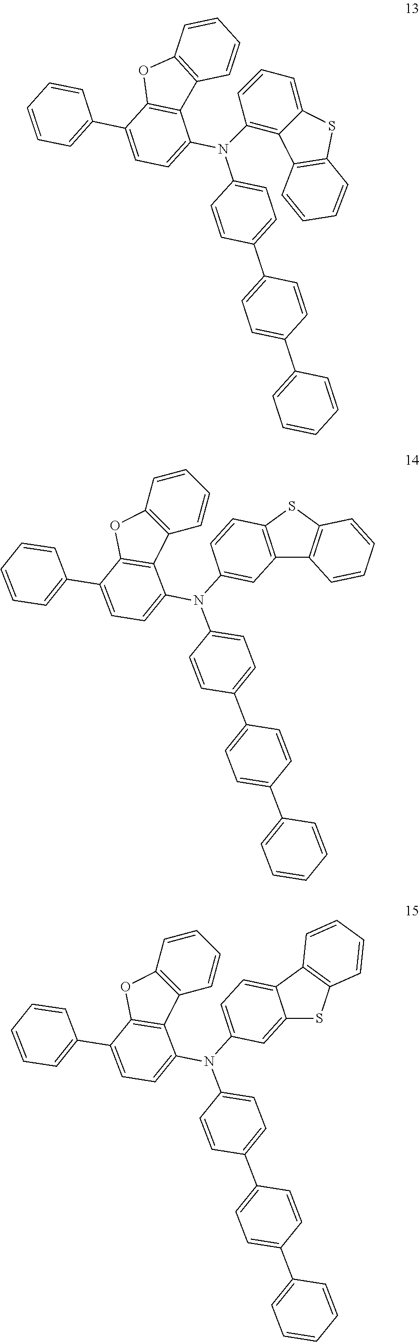 [SMILES: c1ccc(-c2ccc(-c3ccc(N(c4cccc5sc6ccccc6c45)c4ccc(-c5ccccc5)c5oc6ccccc6c45)cc3)cc2)cc1 ; c1ccc(-c2ccc(-c3ccc(N(c4ccc5sc6ccccc6c5c4)c4ccc(-c5ccccc5)c5oc6ccccc6c45)cc3)cc2)cc1 ; c1ccc(-c2ccc(-c3ccc(N(c4ccc5c(c4)sc4ccccc45)c4ccc(-c5ccccc5)c5oc6ccccc6c45)cc3)cc2)cc1]

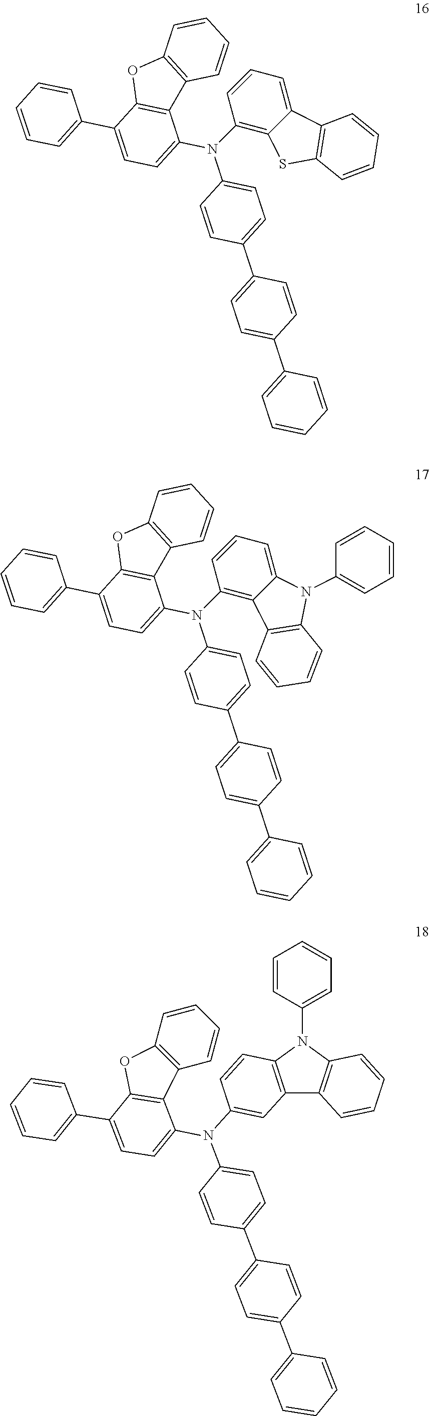

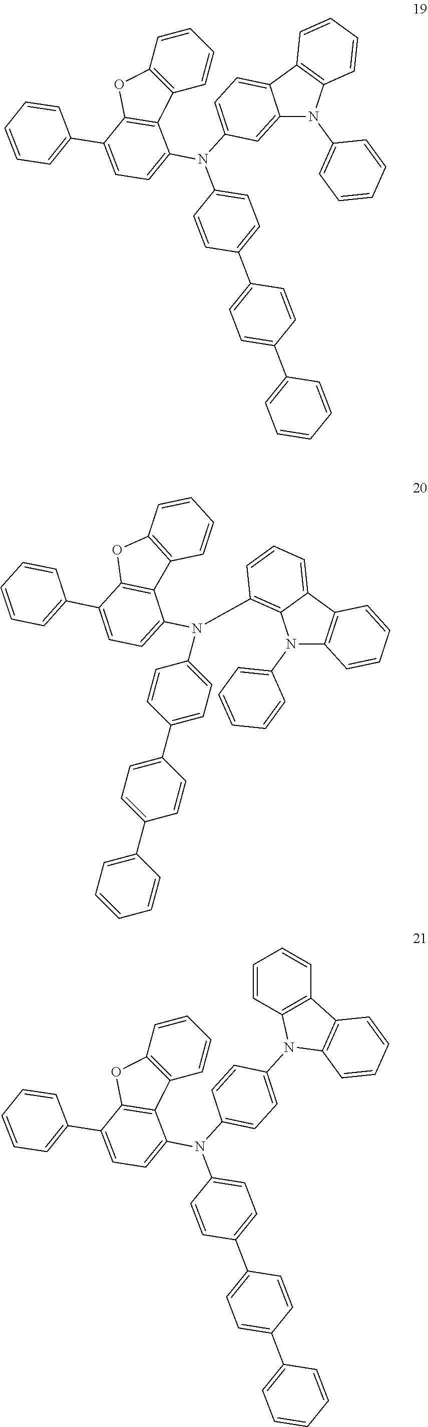 [SMILES: c1ccc(-c2ccc(-c3ccc(N(c4ccc5c6ccccc6n(-c6ccccc6)c5c4)c4ccc(-c5ccccc5)c5oc6ccccc6c45)cc3)cc2)cc1 ; c1ccc(-c2ccc(-c3ccc(N(c4ccc(-c5ccccc5)c5oc6ccccc6c45)c4cccc5c6ccccc6n(-c6ccccc6)c45)cc3)cc2)cc1 ; c1ccc(-c2ccc(-c3ccc(N(c4ccc(-n5c6ccccc6c6ccccc65)cc4)c4ccc(-c5ccccc5)c5oc6ccccc6c45)cc3)cc2)cc1]

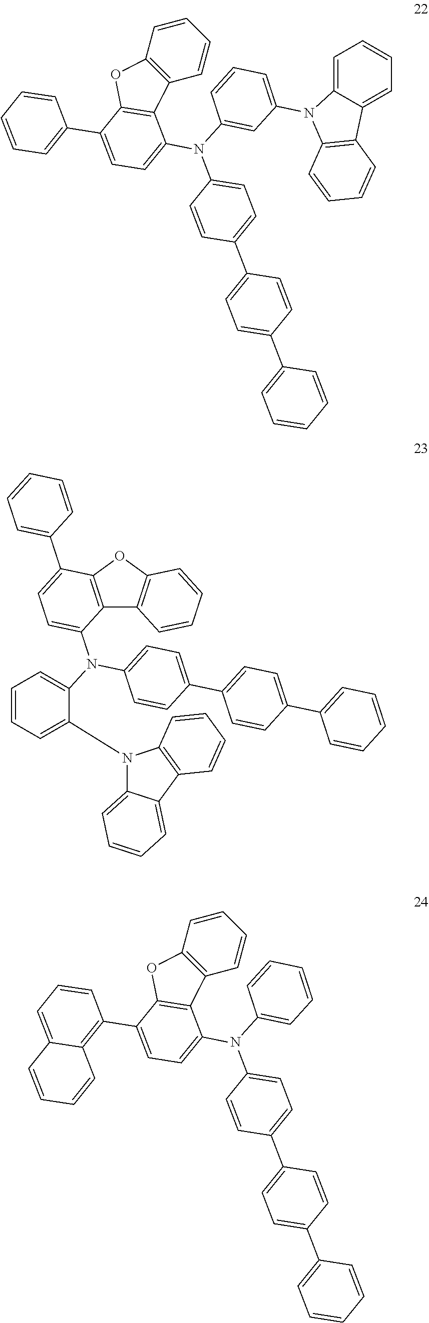 [SMILES: c1ccc(-c2ccc(-c3ccc(N(c4cccc(-n5c6ccccc6c6ccccc65)c4)c4ccc(-c5ccccc5)c5oc6ccccc6c45)cc3)cc2)cc1 ; c1ccc(-c2ccc(-c3ccc(N(c4ccccc4-n4c5ccccc5c5ccccc54)c4ccc(-c5ccccc5)c5oc6ccccc6c45)cc3)cc2)cc1 ; c1ccc(-c2ccc(-c3ccc(N(c4ccccc4)c4ccc(-c5cccc6ccccc56)c5oc6ccccc6c45)cc3)cc2)cc1]

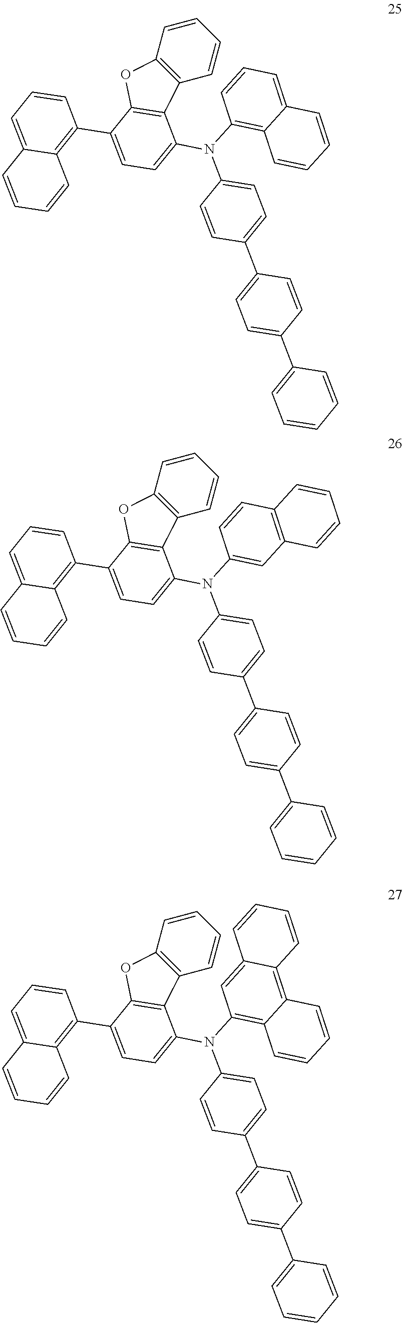 [SMILES: c1ccc(-c2ccc(-c3ccc(N(c4cccc5ccccc45)c4ccc(-c5cccc6ccccc56)c5oc6ccccc6c45)cc3)cc2)cc1 ; c1ccc(-c2ccc(-c3ccc(N(c4ccc5ccccc5c4)c4ccc(-c5cccc6ccccc56)c5oc6ccccc6c45)cc3)cc2)cc1 ; c1ccc(-c2ccc(-c3ccc(N(c4cc5ccccc5c5ccccc45)c4ccc(-c5cccc6ccccc56)c5oc6ccccc6c45)cc3)cc2)cc1]

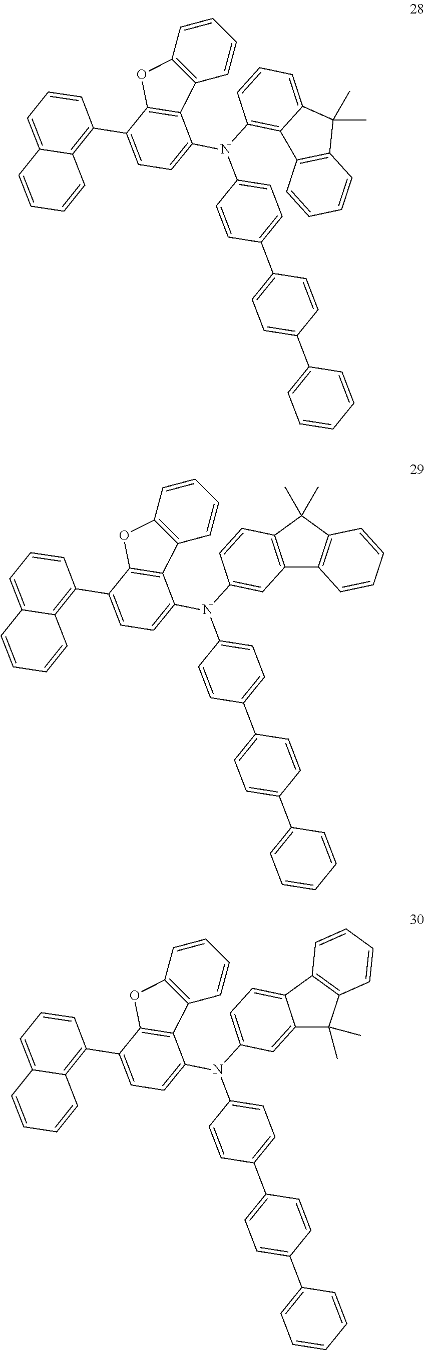 [SMILES: CC1(C)c2ccccc2-c2c(N(c3ccc(-c4ccc(-c5ccccc5)cc4)cc3)c3ccc(-c4cccc5ccccc45)c4oc5ccccc5c34)cccc21 ; CC1(C)c2ccccc2-c2cc(N(c3ccc(-c4ccc(-c5ccccc5)cc4)cc3)c3ccc(-c4cccc5ccccc45)c4oc5ccccc5c34)ccc21 ; CC1(C)c2ccccc2-c2ccc(N(c3ccc(-c4ccc(-c5ccccc5)cc4)cc3)c3ccc(-c4cccc5ccccc45)c4oc5ccccc5c34)cc21]

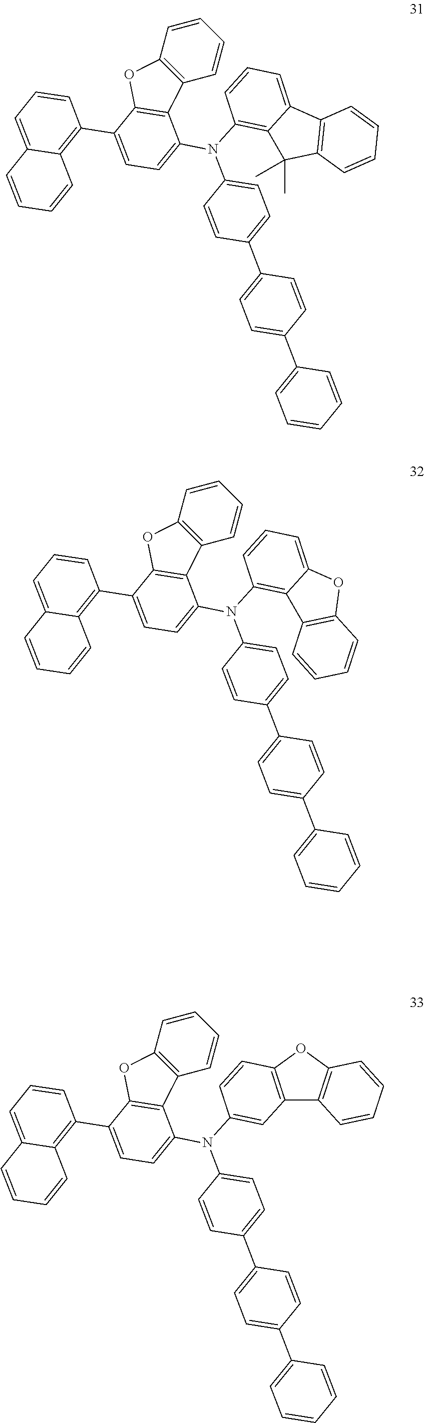 [SMILES: CC1(C)c2ccccc2-c2cccc(N(c3ccc(-c4ccc(-c5ccccc5)cc4)cc3)c3ccc(-c4cccc5ccccc45)c4oc5ccccc5c34)c21 ; c1ccc(-c2ccc(-c3ccc(N(c4cccc5oc6ccccc6c45)c4ccc(-c5cccc6ccccc56)c5oc6ccccc6c45)cc3)cc2)cc1 ; c1ccc(-c2ccc(-c3ccc(N(c4ccc5oc6ccccc6c5c4)c4ccc(-c5cccc6ccccc56)c5oc6ccccc6c45)cc3)cc2)cc1]

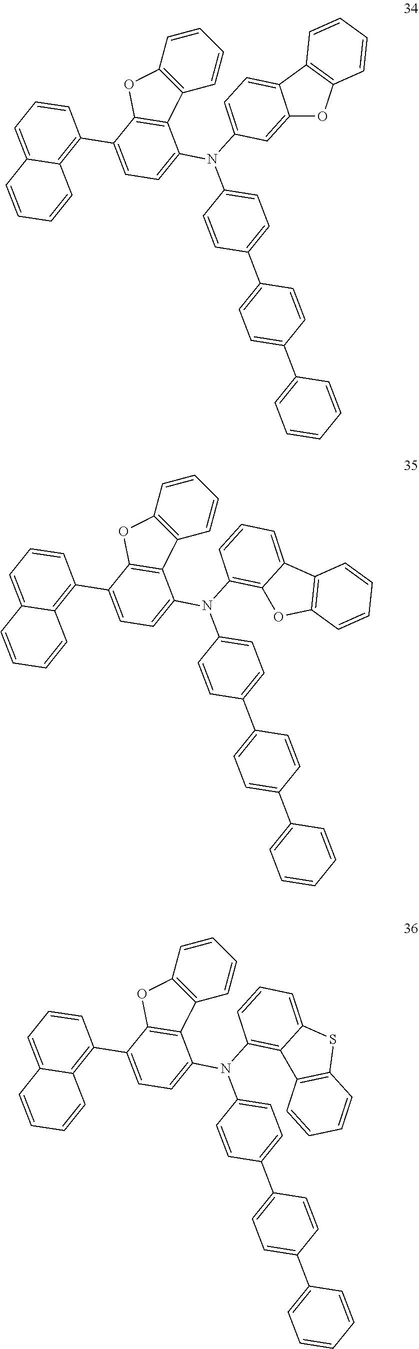 [SMILES: c1ccc(-c2ccc(-c3ccc(N(c4ccc5c(c4)oc4ccccc45)c4ccc(-c5cccc6ccccc56)c5oc6ccccc6c45)cc3)cc2)cc1 ; c1ccc(-c2ccc(-c3ccc(N(c4cccc5c4oc4ccccc45)c4ccc(-c5cccc6ccccc56)c5oc6ccccc6c45)cc3)cc2)cc1 ; c1ccc(-c2ccc(-c3ccc(N(c4cccc5sc6ccccc6c45)c4ccc(-c5cccc6ccccc56)c5oc6ccccc6c45)cc3)cc2)cc1]

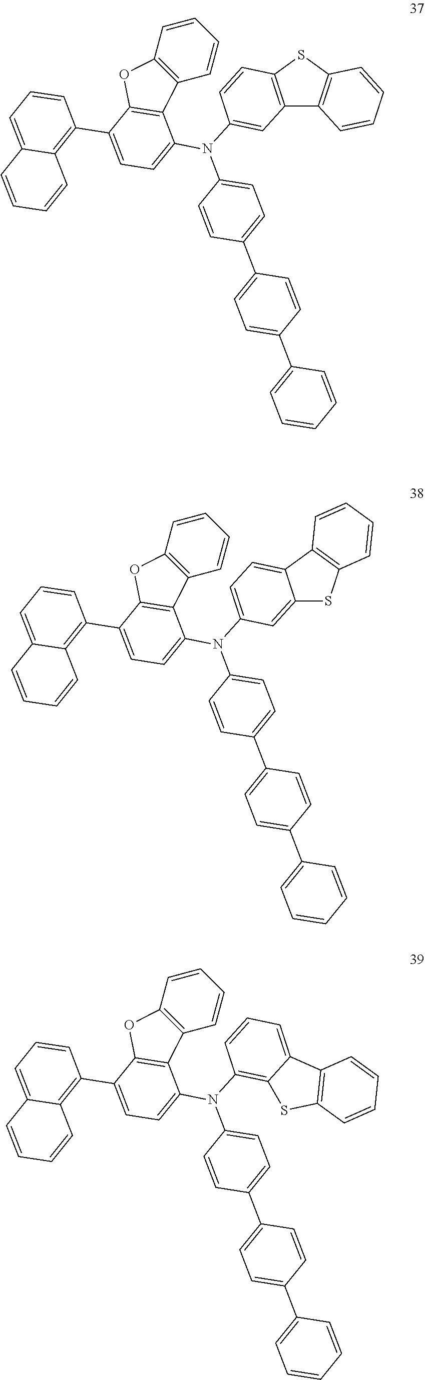 [SMILES: c1ccc(-c2ccc(-c3ccc(N(c4ccc5sc6ccccc6c5c4)c4ccc(-c5cccc6ccccc56)c5oc6ccccc6c45)cc3)cc2)cc1 ; c1ccc(-c2ccc(-c3ccc(N(c4ccc5c(c4)sc4ccccc45)c4ccc(-c5cccc6ccccc56)c5oc6ccccc6c45)cc3)cc2)cc1 ; c1ccc(-c2ccc(-c3ccc(N(c4cccc5c4sc4ccccc45)c4ccc(-c5cccc6ccccc56)c5oc6ccccc6c45)cc3)cc2)cc1]

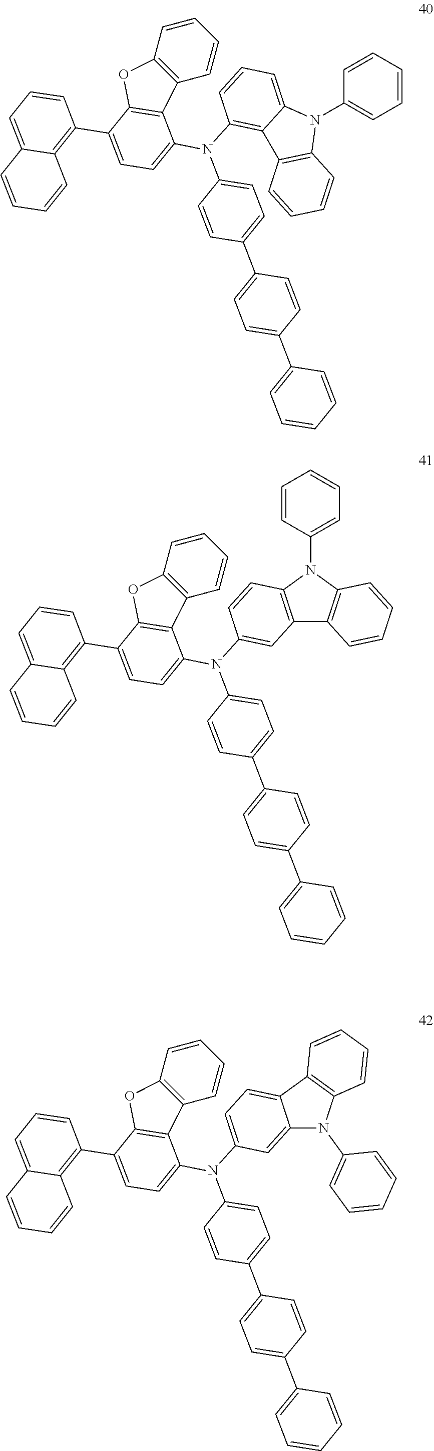

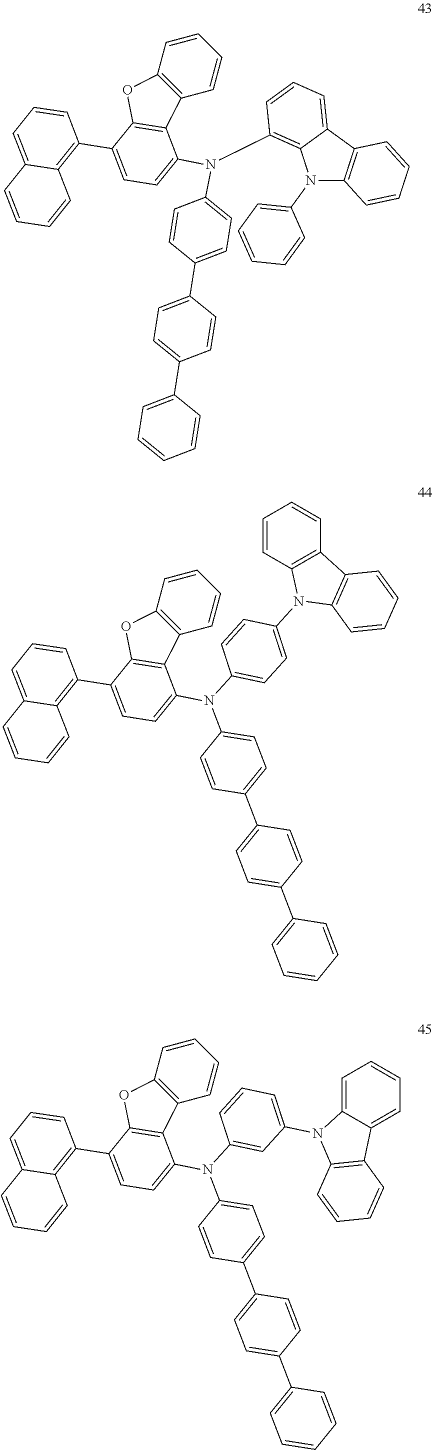 [SMILES: c1ccc(-c2ccc(-c3ccc(N(c4ccc(-c5cccc6ccccc56)c5oc6ccccc6c45)c4cccc5c6ccccc6n(-c6ccccc6)c45)cc3)cc2)cc1 ; c1ccc(-c2ccc(-c3ccc(N(c4ccc(-n5c6ccccc6c6ccccc65)cc4)c4ccc(-c5cccc6ccccc56)c5oc6ccccc6c45)cc3)cc2)cc1 ; c1ccc(-c2ccc(-c3ccc(N(c4cccc(-n5c6ccccc6c6ccccc65)c4)c4ccc(-c5cccc6ccccc56)c5oc6ccccc6c45)cc3)cc2)cc1]

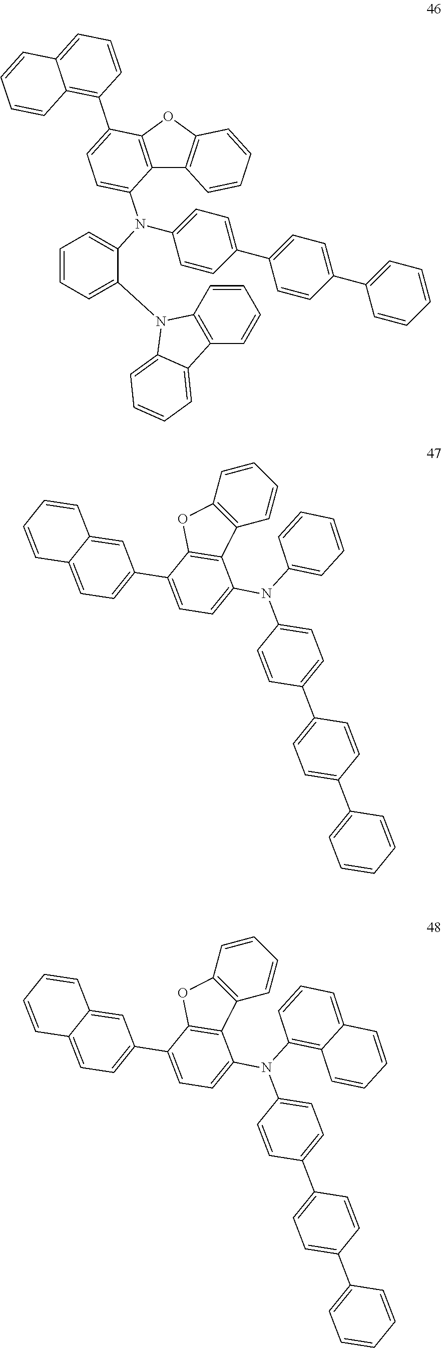 [SMILES: c1ccc(-c2ccc(-c3ccc(N(c4ccccc4-n4c5ccccc5c5ccccc54)c4ccc(-c5cccc6ccccc56)c5oc6ccccc6c45)cc3)cc2)cc1 ; c1ccc(-c2ccc(-c3ccc(N(c4ccccc4)c4ccc(-c5ccc6ccccc6c5)c5oc6ccccc6c45)cc3)cc2)cc1 ; c1ccc(-c2ccc(-c3ccc(N(c4cccc5ccccc45)c4ccc(-c5ccc6ccccc6c5)c5oc6ccccc6c45)cc3)cc2)cc1]

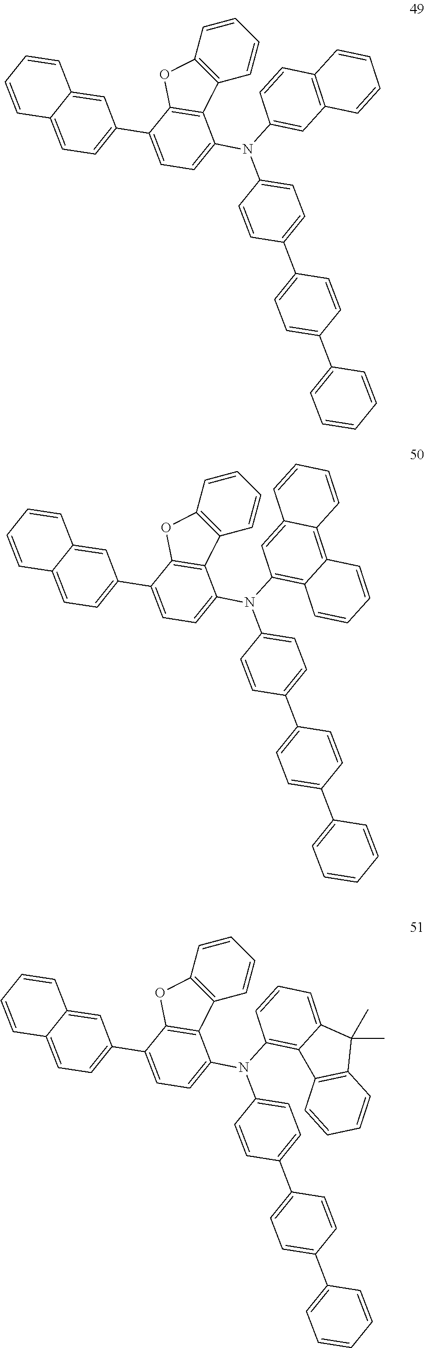 [SMILES: c1ccc(-c2ccc(-c3ccc(N(c4ccc5ccccc5c4)c4ccc(-c5ccc6ccccc6c5)c5oc6ccccc6c45)cc3)cc2)cc1 ; c1ccc(-c2ccc(-c3ccc(N(c4cc5ccccc5c5ccccc45)c4ccc(-c5ccc6ccccc6c5)c5oc6ccccc6c45)cc3)cc2)cc1 ; CC1(C)c2ccccc2-c2c(N(c3ccc(-c4ccc(-c5ccccc5)cc4)cc3)c3ccc(-c4ccc5ccccc5c4)c4oc5ccccc5c34)cccc21]

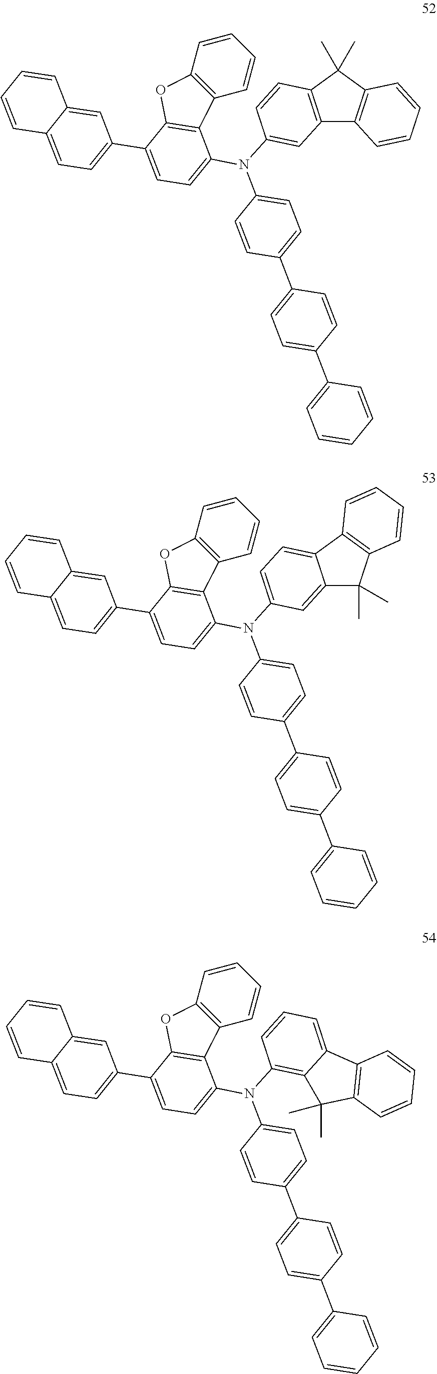 [SMILES: CC1(C)c2ccccc2-c2cc(N(c3ccc(-c4ccc(-c5ccccc5)cc4)cc3)c3ccc(-c4ccc5ccccc5c4)c4oc5ccccc5c34)ccc21 ; CC1(C)c2ccccc2-c2ccc(N(c3ccc(-c4ccc(-c5ccccc5)cc4)cc3)c3ccc(-c4ccc5ccccc5c4)c4oc5ccccc5c34)cc21 ; CC1(C)c2ccccc2-c2cccc(N(c3ccc(-c4ccc(-c5ccccc5)cc4)cc3)c3ccc(-c4ccc5ccccc5c4)c4oc5ccccc5c34)c21]

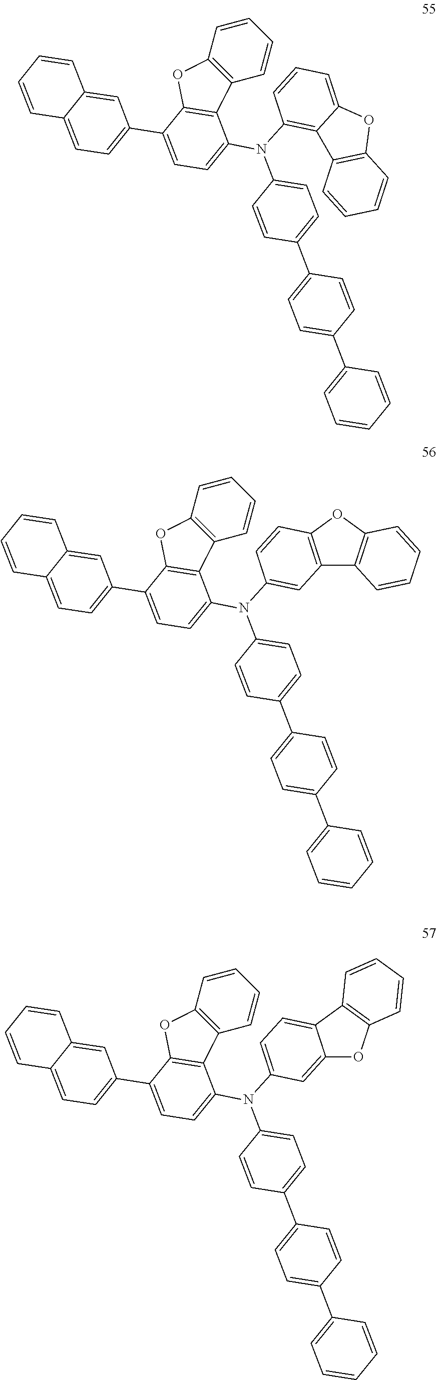 [SMILES: c1ccc(-c2ccc(-c3ccc(N(c4cccc5oc6ccccc6c45)c4ccc(-c5ccc6ccccc6c5)c5oc6ccccc6c45)cc3)cc2)cc1 ; c1ccc(-c2ccc(-c3ccc(N(c4ccc5oc6ccccc6c5c4)c4ccc(-c5ccc6ccccc6c5)c5oc6ccccc6c45)cc3)cc2)cc1 ; c1ccc(-c2ccc(-c3ccc(N(c4ccc5c(c4)oc4ccccc45)c4ccc(-c5ccc6ccccc6c5)c5oc6ccccc6c45)cc3)cc2)cc1]

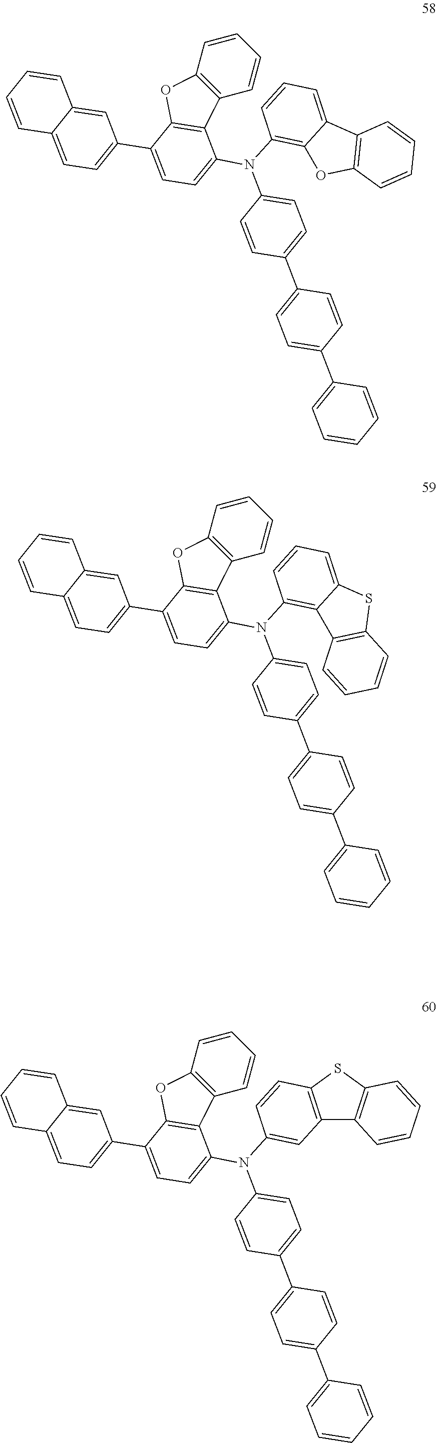 [SMILES: c1ccc(-c2ccc(-c3ccc(N(c4cccc5c4oc4ccccc45)c4ccc(-c5ccc6ccccc6c5)c5oc6ccccc6c45)cc3)cc2)cc1 ; c1ccc(-c2ccc(-c3ccc(N(c4cccc5sc6ccccc6c45)c4ccc(-c5ccc6ccccc6c5)c5oc6ccccc6c45)cc3)cc2)cc1 ; c1ccc(-c2ccc(-c3ccc(N(c4ccc5sc6ccccc6c5c4)c4ccc(-c5ccc6ccccc6c5)c5oc6ccccc6c45)cc3)cc2)cc1]

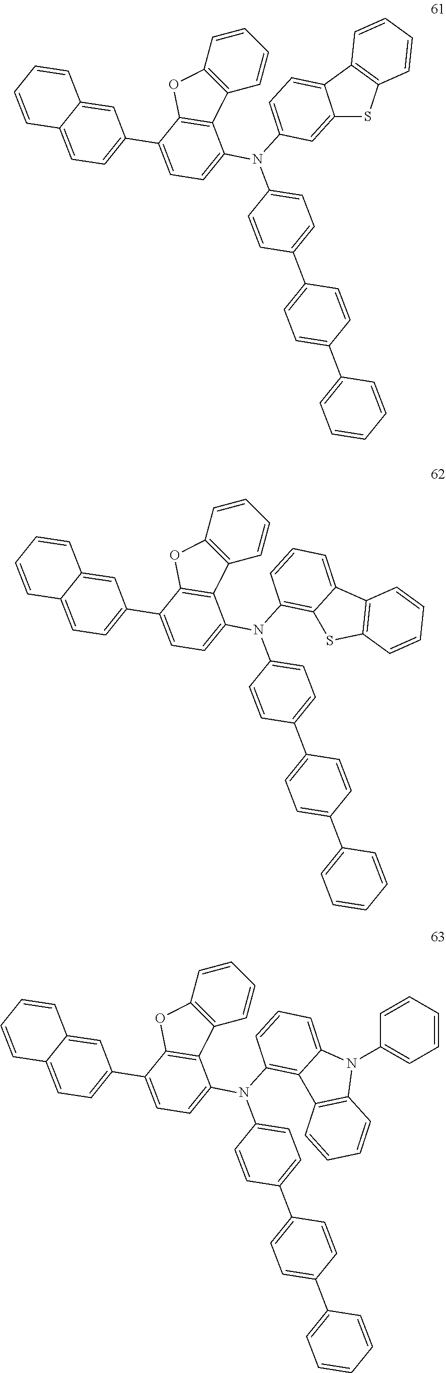 [SMILES: c1ccc(-c2ccc(-c3ccc(N(c4ccc5c(c4)sc4ccccc45)c4ccc(-c5ccc6ccccc6c5)c5oc6ccccc6c45)cc3)cc2)cc1 ; c1ccc(-c2ccc(-c3ccc(N(c4cccc5c4sc4ccccc45)c4ccc(-c5ccc6ccccc6c5)c5oc6ccccc6c45)cc3)cc2)cc1 ; c1ccc(-c2ccc(-c3ccc(N(c4ccc(-c5ccc6ccccc6c5)c5oc6ccccc6c45)c4cccc5c4c4ccccc4n5-c4ccccc4)cc3)cc2)cc1]

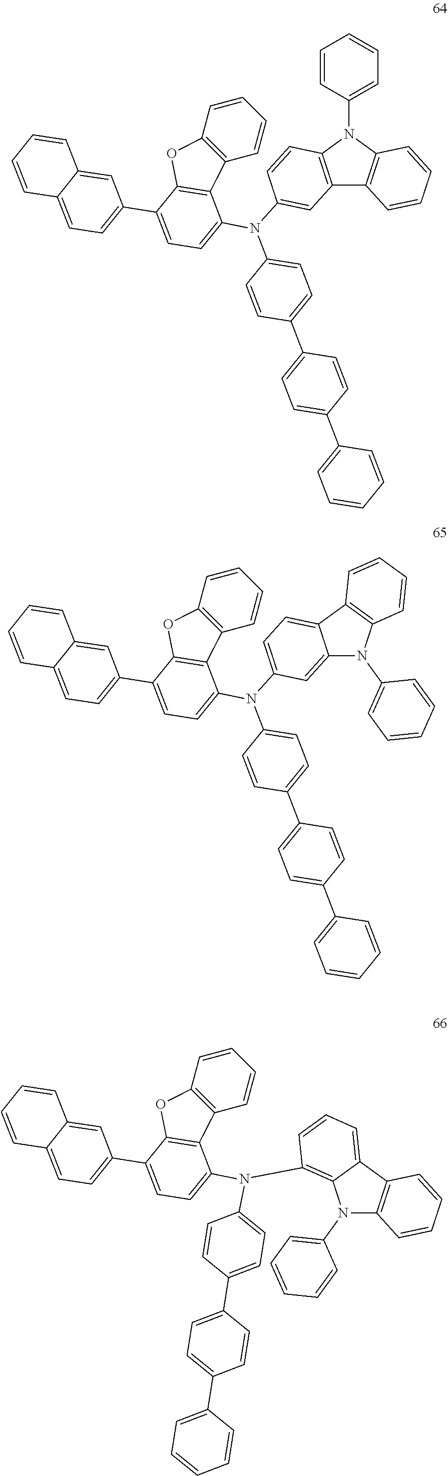 [SMILES: c1ccc(-c2ccc(-c3ccc(N(c4ccc5c(c4)c4ccccc4n5-c4ccccc4)c4ccc(-c5ccc6ccccc6c5)c5oc6ccccc6c45)cc3)cc2)cc1 ; c1ccc(-c2ccc(-c3ccc(N(c4ccc5c6ccccc6n(-c6ccccc6)c5c4)c4ccc(-c5ccc6ccccc6c5)c5oc6ccccc6c45)cc3)cc2)cc1 ; c1ccc(-c2ccc(-c3ccc(N(c4ccc(-c5ccc6ccccc6c5)c5oc6ccccc6c45)c4cccc5c6ccccc6n(-c6ccccc6)c45)cc3)cc2)cc1]

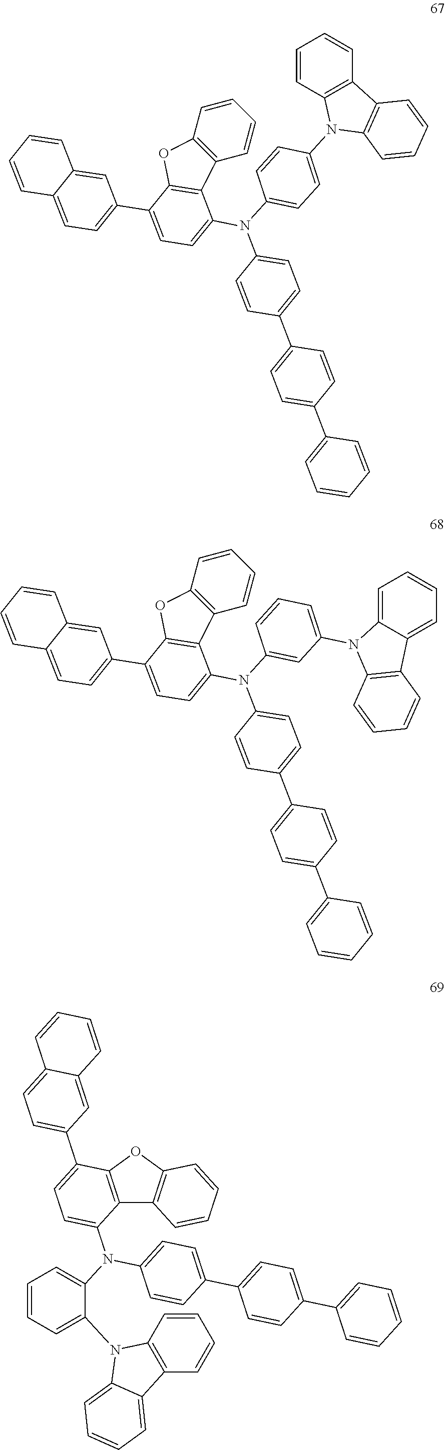 [SMILES: c1ccc(-c2ccc(-c3ccc(N(c4ccc(-n5c6ccccc6c6ccccc65)cc4)c4ccc(-c5ccc6ccccc6c5)c5oc6ccccc6c45)cc3)cc2)cc1 ; c1ccc(-c2ccc(-c3ccc(N(c4cccc(-n5c6ccccc6c6ccccc65)c4)c4ccc(-c5ccc6ccccc6c5)c5oc6ccccc6c45)cc3)cc2)cc1 ; c1ccc(-c2ccc(-c3ccc(N(c4ccccc4-n4c5ccccc5c5ccccc54)c4ccc(-c5ccc6ccccc6c5)c5oc6ccccc6c45)cc3)cc2)cc1]

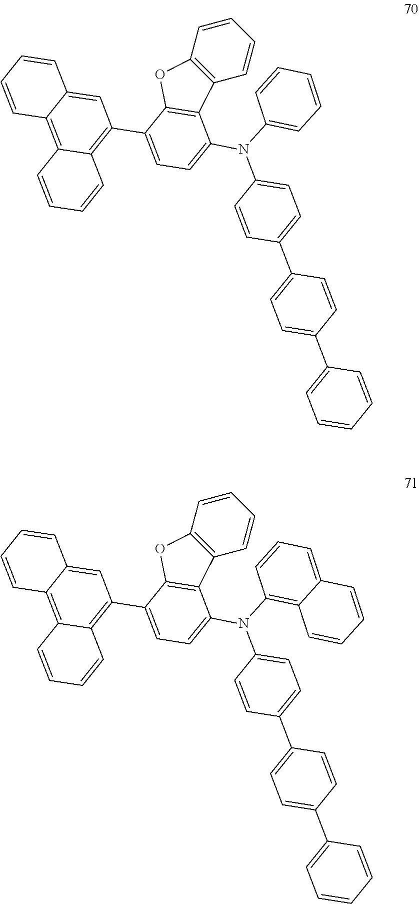 [SMILES: c1ccc(-c2ccc(-c3ccc(N(c4ccccc4)c4ccc(-c5cc6ccccc6c6ccccc56)c5oc6ccccc6c45)cc3)cc2)cc1 ; c1ccc(-c2ccc(-c3ccc(N(c4cccc5ccccc45)c4ccc(-c5cc6ccccc6c6ccccc56)c5oc6ccccc6c45)cc3)cc2)cc1]

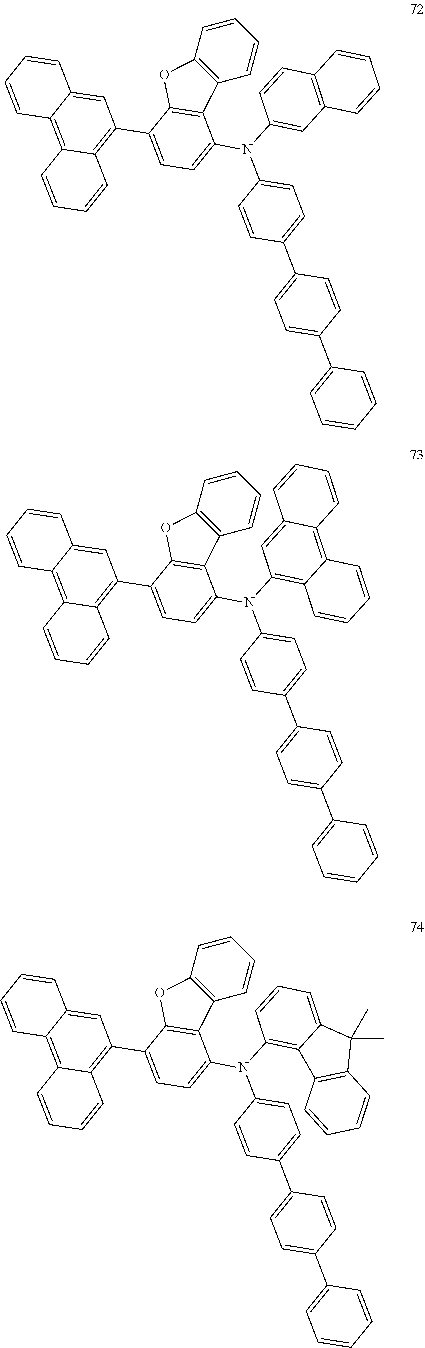 [SMILES: c1ccc(-c2ccc(-c3ccc(N(c4ccc5ccccc5c4)c4ccc(-c5cc6ccccc6c6ccccc56)c5oc6ccccc6c45)cc3)cc2)cc1 ; c1ccc(-c2ccc(-c3ccc(N(c4cc5ccccc5c5ccccc45)c4ccc(-c5cc6ccccc6c6ccccc56)c5oc6ccccc6c45)cc3)cc2)cc1 ; CC1(C)c2ccccc2-c2c(N(c3ccc(-c4ccc(-c5ccccc5)cc4)cc3)c3ccc(-c4cc5ccccc5c5ccccc45)c4oc5ccccc5c34)cccc21]

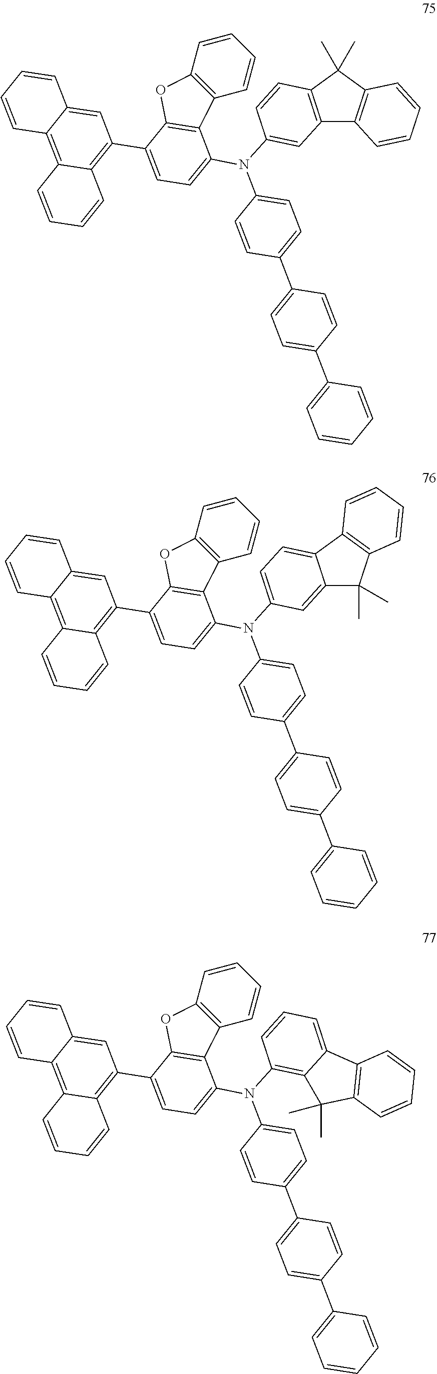 [SMILES: CC1(C)c2ccccc2-c2cc(N(c3ccc(-c4ccc(-c5ccccc5)cc4)cc3)c3ccc(-c4cc5ccccc5c5ccccc45)c4oc5ccccc5c34)ccc21 ; CC1(C)c2ccccc2-c2ccc(N(c3ccc(-c4ccc(-c5ccccc5)cc4)cc3)c3ccc(-c4cc5ccccc5c5ccccc45)c4oc5ccccc5c34)cc21 ; CC1(C)c2ccccc2-c2cccc(N(c3ccc(-c4ccc(-c5ccccc5)cc4)cc3)c3ccc(-c4cc5ccccc5c5ccccc45)c4oc5ccccc5c34)c21]

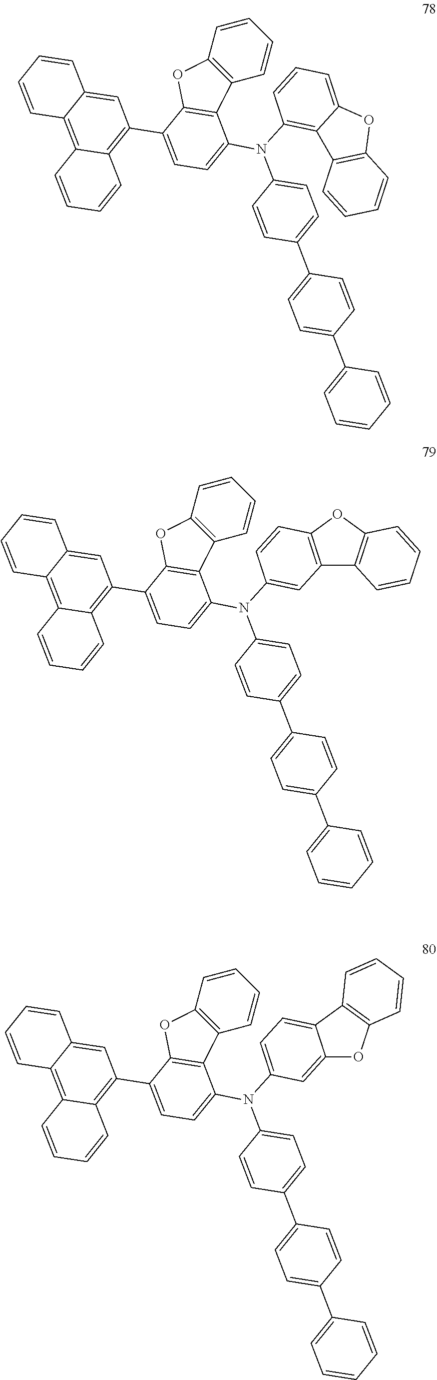 [SMILES: c1ccc(-c2ccc(-c3ccc(N(c4cccc5oc6ccccc6c45)c4ccc(-c5cc6ccccc6c6ccccc56)c5oc6ccccc6c45)cc3)cc2)cc1 ; c1ccc(-c2ccc(-c3ccc(N(c4ccc5oc6ccccc6c5c4)c4ccc(-c5cc6ccccc6c6ccccc56)c5oc6ccccc6c45)cc3)cc2)cc1 ; c1ccc(-c2ccc(-c3ccc(N(c4ccc5c(c4)oc4ccccc45)c4ccc(-c5cc6ccccc6c6ccccc56)c5oc6ccccc6c45)cc3)cc2)cc1]

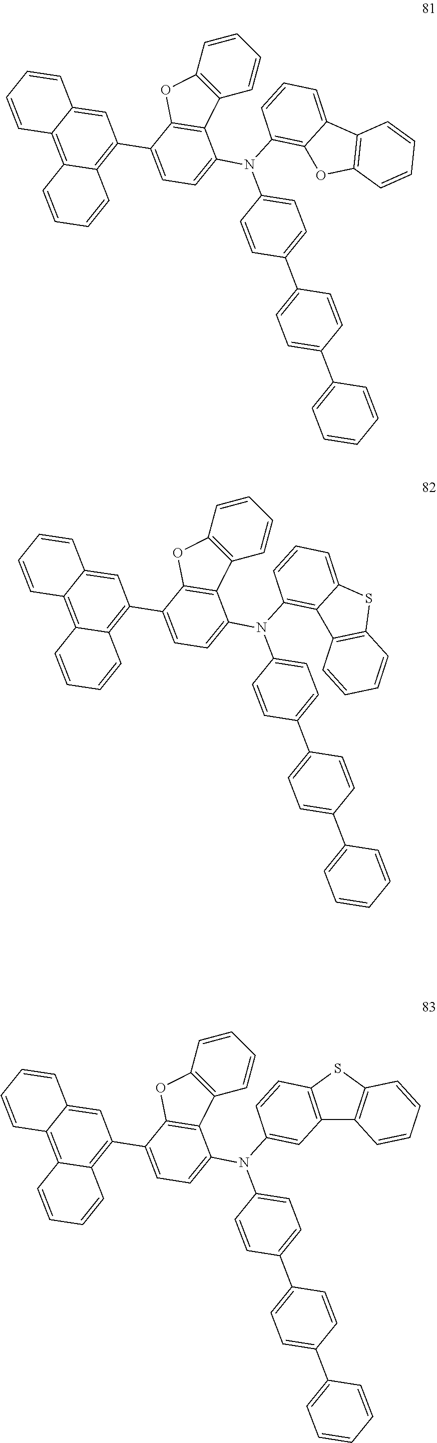 [SMILES: c1ccc(-c2ccc(-c3ccc(N(c4cccc5c4oc4ccccc45)c4ccc(-c5cc6ccccc6c6ccccc56)c5oc6ccccc6c45)cc3)cc2)cc1 ; c1ccc(-c2ccc(-c3ccc(N(c4cccc5sc6ccccc6c45)c4ccc(-c5cc6ccccc6c6ccccc56)c5oc6ccccc6c45)cc3)cc2)cc1 ; c1ccc(-c2ccc(-c3ccc(N(c4ccc5sc6ccccc6c5c4)c4ccc(-c5cc6ccccc6c6ccccc56)c5oc6ccccc6c45)cc3)cc2)cc1]

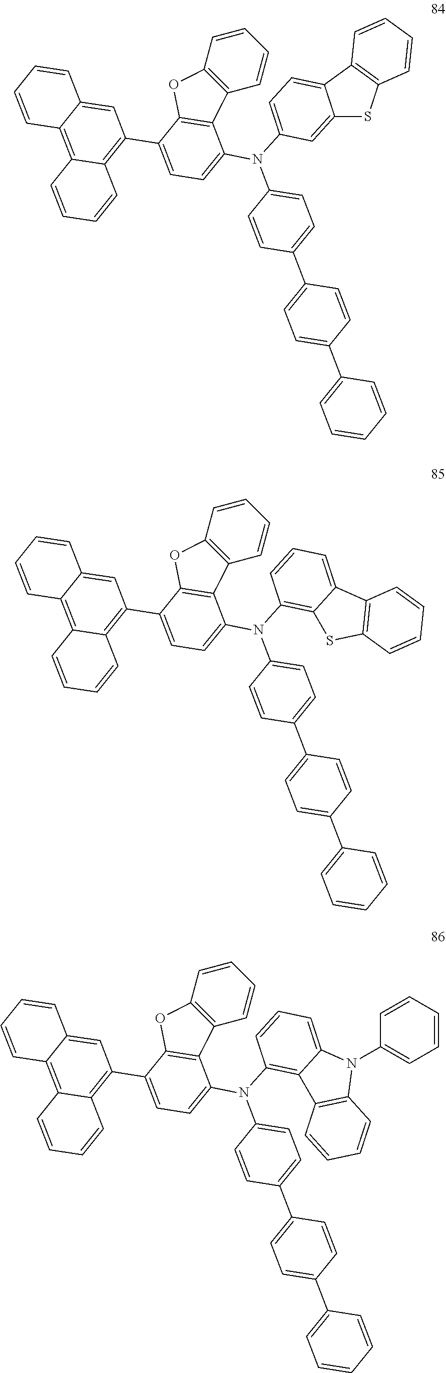 [SMILES: c1ccc(-c2ccc(-c3ccc(N(c4ccc5c(c4)sc4ccccc45)c4ccc(-c5cc6ccccc6c6ccccc56)c5oc6ccccc6c45)cc3)cc2)cc1 ; c1ccc(-c2ccc(-c3ccc(N(c4cccc5c4sc4ccccc45)c4ccc(-c5cc6ccccc6c6ccccc56)c5oc6ccccc6c45)cc3)cc2)cc1 ; c1ccc(-c2ccc(-c3ccc(N(c4ccc(-c5cc6ccccc6c6ccccc56)c5oc6ccccc6c45)c4cccc5c4c4ccccc4n5-c4ccccc4)cc3)cc2)cc1]

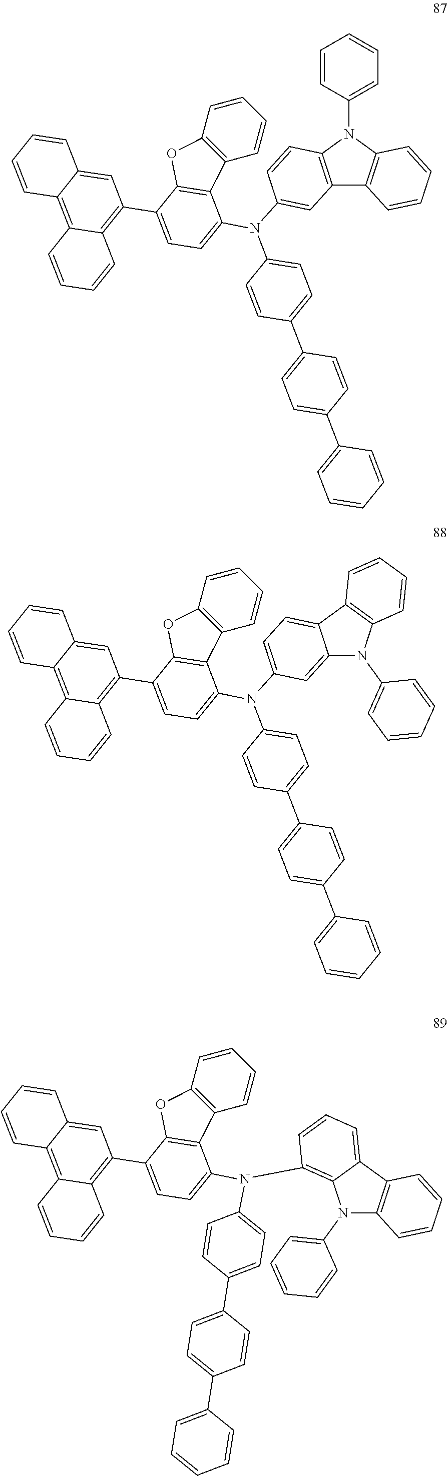 [SMILES: c1ccc(-c2ccc(-c3ccc(N(c4ccc5c(c4)c4ccccc4n5-c4ccccc4)c4ccc(-c5cc6ccccc6c6ccccc56)c5oc6ccccc6c45)cc3)cc2)cc1 ; c1ccc(-c2ccc(-c3ccc(N(c4ccc5c6ccccc6n(-c6ccccc6)c5c4)c4ccc(-c5cc6ccccc6c6ccccc56)c5oc6ccccc6c45)cc3)cc2)cc1 ; c1ccc(-c2ccc(-c3ccc(N(c4ccc(-c5cc6ccccc6c6ccccc56)c5oc6ccccc6c45)c4cccc5c6ccccc6n(-c6ccccc6)c45)cc3)cc2)cc1]

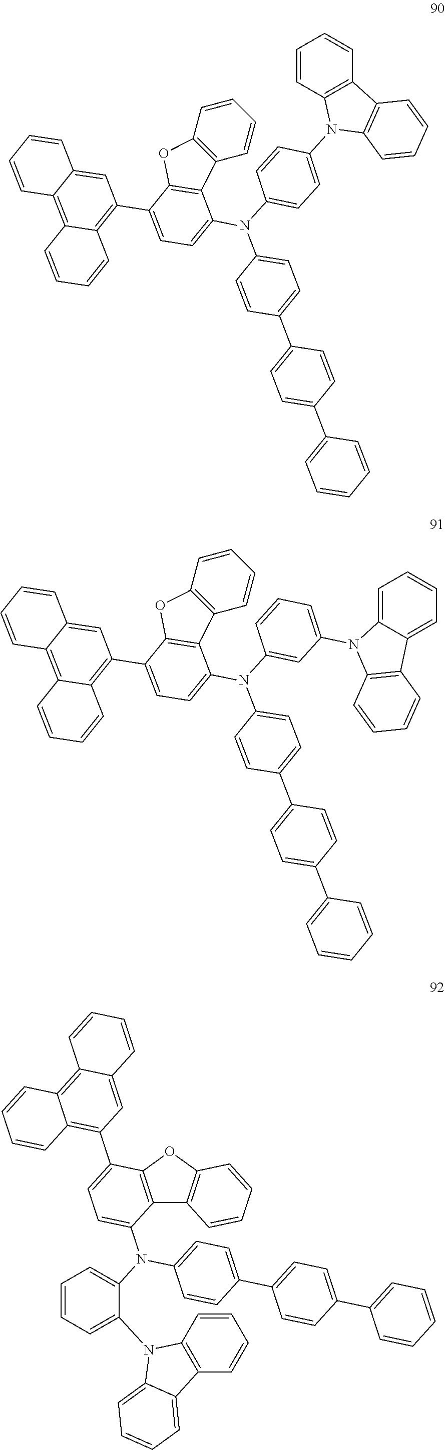 [SMILES: c1ccc(-c2ccc(-c3ccc(N(c4ccc(-n5c6ccccc6c6ccccc65)cc4)c4ccc(-c5cc6ccccc6c6ccccc56)c5oc6ccccc6c45)cc3)cc2)cc1 ; c1ccc(-c2ccc(-c3ccc(N(c4cccc(-n5c6ccccc6c6ccccc65)c4)c4ccc(-c5cc6ccccc6c6ccccc56)c5oc6ccccc6c45)cc3)cc2)cc1 ; c1ccc(-c2ccc(-c3ccc(N(c4ccccc4-n4c5ccccc5c5ccccc54)c4ccc(-c5cc6ccccc6c6ccccc56)c5oc6ccccc6c45)cc3)cc2)cc1]

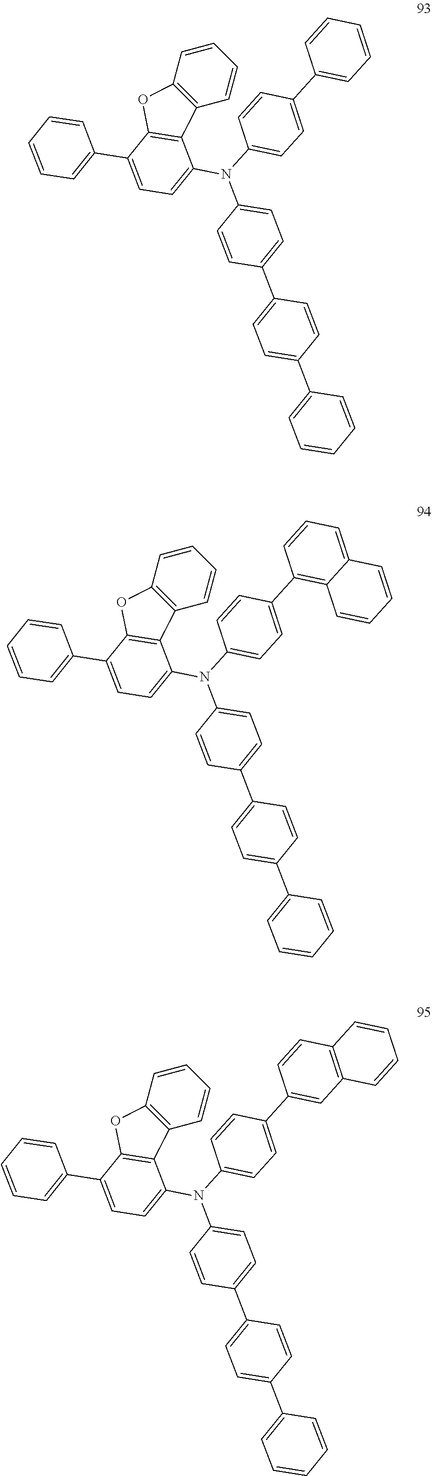 [SMILES: c1ccc(-c2ccc(-c3ccc(N(c4ccc(-c5ccccc5)cc4)c4ccc(-c5ccccc5)c5oc6ccccc6c45)cc3)cc2)cc1 ; c1ccc(-c2ccc(-c3ccc(N(c4ccc(-c5cccc6ccccc56)cc4)c4ccc(-c5ccccc5)c5oc6ccccc6c45)cc3)cc2)cc1 ; c1ccc(-c2ccc(-c3ccc(N(c4ccc(-c5ccc6ccccc6c5)cc4)c4ccc(-c5ccccc5)c5oc6ccccc6c45)cc3)cc2)cc1]

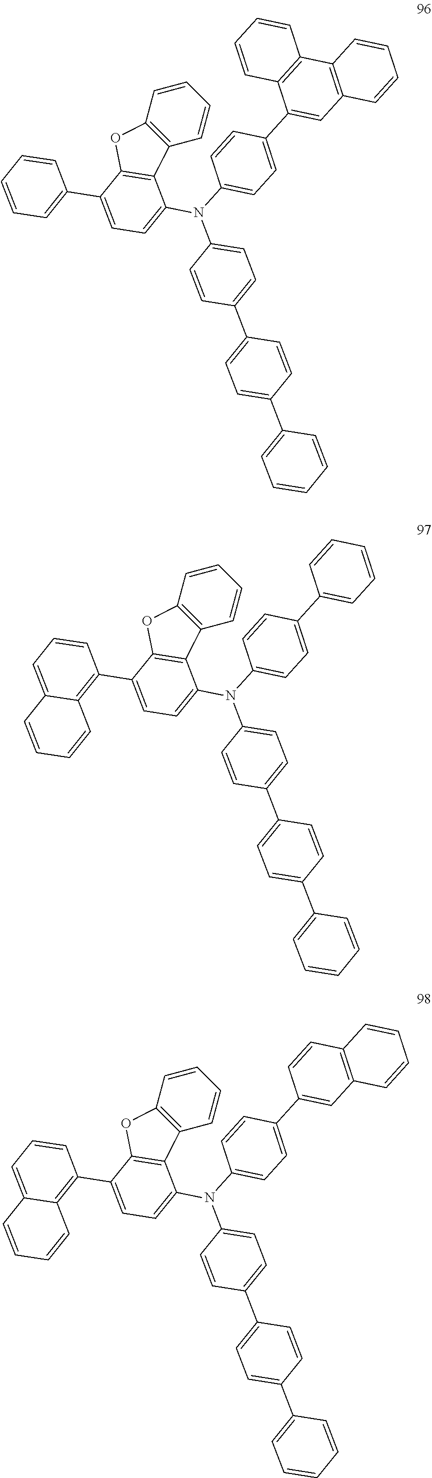 [SMILES: c1ccc(-c2ccc(-c3ccc(N(c4ccc(-c5cc6ccccc6c6ccccc56)cc4)c4ccc(-c5ccccc5)c5oc6ccccc6c45)cc3)cc2)cc1 ; c1ccc(-c2ccc(-c3ccc(N(c4ccc(-c5ccccc5)cc4)c4ccc(-c5cccc6ccccc56)c5oc6ccccc6c45)cc3)cc2)cc1 ; c1ccc(-c2ccc(-c3ccc(N(c4ccc(-c5ccc6ccccc6c5)cc4)c4ccc(-c5cccc6ccccc56)c5oc6ccccc6c45)cc3)cc2)cc1]

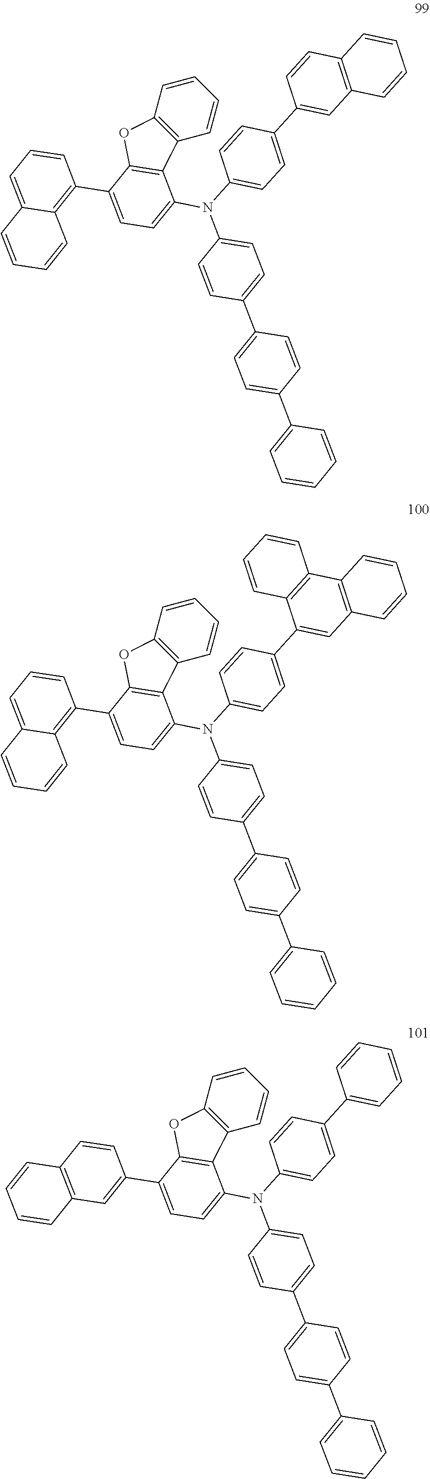 [SMILES: c1ccc(-c2ccc(-c3ccc(N(c4ccc(-c5ccc6ccccc6c5)cc4)c4ccc(-c5cccc6ccccc56)c5oc6ccccc6c45)cc3)cc2)cc1 ; c1ccc(-c2ccc(-c3ccc(N(c4ccc(-c5cc6ccccc6c6ccccc56)cc4)c4ccc(-c5cccc6ccccc56)c5oc6ccccc6c45)cc3)cc2)cc1 ; c1ccc(-c2ccc(-c3ccc(N(c4ccc(-c5ccccc5)cc4)c4ccc(-c5ccc6ccccc6c5)c5oc6ccccc6c45)cc3)cc2)cc1]

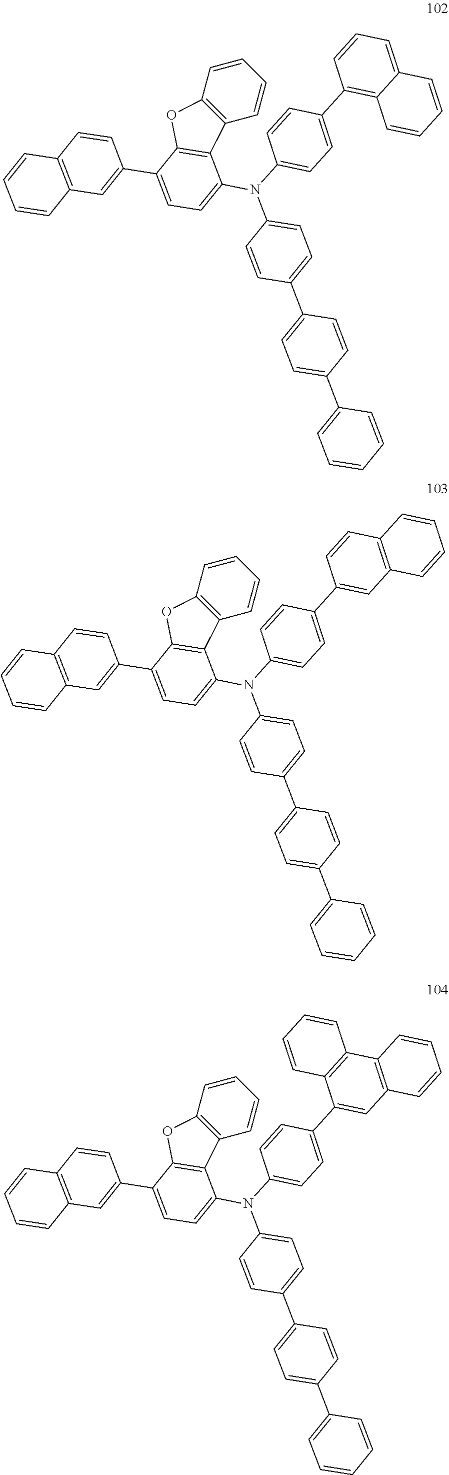 [SMILES: c1ccc(-c2ccc(-c3ccc(N(c4ccc(-c5cccc6ccccc56)cc4)c4ccc(-c5ccc6ccccc6c5)c5oc6ccccc6c45)cc3)cc2)cc1 ; c1ccc(-c2ccc(-c3ccc(N(c4ccc(-c5ccc6ccccc6c5)cc4)c4ccc(-c5ccc6ccccc6c5)c5oc6ccccc6c45)cc3)cc2)cc1 ; c1ccc(-c2ccc(-c3ccc(N(c4ccc(-c5cc6ccccc6c6ccccc56)cc4)c4ccc(-c5ccc6ccccc6c5)c5oc6ccccc6c45)cc3)cc2)cc1]

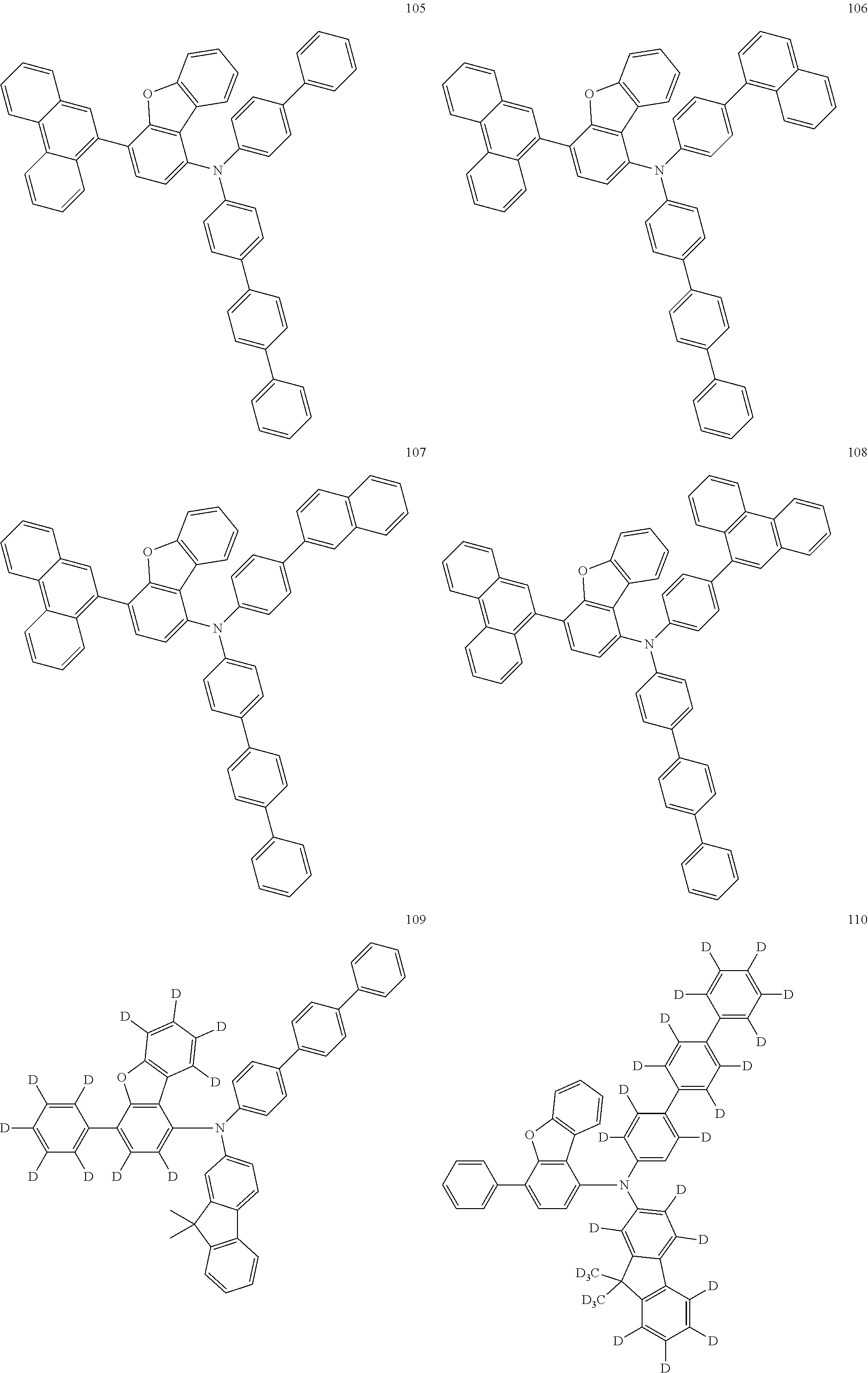

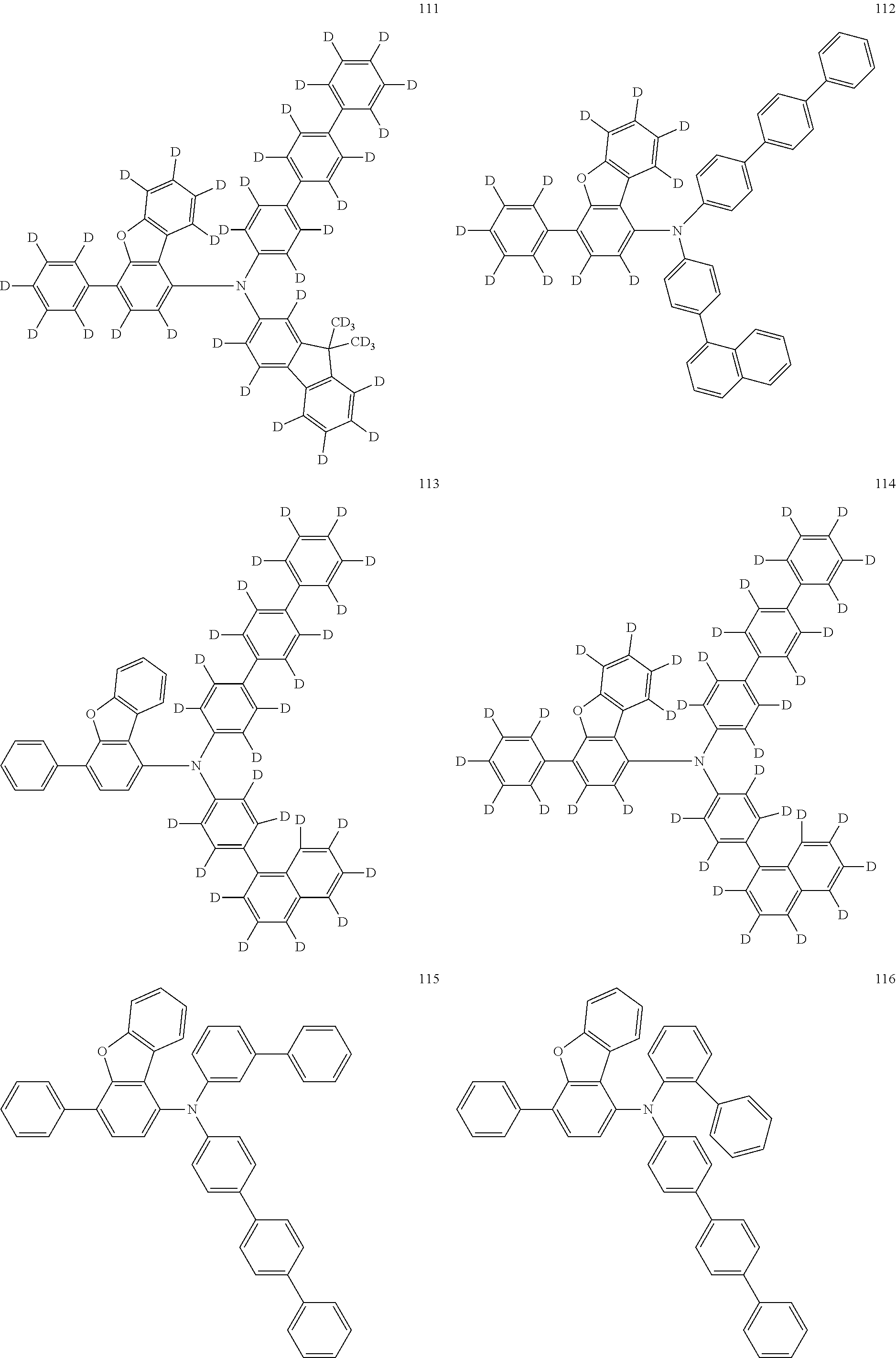

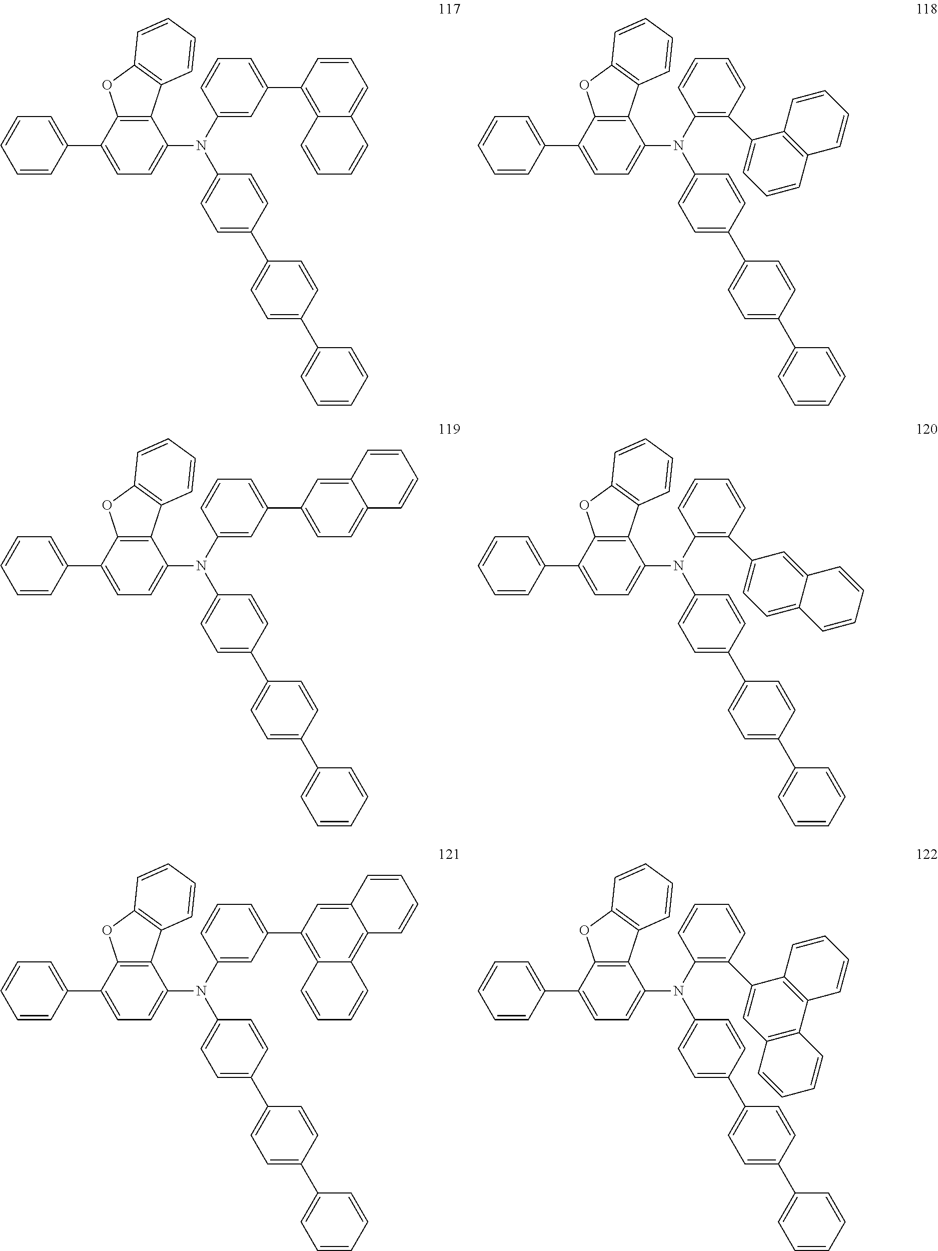

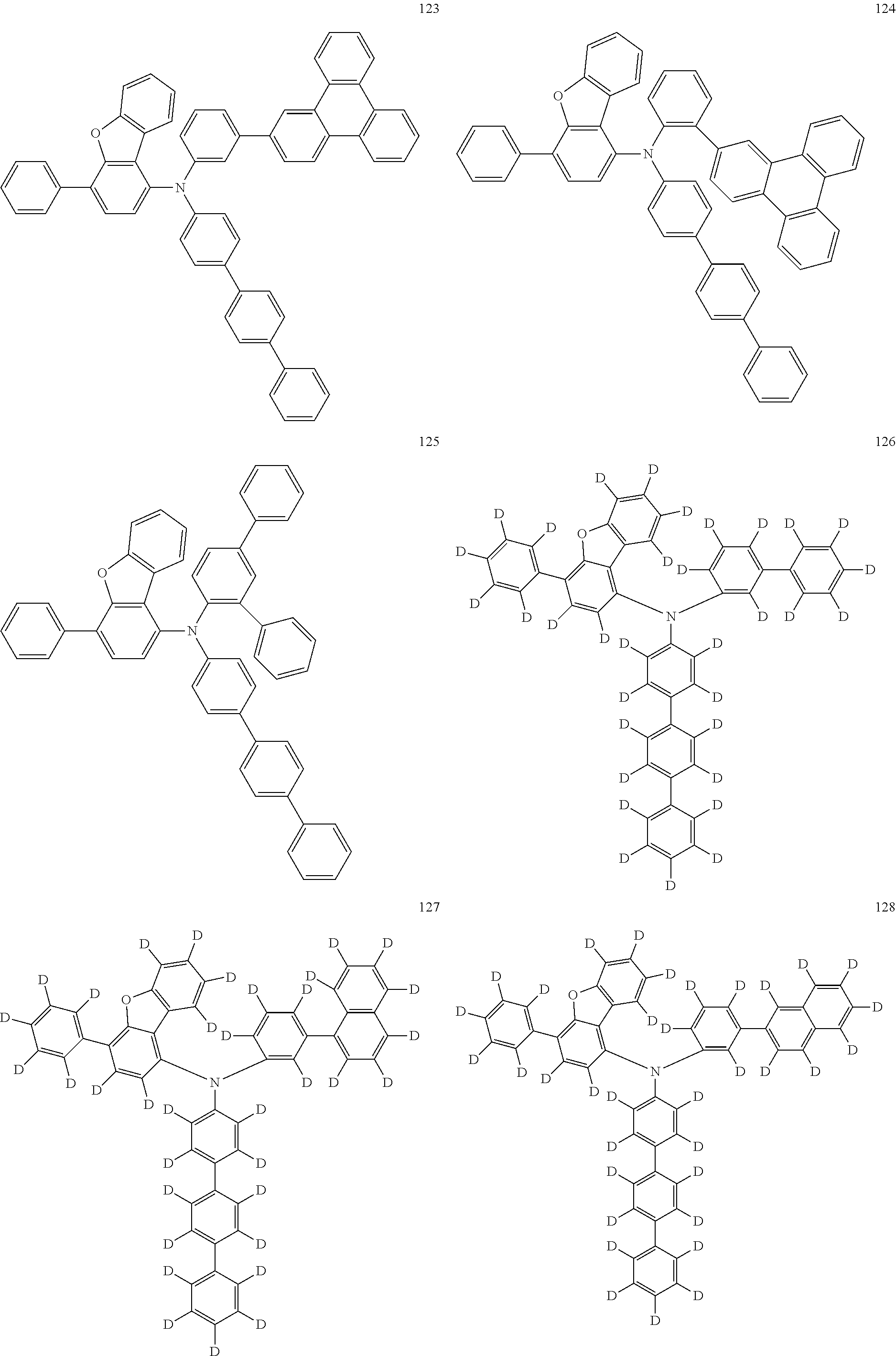

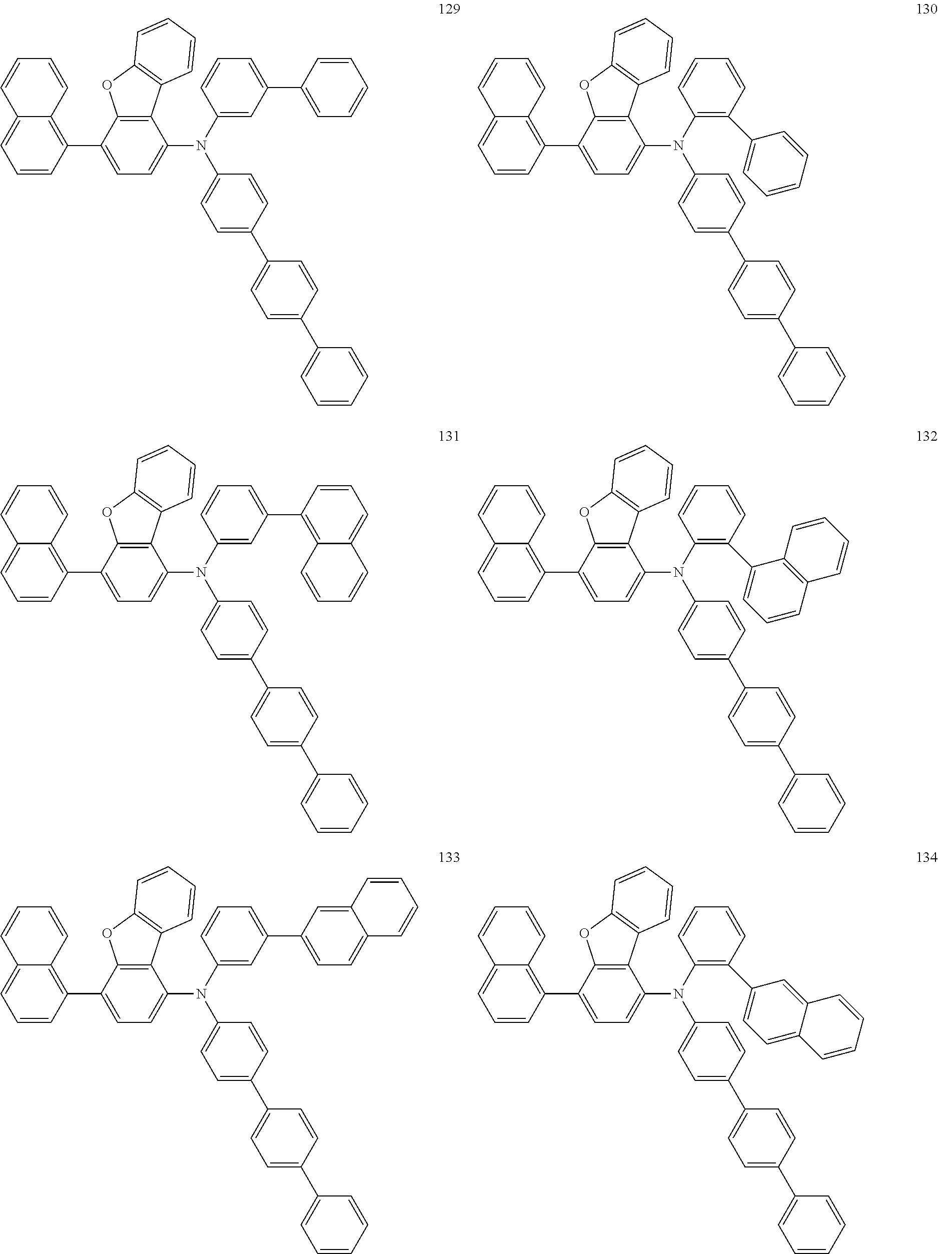

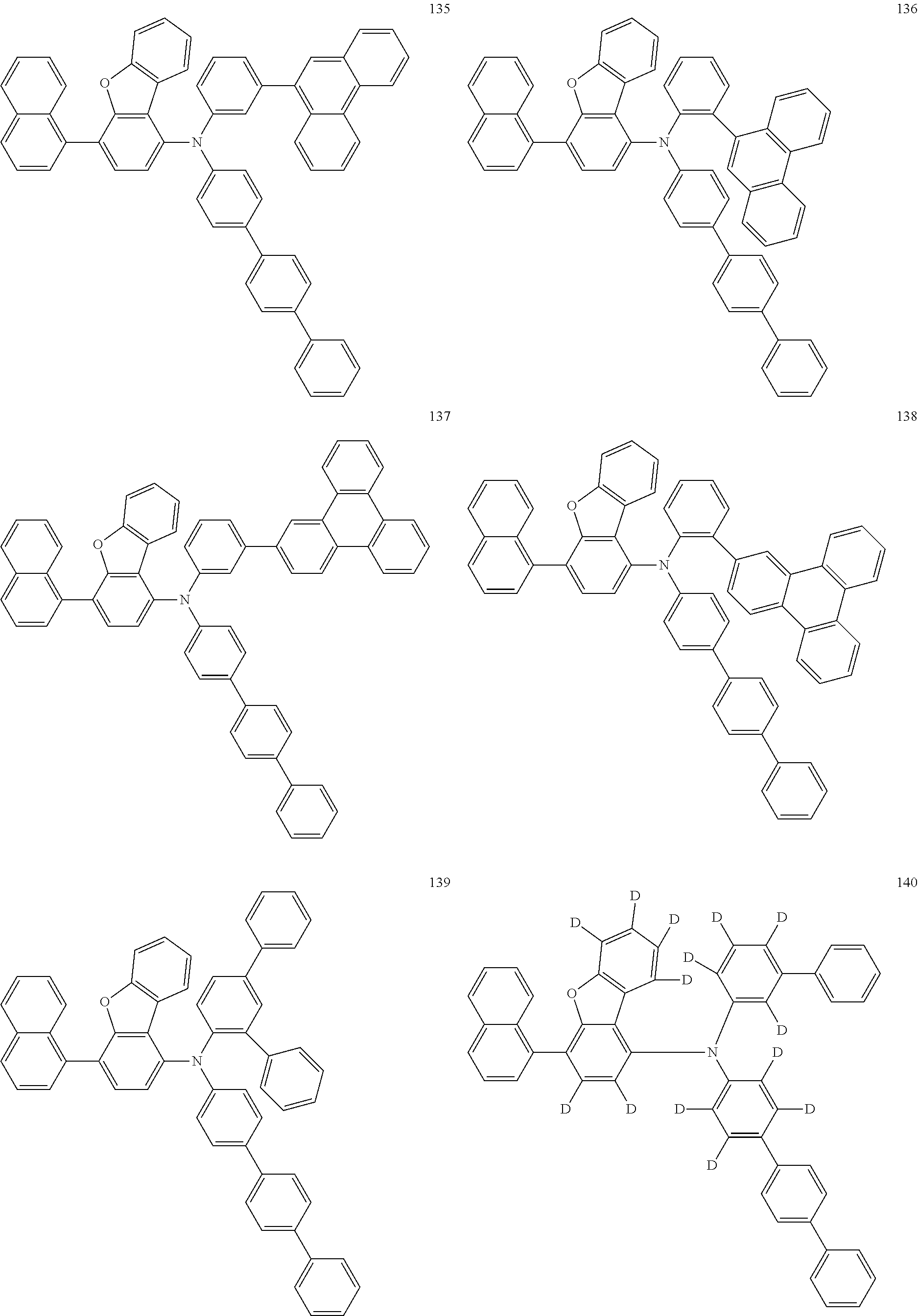

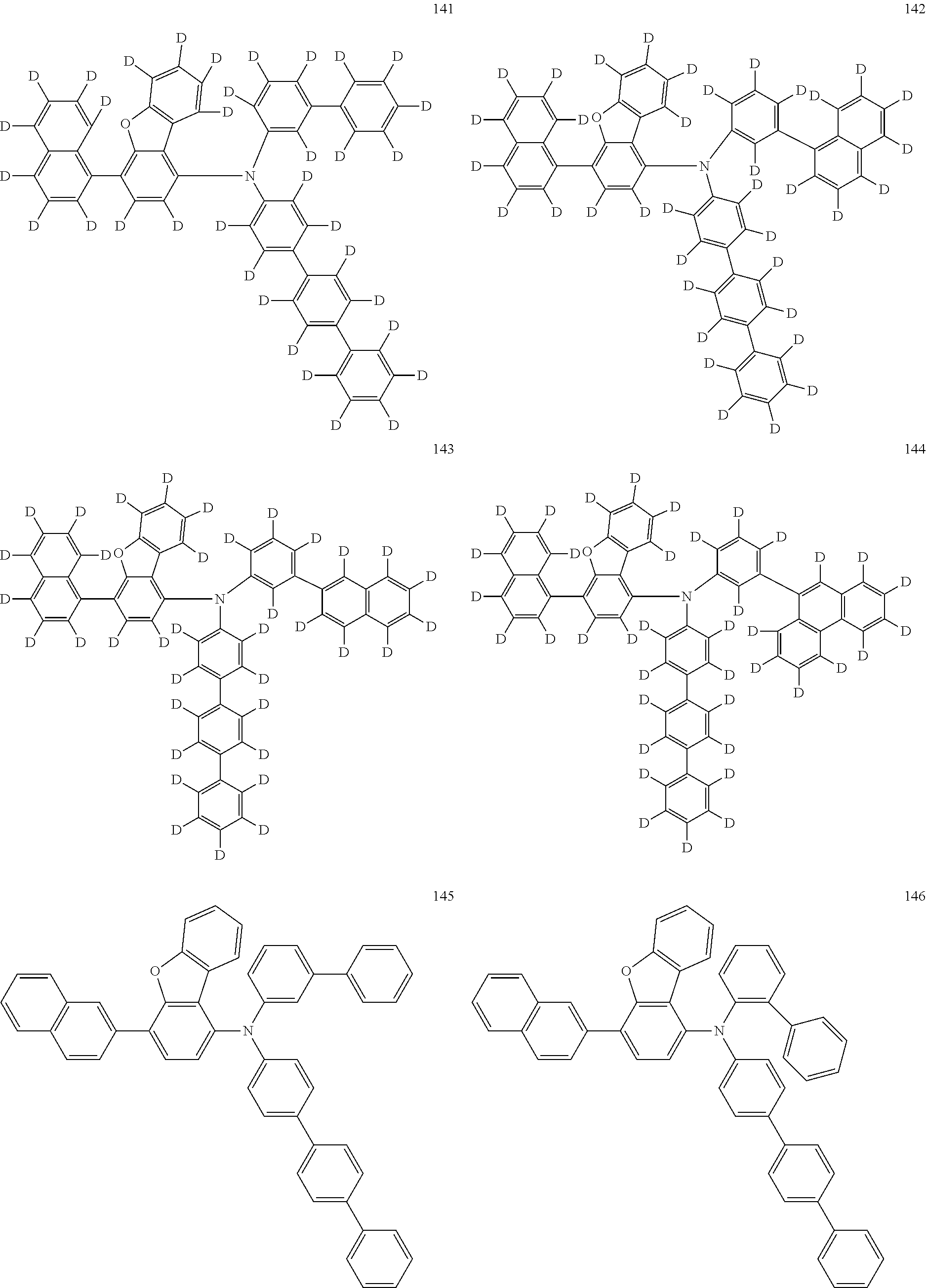

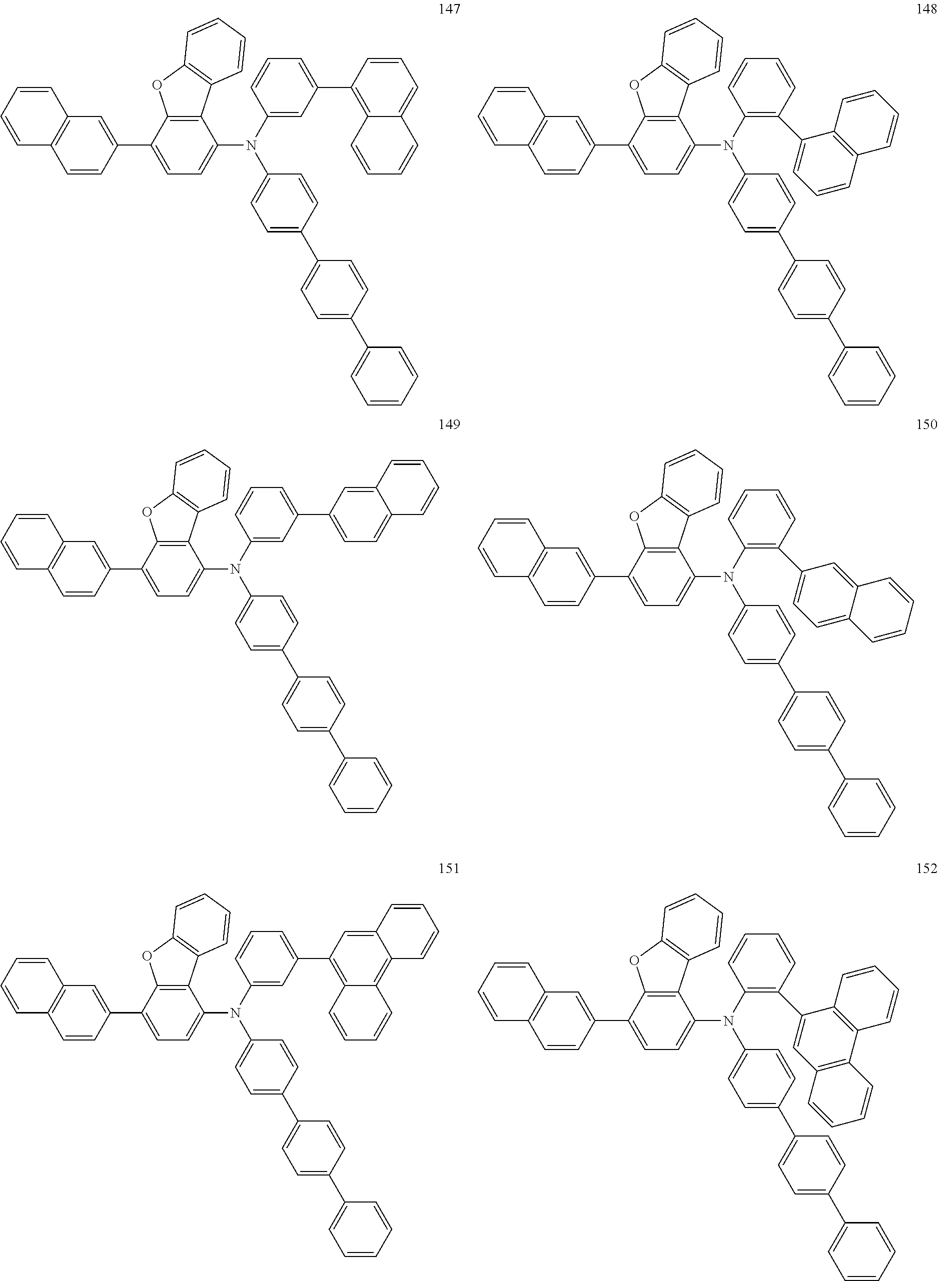

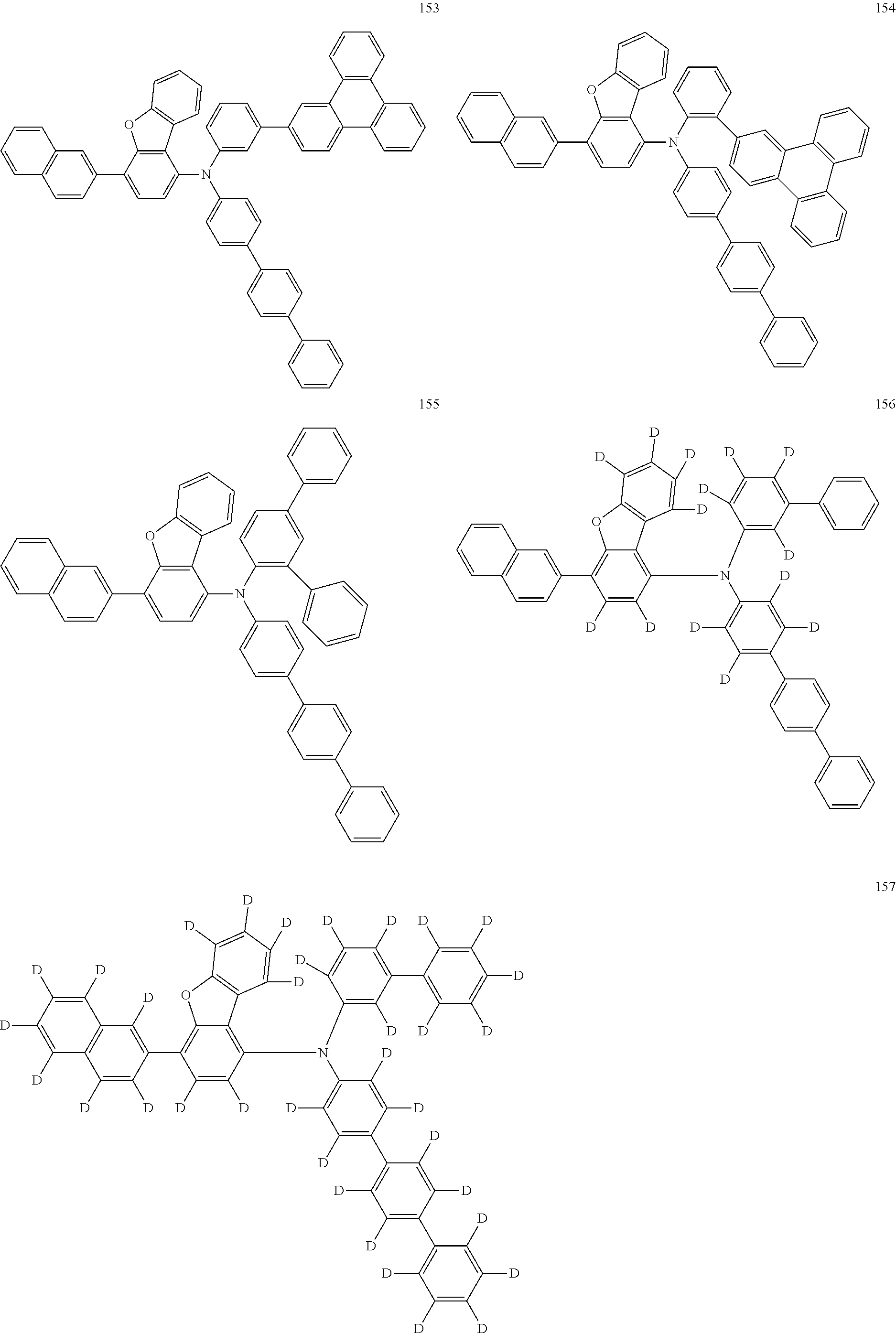

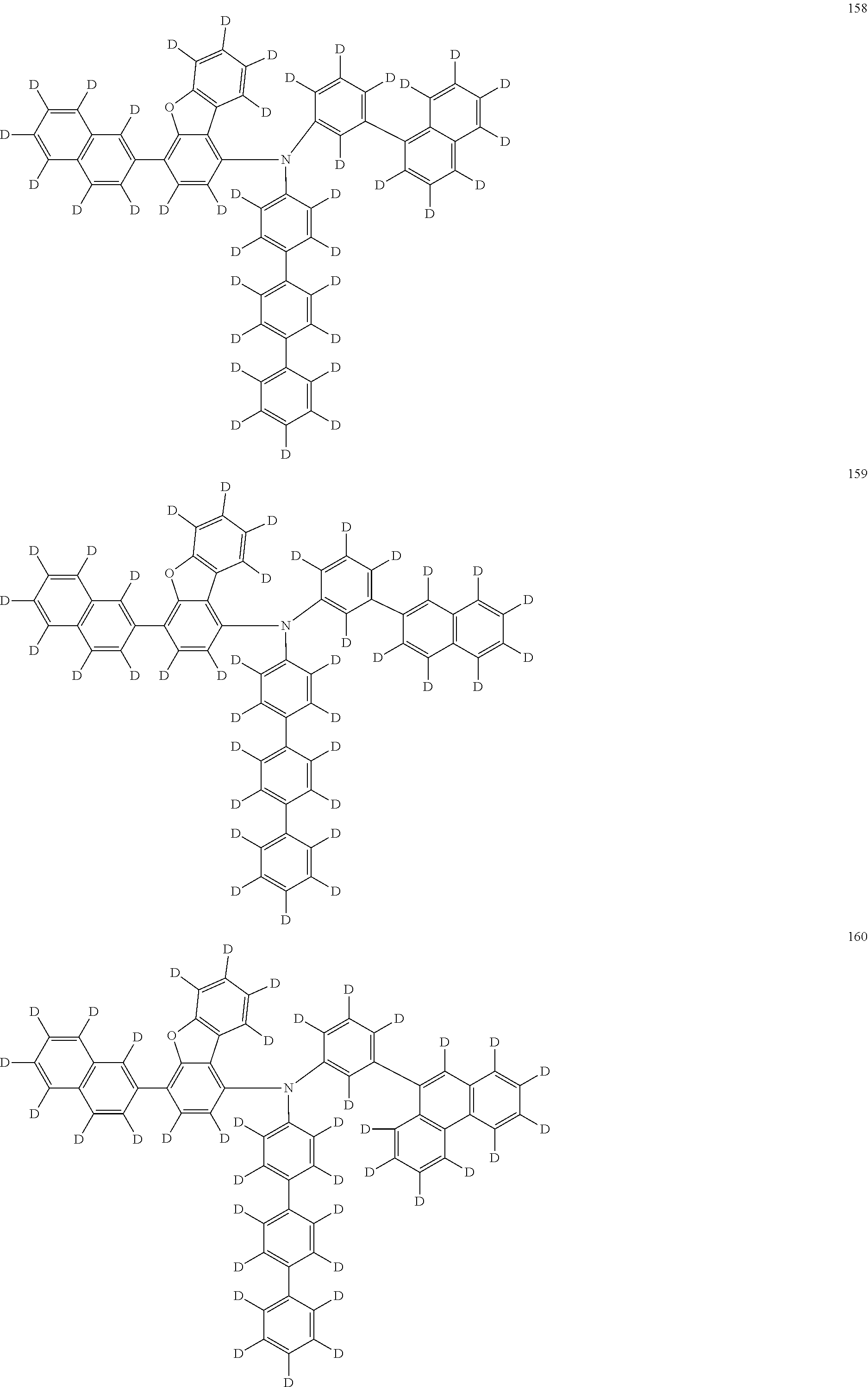

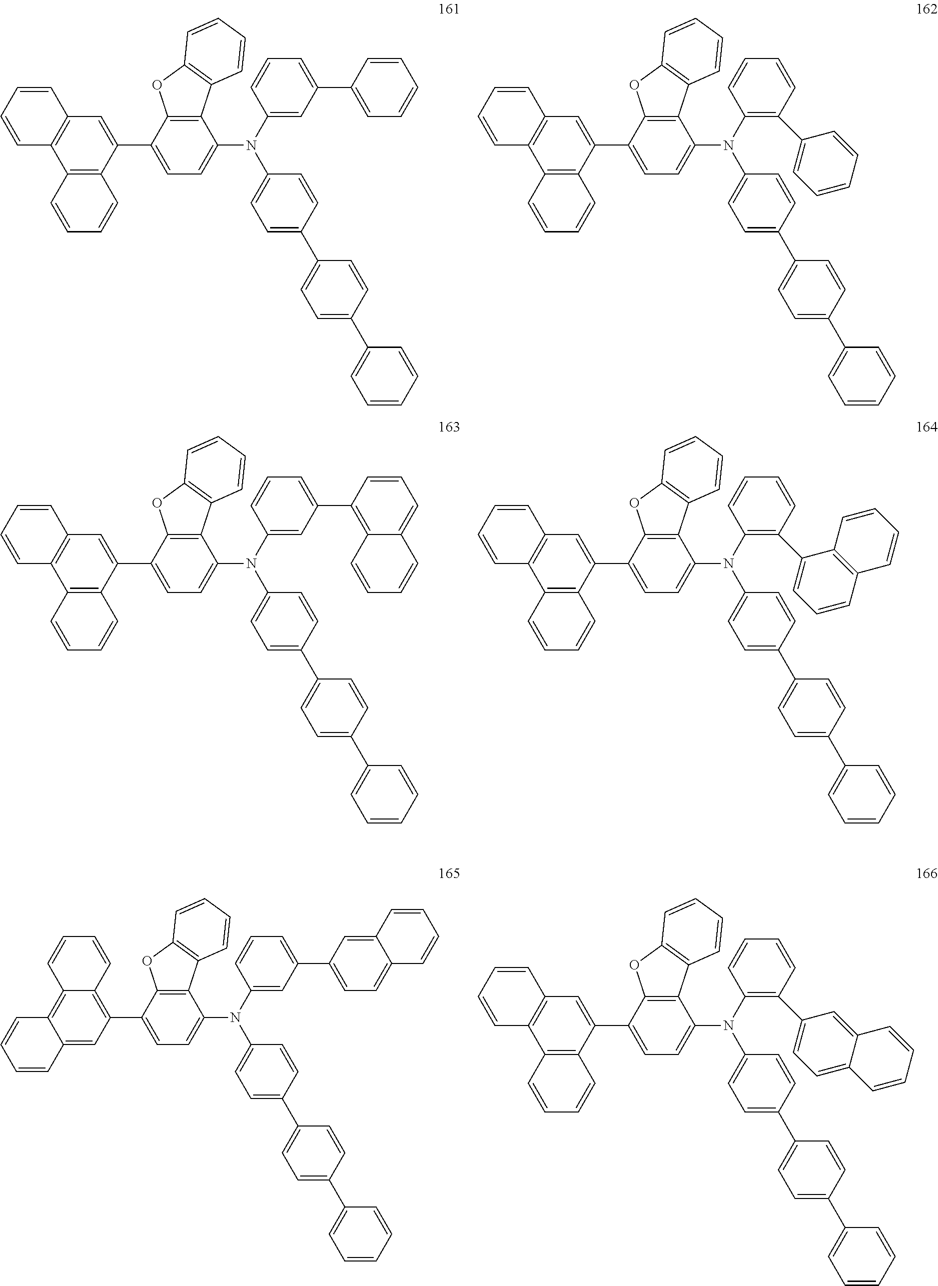

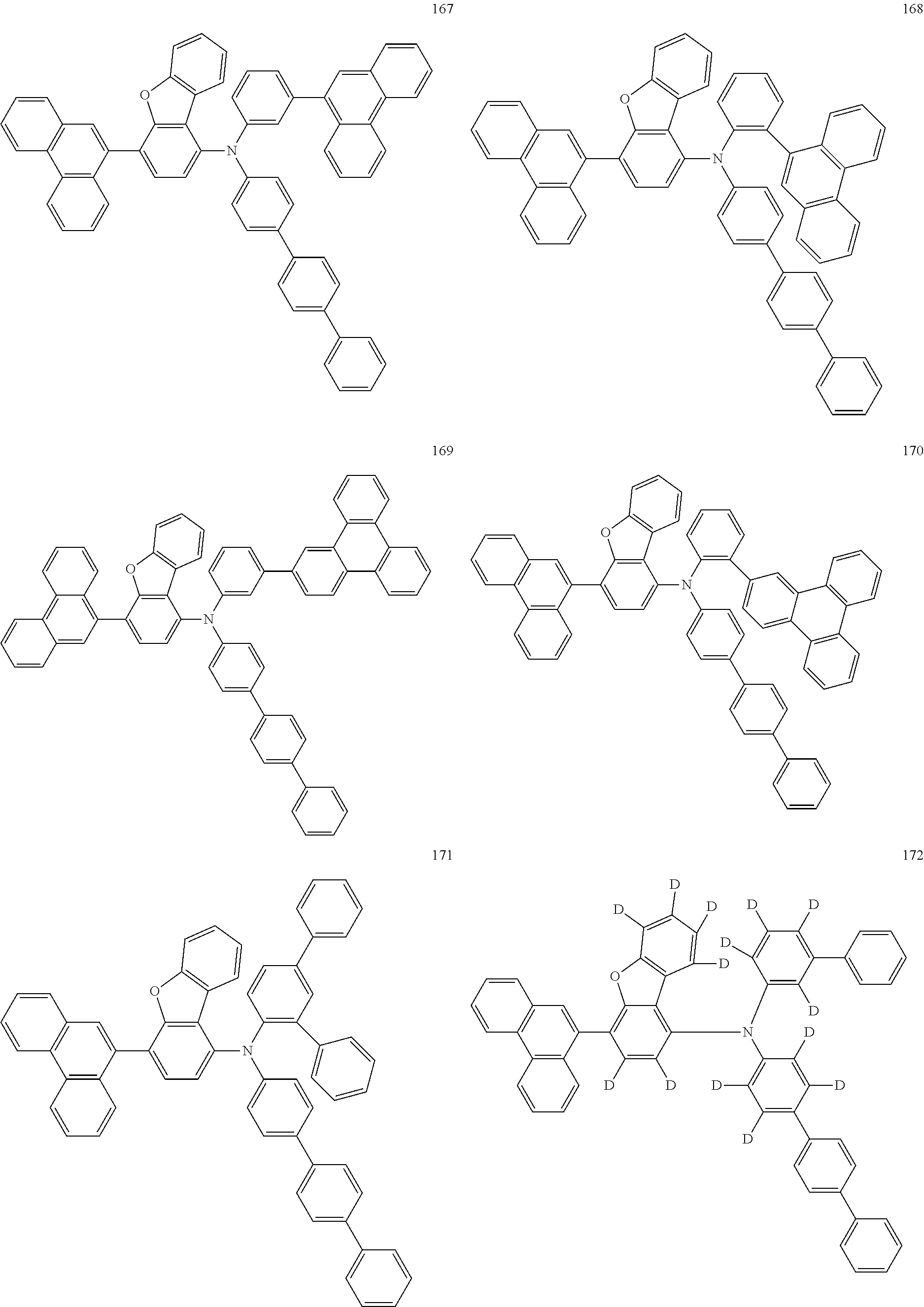

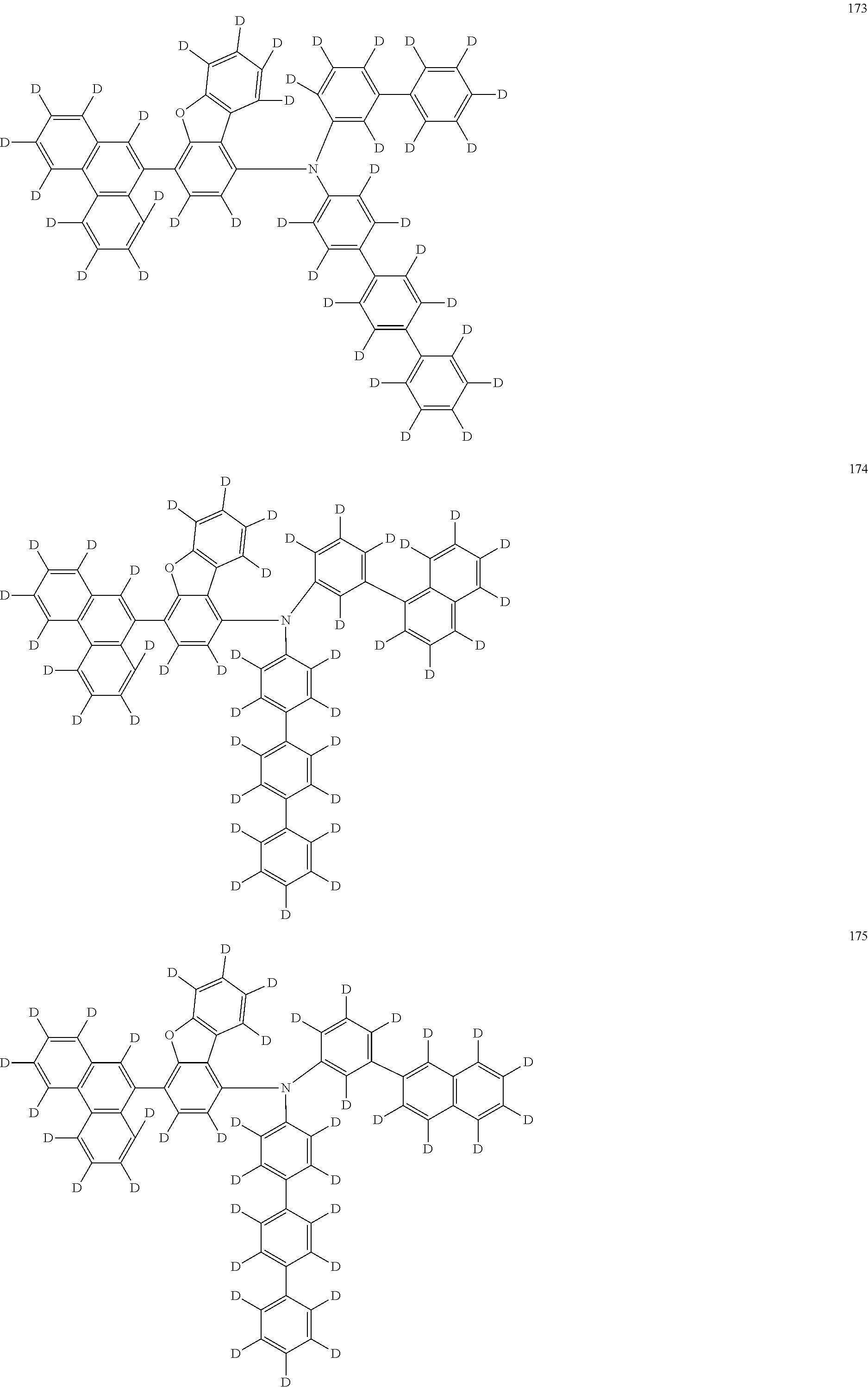

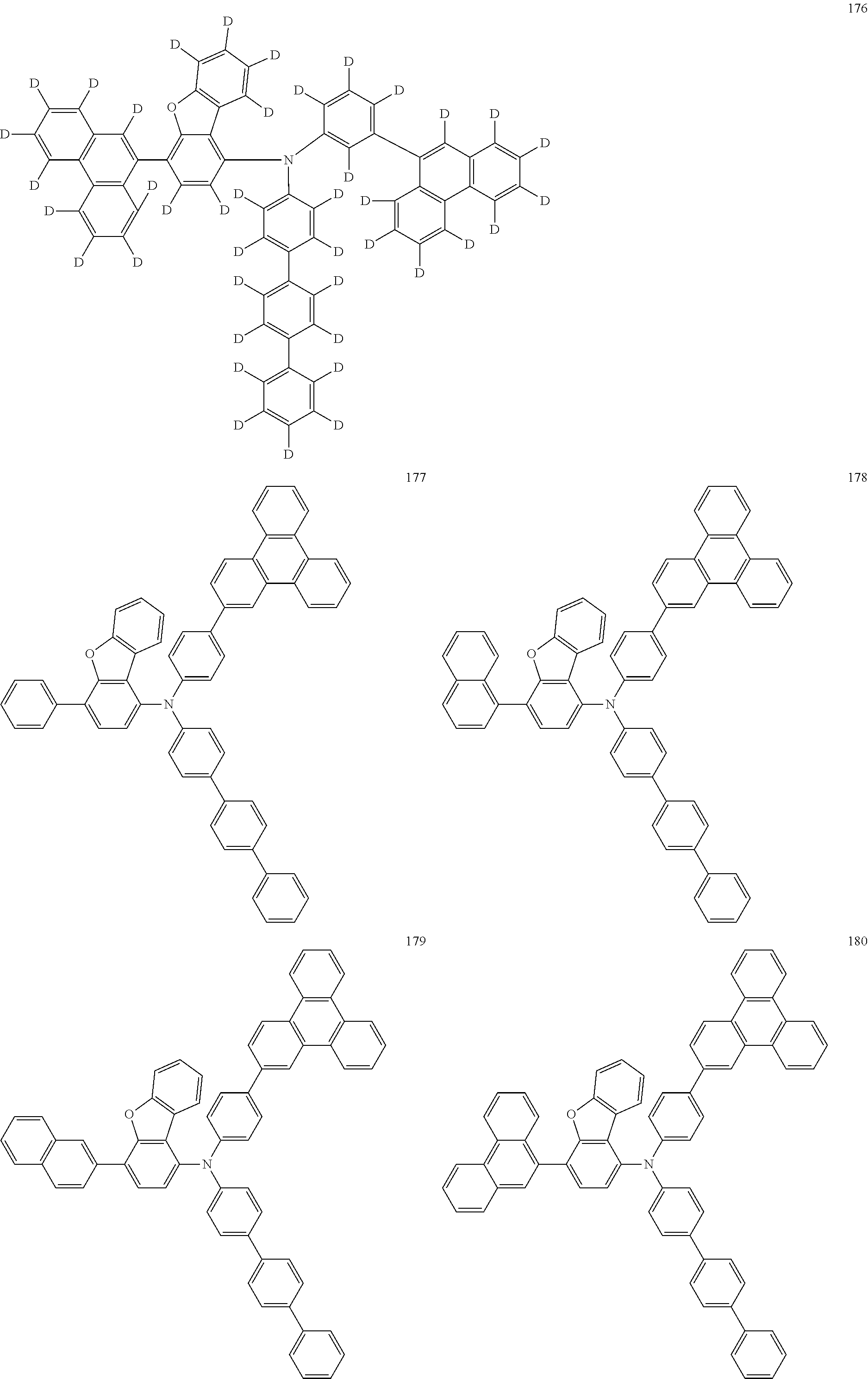

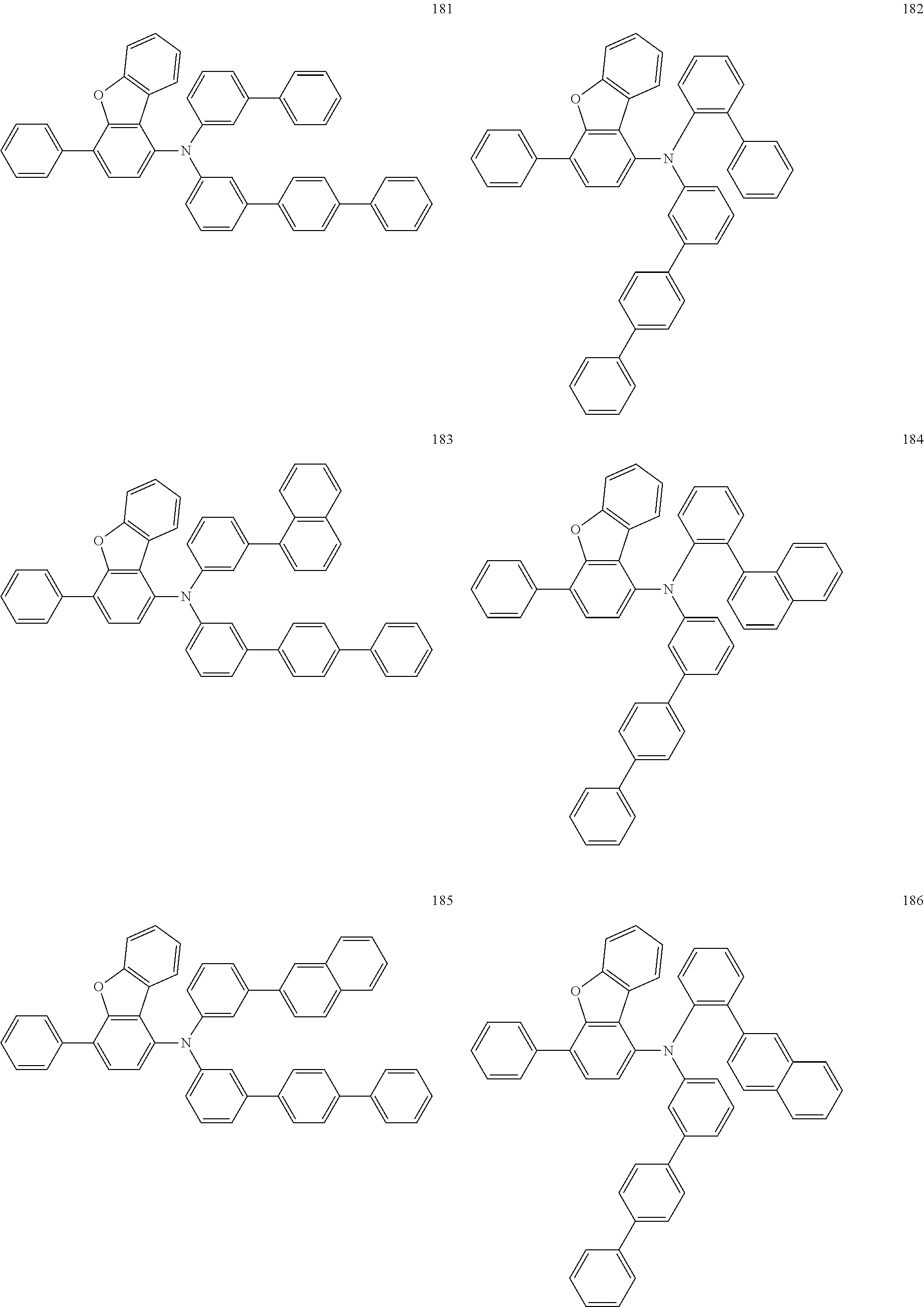

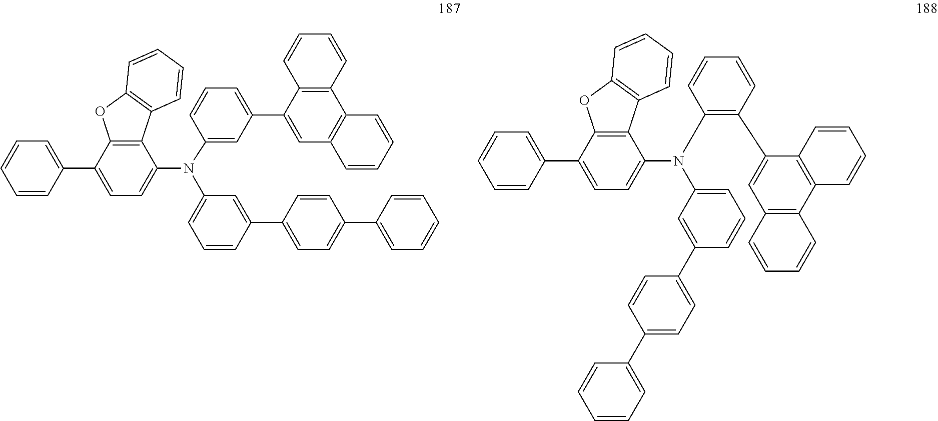 [SMILES: c1ccc(-c2ccc(-c3cccc(N(c4cccc(-c5cc6ccccc6c6ccccc56)c4)c4ccc(-c5ccccc5)c5oc6ccccc6c45)c3)cc2)cc1 ; c1ccc(-c2ccc(-c3cccc(N(c4ccccc4-c4cc5ccccc5c5ccccc45)c4ccc(-c5ccccc5)c5oc6ccccc6c45)c3)cc2)cc1]

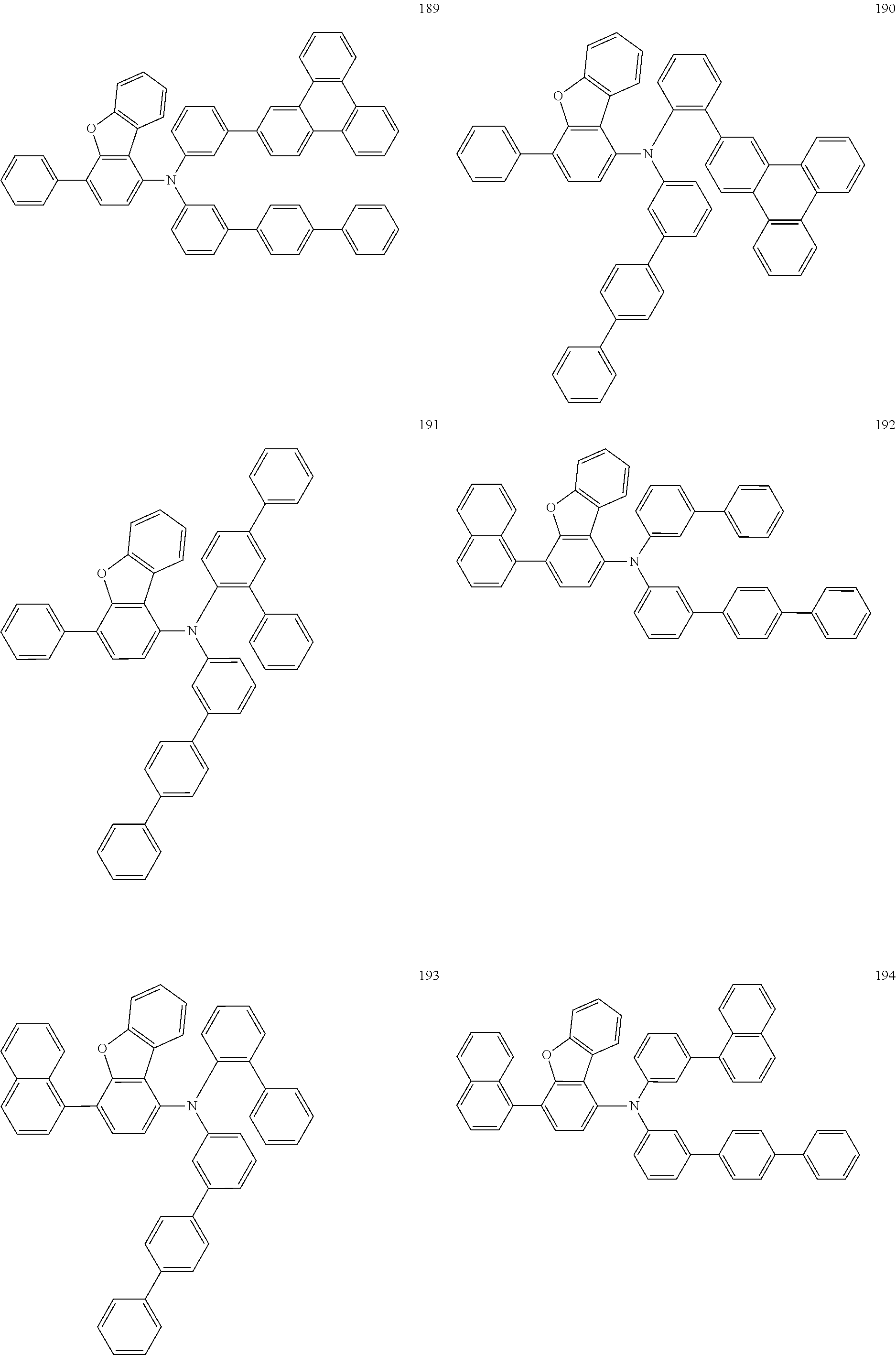

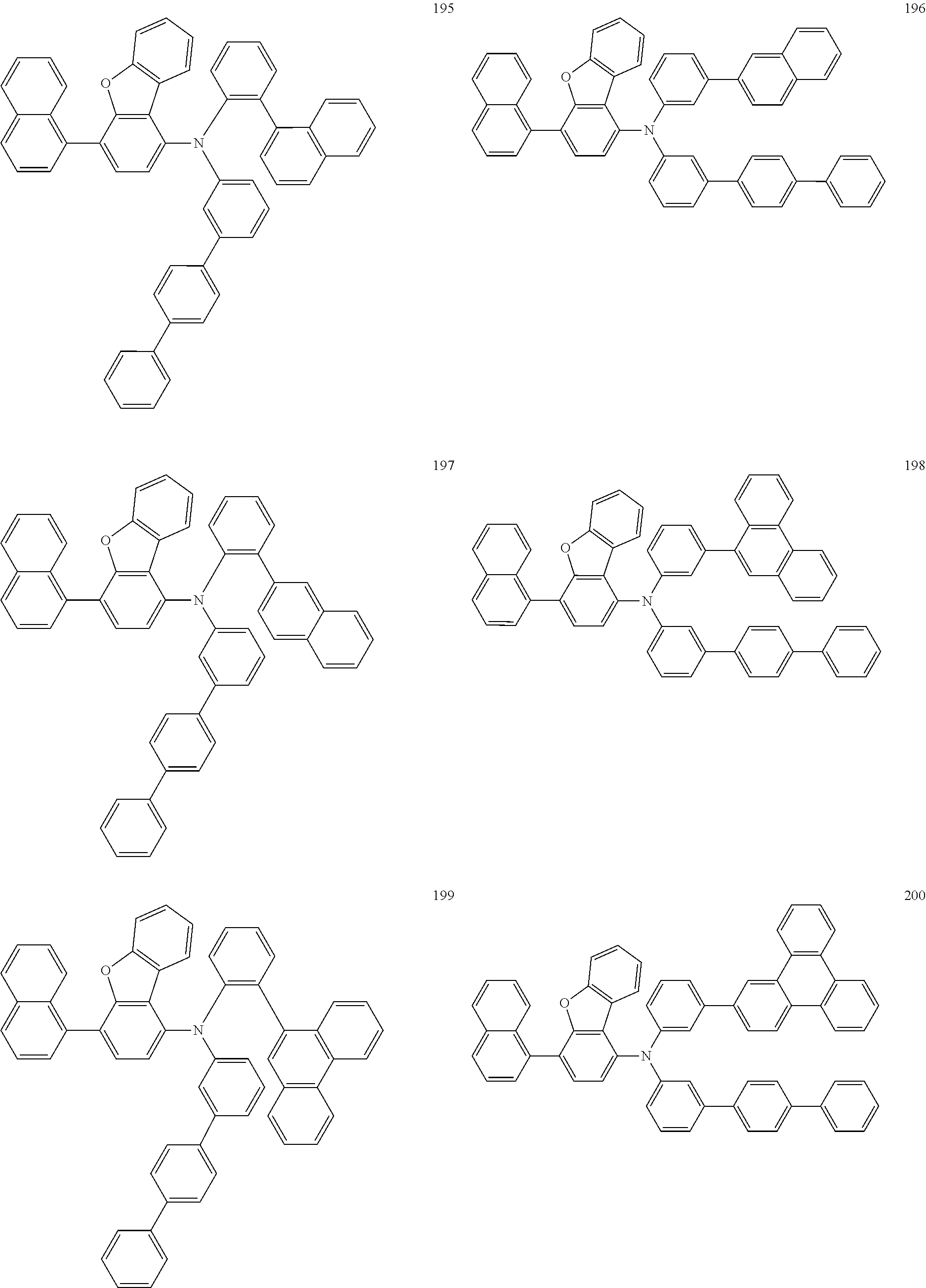

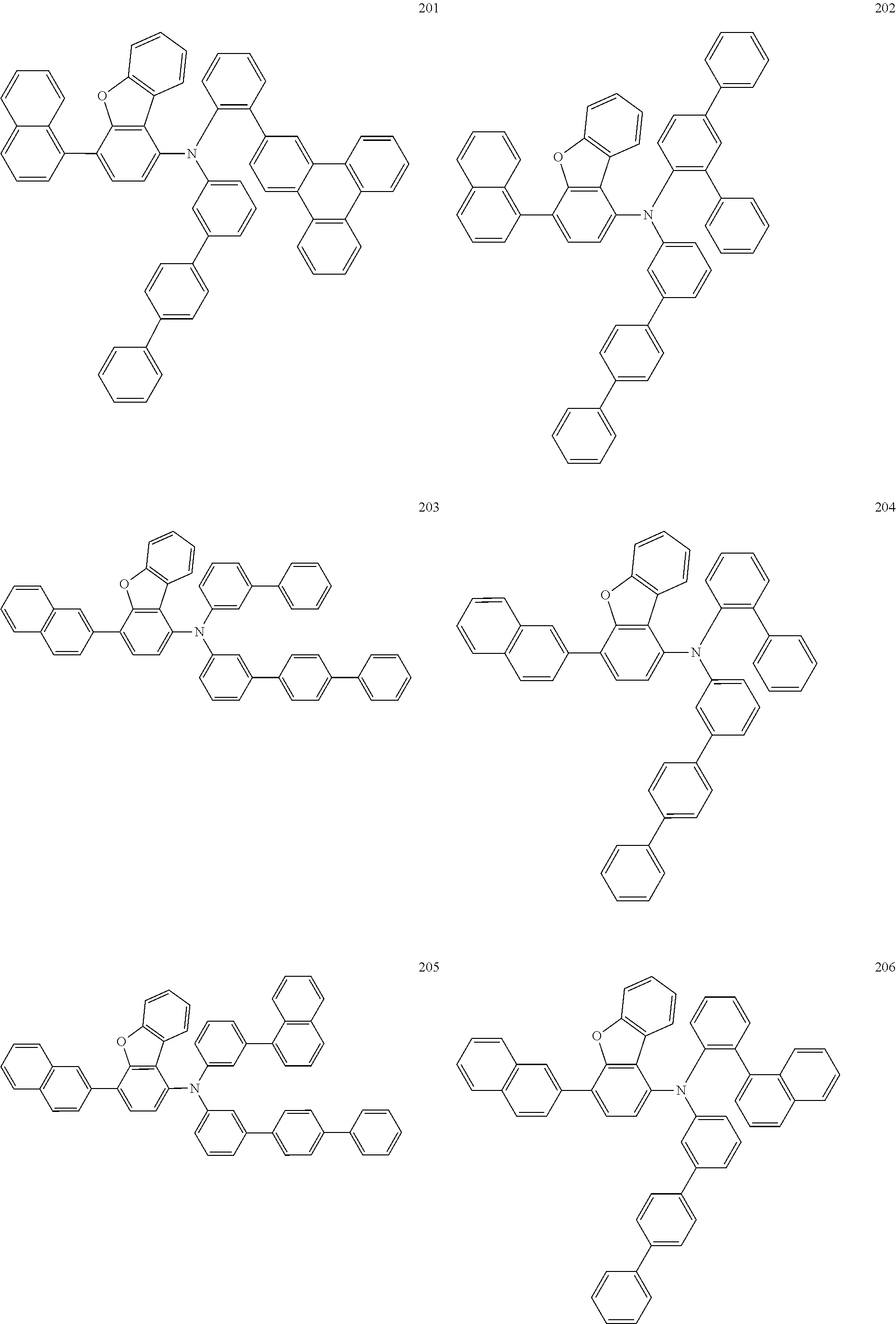

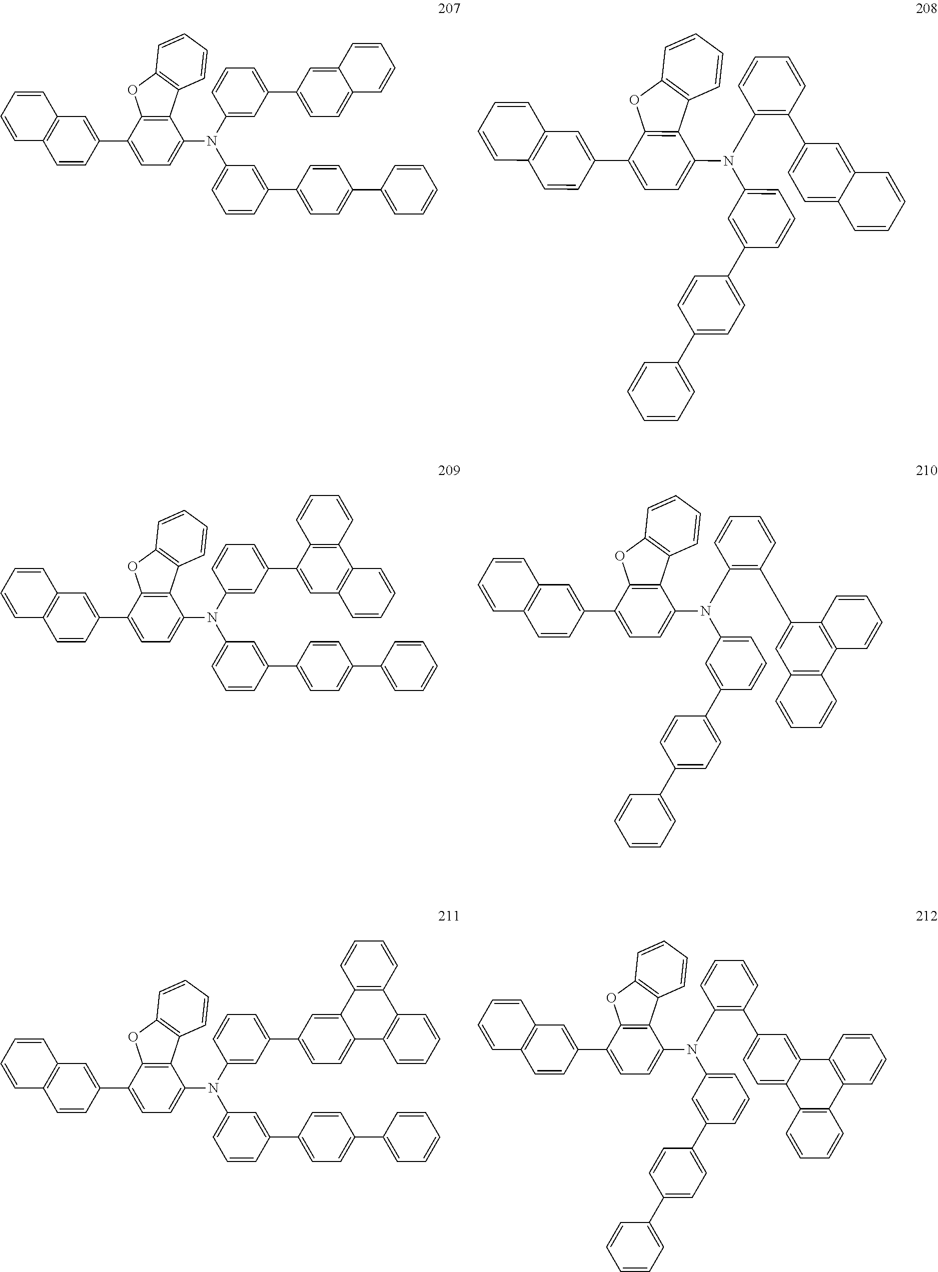

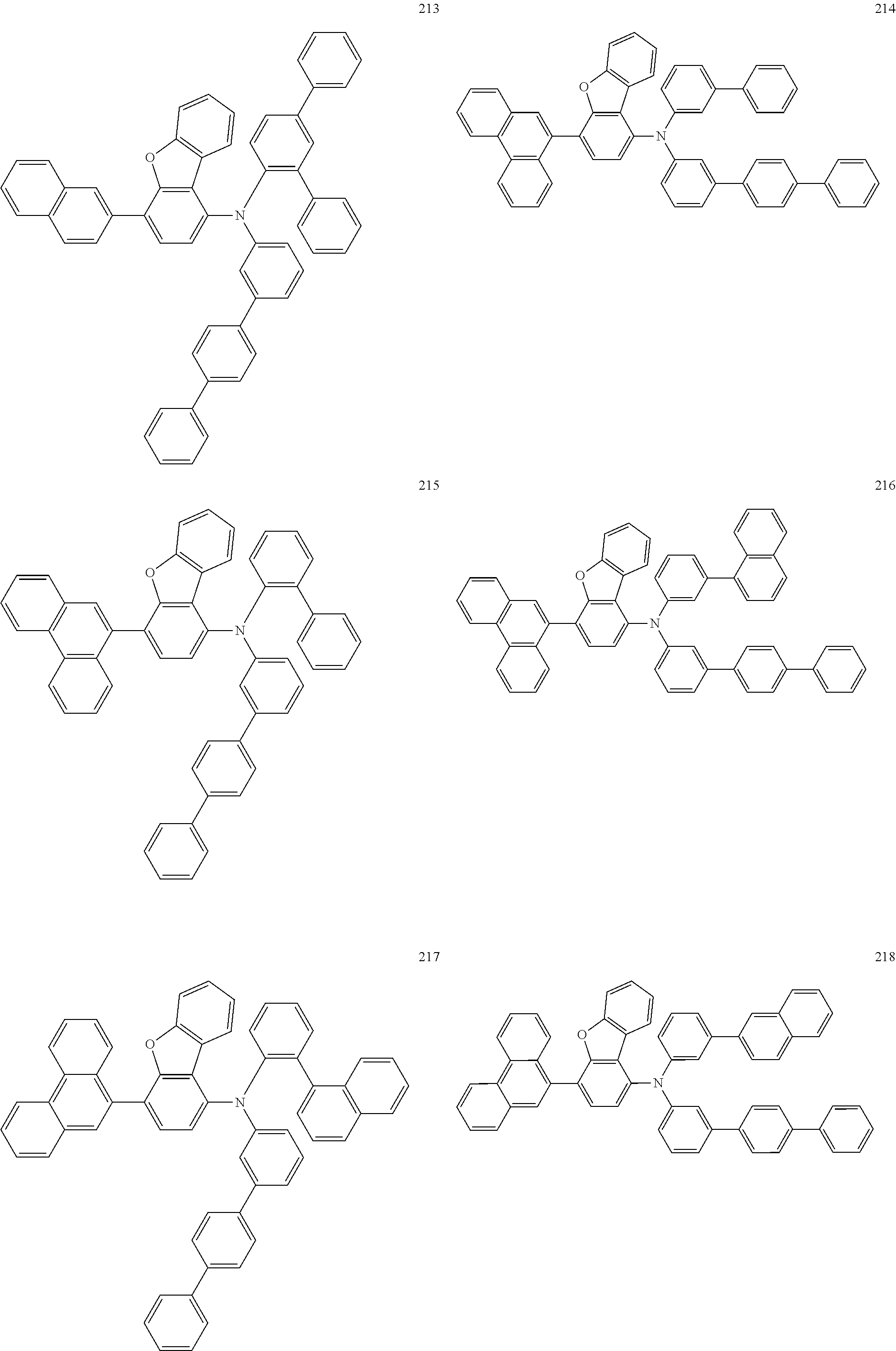

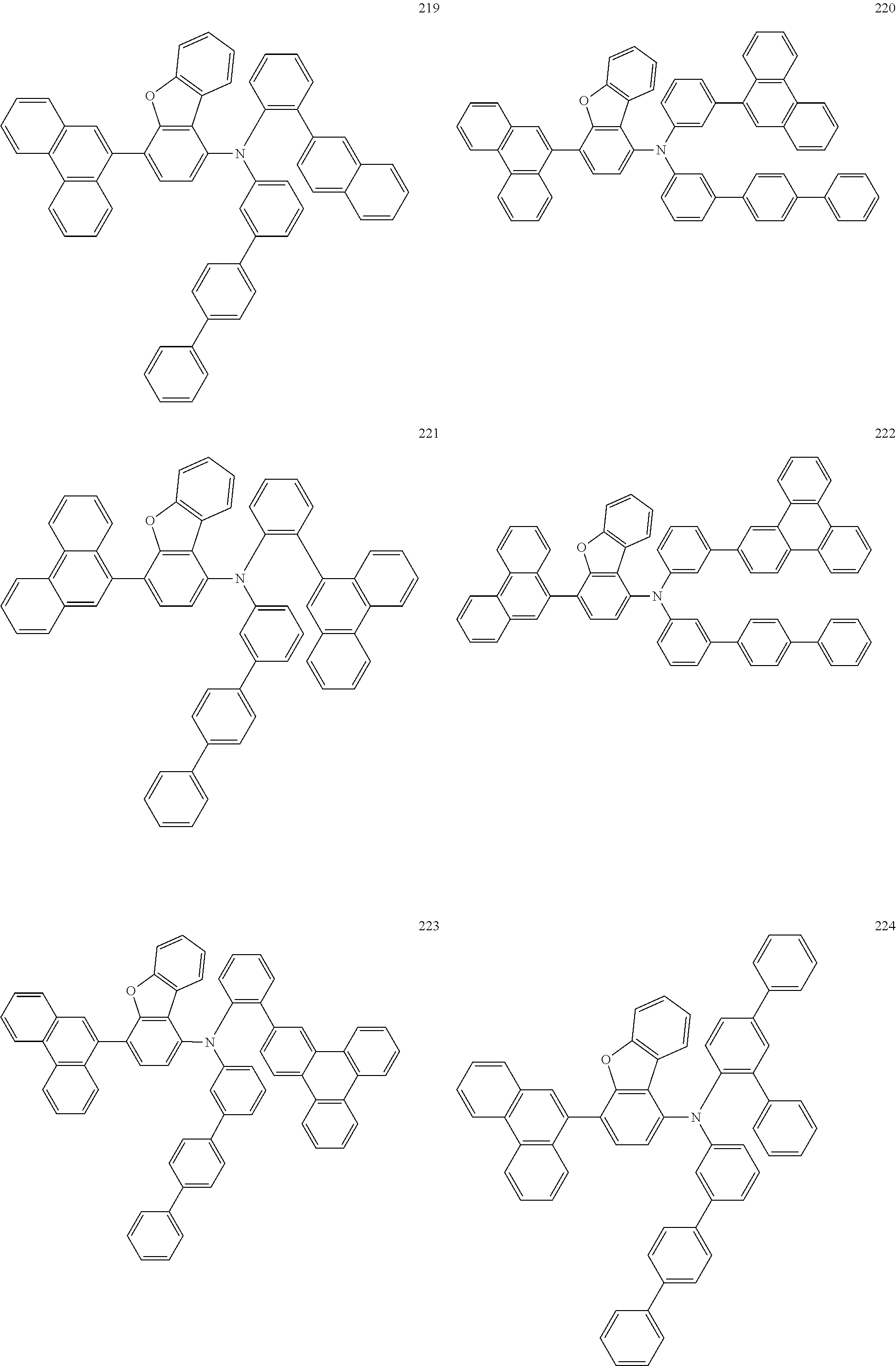

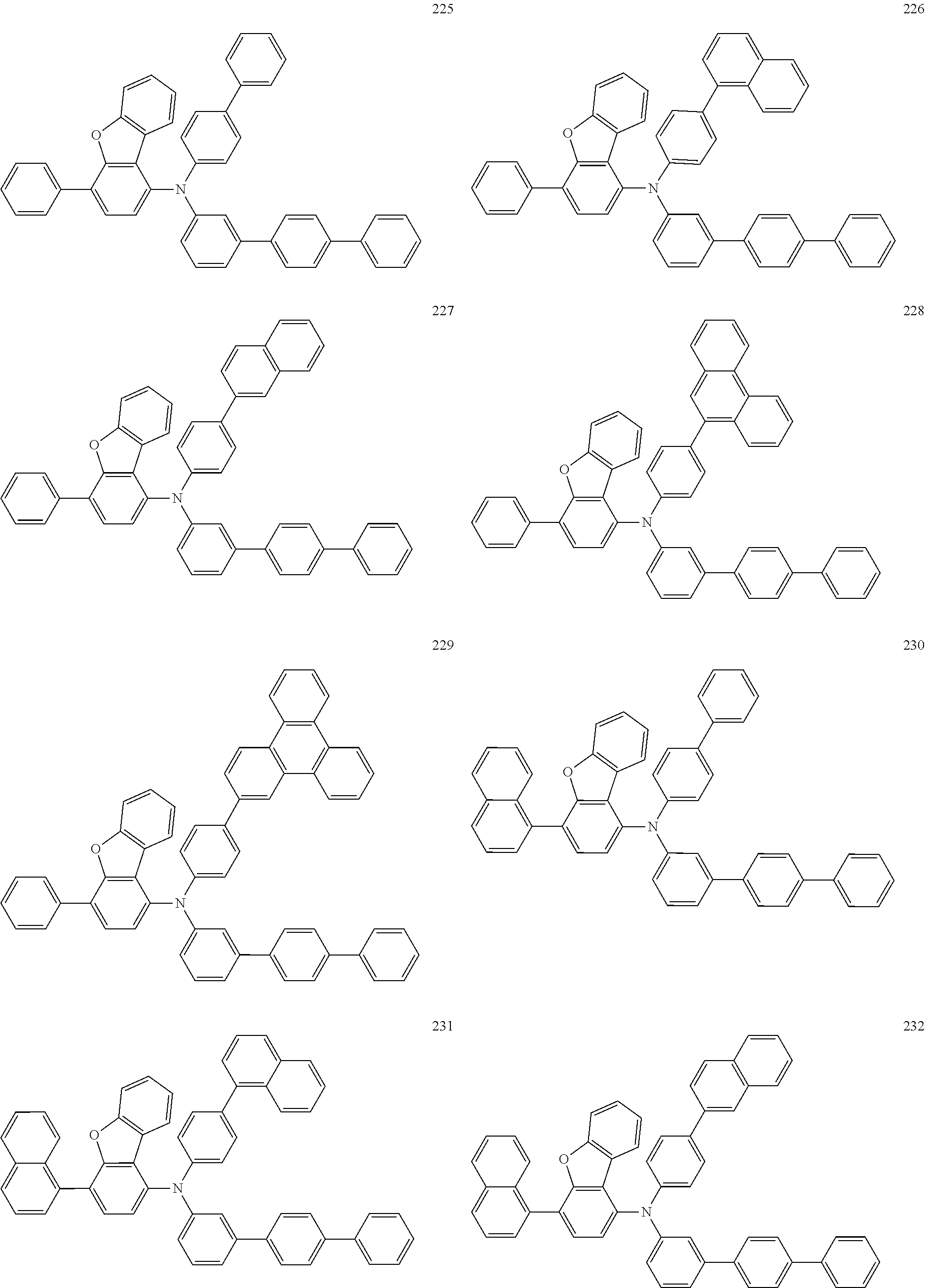

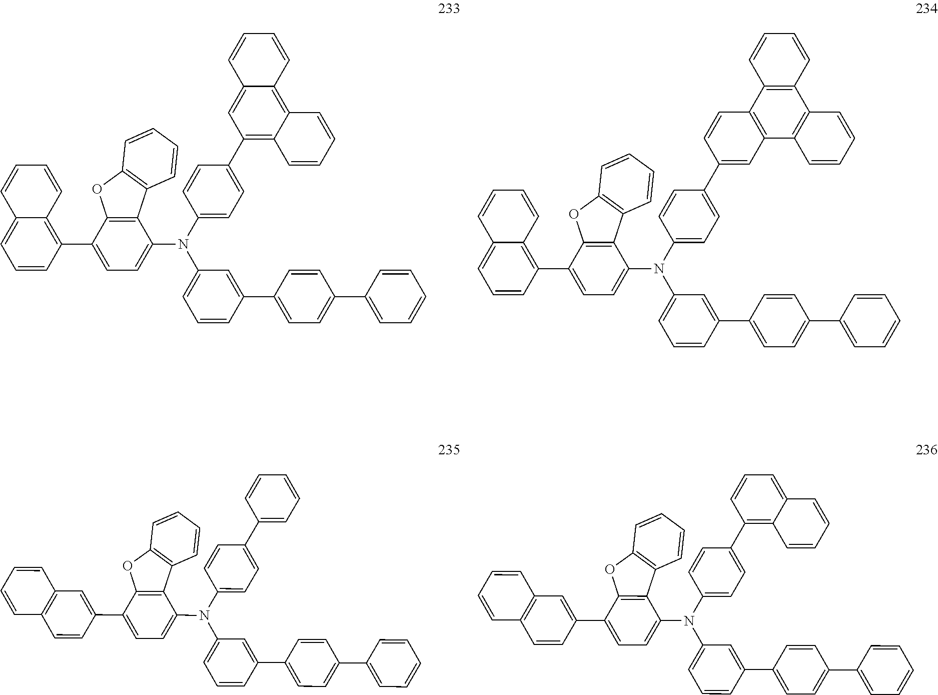 [SMILES: c1ccc(-c2ccc(-c3cccc(N(c4ccc(-c5cc6ccccc6c6ccccc56)cc4)c4ccc(-c5cccc6ccccc56)c5oc6ccccc6c45)c3)cc2)cc1 ; c1ccc(-c2ccc(-c3cccc(N(c4ccc(-c5ccc6c7ccccc7c7ccccc7c6c5)cc4)c4ccc(-c5cccc6ccccc56)c5oc6ccccc6c45)c3)cc2)cc1 ; c1ccc(-c2ccc(-c3cccc(N(c4ccc(-c5ccccc5)cc4)c4ccc(-c5ccc6ccccc6c5)c5oc6ccccc6c45)c3)cc2)cc1 ; c1ccc(-c2ccc(-c3cccc(N(c4ccc(-c5cccc6ccccc56)cc4)c4ccc(-c5ccc6ccccc6c5)c5oc6ccccc6c45)c3)cc2)cc1]

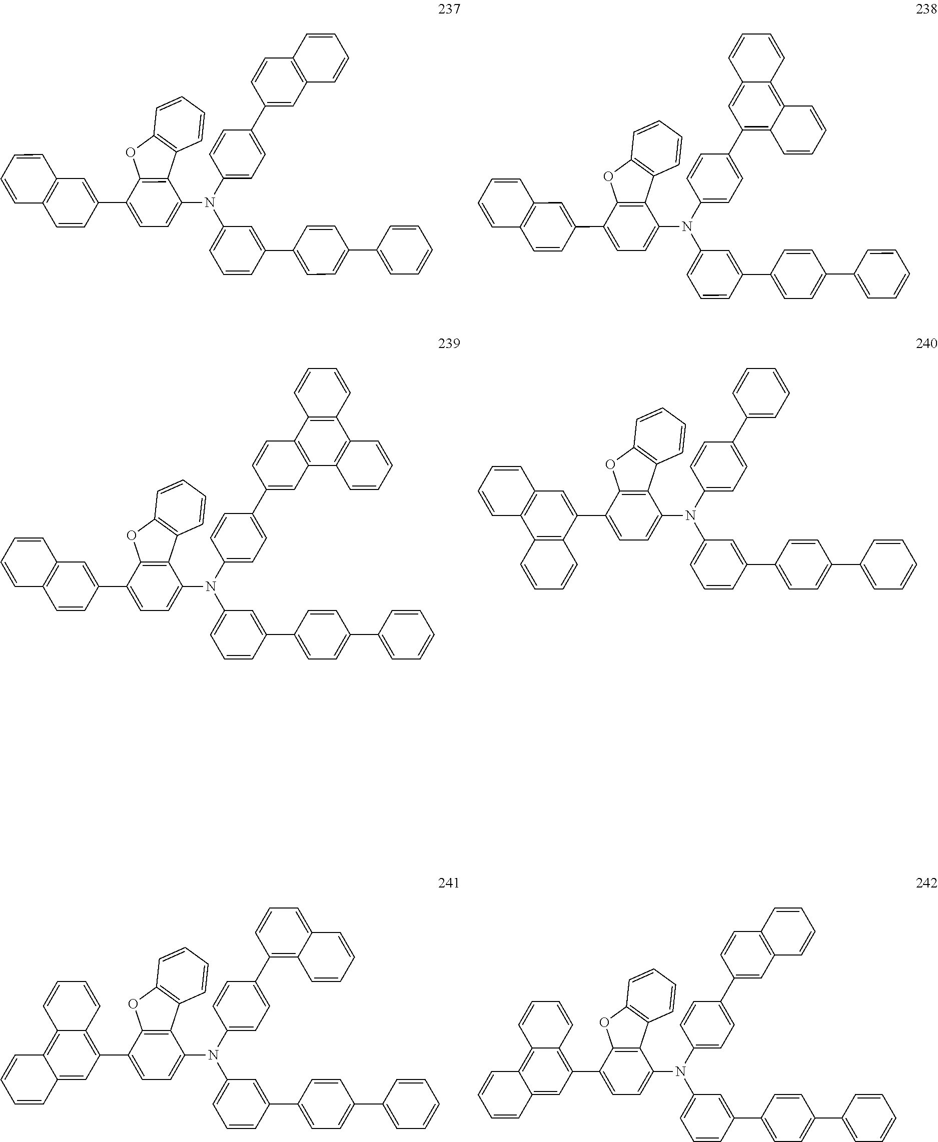

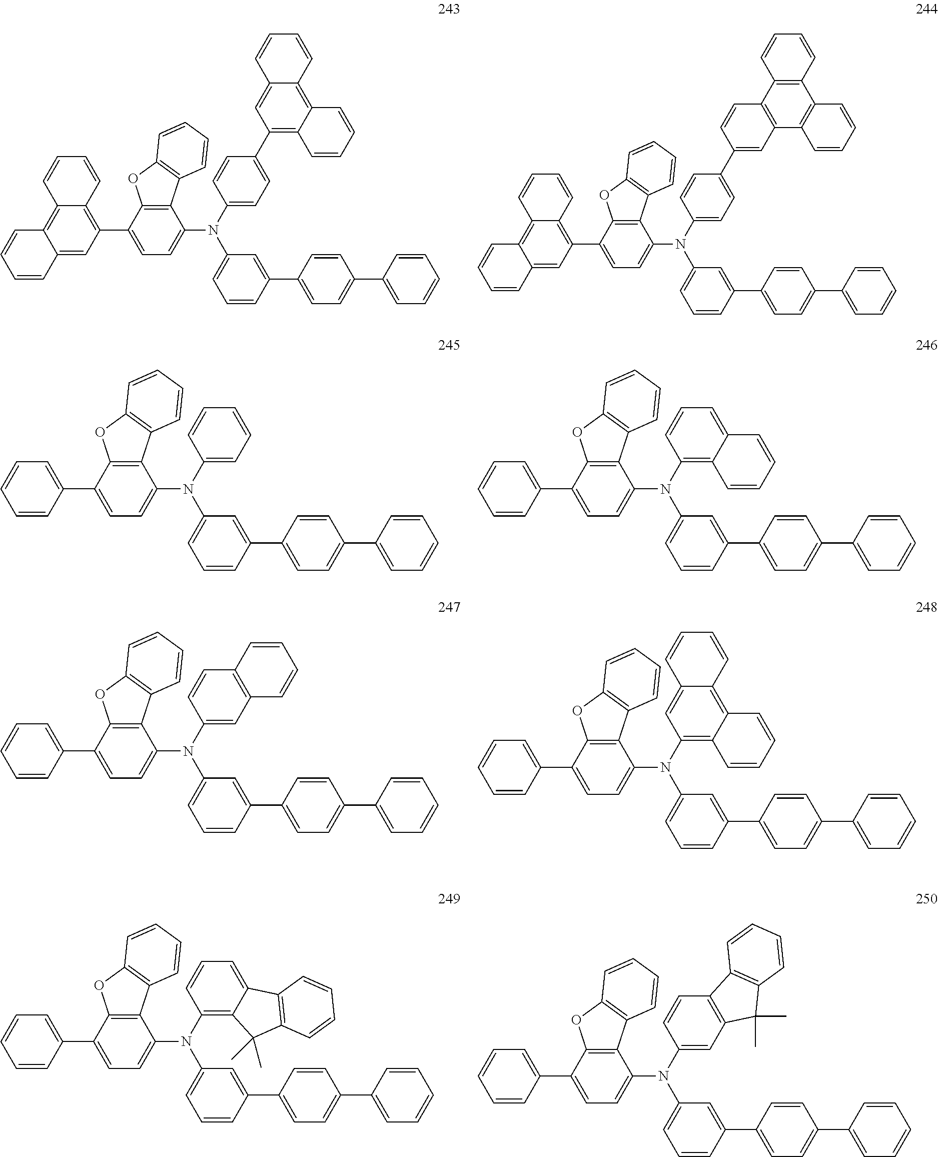

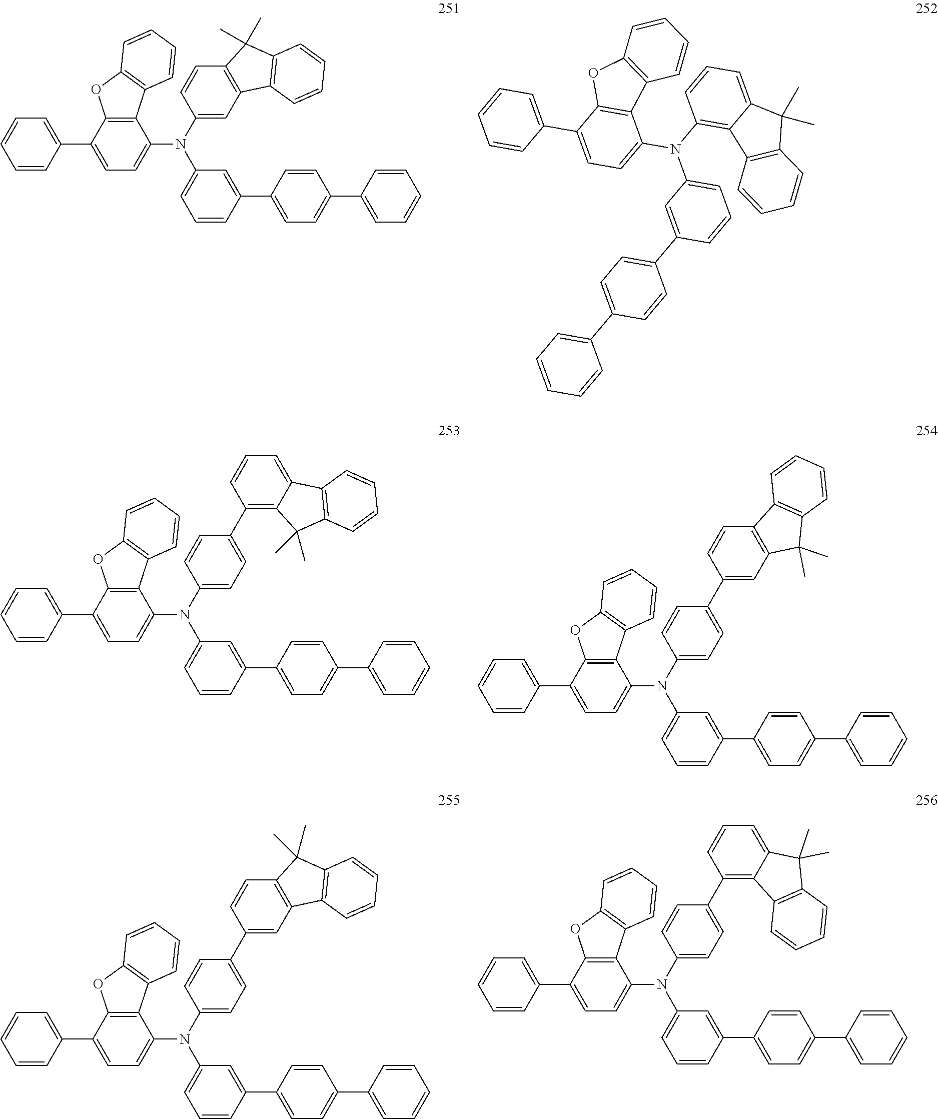

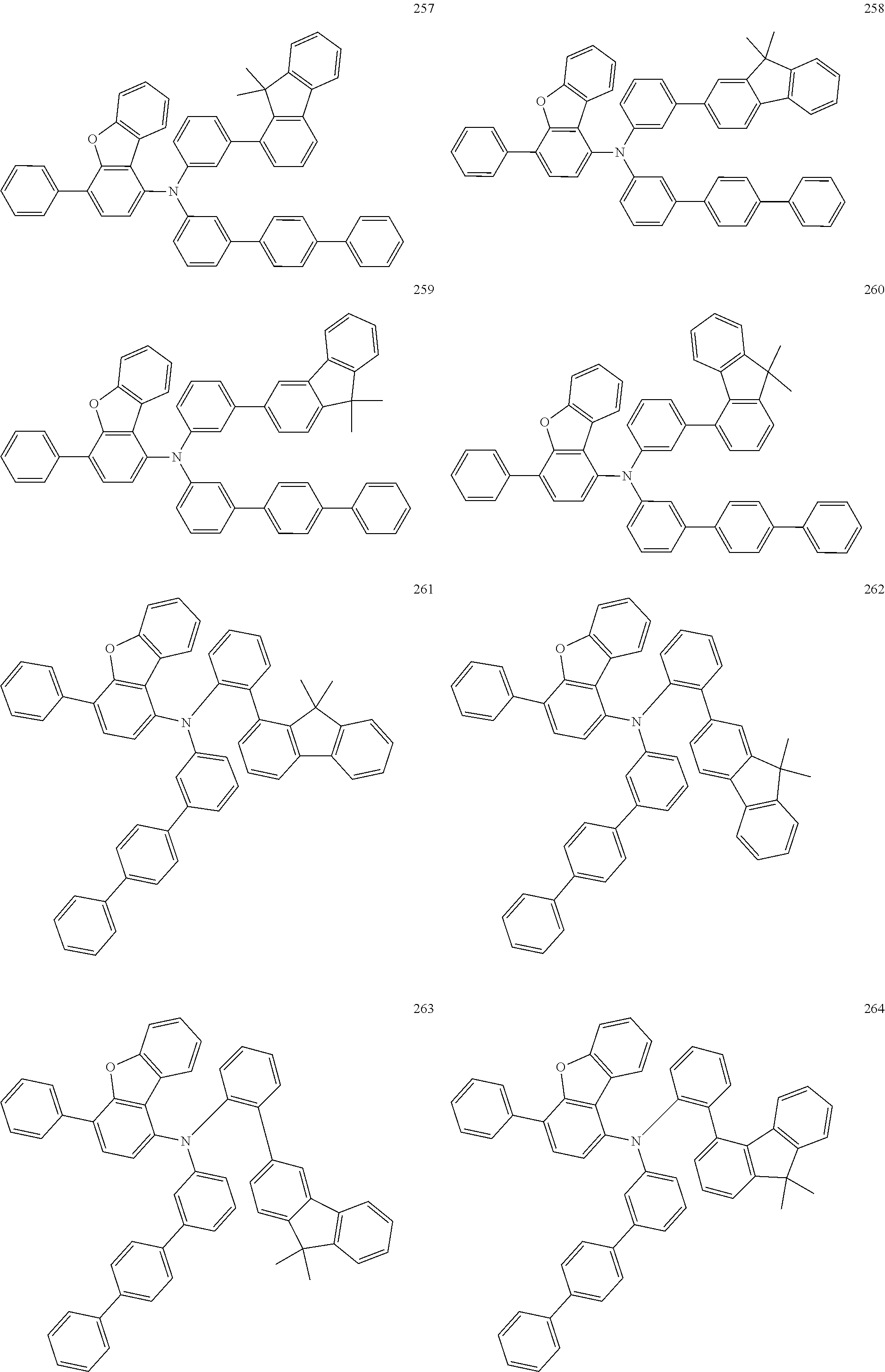

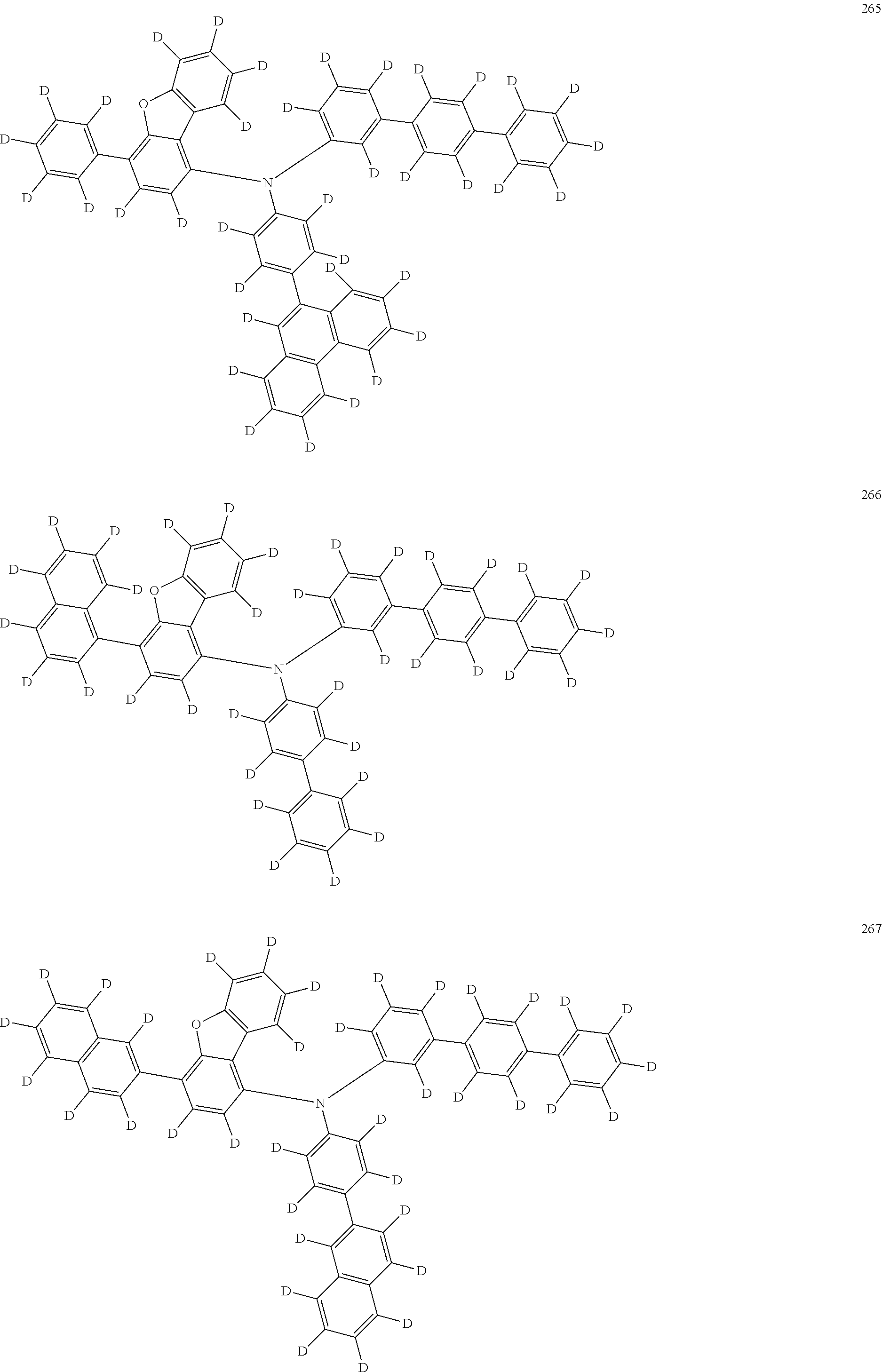

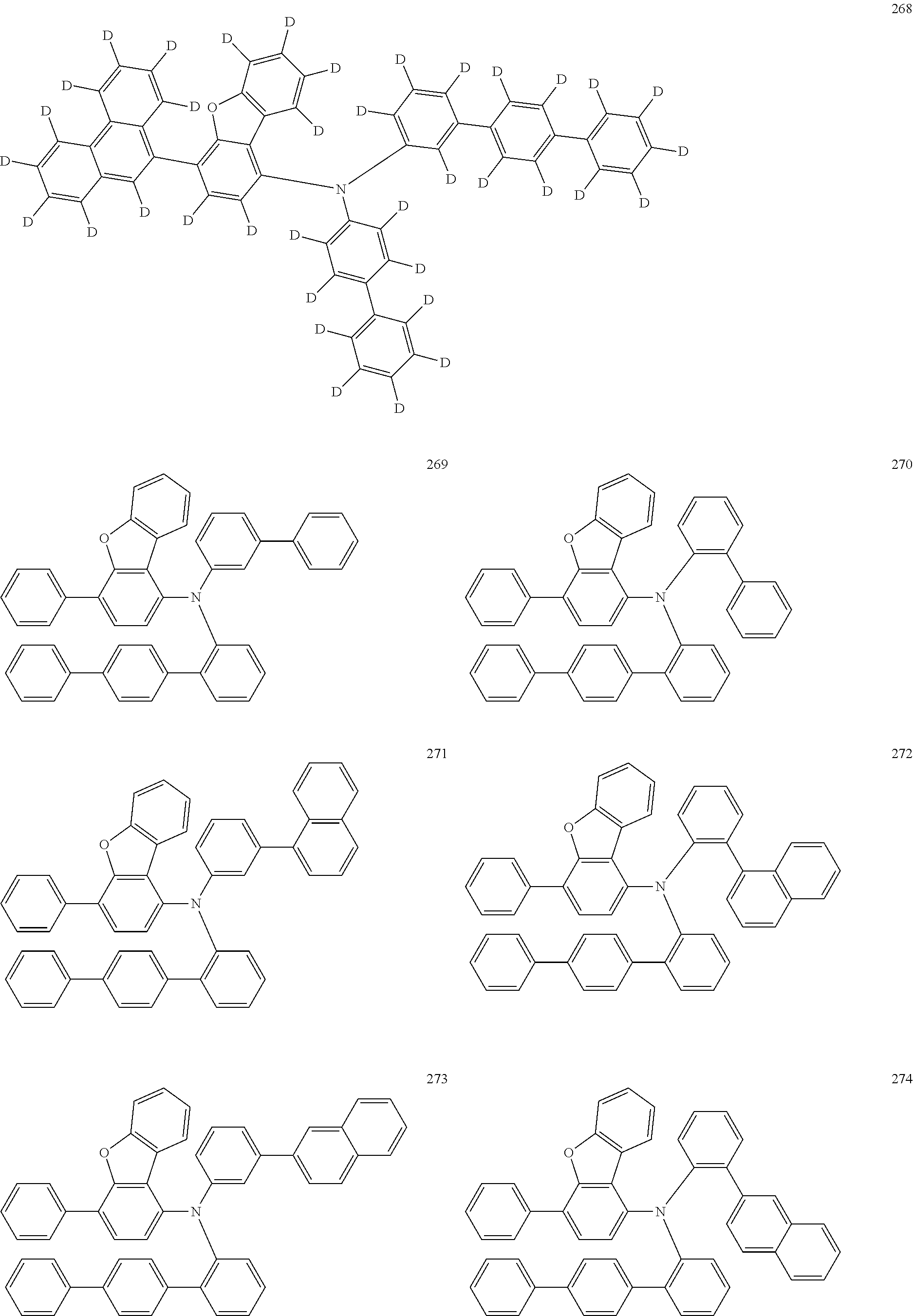

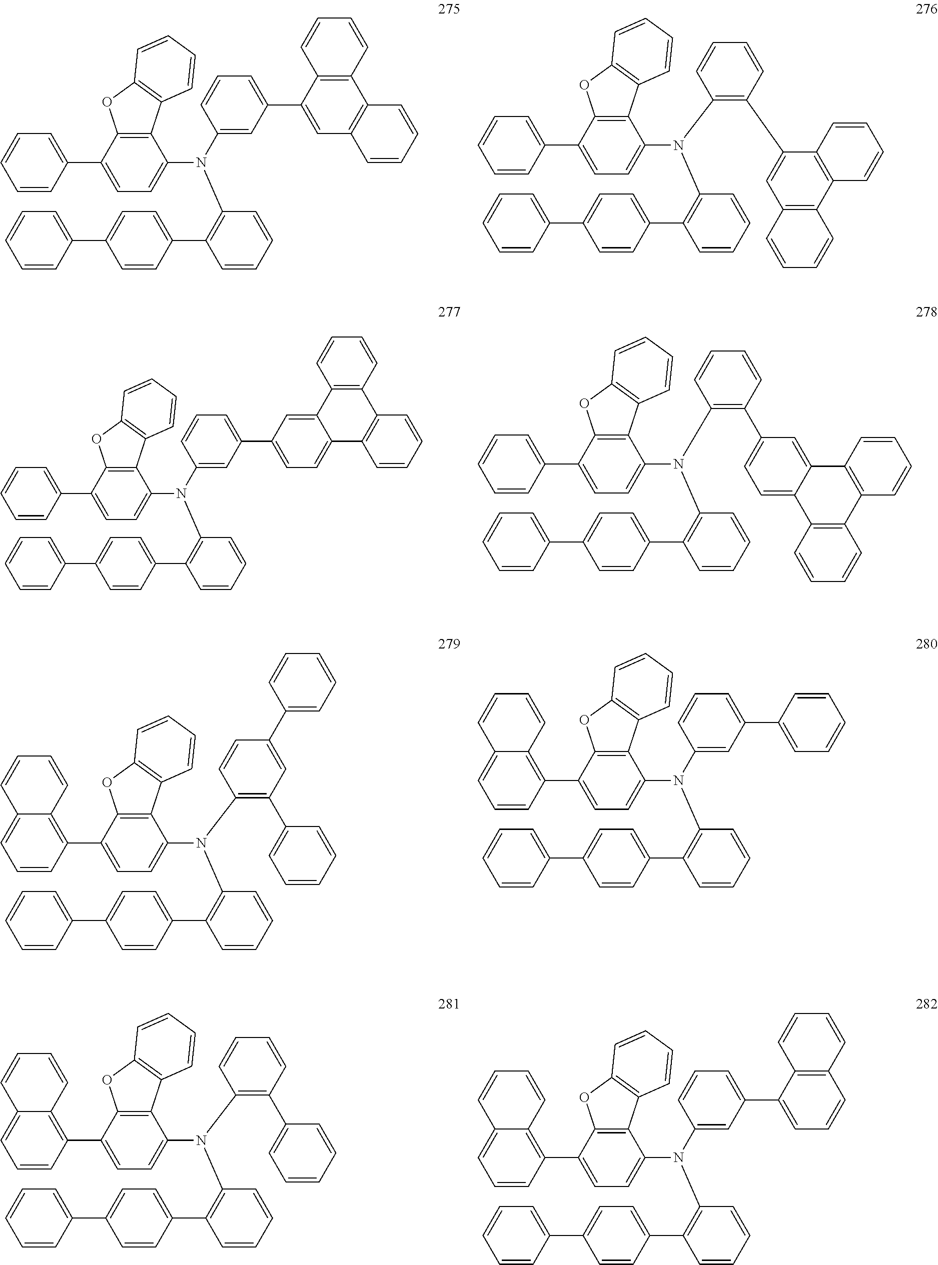

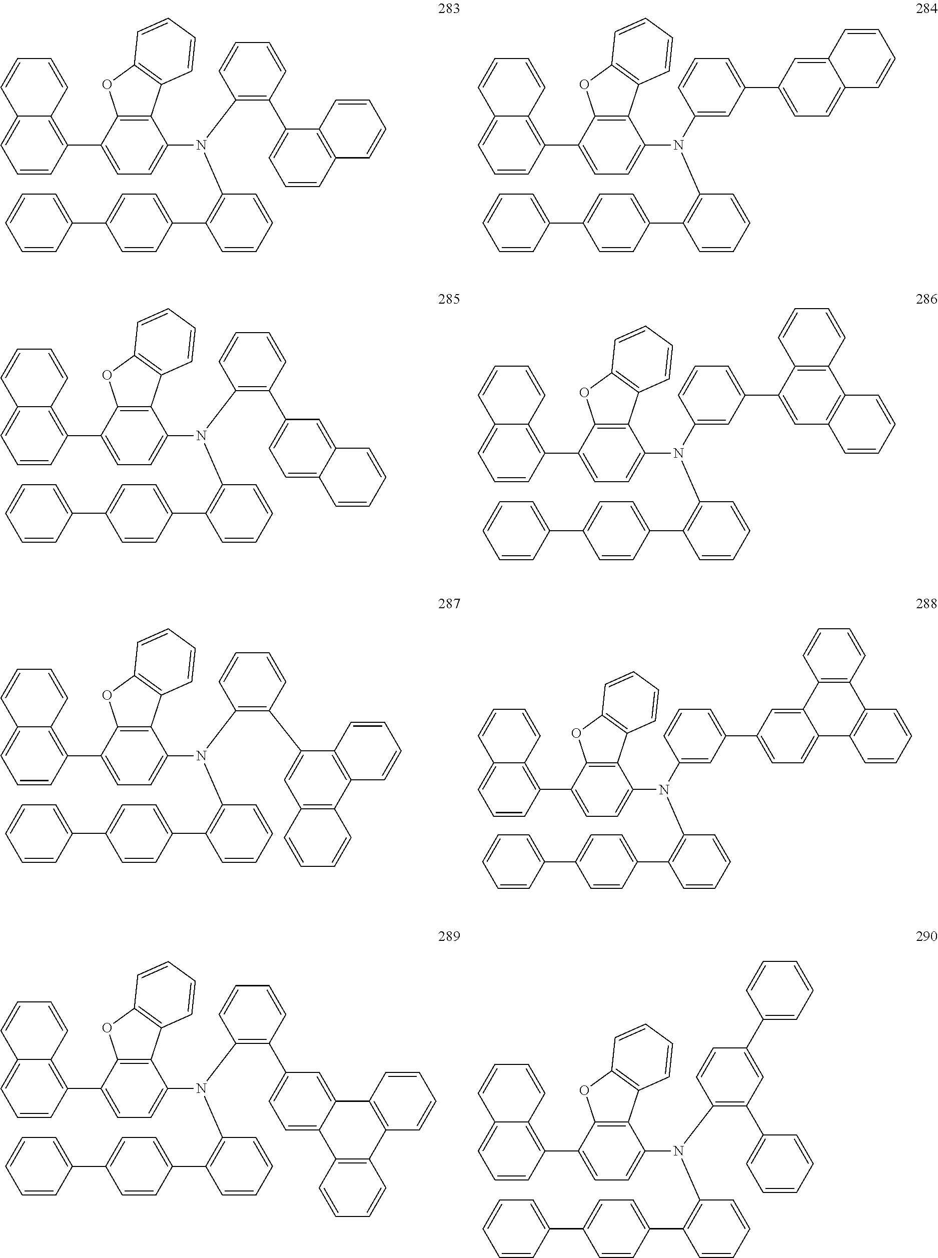

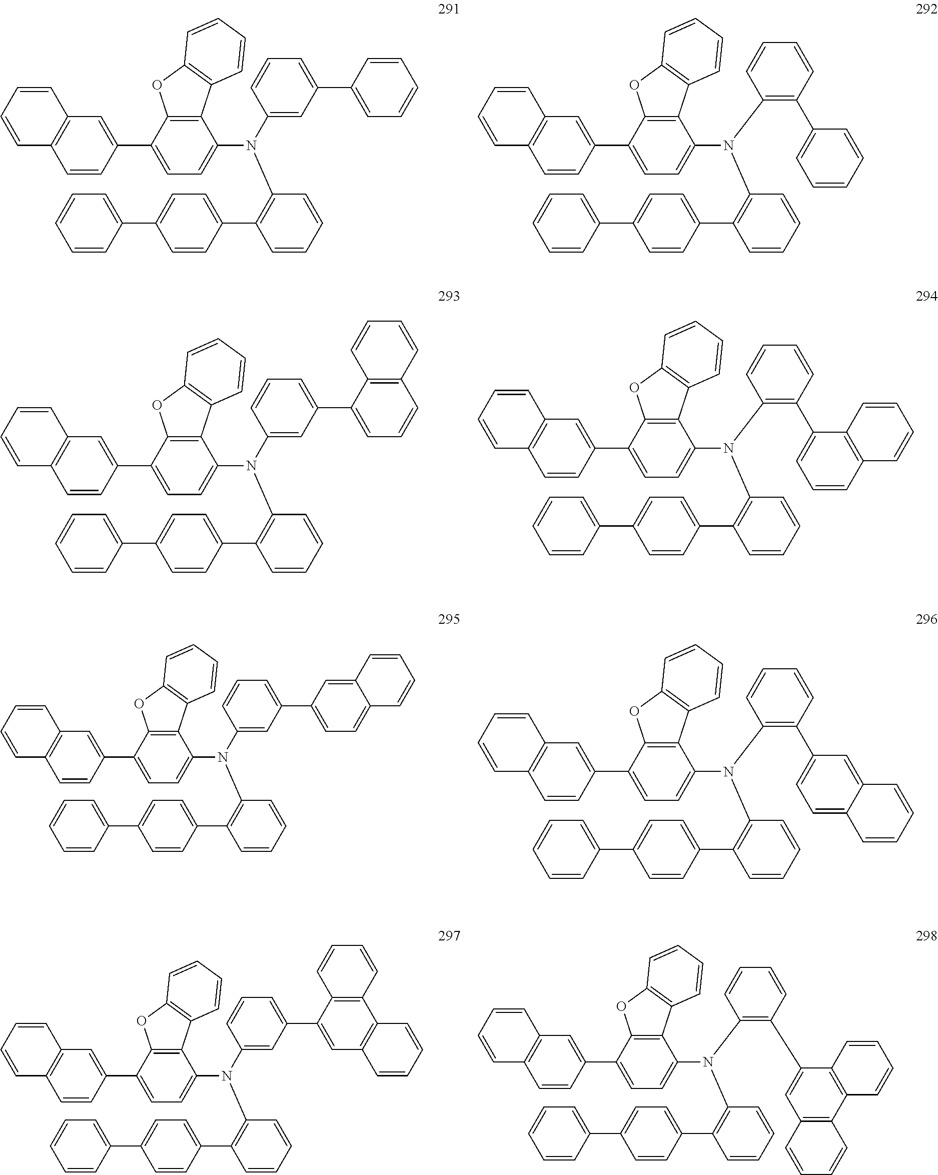

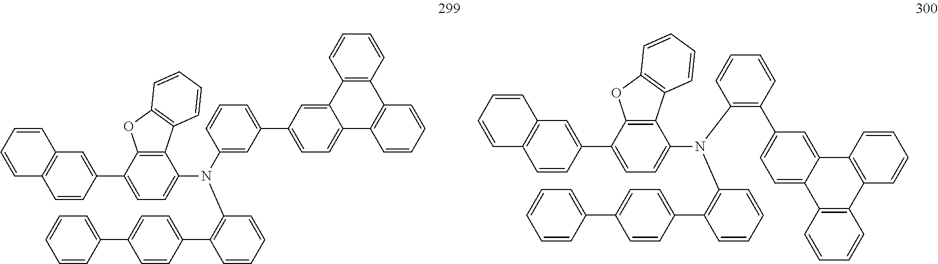 [SMILES: c1ccc(-c2ccc(-c3ccccc3N(c3cccc(-c4ccc5c6ccccc6c6ccccc6c5c4)c3)c3ccc(-c4ccc5ccccc5c4)c4oc5ccccc5c34)cc2)cc1 ; c1ccc(-c2ccc(-c3ccccc3N(c3ccccc3-c3ccc4c5ccccc5c5ccccc5c4c3)c3ccc(-c4ccc5ccccc5c4)c4oc5ccccc5c34)cc2)cc1]

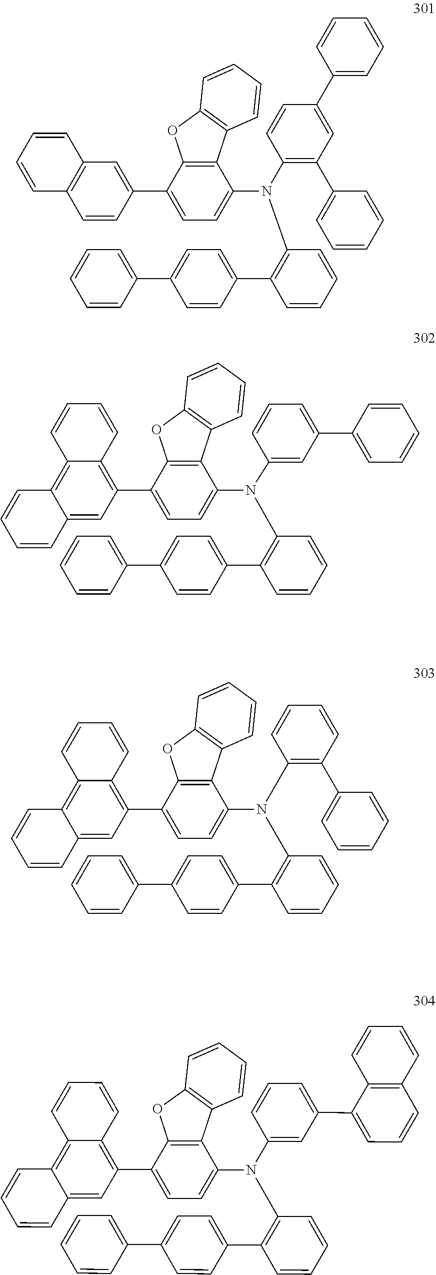 [SMILES: c1ccc(-c2ccc(-c3ccccc3N(c3ccc(-c4ccccc4)cc3-c3ccccc3)c3ccc(-c4ccc5ccccc5c4)c4oc5ccccc5c34)cc2)cc1 ; c1ccc(-c2ccc(-c3ccccc3N(c3cccc(-c4ccccc4)c3)c3ccc(-c4cc5ccccc5c5ccccc45)c4oc5ccccc5c34)cc2)cc1 ; c1ccc(-c2ccc(-c3ccccc3N(c3ccccc3-c3ccccc3)c3ccc(-c4cc5ccccc5c5ccccc45)c4oc5ccccc5c34)cc2)cc1 ; c1ccc(-c2ccc(-c3ccccc3N(c3cccc(-c4cccc5ccccc45)c3)c3ccc(-c4cc5ccccc5c5ccccc45)c4oc5ccccc5c34)cc2)cc1]

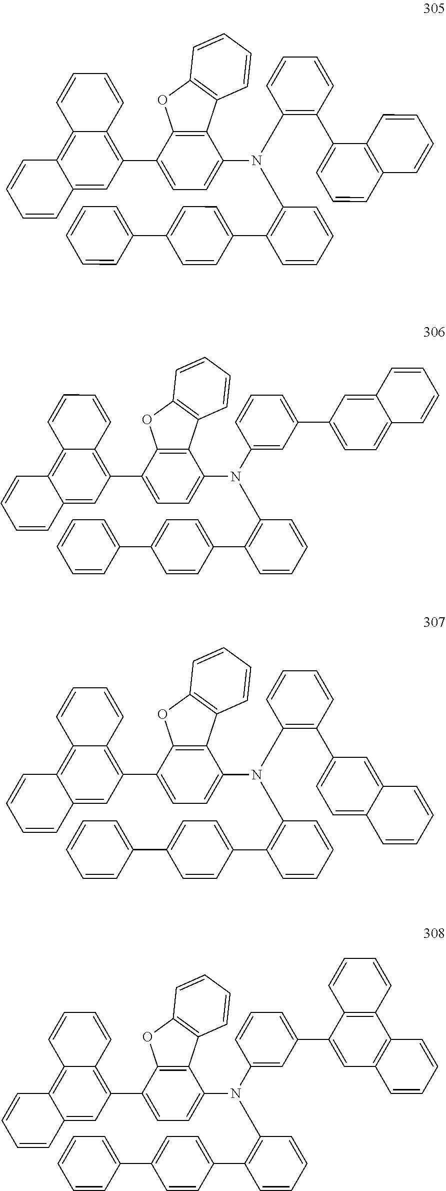 [SMILES: c1ccc(-c2ccc(-c3ccccc3N(c3ccccc3-c3cccc4ccccc34)c3ccc(-c4cc5ccccc5c5ccccc45)c4oc5ccccc5c34)cc2)cc1 ; c1ccc(-c2ccc(-c3ccccc3N(c3cccc(-c4ccc5ccccc5c4)c3)c3ccc(-c4cc5ccccc5c5ccccc45)c4oc5ccccc5c34)cc2)cc1 ; c1ccc(-c2ccc(-c3ccccc3N(c3ccccc3-c3ccc4ccccc4c3)c3ccc(-c4cc5ccccc5c5ccccc45)c4oc5ccccc5c34)cc2)cc1 ; c1ccc(-c2ccc(-c3ccccc3N(c3cccc(-c4cc5ccccc5c5ccccc45)c3)c3ccc(-c4cc5ccccc5c5ccccc45)c4oc5ccccc5c34)cc2)cc1]

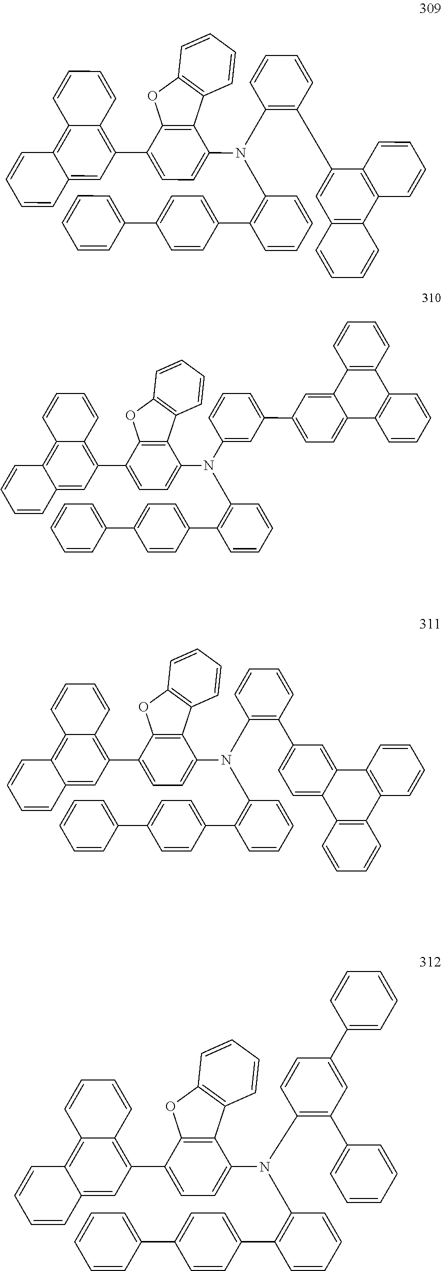 [SMILES: c1ccc(-c2ccc(-c3ccccc3N(c3ccccc3-c3cc4ccccc4c4ccccc34)c3ccc(-c4cc5ccccc5c5ccccc45)c4oc5ccccc5c34)cc2)cc1 ; c1ccc(-c2ccc(-c3ccccc3N(c3cccc(-c4ccc5c6ccccc6c6ccccc6c5c4)c3)c3ccc(-c4cc5ccccc5c5ccccc45)c4oc5ccccc5c34)cc2)cc1 ; c1ccc(-c2ccc(-c3ccccc3N(c3ccccc3-c3ccc4c5ccccc5c5ccccc5c4c3)c3ccc(-c4cc5ccccc5c5ccccc45)c4oc5ccccc5c34)cc2)cc1 ; c1ccc(-c2ccc(-c3ccccc3N(c3ccc(-c4ccccc4)cc3-c3ccccc3)c3ccc(-c4cc5ccccc5c5ccccc45)c4oc5ccccc5c34)cc2)cc1]

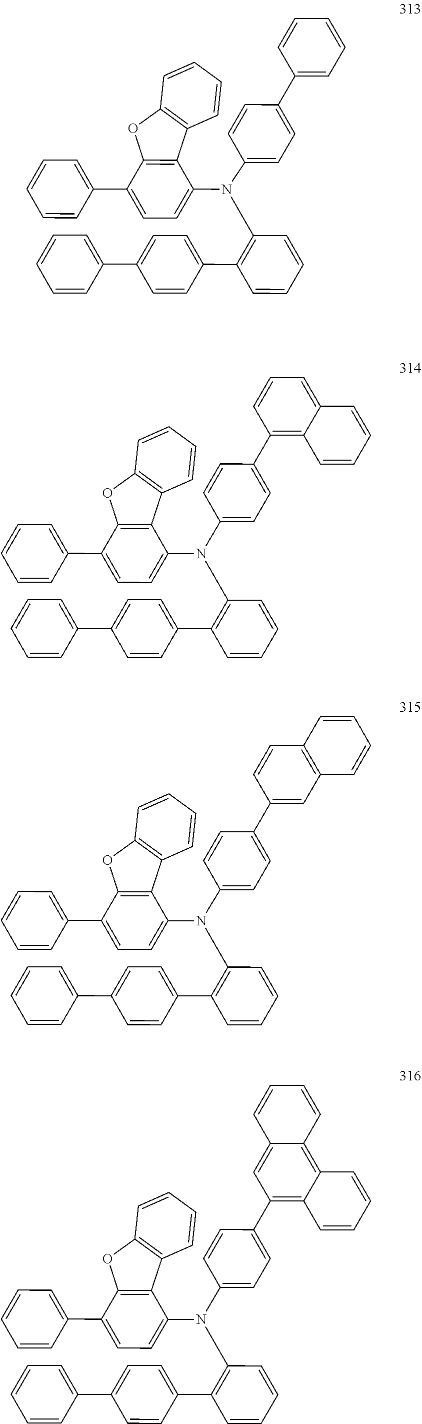 [SMILES: c1ccc(-c2ccc(-c3ccccc3N(c3ccc(-c4ccccc4)cc3)c3ccc(-c4ccccc4)c4oc5ccccc5c34)cc2)cc1 ; c1ccc(-c2ccc(-c3ccccc3N(c3ccc(-c4cccc5ccccc45)cc3)c3ccc(-c4ccccc4)c4oc5ccccc5c34)cc2)cc1 ; c1ccc(-c2ccc(-c3ccccc3N(c3ccc(-c4ccc5ccccc5c4)cc3)c3ccc(-c4ccccc4)c4oc5ccccc5c34)cc2)cc1 ; c1ccc(-c2ccc(-c3ccccc3N(c3ccc(-c4cc5ccccc5c5ccccc45)cc3)c3ccc(-c4ccccc4)c4oc5ccccc5c34)cc2)cc1]

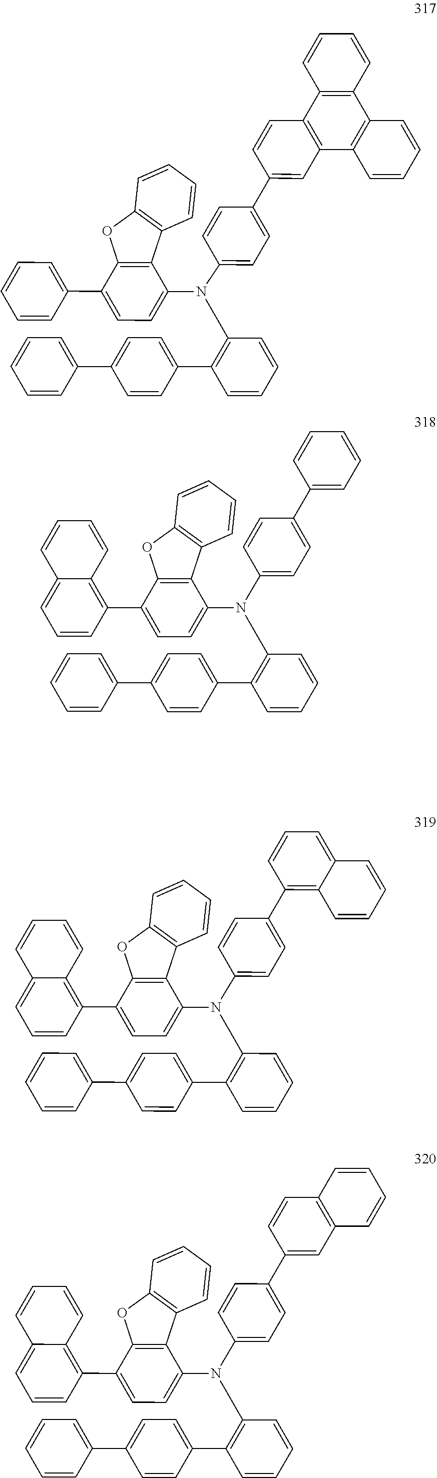 [SMILES: c1ccc(-c2ccc(-c3ccccc3N(c3ccc(-c4ccc5c6ccccc6c6ccccc6c5c4)cc3)c3ccc(-c4ccccc4)c4oc5ccccc5c34)cc2)cc1 ; c1ccc(-c2ccc(-c3ccccc3N(c3ccc(-c4ccccc4)cc3)c3ccc(-c4cccc5ccccc45)c4oc5ccccc5c34)cc2)cc1 ; c1ccc(-c2ccc(-c3ccccc3N(c3ccc(-c4cccc5ccccc45)cc3)c3ccc(-c4cccc5ccccc45)c4oc5ccccc5c34)cc2)cc1 ; c1ccc(-c2ccc(-c3ccccc3N(c3ccc(-c4ccc5ccccc5c4)cc3)c3ccc(-c4cccc5ccccc45)c4oc5ccccc5c34)cc2)cc1]

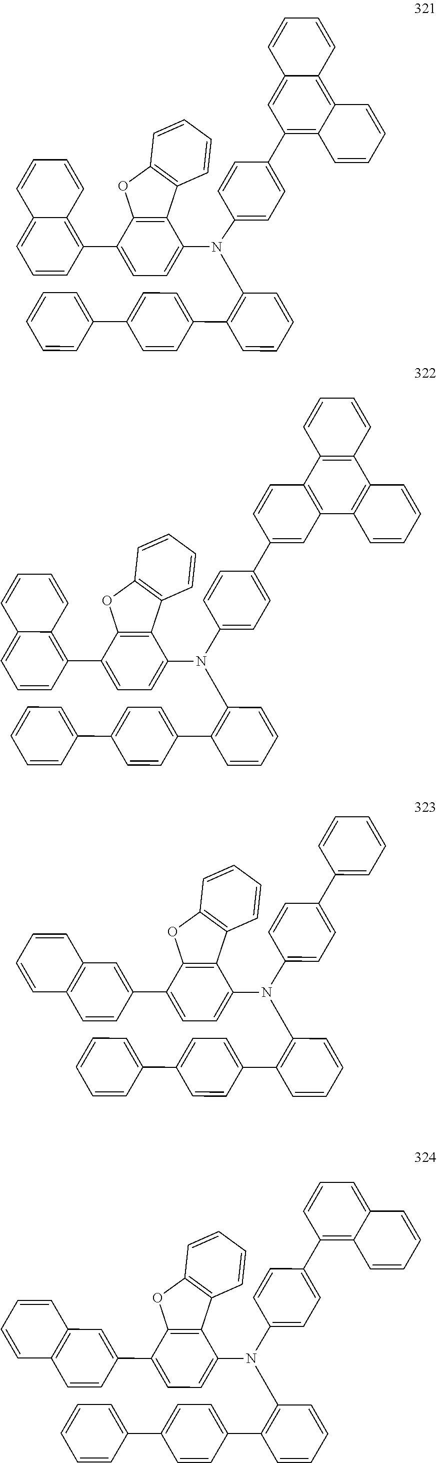 [SMILES: c1ccc(-c2ccc(-c3ccccc3N(c3ccc(-c4cc5ccccc5c5ccccc45)cc3)c3ccc(-c4cccc5ccccc45)c4oc5ccccc5c34)cc2)cc1 ; c1ccc(-c2ccc(-c3ccccc3N(c3ccc(-c4ccc5c6ccccc6c6ccccc6c5c4)cc3)c3ccc(-c4cccc5ccccc45)c4oc5ccccc5c34)cc2)cc1 ; c1ccc(-c2ccc(-c3ccccc3N(c3ccc(-c4ccccc4)cc3)c3ccc(-c4ccc5ccccc5c4)c4oc5ccccc5c34)cc2)cc1 ; c1ccc(-c2ccc(-c3ccccc3N(c3ccc(-c4cccc5ccccc45)cc3)c3ccc(-c4ccc5ccccc5c4)c4oc5ccccc5c34)cc2)cc1]

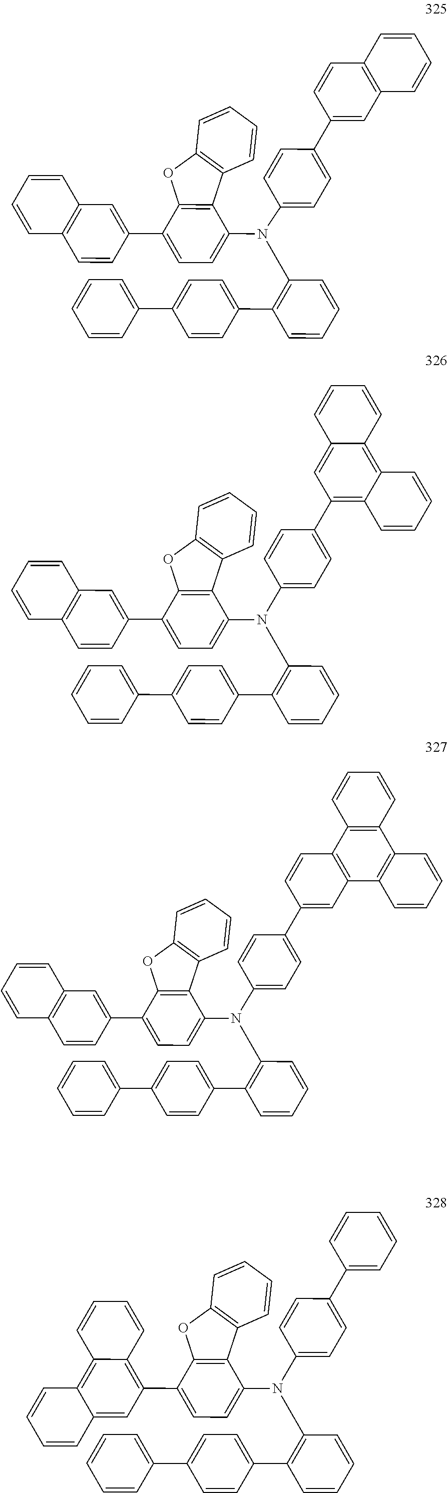 [SMILES: c1ccc(-c2ccc(-c3ccccc3N(c3ccc(-c4ccc5ccccc5c4)cc3)c3ccc(-c4ccc5ccccc5c4)c4oc5ccccc5c34)cc2)cc1 ; c1ccc(-c2ccc(-c3ccccc3N(c3ccc(-c4cc5ccccc5c5ccccc45)cc3)c3ccc(-c4ccc5ccccc5c4)c4oc5ccccc5c34)cc2)cc1 ; c1ccc(-c2ccc(-c3ccccc3N(c3ccc(-c4ccc5c6ccccc6c6ccccc6c5c4)cc3)c3ccc(-c4ccc5ccccc5c4)c4oc5ccccc5c34)cc2)cc1 ; c1ccc(-c2ccc(-c3ccccc3N(c3ccc(-c4ccccc4)cc3)c3ccc(-c4cc5ccccc5c5ccccc45)c4oc5ccccc5c34)cc2)cc1]

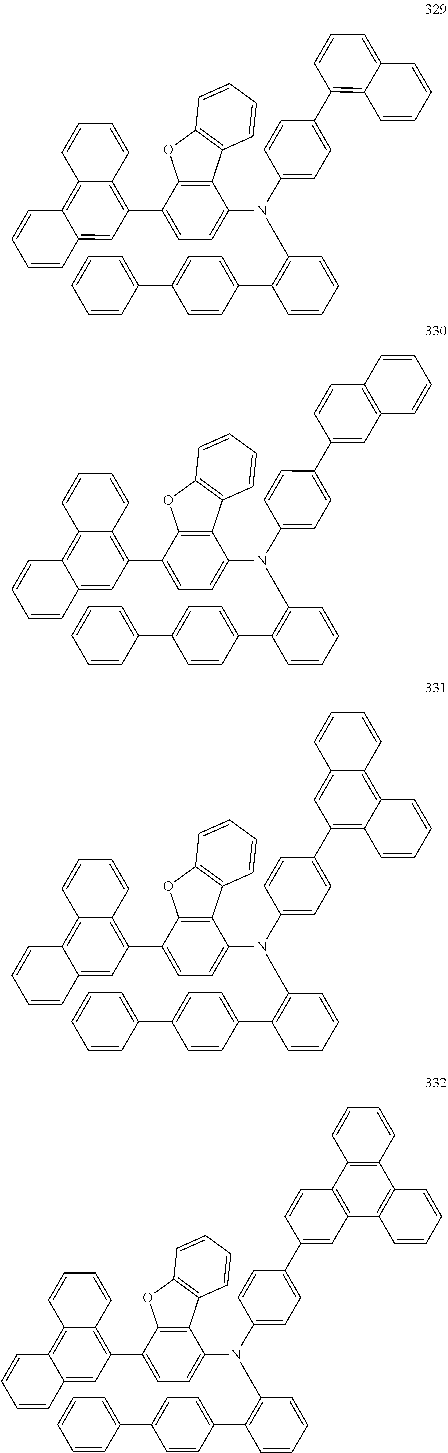 [SMILES: c1ccc(-c2ccc(-c3ccccc3N(c3ccc(-c4cccc5ccccc45)cc3)c3ccc(-c4cc5ccccc5c5ccccc45)c4oc5ccccc5c34)cc2)cc1 ; c1ccc(-c2ccc(-c3ccccc3N(c3ccc(-c4ccc5ccccc5c4)cc3)c3ccc(-c4cc5ccccc5c5ccccc45)c4oc5ccccc5c34)cc2)cc1 ; c1ccc(-c2ccc(-c3ccccc3N(c3ccc(-c4cc5ccccc5c5ccccc45)cc3)c3ccc(-c4cc5ccccc5c5ccccc45)c4oc5ccccc5c34)cc2)cc1 ; c1ccc(-c2ccc(-c3ccccc3N(c3ccc(-c4ccc5c6ccccc6c6ccccc6c5c4)cc3)c3ccc(-c4cc5ccccc5c5ccccc45)c4oc5ccccc5c34)cc2)cc1]

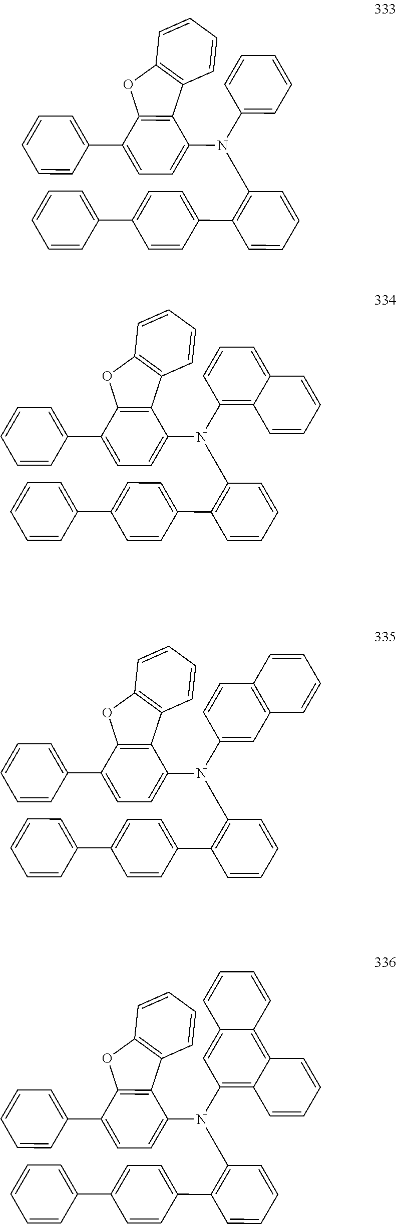 [SMILES: c1ccc(-c2ccc(-c3ccccc3N(c3ccccc3)c3ccc(-c4ccccc4)c4oc5ccccc5c34)cc2)cc1 ; c1ccc(-c2ccc(-c3ccccc3N(c3cccc4ccccc34)c3ccc(-c4ccccc4)c4oc5ccccc5c34)cc2)cc1 ; c1ccc(-c2ccc(-c3ccccc3N(c3ccc4ccccc4c3)c3ccc(-c4ccccc4)c4oc5ccccc5c34)cc2)cc1 ; c1ccc(-c2ccc(-c3ccccc3N(c3cc4ccccc4c4ccccc34)c3ccc(-c4ccccc4)c4oc5ccccc5c34)cc2)cc1]

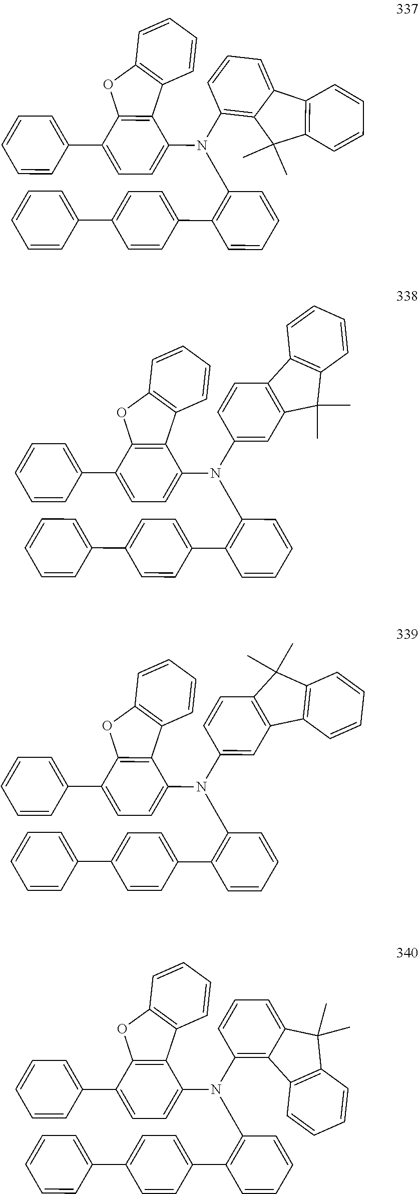 [SMILES: CC1(C)c2ccccc2-c2cccc(N(c3ccccc3-c3ccc(-c4ccccc4)cc3)c3ccc(-c4ccccc4)c4oc5ccccc5c34)c21 ; CC1(C)c2ccccc2-c2ccc(N(c3ccccc3-c3ccc(-c4ccccc4)cc3)c3ccc(-c4ccccc4)c4oc5ccccc5c34)cc21 ; CC1(C)c2ccccc2-c2cc(N(c3ccccc3-c3ccc(-c4ccccc4)cc3)c3ccc(-c4ccccc4)c4oc5ccccc5c34)ccc21 ; CC1(C)c2ccccc2-c2c(N(c3ccccc3-c3ccc(-c4ccccc4)cc3)c3ccc(-c4ccccc4)c4oc5ccccc5c34)cccc21]

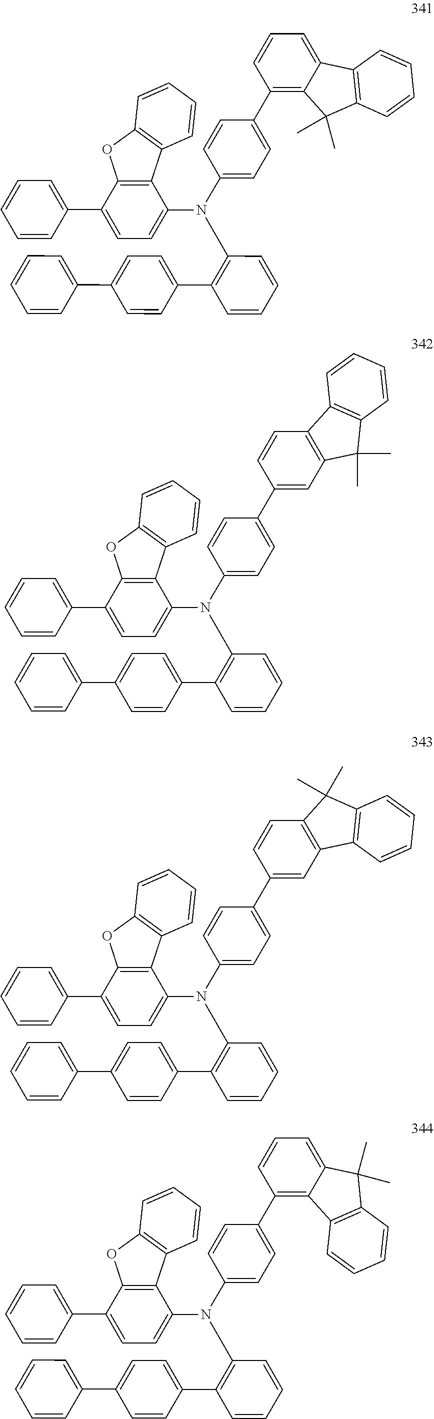

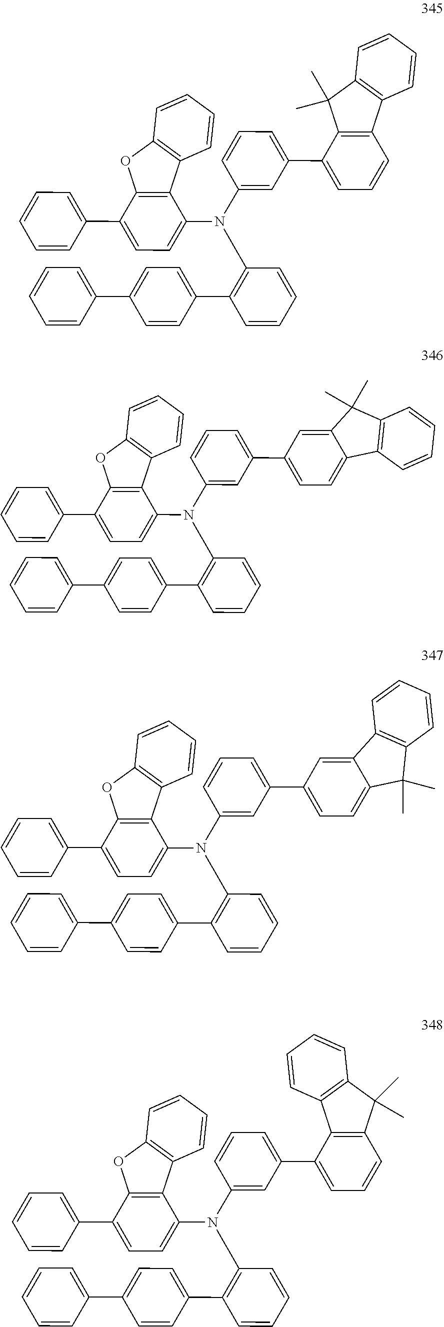 [SMILES: CC1(C)c2ccccc2-c2cccc(-c3cccc(N(c4ccccc4-c4ccc(-c5ccccc5)cc4)c4ccc(-c5ccccc5)c5oc6ccccc6c45)c3)c21 ; CC1(C)c2ccccc2-c2ccc(-c3cccc(N(c4ccccc4-c4ccc(-c5ccccc5)cc4)c4ccc(-c5ccccc5)c5oc6ccccc6c45)c3)cc21 ; CC1(C)c2ccccc2-c2cc(-c3cccc(N(c4ccccc4-c4ccc(-c5ccccc5)cc4)c4ccc(-c5ccccc5)c5oc6ccccc6c45)c3)ccc21 ; CC1(C)c2ccccc2-c2c(-c3cccc(N(c4ccccc4-c4ccc(-c5ccccc5)cc4)c4ccc(-c5ccccc5)c5oc6ccccc6c45)c3)cccc21]

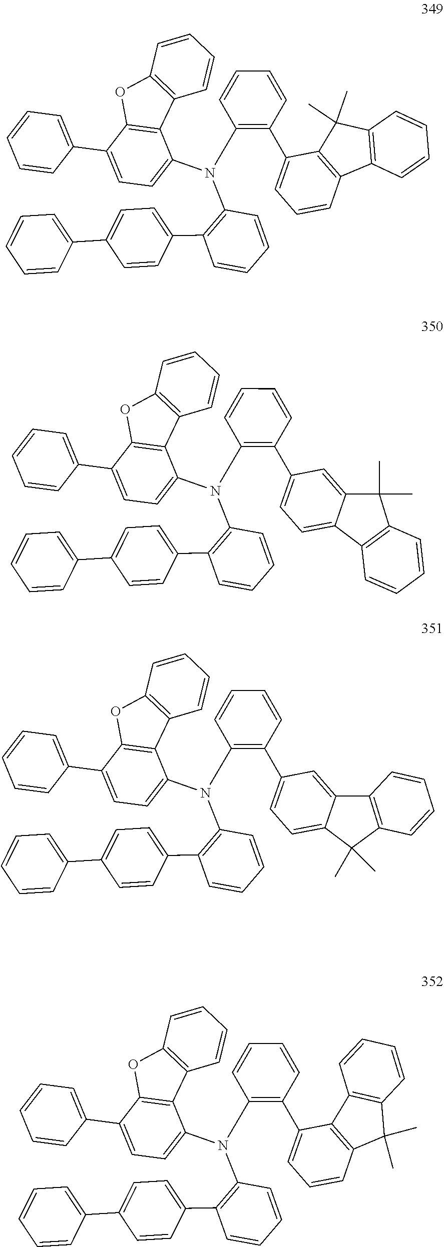 [SMILES: CC1(C)c2ccccc2-c2cccc(-c3ccccc3N(c3ccccc3-c3ccc(-c4ccccc4)cc3)c3ccc(-c4ccccc4)c4oc5ccccc5c34)c21 ; CC1(C)c2ccccc2-c2ccc(-c3ccccc3N(c3ccccc3-c3ccc(-c4ccccc4)cc3)c3ccc(-c4ccccc4)c4oc5ccccc5c34)cc21 ; CC1(C)c2ccccc2-c2cc(-c3ccccc3N(c3ccccc3-c3ccc(-c4ccccc4)cc3)c3ccc(-c4ccccc4)c4oc5ccccc5c34)ccc21 ; CC1(C)c2ccccc2-c2c(-c3ccccc3N(c3ccccc3-c3ccc(-c4ccccc4)cc3)c3ccc(-c4ccccc4)c4oc5ccccc5c34)cccc21]

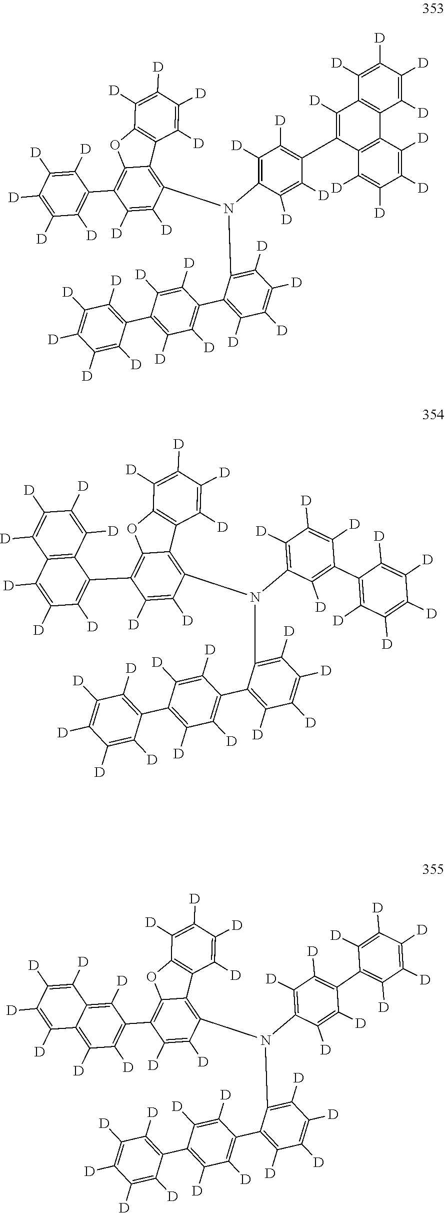

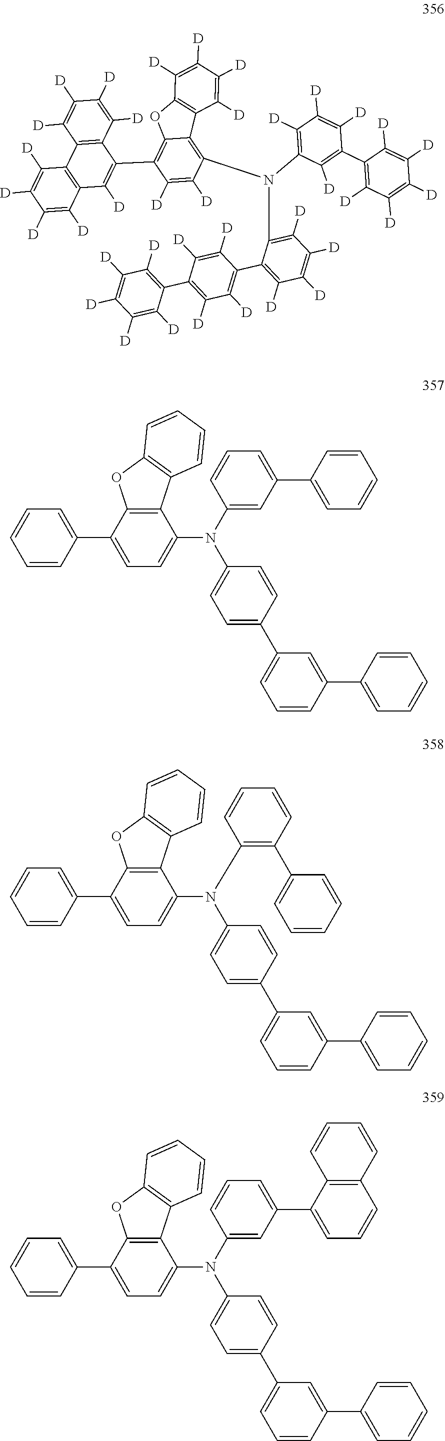

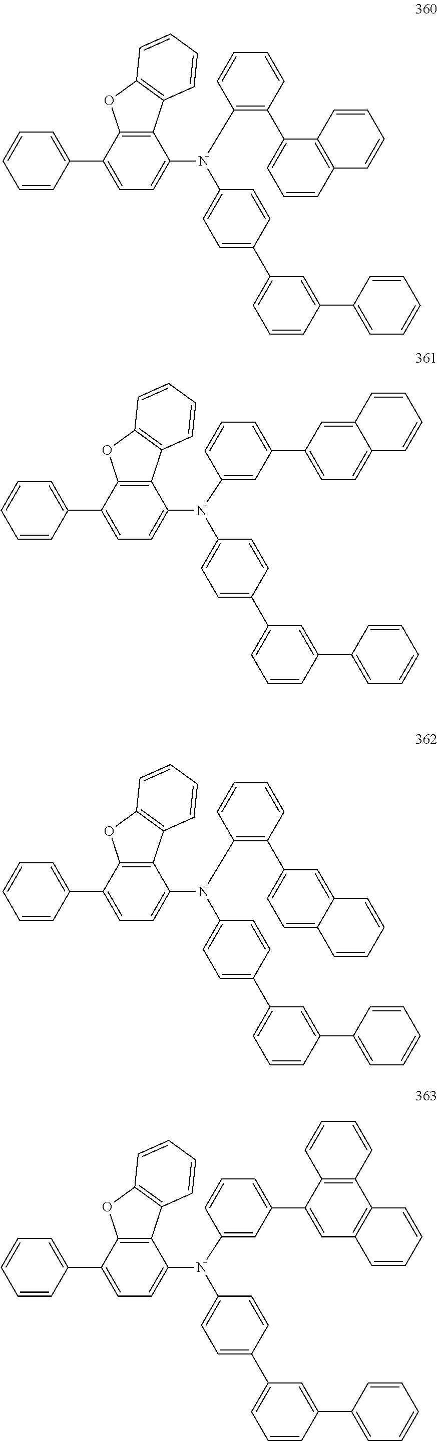 [SMILES: c1ccc(-c2cccc(-c3ccc(N(c4ccccc4-c4cccc5ccccc45)c4ccc(-c5ccccc5)c5oc6ccccc6c45)cc3)c2)cc1 ; c1ccc(-c2cccc(-c3ccc(N(c4cccc(-c5ccc6ccccc6c5)c4)c4ccc(-c5ccccc5)c5oc6ccccc6c45)cc3)c2)cc1 ; c1ccc(-c2cccc(-c3ccc(N(c4ccccc4-c4ccc5ccccc5c4)c4ccc(-c5ccccc5)c5oc6ccccc6c45)cc3)c2)cc1 ; c1ccc(-c2cccc(-c3ccc(N(c4cccc(-c5cc6ccccc6c6ccccc56)c4)c4ccc(-c5ccccc5)c5oc6ccccc6c45)cc3)c2)cc1]

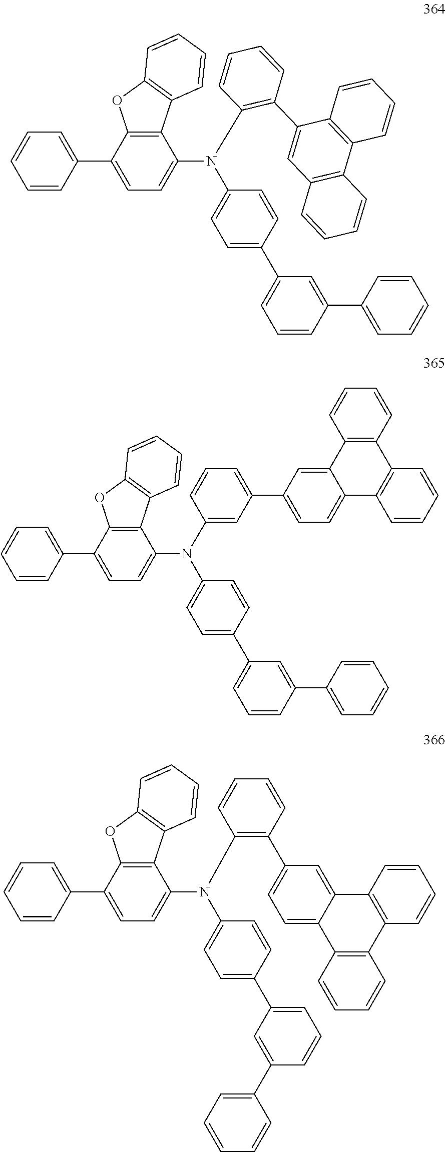 [SMILES: c1ccc(-c2cccc(-c3ccc(N(c4ccccc4-c4cc5ccccc5c5ccccc45)c4ccc(-c5ccccc5)c5oc6ccccc6c45)cc3)c2)cc1 ; c1ccc(-c2cccc(-c3ccc(N(c4cccc(-c5ccc6c7ccccc7c7ccccc7c6c5)c4)c4ccc(-c5ccccc5)c5oc6ccccc6c45)cc3)c2)cc1 ; c1ccc(-c2cccc(-c3ccc(N(c4ccccc4-c4ccc5c6ccccc6c6ccccc6c5c4)c4ccc(-c5ccccc5)c5oc6ccccc6c45)cc3)c2)cc1]

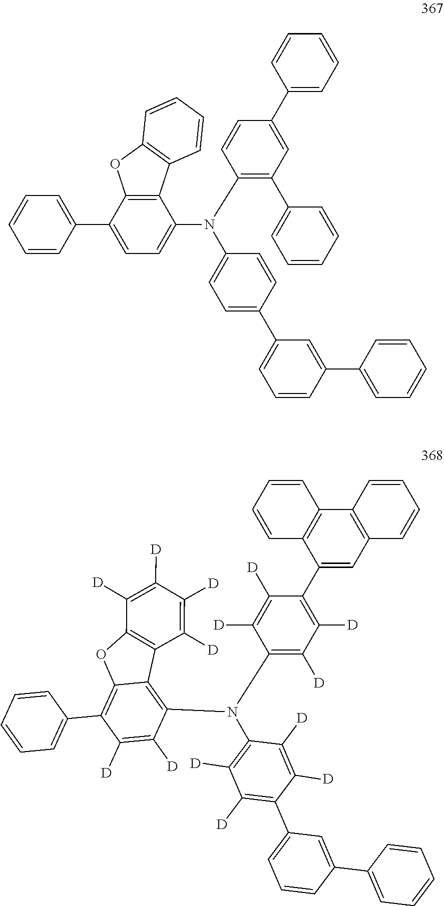 [SMILES: c1ccc(-c2cccc(-c3ccc(N(c4ccc(-c5ccccc5)cc4-c4ccccc4)c4ccc(-c5ccccc5)c5oc6ccccc6c45)cc3)c2)cc1 ; [2H]c1c([2H])c(N(c2c([2H])c([2H])c(-c3cc4ccccc4c4ccccc34)c([2H])c2[2H])c2c([2H])c([2H])c(-c3ccccc3)c3oc4c([2H])c([2H])c([2H])c([2H])c4c23)c([2H])c([2H])c1-c1cccc(-c2ccccc2)c1]

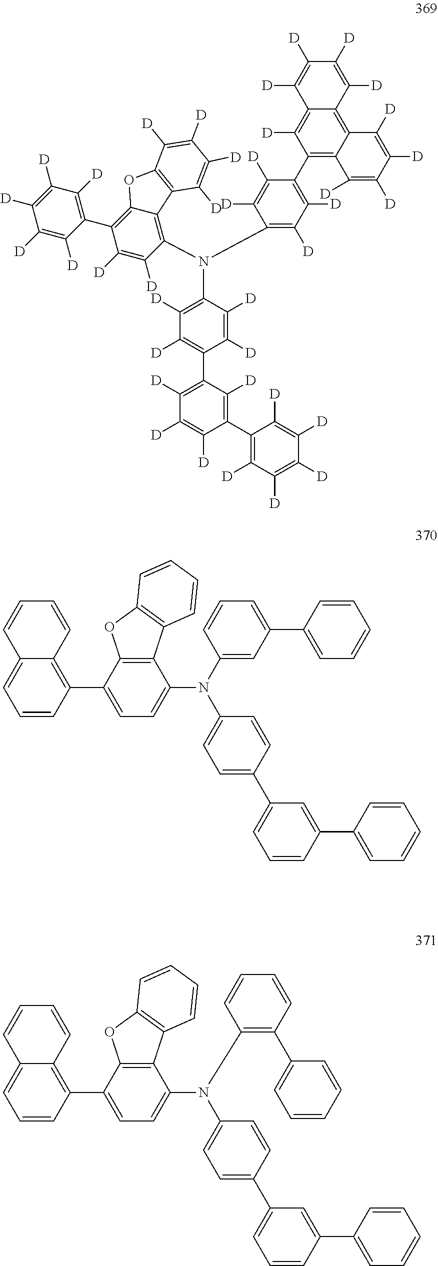 [SMILES: [2H]c1c([2H])c([2H])c(-c2c([2H])c([2H])c([2H])c(-c3c([2H])c([2H])c(N(c4c([2H])c([2H])c(-c5c([2H])c6c([2H])c([2H])c([2H])c([2H])c6c6c([2H])c([2H])c([2H])c([2H])c56)c([2H])c4[2H])c4c([2H])c([2H])c(-c5c([2H])c([2H])c([2H])c([2H])c5[2H])c5oc6c([2H])c([2H])c([2H])c([2H])c6c45)c([2H])c3[2H])c2[2H])c([2H])c1[2H] ; c1ccc(-c2cccc(-c3ccc(N(c4cccc(-c5ccccc5)c4)c4ccc(-c5cccc6ccccc56)c5oc6ccccc6c45)cc3)c2)cc1 ; c1ccc(-c2cccc(-c3ccc(N(c4ccccc4-c4ccccc4)c4ccc(-c5cccc6ccccc56)c5oc6ccccc6c45)cc3)c2)cc1]

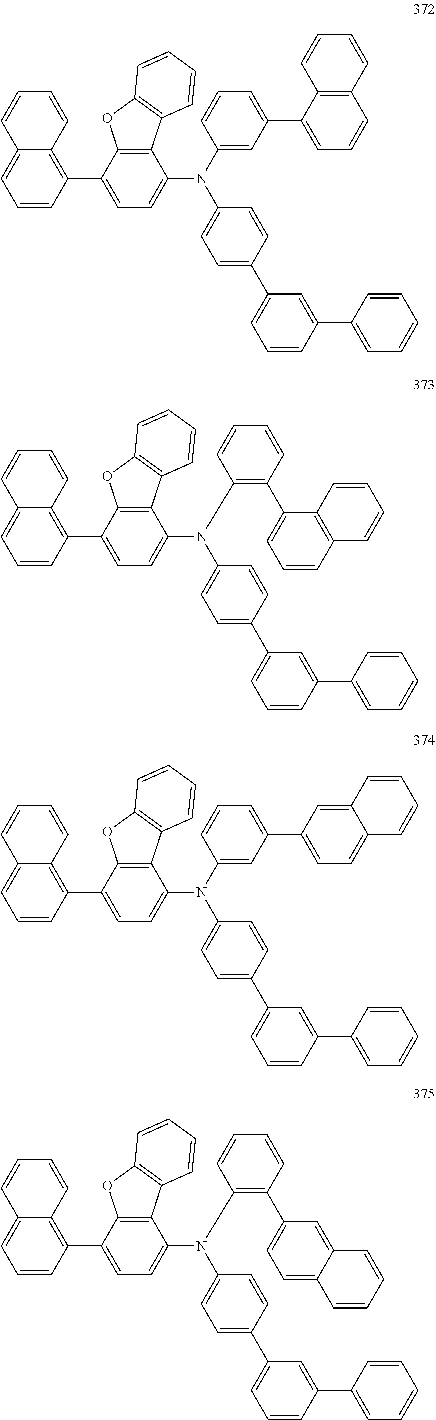 [SMILES: c1ccc(-c2cccc(-c3ccc(N(c4cccc(-c5cccc6ccccc56)c4)c4ccc(-c5cccc6ccccc56)c5oc6ccccc6c45)cc3)c2)cc1 ; c1ccc(-c2cccc(-c3ccc(N(c4ccccc4-c4cccc5ccccc45)c4ccc(-c5cccc6ccccc56)c5oc6ccccc6c45)cc3)c2)cc1 ; c1ccc(-c2cccc(-c3ccc(N(c4cccc(-c5ccc6ccccc6c5)c4)c4ccc(-c5cccc6ccccc56)c5oc6ccccc6c45)cc3)c2)cc1 ; c1ccc(-c2cccc(-c3ccc(N(c4ccccc4-c4ccc5ccccc5c4)c4ccc(-c5cccc6ccccc56)c5oc6ccccc6c45)cc3)c2)cc1]

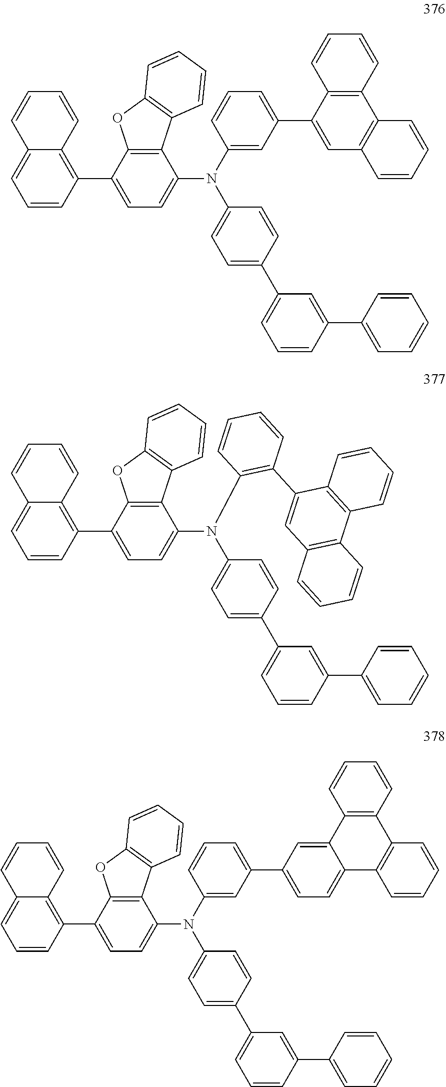 [SMILES: c1ccc(-c2cccc(-c3ccc(N(c4cccc(-c5cc6ccccc6c6ccccc56)c4)c4ccc(-c5cccc6ccccc56)c5oc6ccccc6c45)cc3)c2)cc1 ; c1ccc(-c2cccc(-c3ccc(N(c4ccccc4-c4cc5ccccc5c5ccccc45)c4ccc(-c5cccc6ccccc56)c5oc6ccccc6c45)cc3)c2)cc1 ; c1ccc(-c2cccc(-c3ccc(N(c4cccc(-c5ccc6c7ccccc7c7ccccc7c6c5)c4)c4ccc(-c5cccc6ccccc56)c5oc6ccccc6c45)cc3)c2)cc1]

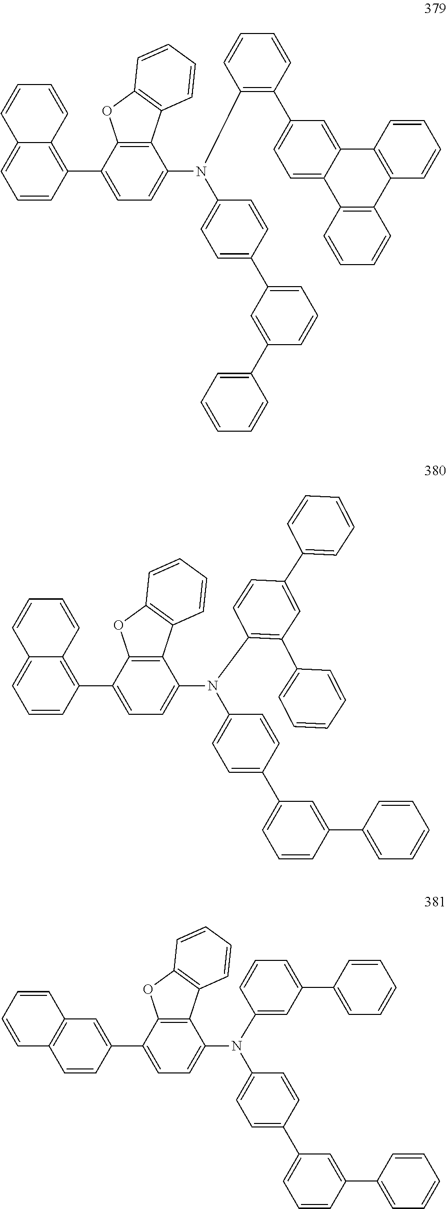 [SMILES: c1ccc(-c2cccc(-c3ccc(N(c4ccccc4-c4ccc5c6ccccc6c6ccccc6c5c4)c4ccc(-c5cccc6ccccc56)c5oc6ccccc6c45)cc3)c2)cc1 ; c1ccc(-c2cccc(-c3ccc(N(c4ccc(-c5ccccc5)cc4-c4ccccc4)c4ccc(-c5cccc6ccccc56)c5oc6ccccc6c45)cc3)c2)cc1 ; c1ccc(-c2cccc(-c3ccc(N(c4cccc(-c5ccccc5)c4)c4ccc(-c5ccc6ccccc6c5)c5oc6ccccc6c45)cc3)c2)cc1]

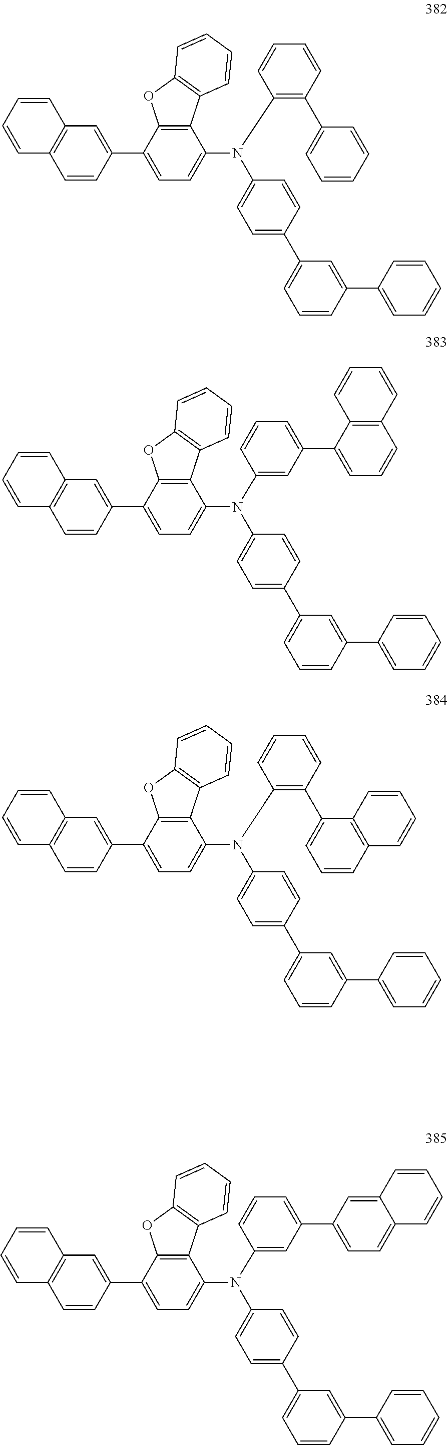 [SMILES: c1ccc(-c2cccc(-c3ccc(N(c4ccccc4-c4ccccc4)c4ccc(-c5ccc6ccccc6c5)c5oc6ccccc6c45)cc3)c2)cc1 ; c1ccc(-c2cccc(-c3ccc(N(c4cccc(-c5cccc6ccccc56)c4)c4ccc(-c5ccc6ccccc6c5)c5oc6ccccc6c45)cc3)c2)cc1 ; c1ccc(-c2cccc(-c3ccc(N(c4ccccc4-c4cccc5ccccc45)c4ccc(-c5ccc6ccccc6c5)c5oc6ccccc6c45)cc3)c2)cc1 ; c1ccc(-c2cccc(-c3ccc(N(c4cccc(-c5ccc6ccccc6c5)c4)c4ccc(-c5ccc6ccccc6c5)c5oc6ccccc6c45)cc3)c2)cc1]

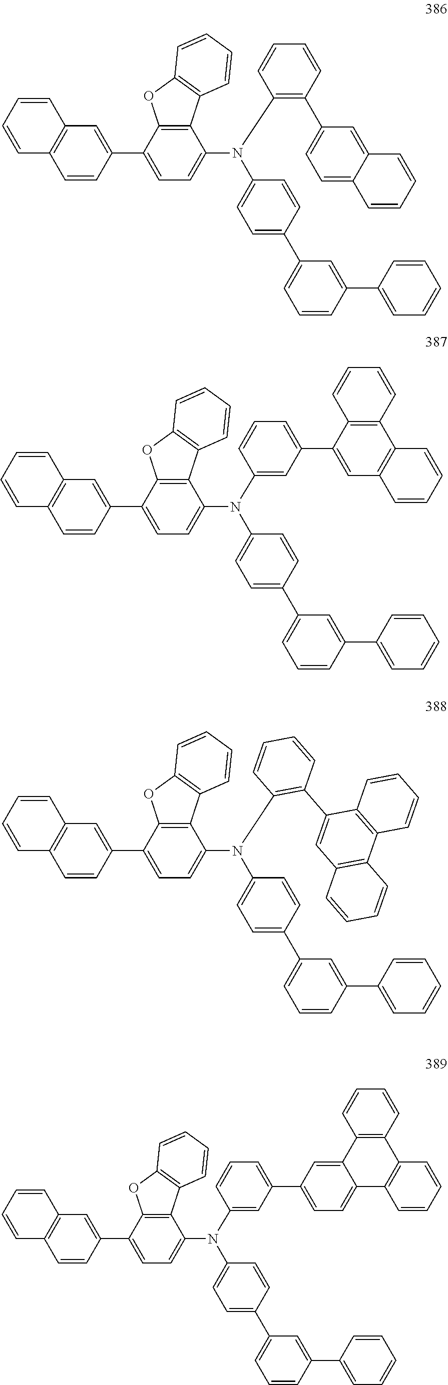 [SMILES: c1ccc(-c2cccc(-c3ccc(N(c4ccccc4-c4ccc5ccccc5c4)c4ccc(-c5ccc6ccccc6c5)c5oc6ccccc6c45)cc3)c2)cc1 ; c1ccc(-c2cccc(-c3ccc(N(c4cccc(-c5cc6ccccc6c6ccccc56)c4)c4ccc(-c5ccc6ccccc6c5)c5oc6ccccc6c45)cc3)c2)cc1 ; c1ccc(-c2cccc(-c3ccc(N(c4ccccc4-c4cc5ccccc5c5ccccc45)c4ccc(-c5ccc6ccccc6c5)c5oc6ccccc6c45)cc3)c2)cc1 ; c1ccc(-c2cccc(-c3ccc(N(c4cccc(-c5ccc6c7ccccc7c7ccccc7c6c5)c4)c4ccc(-c5ccc6ccccc6c5)c5oc6ccccc6c45)cc3)c2)cc1]

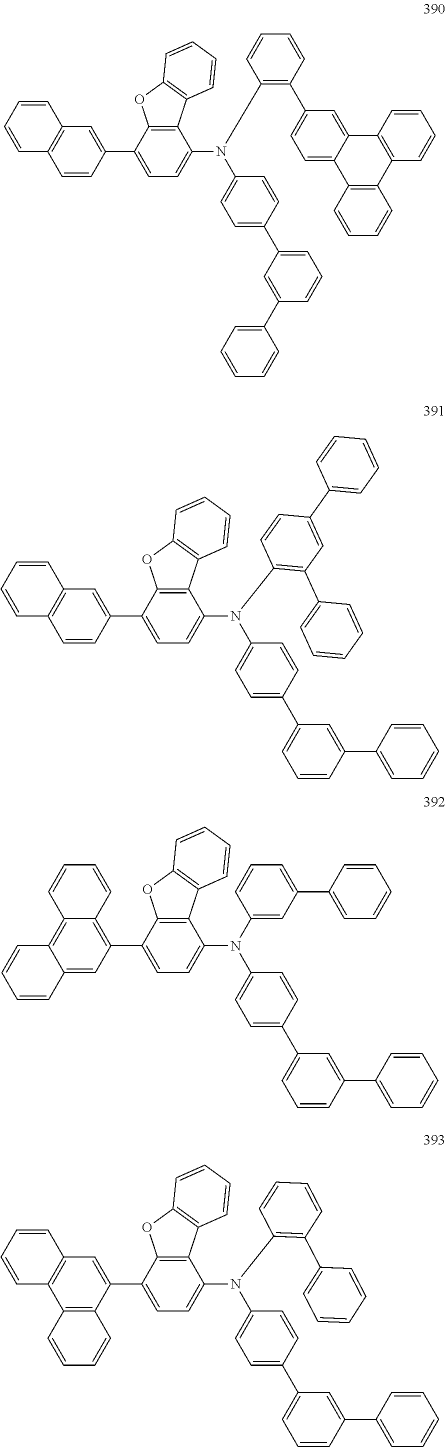 [SMILES: c1ccc(-c2cccc(-c3ccc(N(c4ccccc4-c4ccc5c6ccccc6c6ccccc6c5c4)c4ccc(-c5ccc6ccccc6c5)c5oc6ccccc6c45)cc3)c2)cc1 ; c1ccc(-c2cccc(-c3ccc(N(c4ccc(-c5ccccc5)cc4-c4ccccc4)c4ccc(-c5ccc6ccccc6c5)c5oc6ccccc6c45)cc3)c2)cc1 ; c1ccc(-c2cccc(-c3ccc(N(c4cccc(-c5ccccc5)c4)c4ccc(-c5cc6ccccc6c6ccccc56)c5oc6ccccc6c45)cc3)c2)cc1 ; c1ccc(-c2cccc(-c3ccc(N(c4ccccc4-c4ccccc4)c4ccc(-c5cc6ccccc6c6ccccc56)c5oc6ccccc6c45)cc3)c2)cc1]

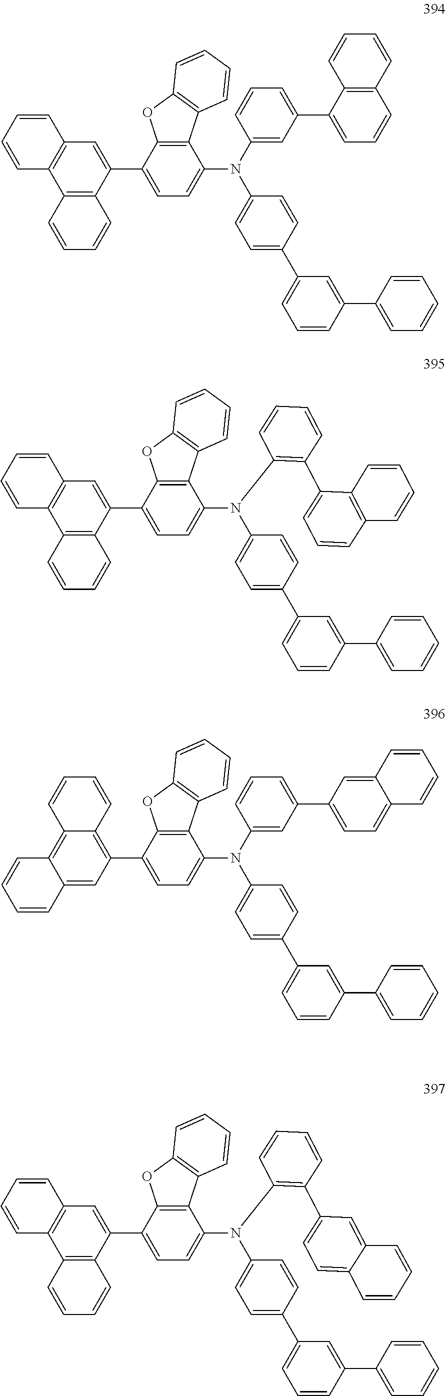 [SMILES: c1ccc(-c2cccc(-c3ccc(N(c4cccc(-c5cccc6ccccc56)c4)c4ccc(-c5cc6ccccc6c6ccccc56)c5oc6ccccc6c45)cc3)c2)cc1 ; c1ccc(-c2cccc(-c3ccc(N(c4ccccc4-c4cccc5ccccc45)c4ccc(-c5cc6ccccc6c6ccccc56)c5oc6ccccc6c45)cc3)c2)cc1 ; c1ccc(-c2cccc(-c3ccc(N(c4cccc(-c5ccc6ccccc6c5)c4)c4ccc(-c5cc6ccccc6c6ccccc56)c5oc6ccccc6c45)cc3)c2)cc1 ; c1ccc(-c2cccc(-c3ccc(N(c4ccccc4-c4ccc5ccccc5c4)c4ccc(-c5cc6ccccc6c6ccccc56)c5oc6ccccc6c45)cc3)c2)cc1]

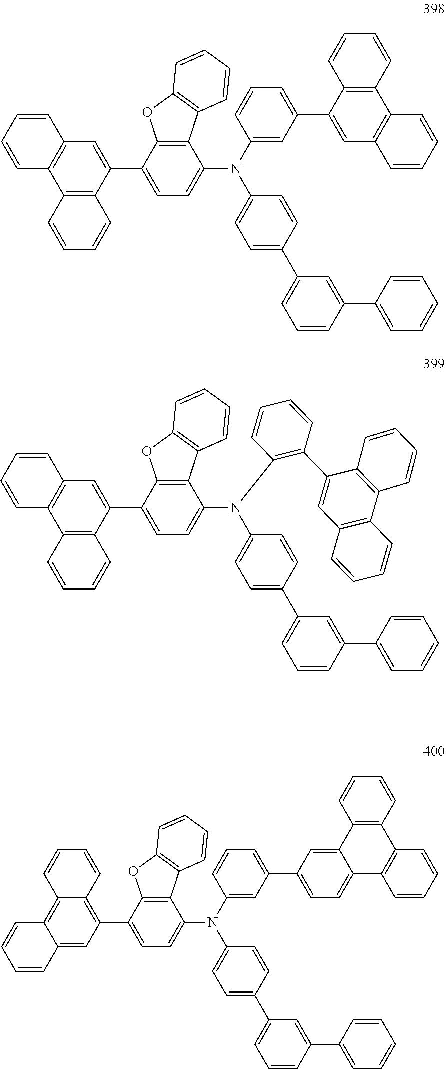 [SMILES: c1ccc(-c2cccc(-c3ccc(N(c4cccc(-c5cc6ccccc6c6ccccc56)c4)c4ccc(-c5cc6ccccc6c6ccccc56)c5oc6ccccc6c45)cc3)c2)cc1 ; c1ccc(-c2cccc(-c3ccc(N(c4ccccc4-c4cc5ccccc5c5ccccc45)c4ccc(-c5cc6ccccc6c6ccccc56)c5oc6ccccc6c45)cc3)c2)cc1 ; c1ccc(-c2cccc(-c3ccc(N(c4cccc(-c5ccc6c7ccccc7c7ccccc7c6c5)c4)c4ccc(-c5cc6ccccc6c6ccccc56)c5oc6ccccc6c45)cc3)c2)cc1]

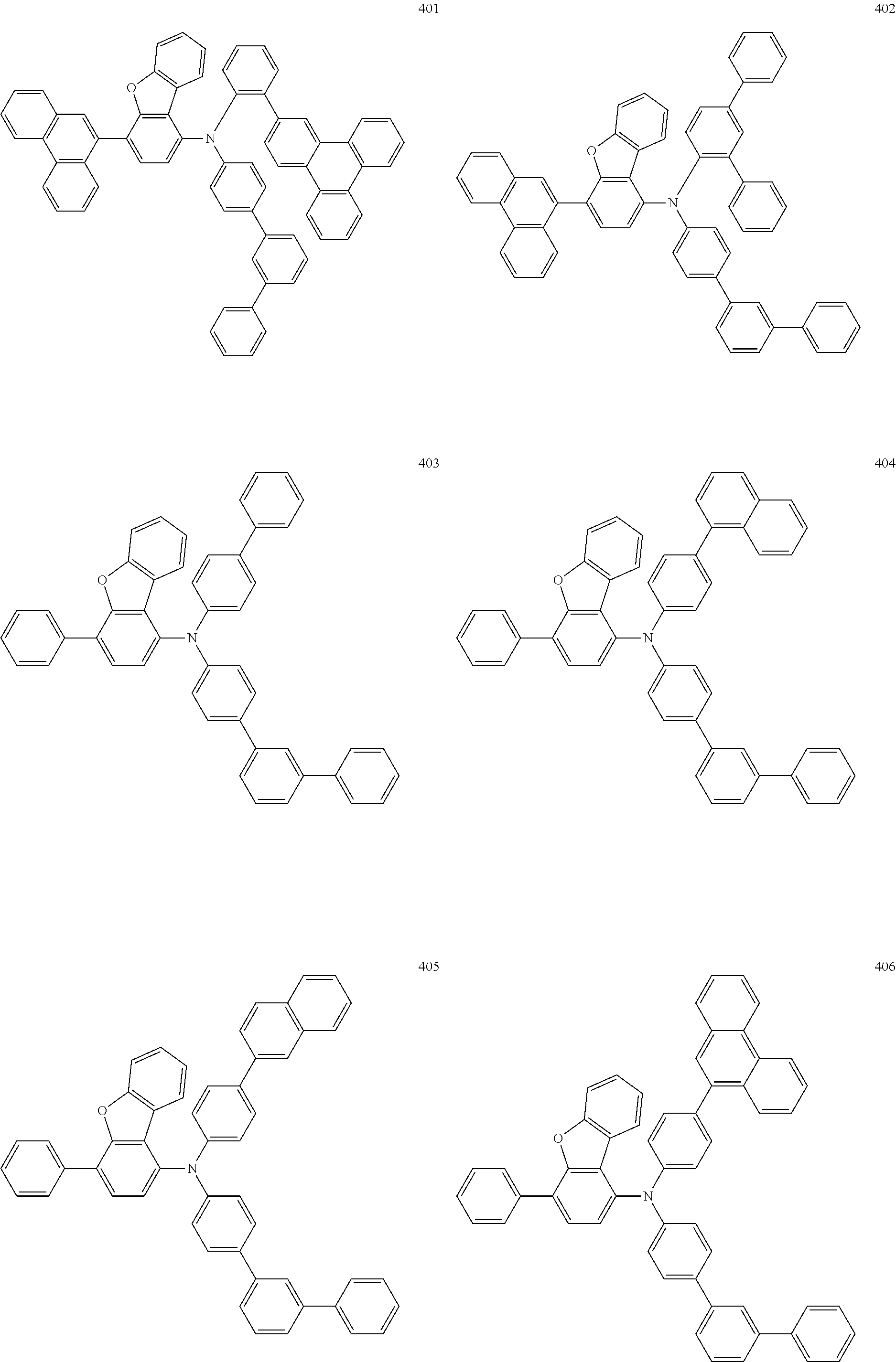

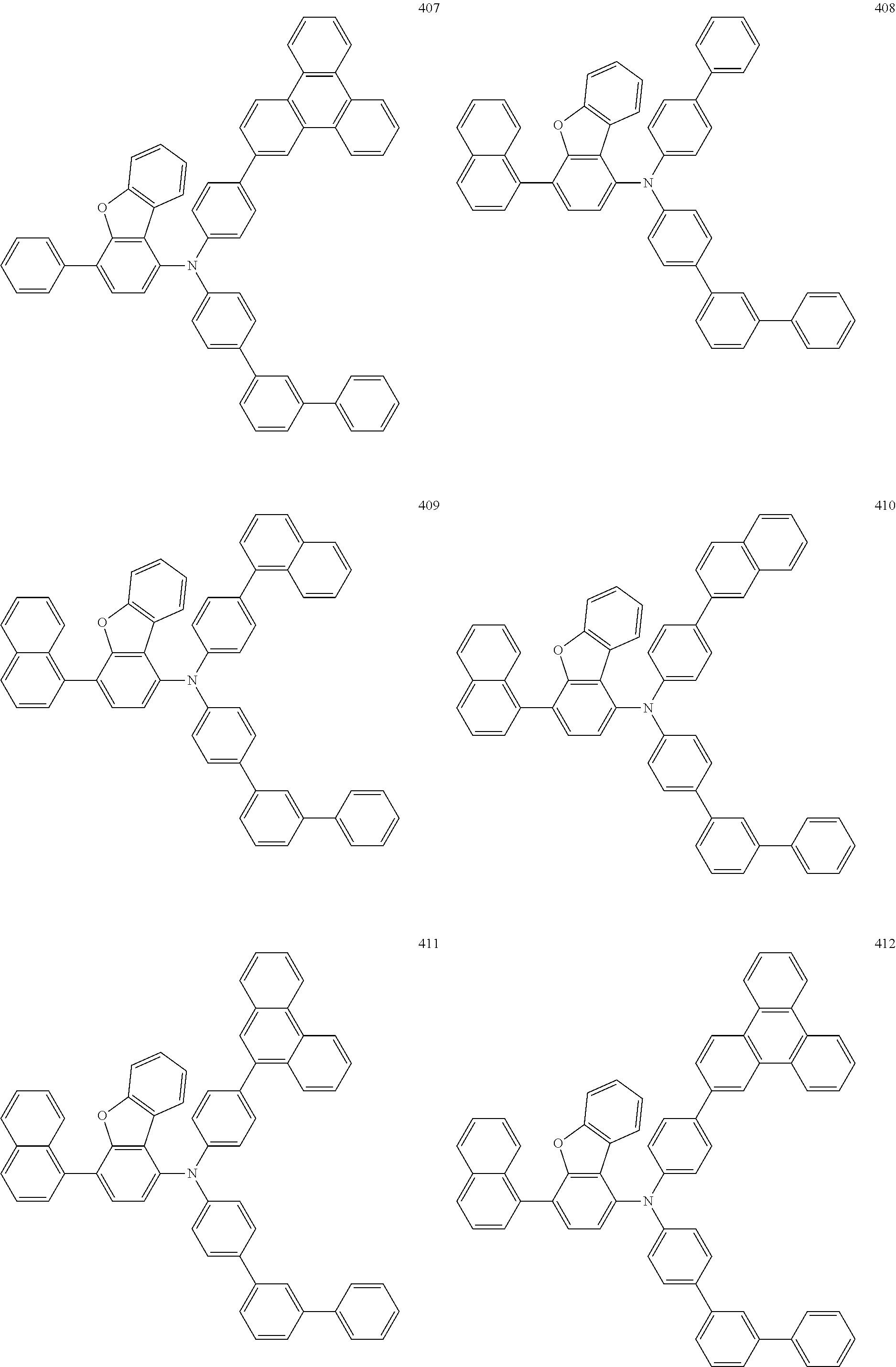

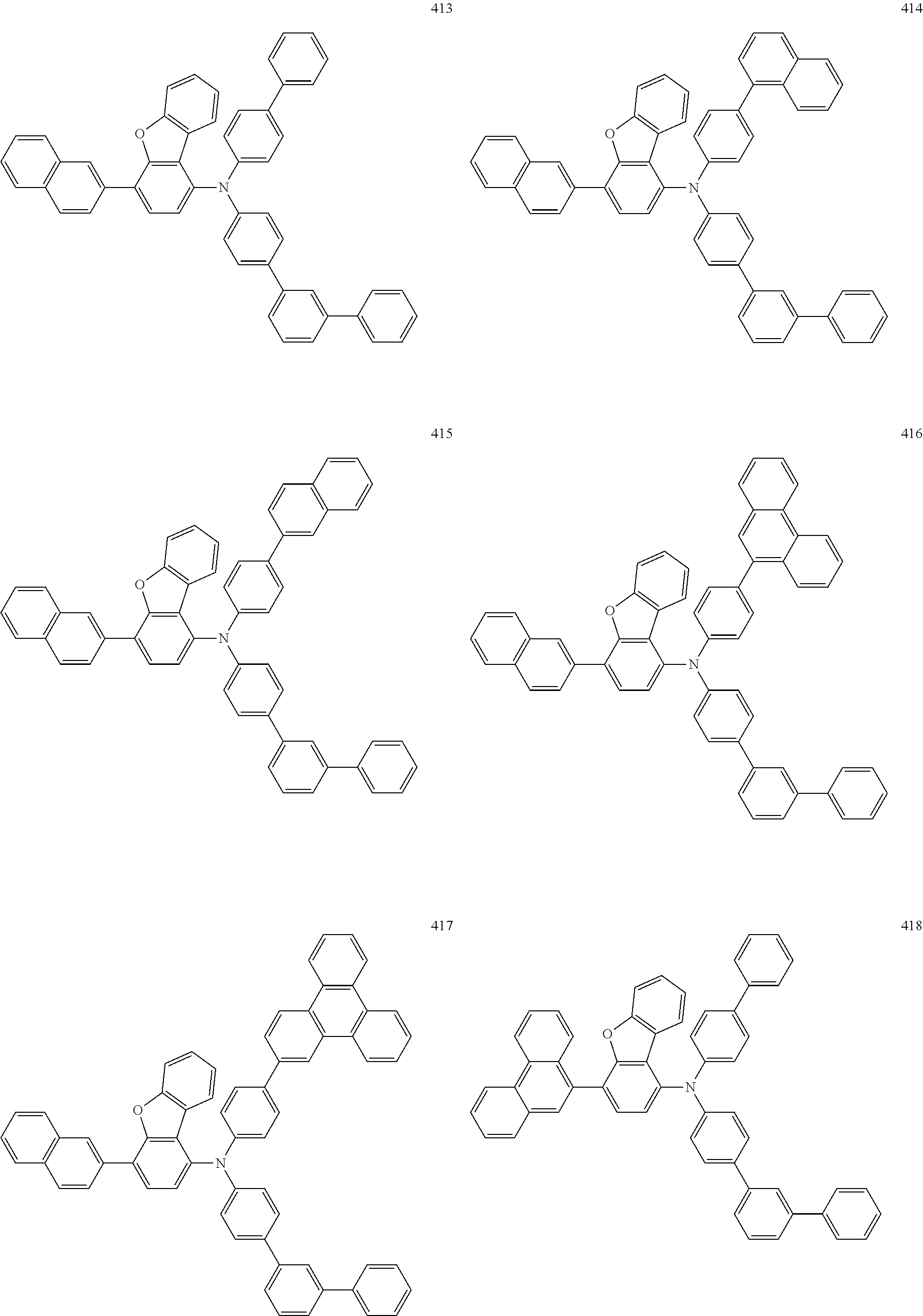

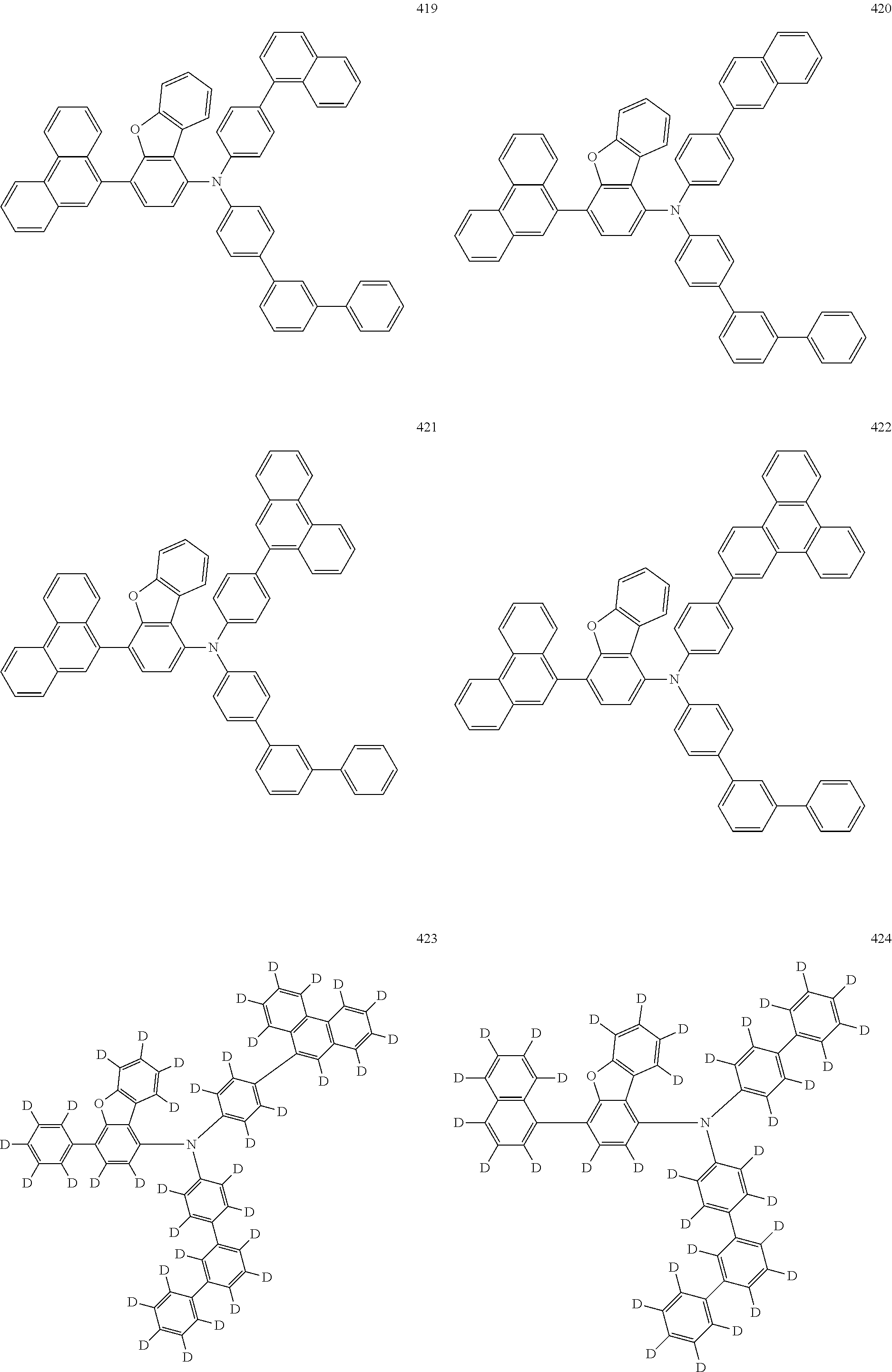

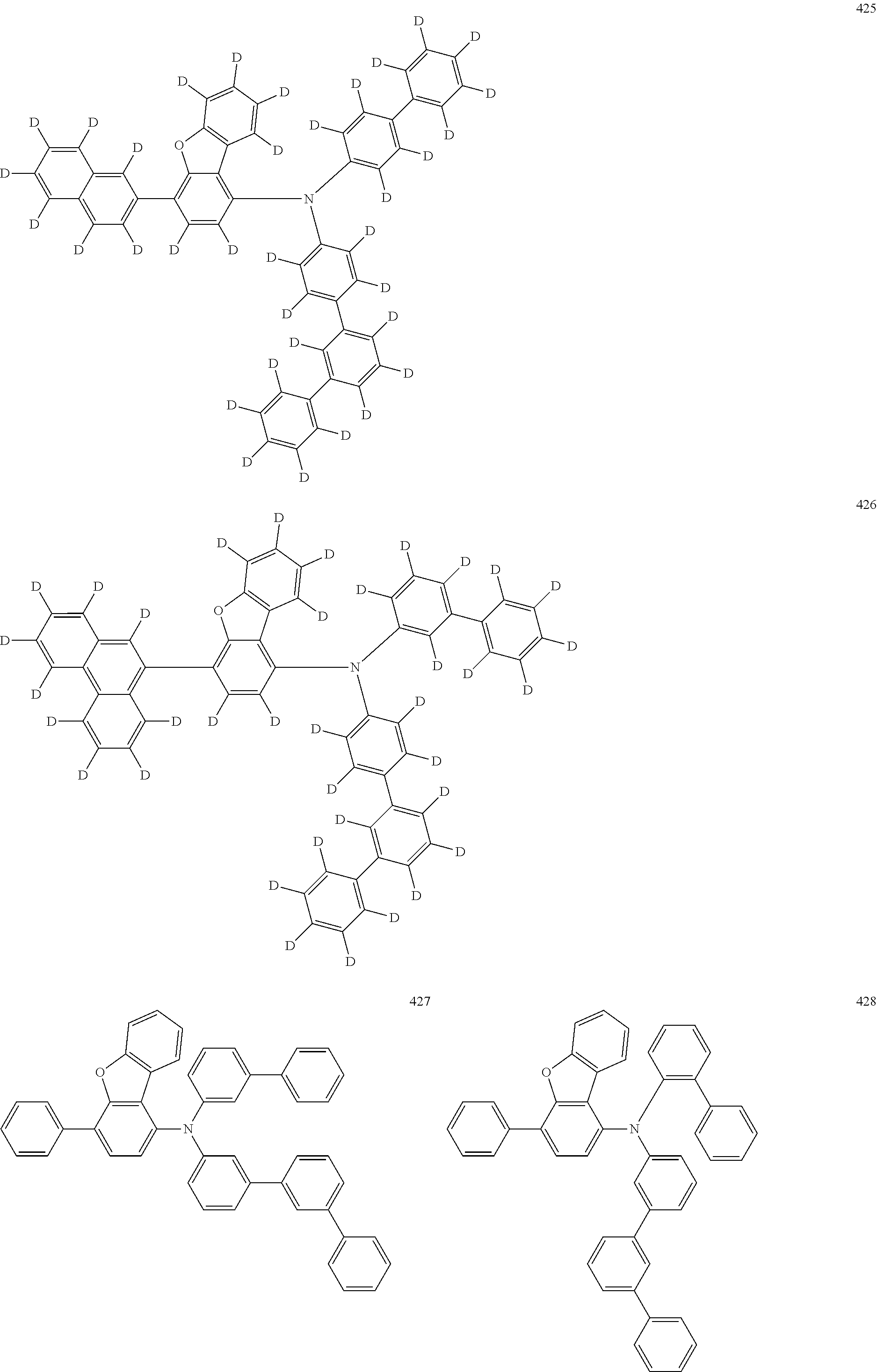

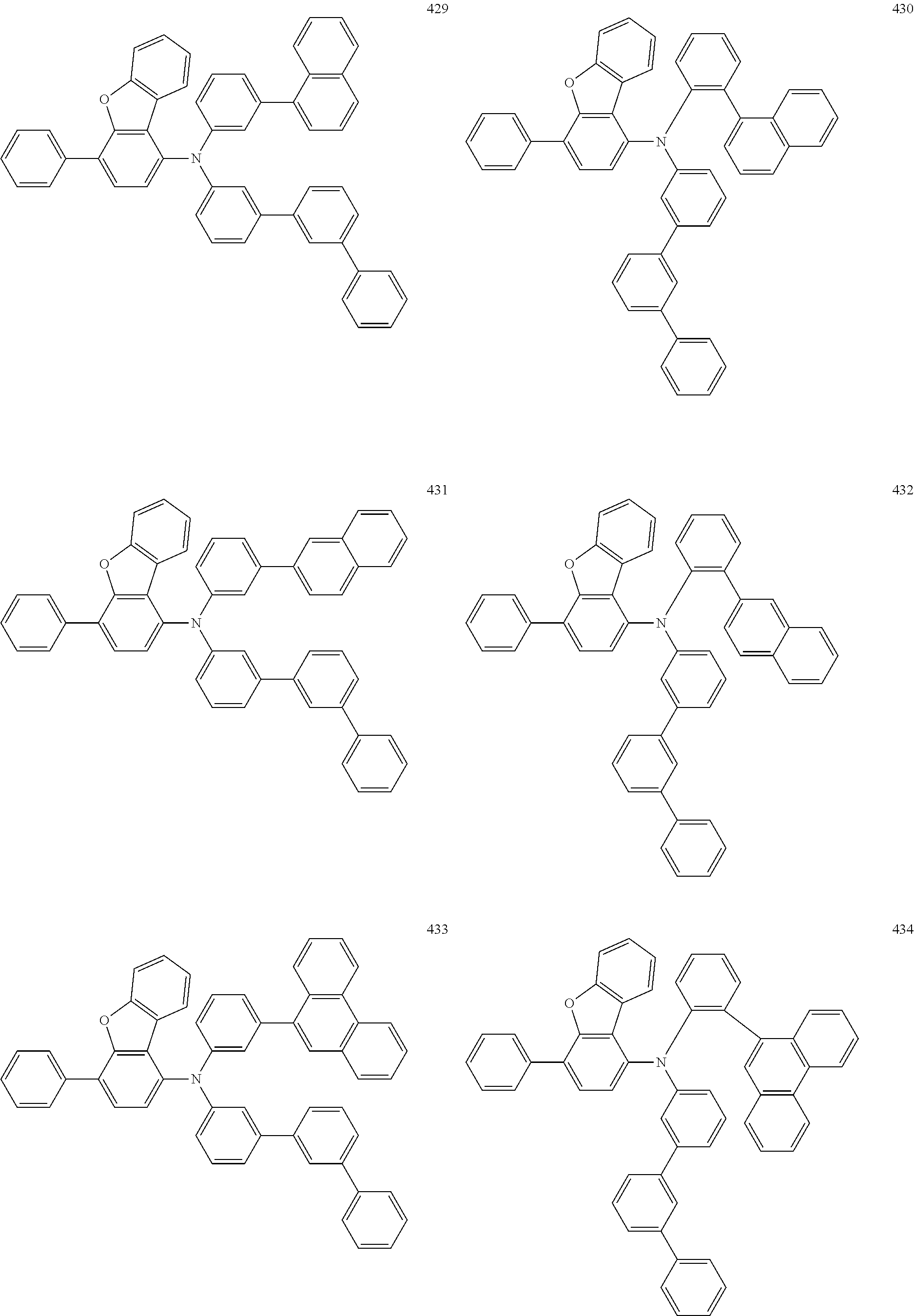

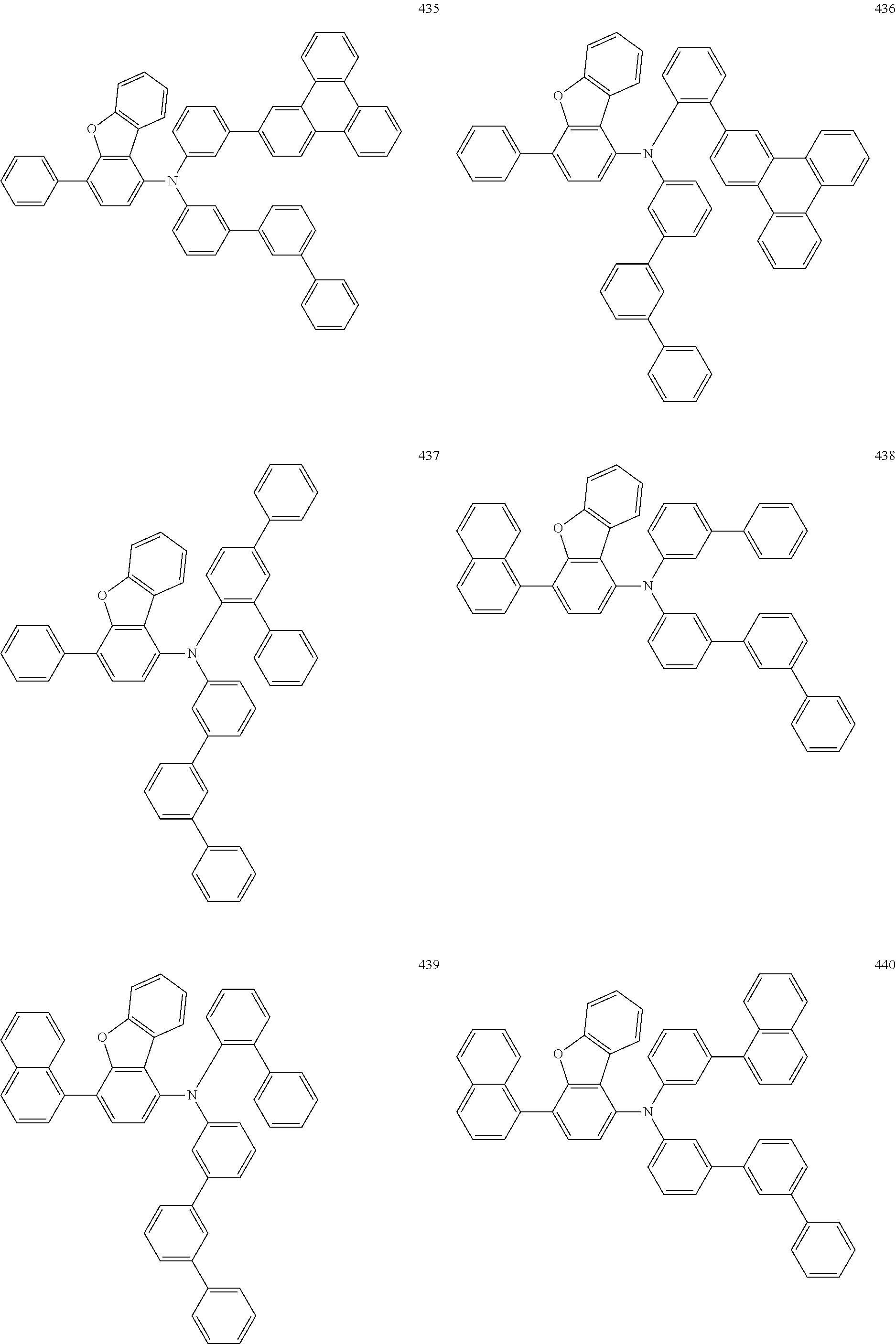

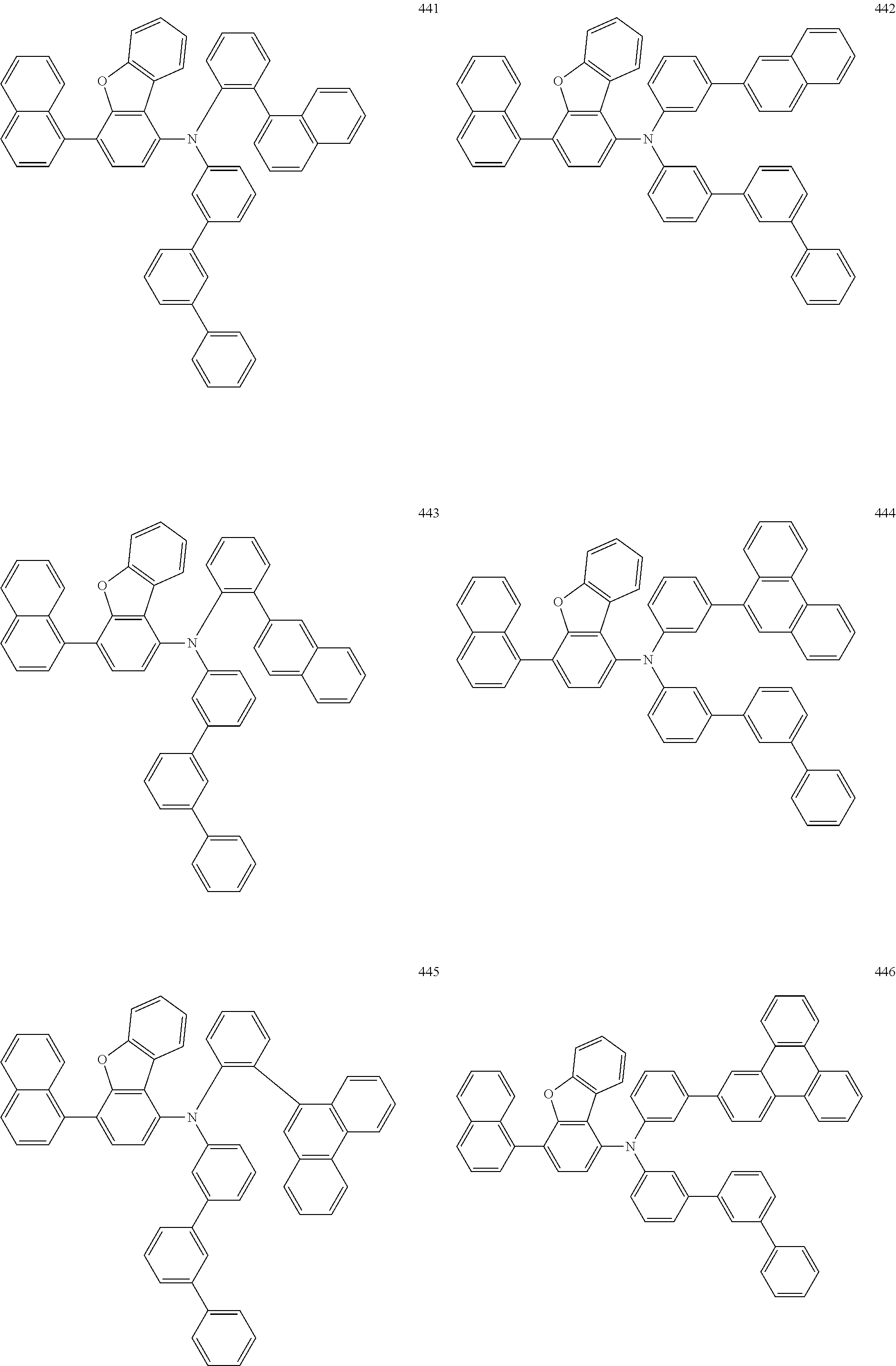

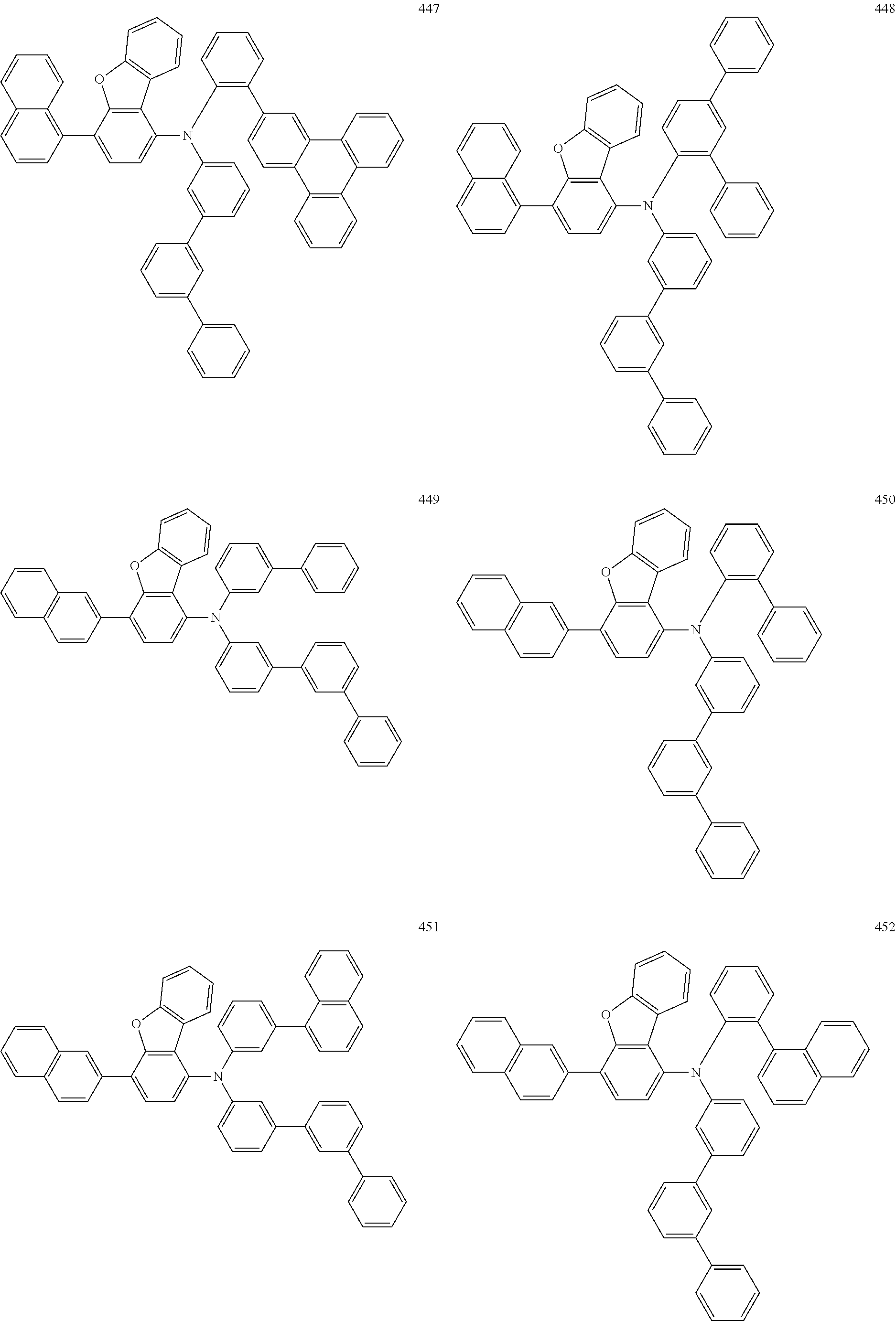

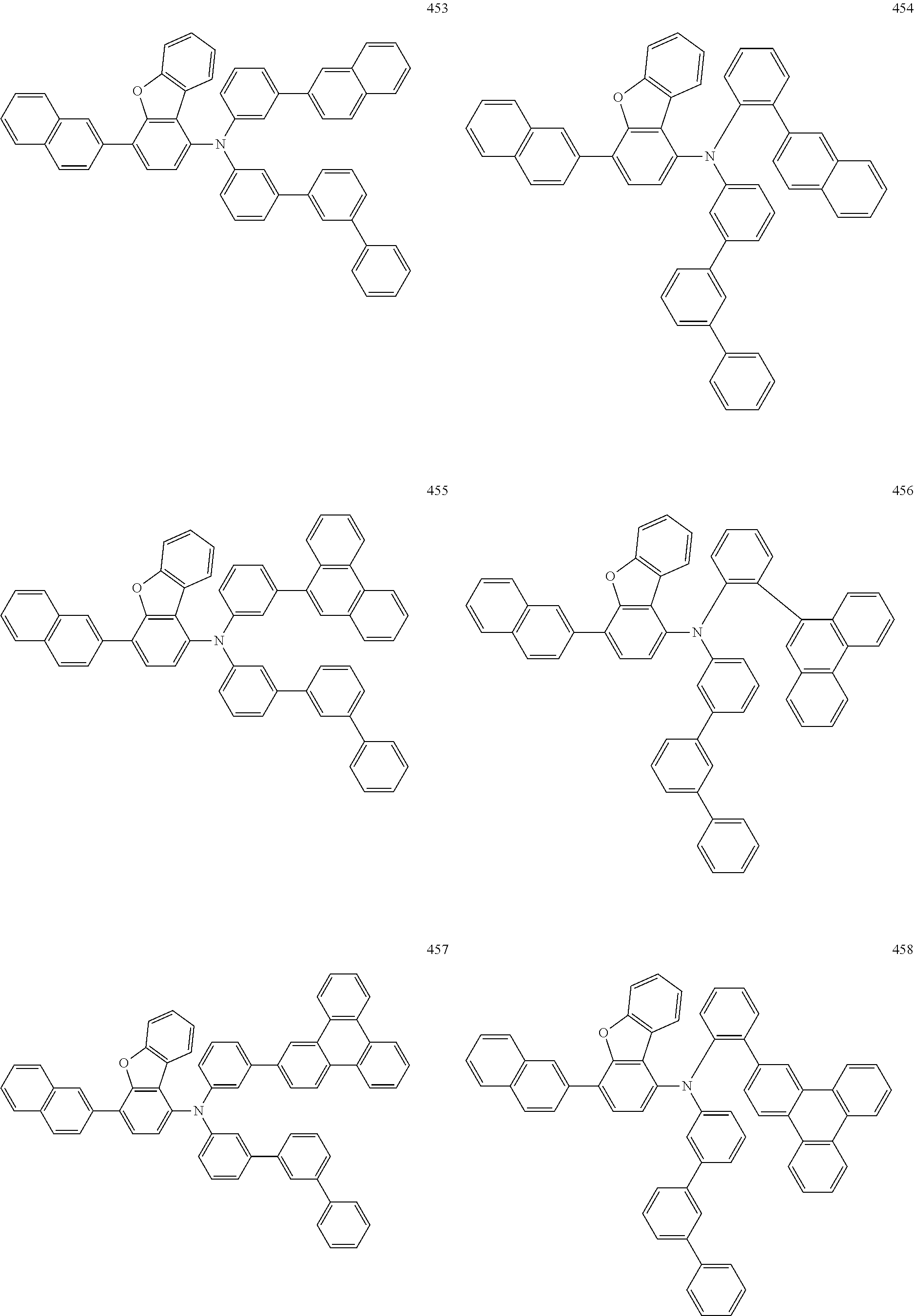

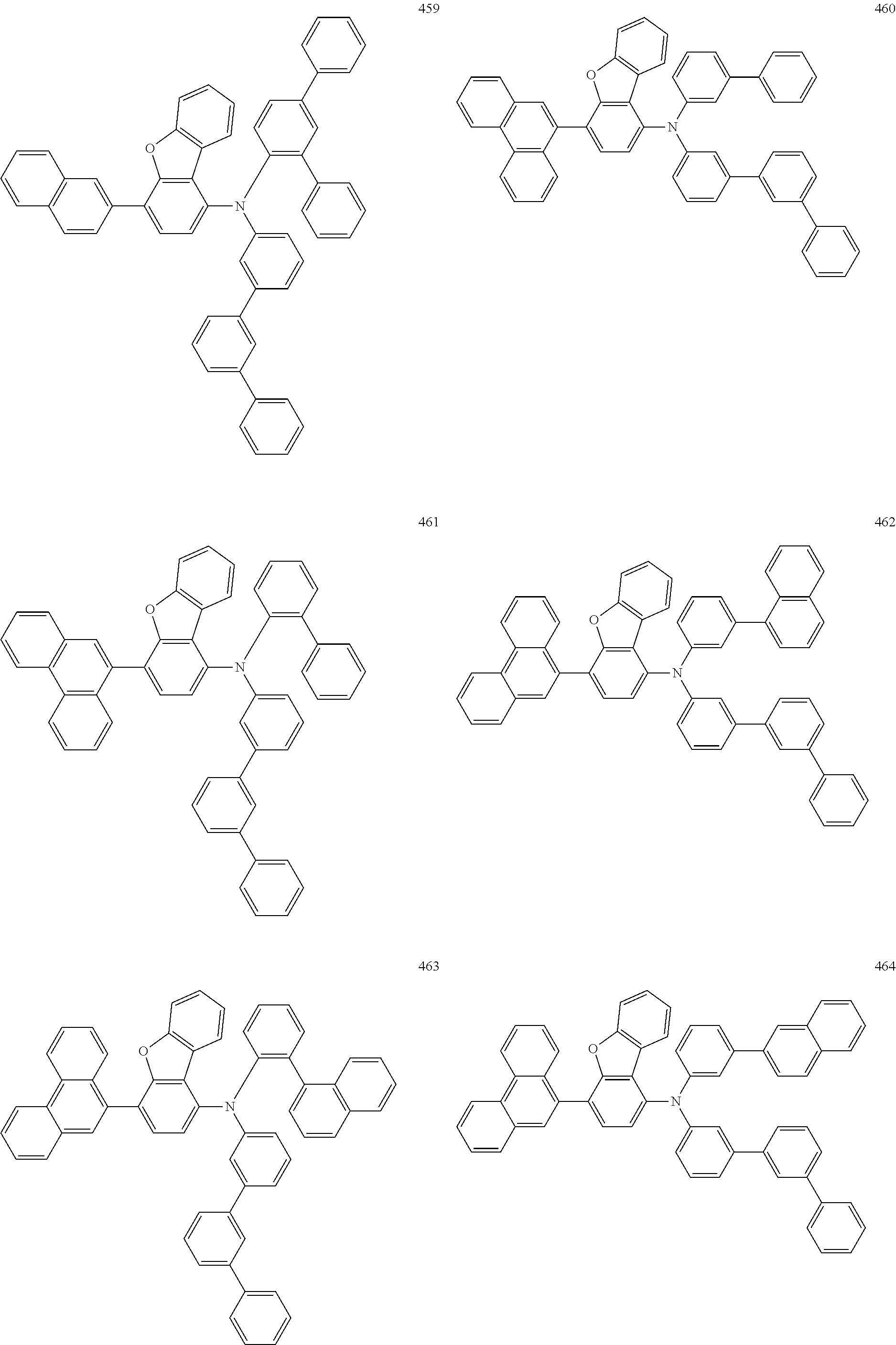

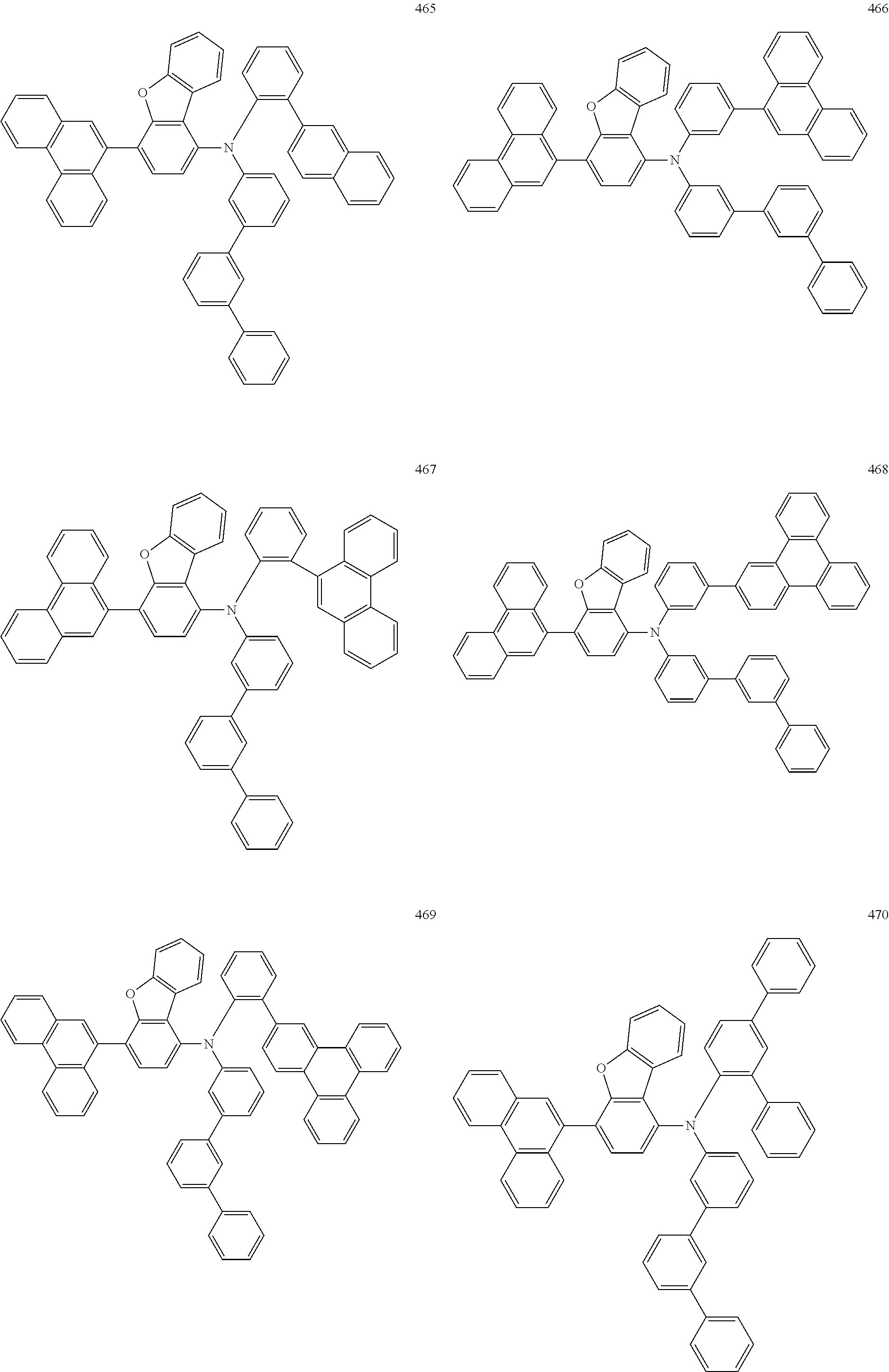

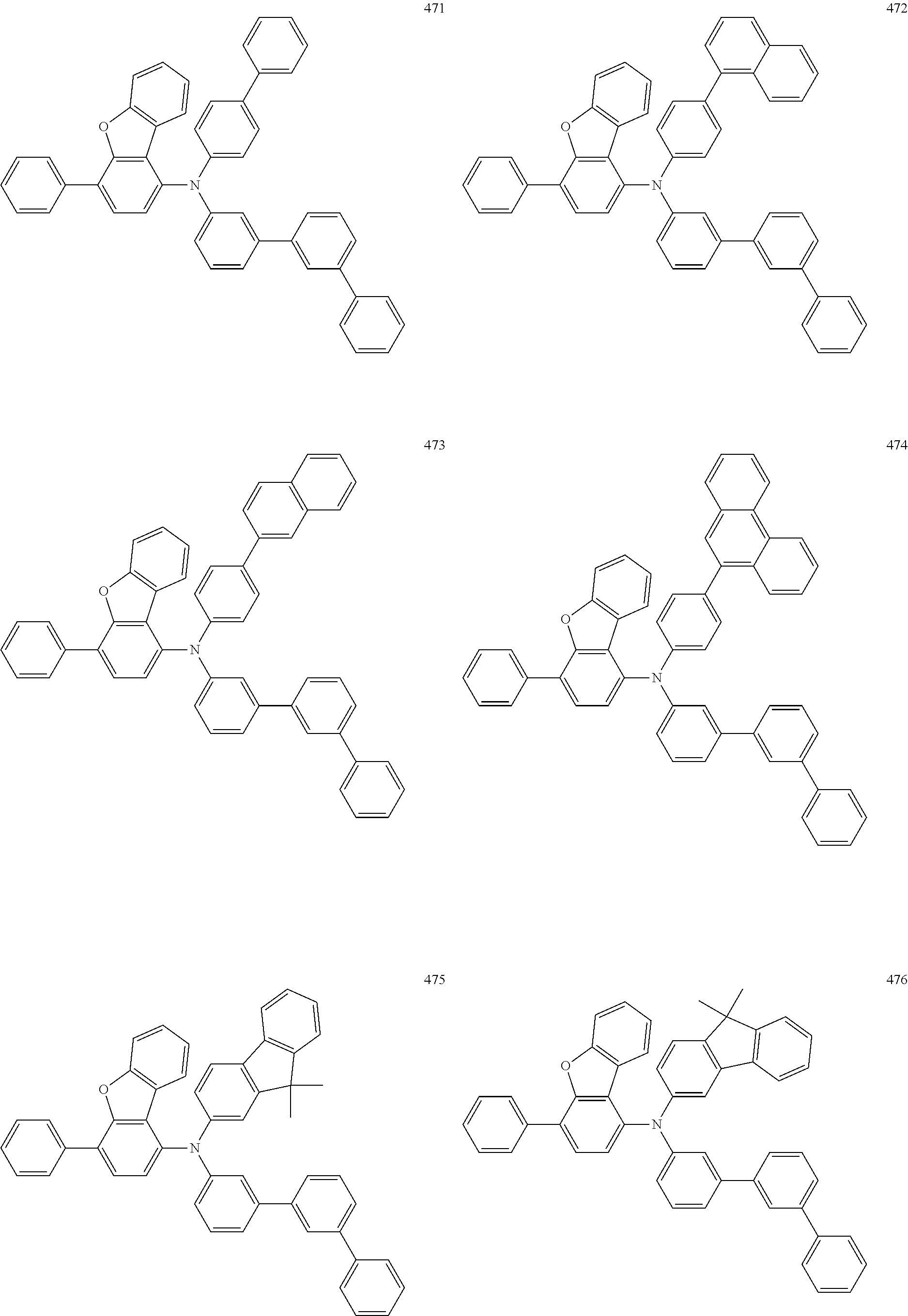

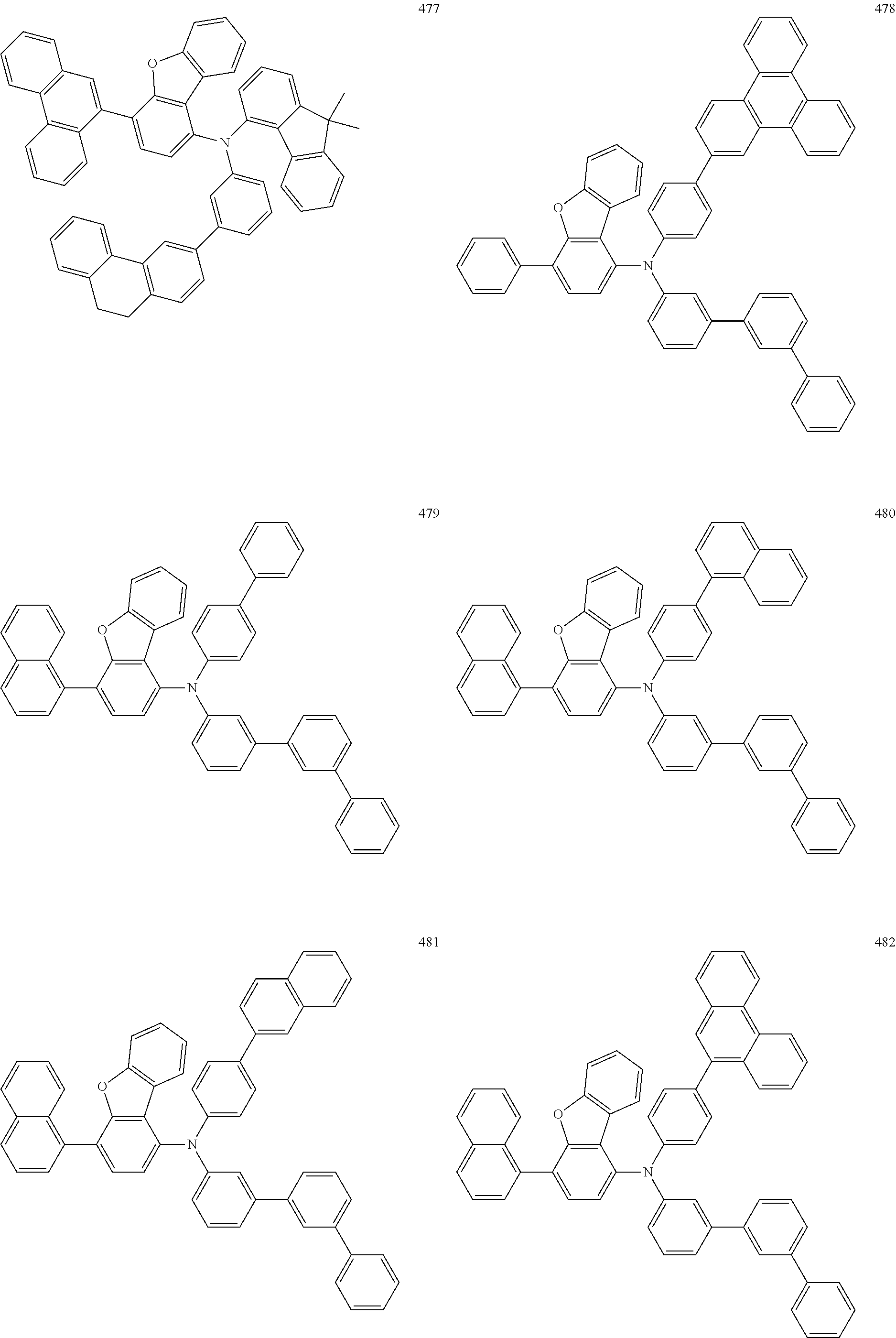

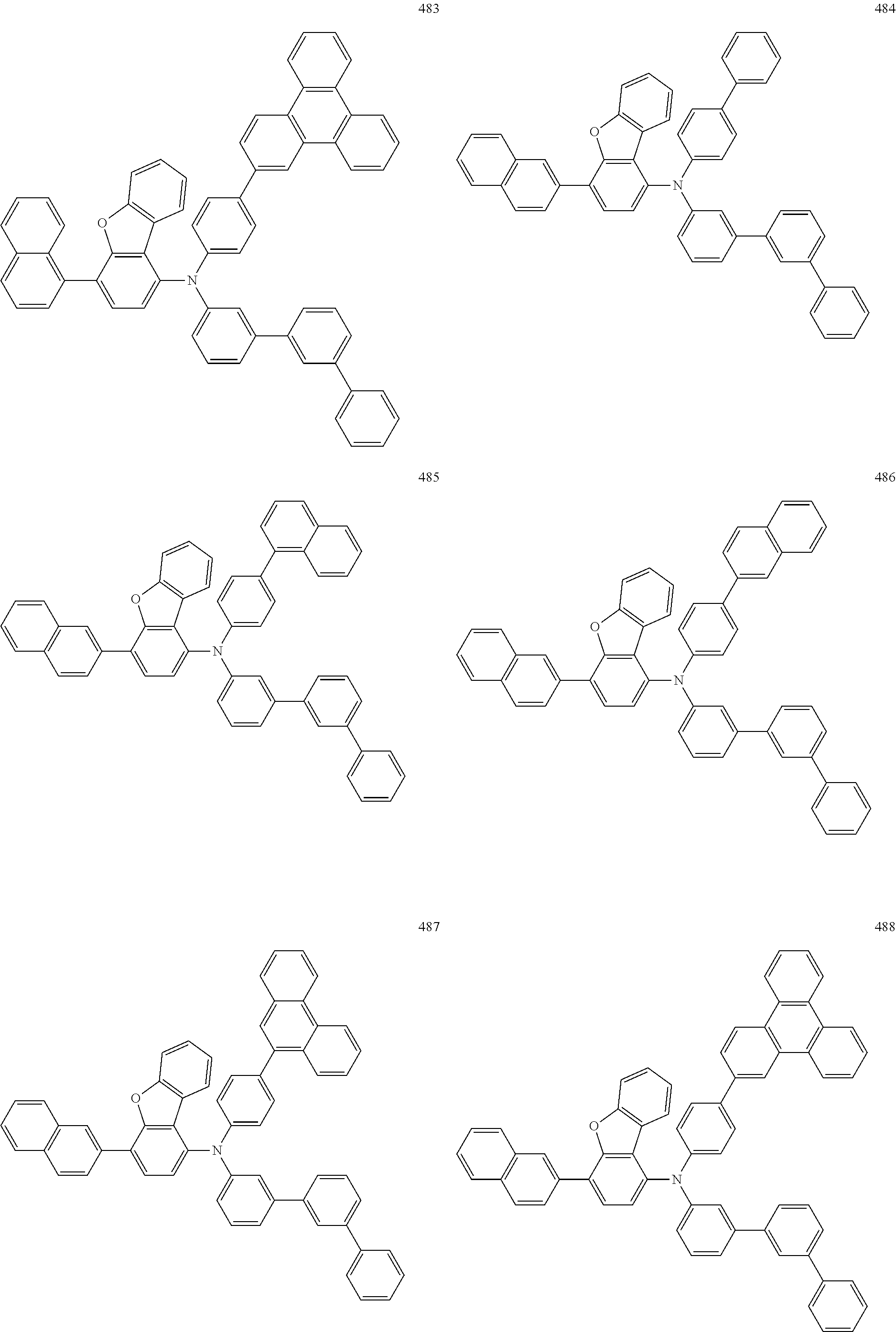

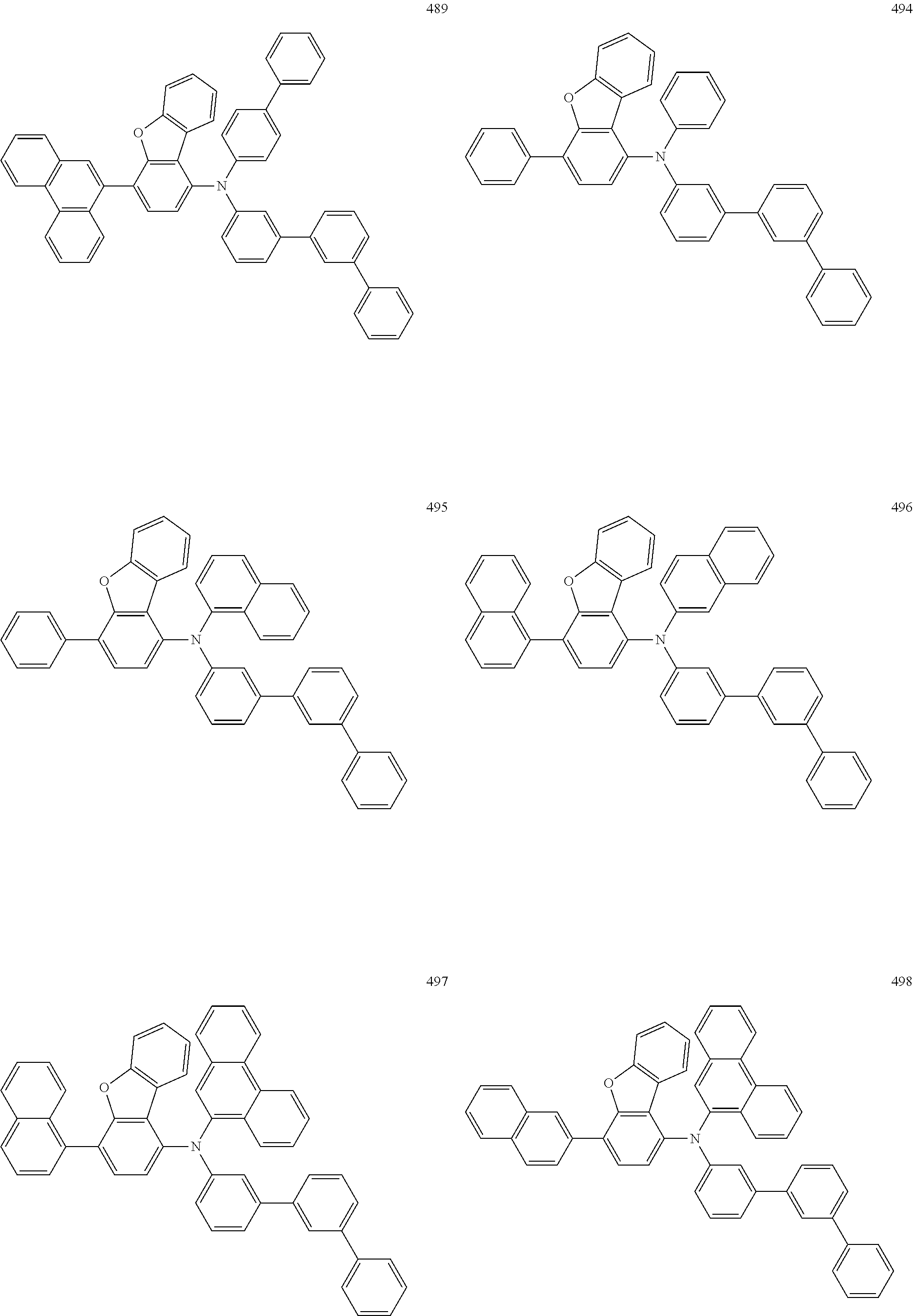

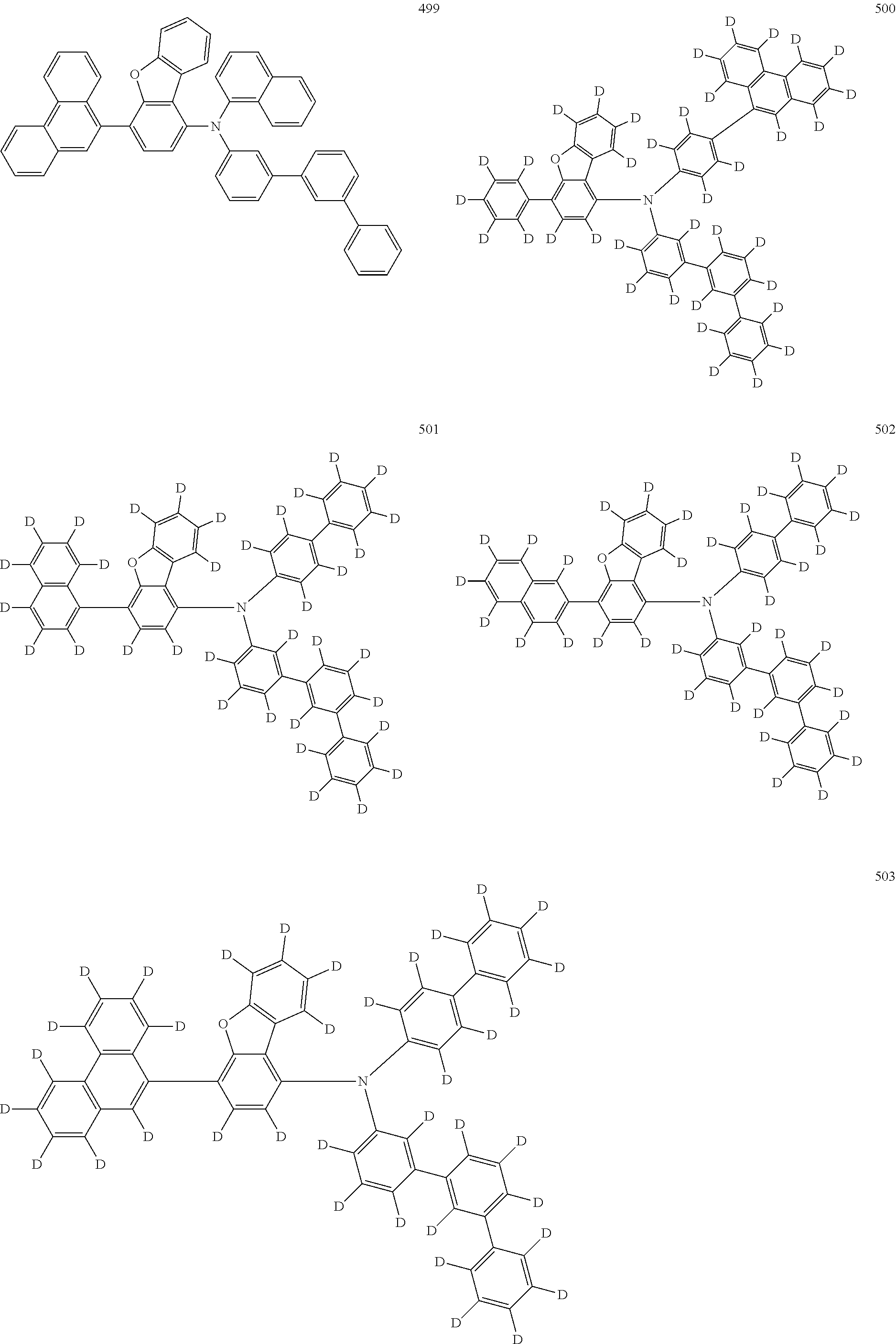

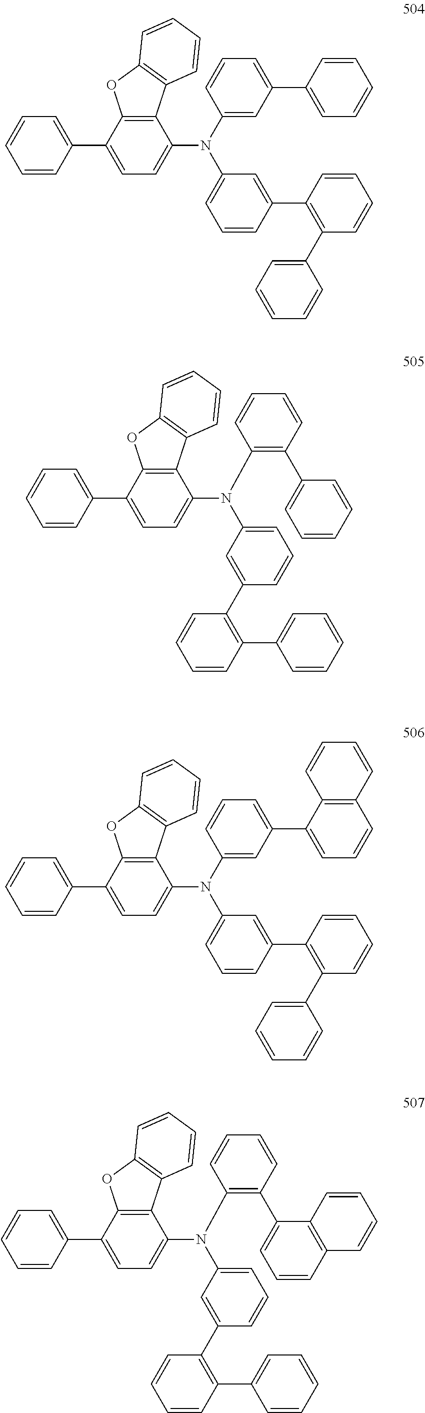 [SMILES: c1ccc(-c2cccc(N(c3cccc(-c4ccccc4-c4ccccc4)c3)c3ccc(-c4ccccc4)c4oc5ccccc5c34)c2)cc1 ; c1ccc(-c2ccccc2-c2cccc(N(c3ccccc3-c3ccccc3)c3ccc(-c4ccccc4)c4oc5ccccc5c34)c2)cc1 ; c1ccc(-c2ccccc2-c2cccc(N(c3cccc(-c4cccc5ccccc45)c3)c3ccc(-c4ccccc4)c4oc5ccccc5c34)c2)cc1 ; c1ccc(-c2ccccc2-c2cccc(N(c3ccccc3-c3cccc4ccccc34)c3ccc(-c4ccccc4)c4oc5ccccc5c34)c2)cc1]

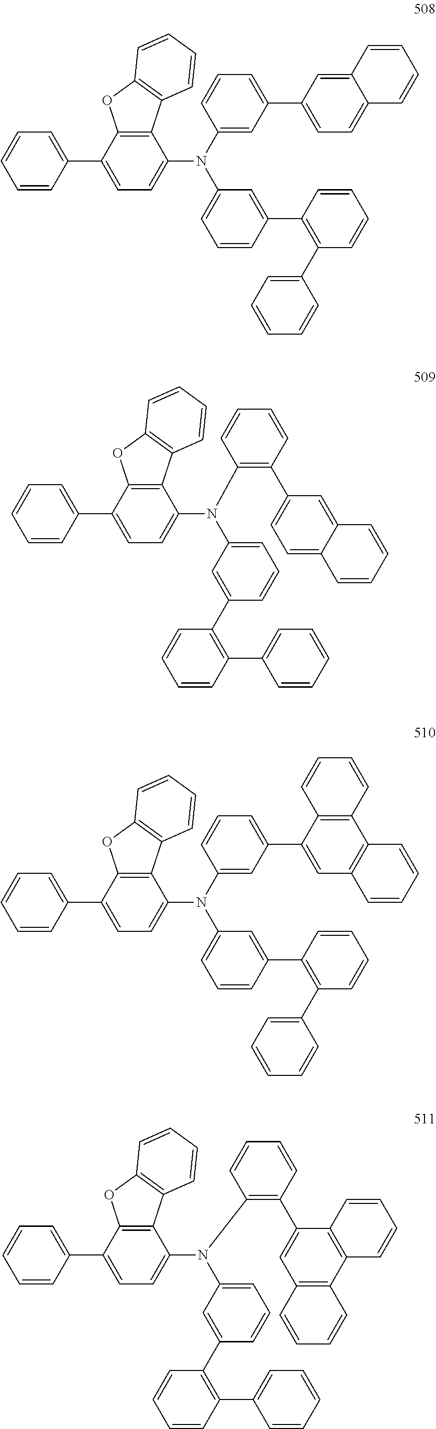 [SMILES: c1ccc(-c2ccccc2-c2cccc(N(c3cccc(-c4ccc5ccccc5c4)c3)c3ccc(-c4ccccc4)c4oc5ccccc5c34)c2)cc1 ; c1ccc(-c2ccccc2-c2cccc(N(c3ccccc3-c3ccc4ccccc4c3)c3ccc(-c4ccccc4)c4oc5ccccc5c34)c2)cc1 ; c1ccc(-c2ccccc2-c2cccc(N(c3cccc(-c4cc5ccccc5c5ccccc45)c3)c3ccc(-c4ccccc4)c4oc5ccccc5c34)c2)cc1 ; c1ccc(-c2ccccc2-c2cccc(N(c3ccccc3-c3cc4ccccc4c4ccccc34)c3ccc(-c4ccccc4)c4oc5ccccc5c34)c2)cc1]

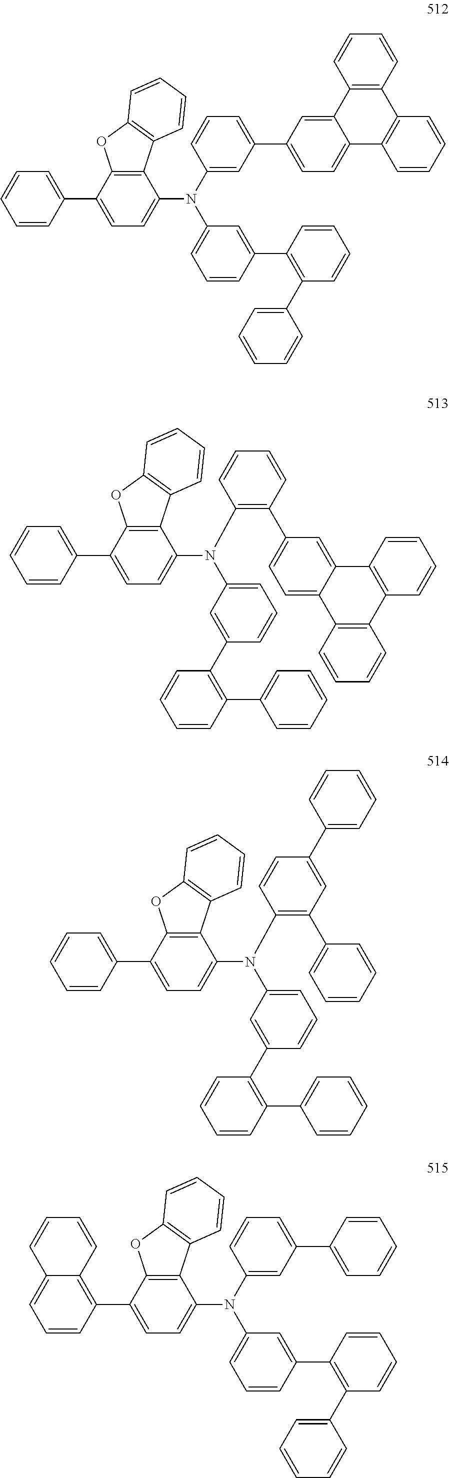

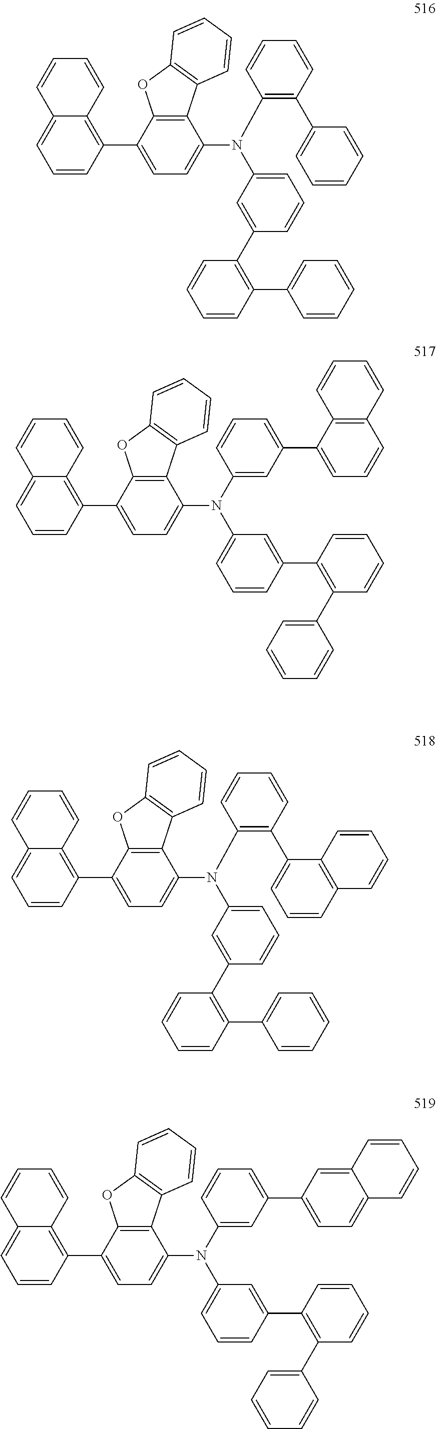

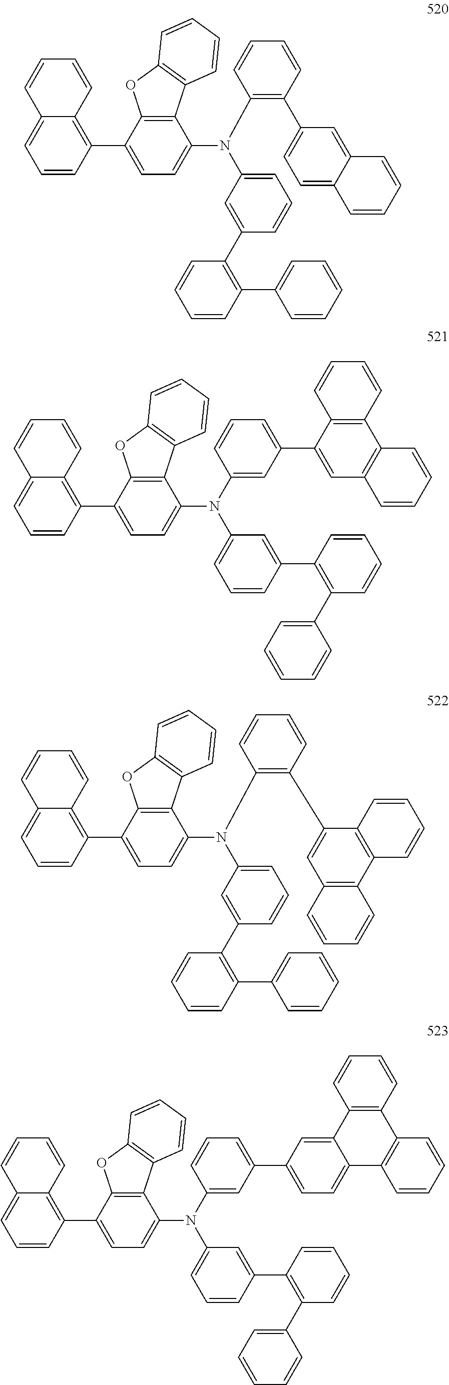

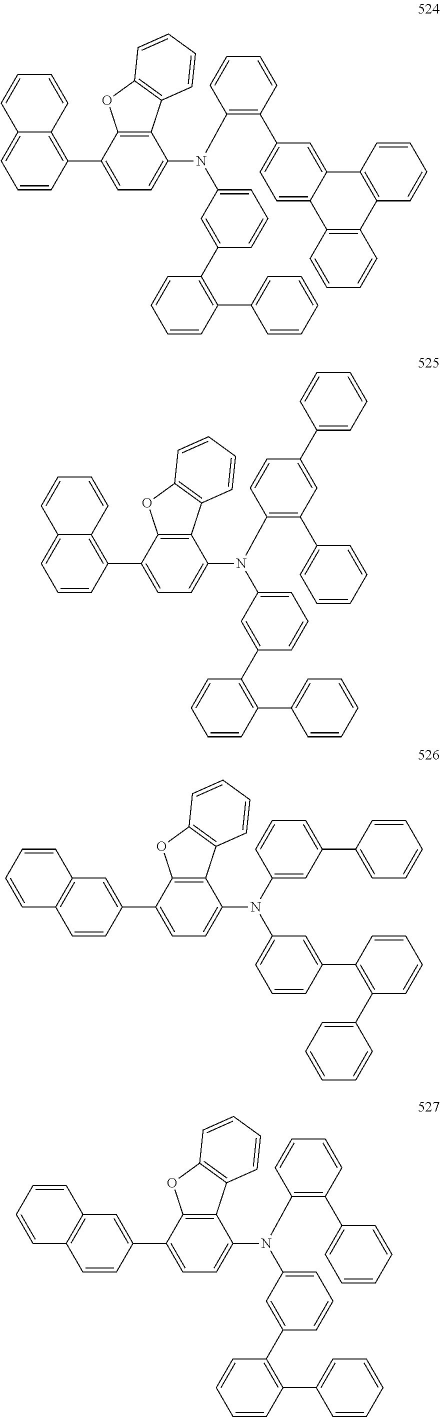

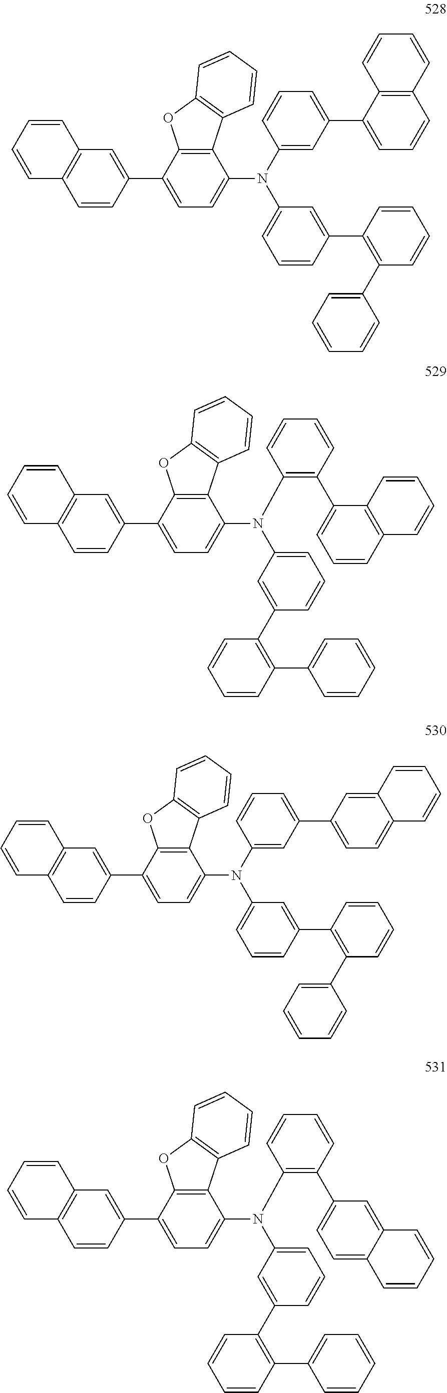 [SMILES: c1ccc(-c2ccccc2-c2cccc(N(c3cccc(-c4cccc5ccccc45)c3)c3ccc(-c4ccc5ccccc5c4)c4oc5ccccc5c34)c2)cc1 ; c1ccc(-c2ccccc2-c2cccc(N(c3ccccc3-c3cccc4ccccc34)c3ccc(-c4ccc5ccccc5c4)c4oc5ccccc5c34)c2)cc1 ; c1ccc(-c2ccccc2-c2cccc(N(c3cccc(-c4ccc5ccccc5c4)c3)c3ccc(-c4ccc5ccccc5c4)c4oc5ccccc5c34)c2)cc1 ; c1ccc(-c2ccccc2-c2cccc(N(c3ccccc3-c3ccc4ccccc4c3)c3ccc(-c4ccc5ccccc5c4)c4oc5ccccc5c34)c2)cc1]

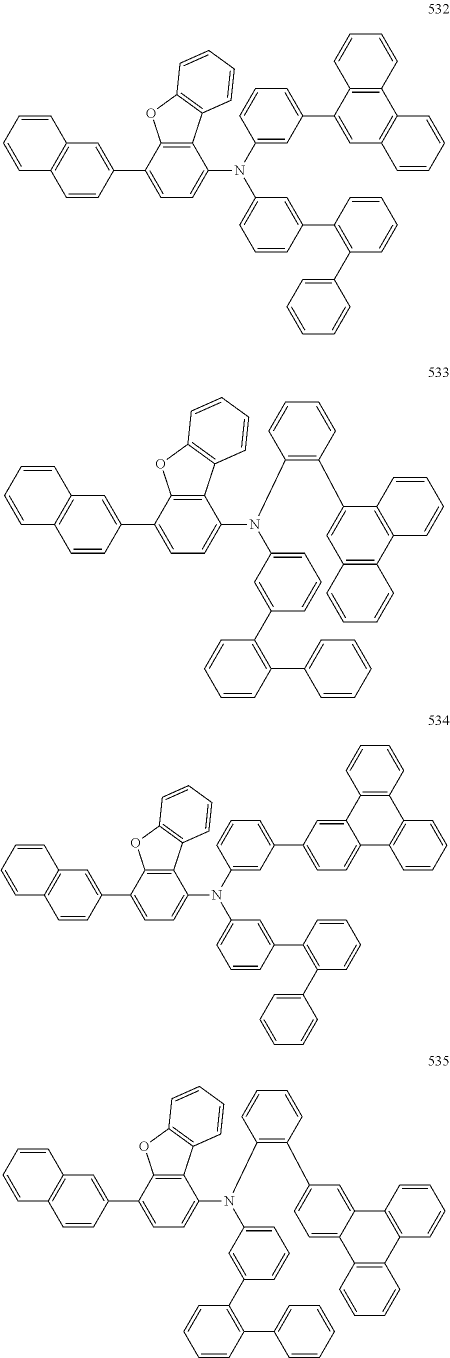 [SMILES: c1ccc(-c2ccccc2-c2cccc(N(c3cccc(-c4cc5ccccc5c5ccccc45)c3)c3ccc(-c4ccc5ccccc5c4)c4oc5ccccc5c34)c2)cc1 ; c1ccc(-c2ccccc2-c2cccc(N(c3ccccc3-c3cc4ccccc4c4ccccc34)c3ccc(-c4ccc5ccccc5c4)c4oc5ccccc5c34)c2)cc1 ; c1ccc(-c2ccccc2-c2cccc(N(c3cccc(-c4ccc5c6ccccc6c6ccccc6c5c4)c3)c3ccc(-c4ccc5ccccc5c4)c4oc5ccccc5c34)c2)cc1 ; c1ccc(-c2ccccc2-c2cccc(N(c3ccccc3-c3ccc4c5ccccc5c5ccccc5c4c3)c3ccc(-c4ccc5ccccc5c4)c4oc5ccccc5c34)c2)cc1]

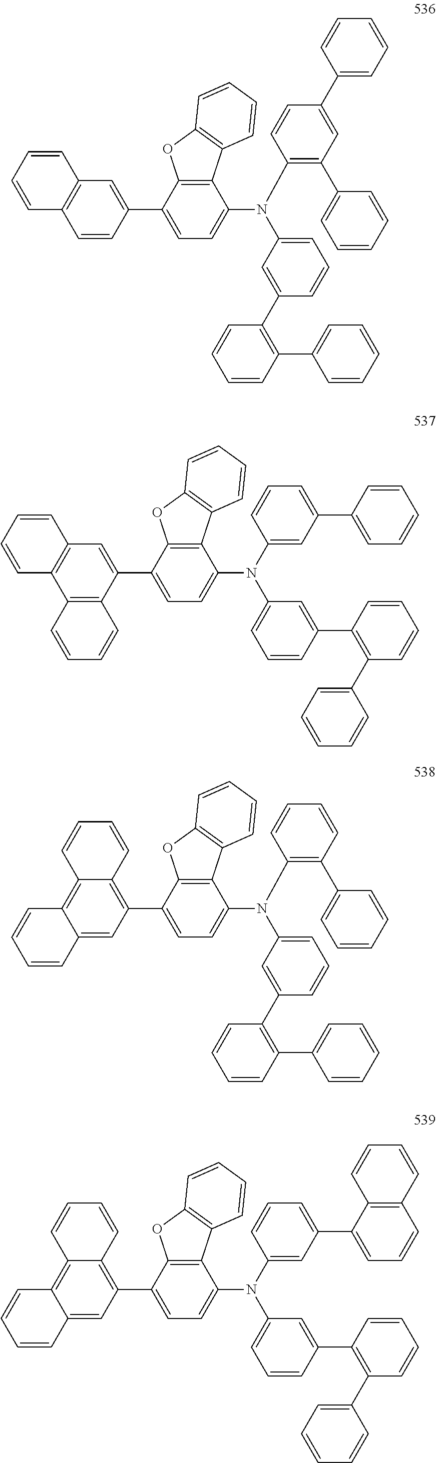 [SMILES: c1ccc(-c2ccc(N(c3cccc(-c4ccccc4-c4ccccc4)c3)c3ccc(-c4ccc5ccccc5c4)c4oc5ccccc5c34)c(-c3ccccc3)c2)cc1 ; c1ccc(-c2cccc(N(c3cccc(-c4ccccc4-c4ccccc4)c3)c3ccc(-c4cc5ccccc5c5ccccc45)c4oc5ccccc5c34)c2)cc1 ; c1ccc(-c2ccccc2-c2cccc(N(c3ccccc3-c3ccccc3)c3ccc(-c4cc5ccccc5c5ccccc45)c4oc5ccccc5c34)c2)cc1 ; c1ccc(-c2ccccc2-c2cccc(N(c3cccc(-c4cccc5ccccc45)c3)c3ccc(-c4cc5ccccc5c5ccccc45)c4oc5ccccc5c34)c2)cc1]

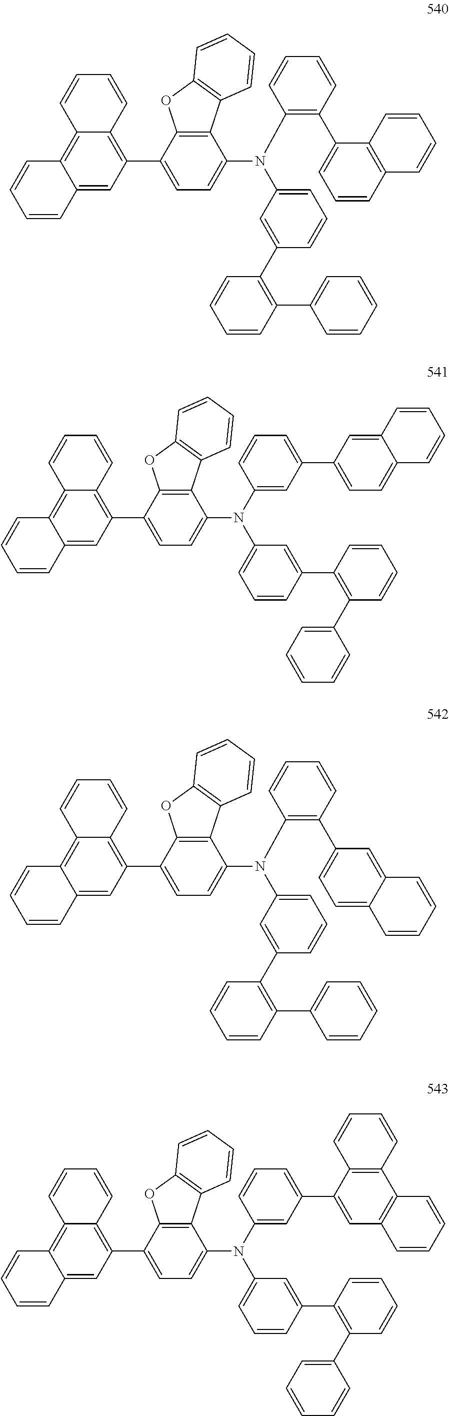 [SMILES: c1ccc(-c2ccccc2-c2cccc(N(c3ccccc3-c3cccc4ccccc34)c3ccc(-c4cc5ccccc5c5ccccc45)c4oc5ccccc5c34)c2)cc1 ; c1ccc(-c2ccccc2-c2cccc(N(c3cccc(-c4ccc5ccccc5c4)c3)c3ccc(-c4cc5ccccc5c5ccccc45)c4oc5ccccc5c34)c2)cc1 ; c1ccc(-c2ccccc2-c2cccc(N(c3ccccc3-c3ccc4ccccc4c3)c3ccc(-c4cc5ccccc5c5ccccc45)c4oc5ccccc5c34)c2)cc1 ; c1ccc(-c2ccccc2-c2cccc(N(c3cccc(-c4cc5ccccc5c5ccccc45)c3)c3ccc(-c4cc5ccccc5c5ccccc45)c4oc5ccccc5c34)c2)cc1]

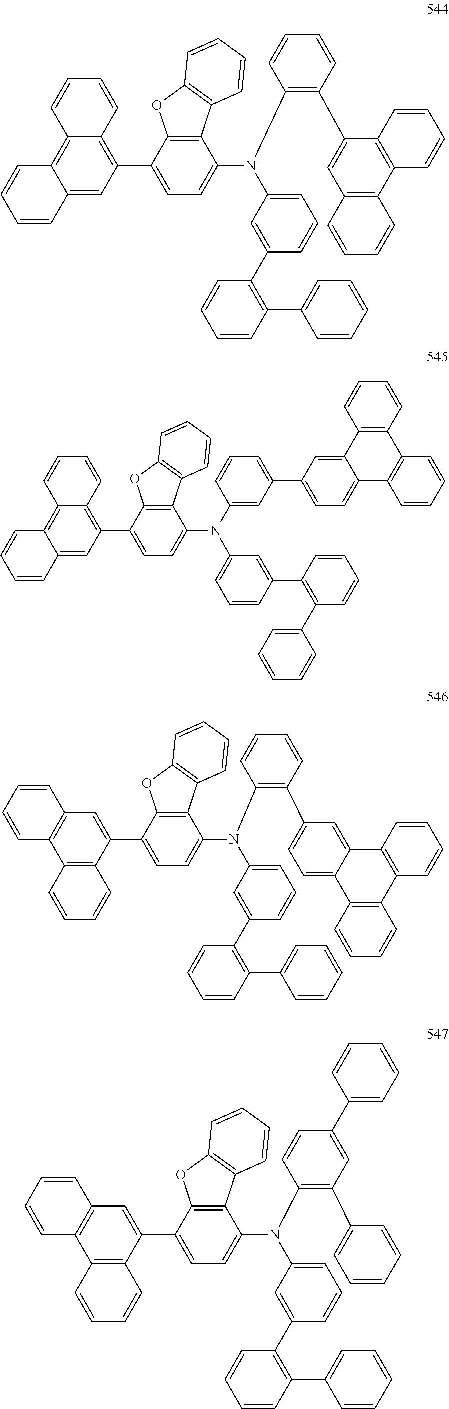 [SMILES: c1ccc(-c2ccccc2-c2cccc(N(c3ccccc3-c3cc4ccccc4c4ccccc34)c3ccc(-c4cc5ccccc5c5ccccc45)c4oc5ccccc5c34)c2)cc1 ; c1ccc(-c2ccccc2-c2cccc(N(c3cccc(-c4ccc5c6ccccc6c6ccccc6c5c4)c3)c3ccc(-c4cc5ccccc5c5ccccc45)c4oc5ccccc5c34)c2)cc1 ; c1ccc(-c2ccccc2-c2cccc(N(c3ccccc3-c3ccc4c5ccccc5c5ccccc5c4c3)c3ccc(-c4cc5ccccc5c5ccccc45)c4oc5ccccc5c34)c2)cc1 ; c1ccc(-c2ccc(N(c3cccc(-c4ccccc4-c4ccccc4)c3)c3ccc(-c4cc5ccccc5c5ccccc45)c4oc5ccccc5c34)c(-c3ccccc3)c2)cc1]

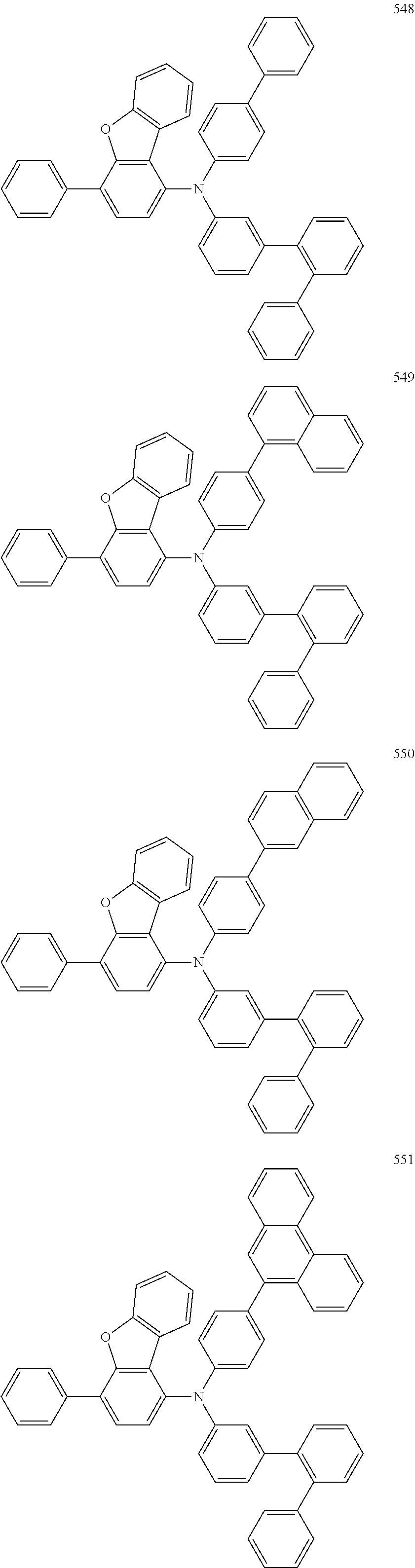

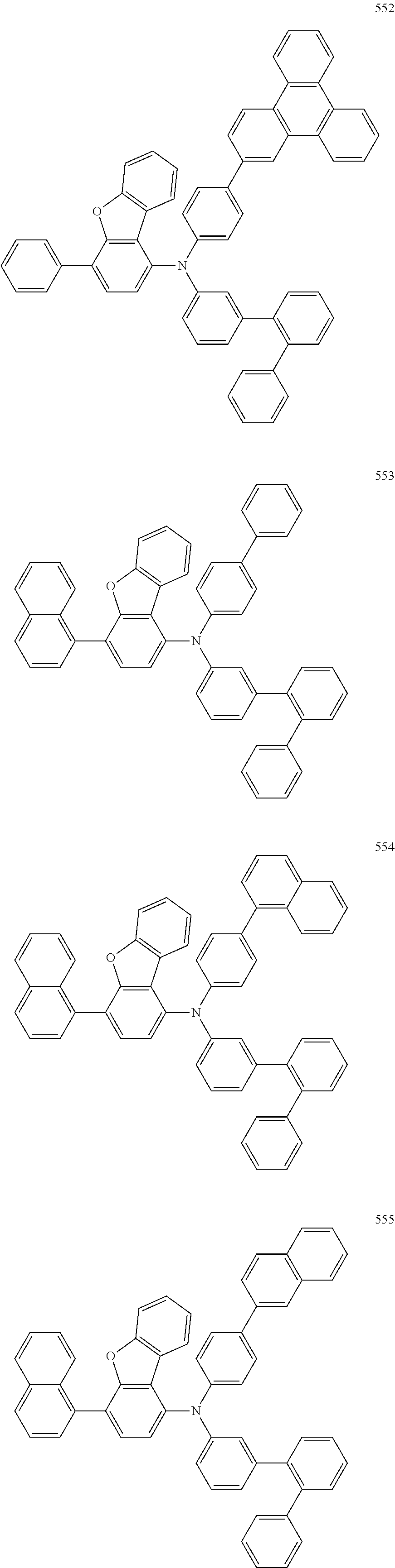 [SMILES: c1ccc(-c2ccccc2-c2cccc(N(c3ccc(-c4ccc5c6ccccc6c6ccccc6c5c4)cc3)c3ccc(-c4ccccc4)c4oc5ccccc5c34)c2)cc1 ; c1ccc(-c2ccc(N(c3cccc(-c4ccccc4-c4ccccc4)c3)c3ccc(-c4cccc5ccccc45)c4oc5ccccc5c34)cc2)cc1 ; c1ccc(-c2ccccc2-c2cccc(N(c3ccc(-c4cccc5ccccc45)cc3)c3ccc(-c4cccc5ccccc45)c4oc5ccccc5c34)c2)cc1 ; c1ccc(-c2ccccc2-c2cccc(N(c3ccc(-c4ccc5ccccc5c4)cc3)c3ccc(-c4cccc5ccccc45)c4oc5ccccc5c34)c2)cc1]

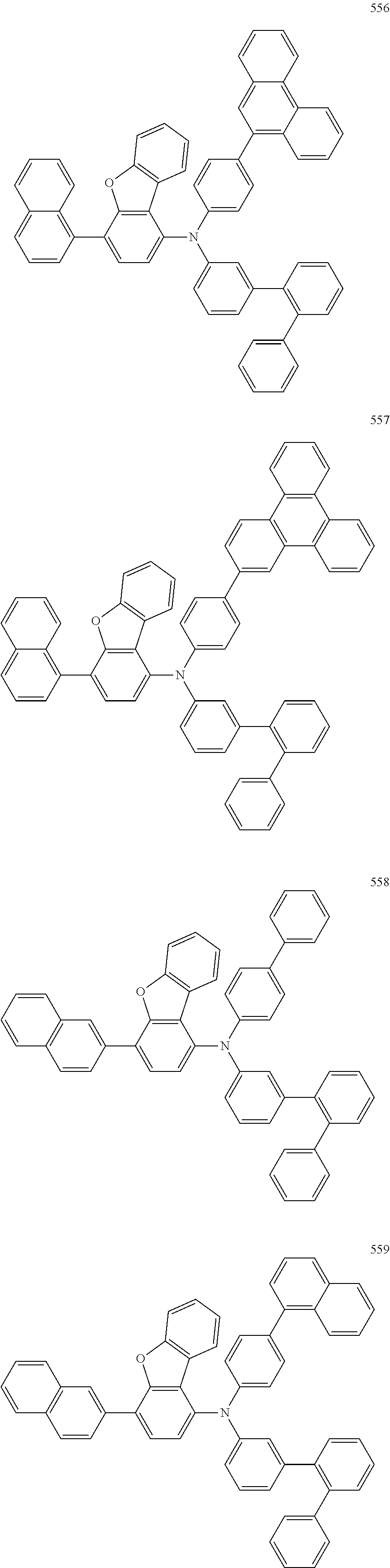 [SMILES: c1ccc(-c2ccccc2-c2cccc(N(c3ccc(-c4cc5ccccc5c5ccccc45)cc3)c3ccc(-c4cccc5ccccc45)c4oc5ccccc5c34)c2)cc1 ; c1ccc(-c2ccccc2-c2cccc(N(c3ccc(-c4ccc5c6ccccc6c6ccccc6c5c4)cc3)c3ccc(-c4cccc5ccccc45)c4oc5ccccc5c34)c2)cc1 ; c1ccc(-c2ccc(N(c3cccc(-c4ccccc4-c4ccccc4)c3)c3ccc(-c4ccc5ccccc5c4)c4oc5ccccc5c34)cc2)cc1 ; c1ccc(-c2ccccc2-c2cccc(N(c3ccc(-c4cccc5ccccc45)cc3)c3ccc(-c4ccc5ccccc5c4)c4oc5ccccc5c34)c2)cc1]

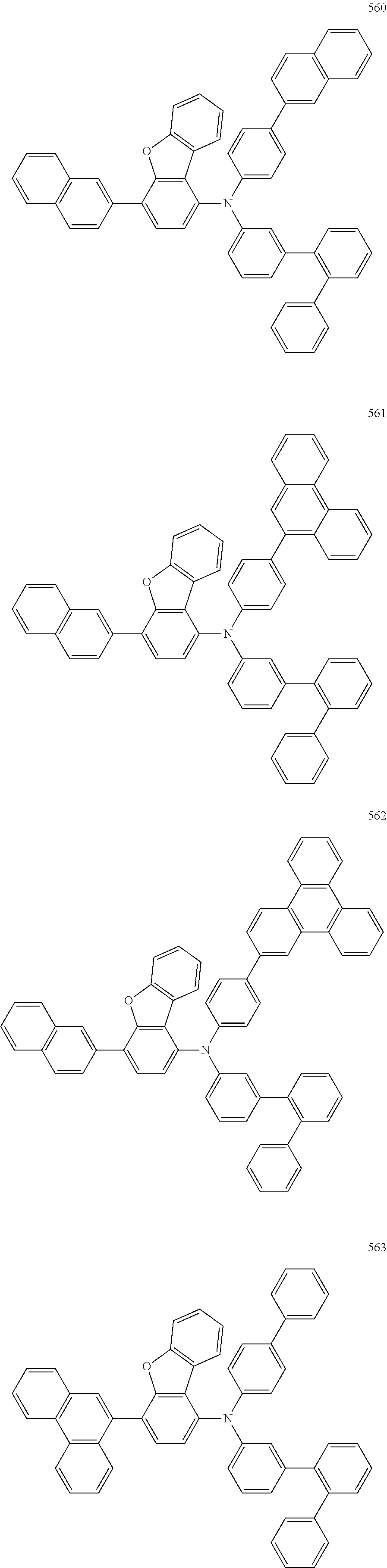 [SMILES: c1ccc(-c2ccccc2-c2cccc(N(c3ccc(-c4ccc5ccccc5c4)cc3)c3ccc(-c4ccc5ccccc5c4)c4oc5ccccc5c34)c2)cc1 ; c1ccc(-c2ccccc2-c2cccc(N(c3ccc(-c4cc5ccccc5c5ccccc45)cc3)c3ccc(-c4ccc5ccccc5c4)c4oc5ccccc5c34)c2)cc1 ; c1ccc(-c2ccccc2-c2cccc(N(c3ccc(-c4ccc5c6ccccc6c6ccccc6c5c4)cc3)c3ccc(-c4ccc5ccccc5c4)c4oc5ccccc5c34)c2)cc1 ; c1ccc(-c2ccc(N(c3cccc(-c4ccccc4-c4ccccc4)c3)c3ccc(-c4cc5ccccc5c5ccccc45)c4oc5ccccc5c34)cc2)cc1]

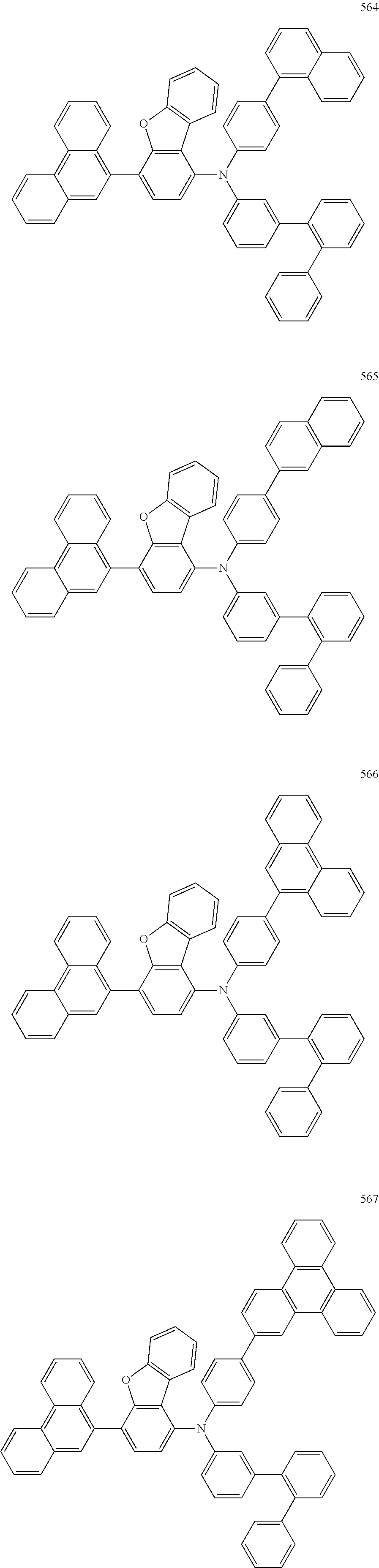 [SMILES: c1ccc(-c2ccccc2-c2cccc(N(c3ccc(-c4cccc5ccccc45)cc3)c3ccc(-c4cc5ccccc5c5ccccc45)c4oc5ccccc5c34)c2)cc1 ; c1ccc(-c2ccccc2-c2cccc(N(c3ccc(-c4ccc5ccccc5c4)cc3)c3ccc(-c4cc5ccccc5c5ccccc45)c4oc5ccccc5c34)c2)cc1 ; c1ccc(-c2ccccc2-c2cccc(N(c3ccc(-c4cc5ccccc5c5ccccc45)cc3)c3ccc(-c4cc5ccccc5c5ccccc45)c4oc5ccccc5c34)c2)cc1 ; c1ccc(-c2ccccc2-c2cccc(N(c3ccc(-c4ccc5c6ccccc6c6ccccc6c5c4)cc3)c3ccc(-c4cc5ccccc5c5ccccc45)c4oc5ccccc5c34)c2)cc1]

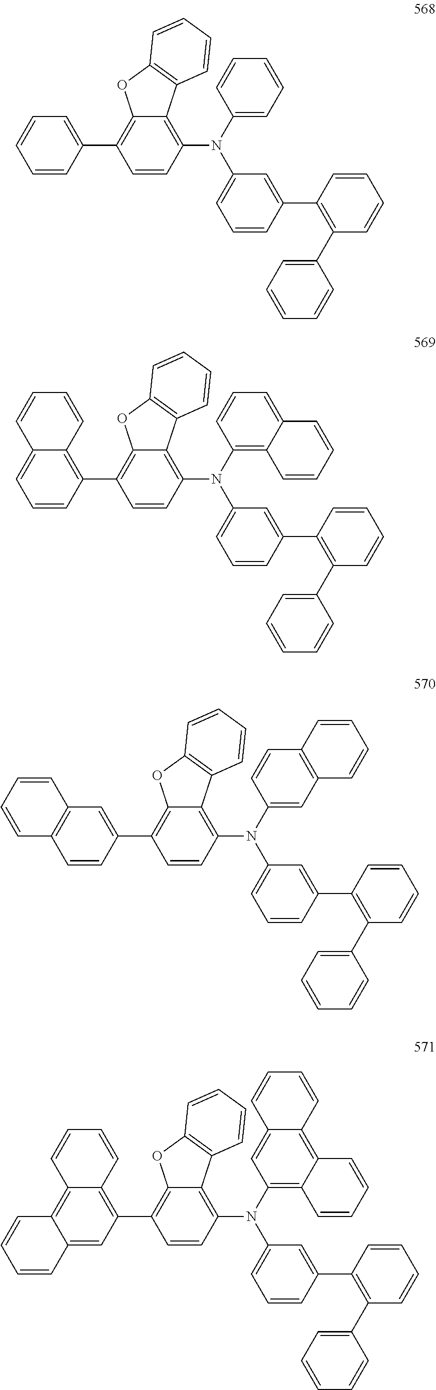 [SMILES: c1ccc(-c2ccccc2-c2cccc(N(c3ccccc3)c3ccc(-c4ccccc4)c4oc5ccccc5c34)c2)cc1 ; c1ccc(-c2ccccc2-c2cccc(N(c3cccc4ccccc34)c3ccc(-c4cccc5ccccc45)c4oc5ccccc5c34)c2)cc1 ; c1ccc(-c2ccccc2-c2cccc(N(c3ccc4ccccc4c3)c3ccc(-c4ccc5ccccc5c4)c4oc5ccccc5c34)c2)cc1 ; c1ccc(-c2ccccc2-c2cccc(N(c3cc4ccccc4c4ccccc34)c3ccc(-c4cc5ccccc5c5ccccc45)c4oc5ccccc5c34)c2)cc1]

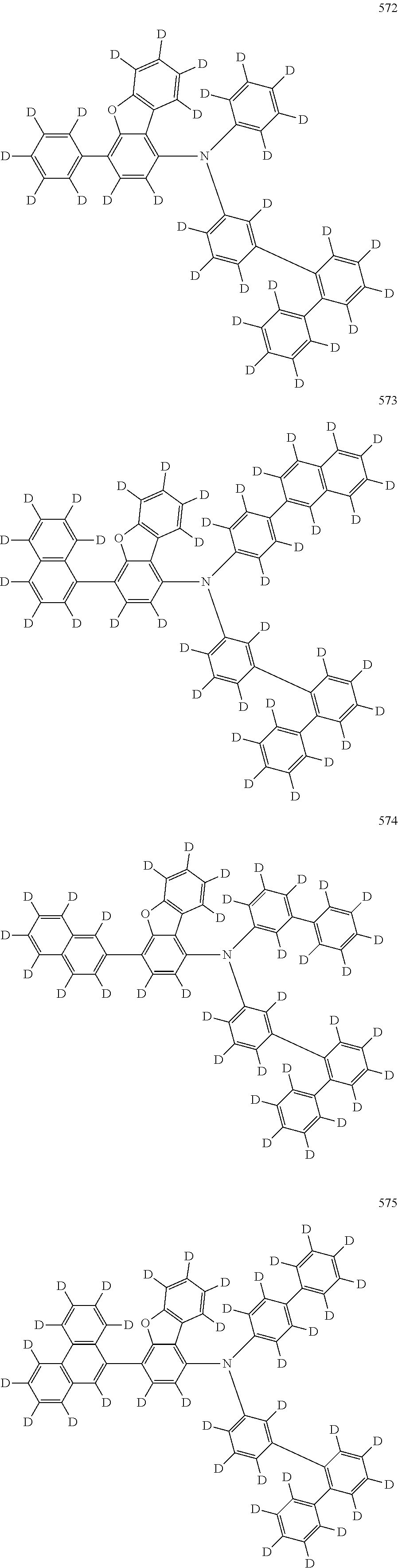

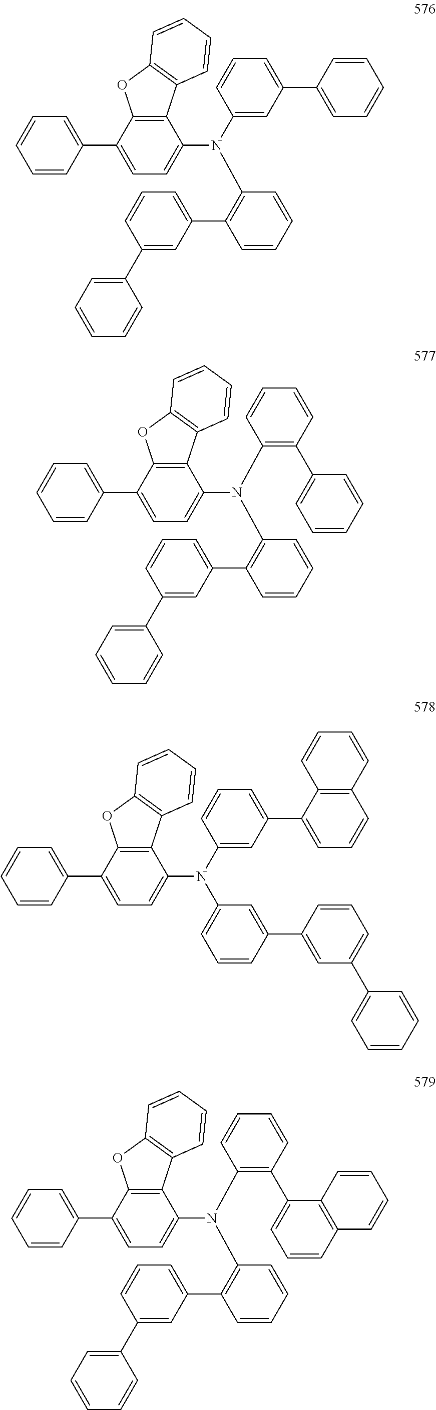 [SMILES: c1ccc(-c2cccc(-c3ccccc3N(c3cccc(-c4ccccc4)c3)c3ccc(-c4ccccc4)c4oc5ccccc5c34)c2)cc1 ; c1ccc(-c2cccc(-c3ccccc3N(c3ccccc3-c3ccccc3)c3ccc(-c4ccccc4)c4oc5ccccc5c34)c2)cc1 ; c1ccc(-c2cccc(-c3cccc(N(c4cccc(-c5cccc6ccccc56)c4)c4ccc(-c5ccccc5)c5oc6ccccc6c45)c3)c2)cc1 ; c1ccc(-c2cccc(-c3ccccc3N(c3ccccc3-c3cccc4ccccc34)c3ccc(-c4ccccc4)c4oc5ccccc5c34)c2)cc1]

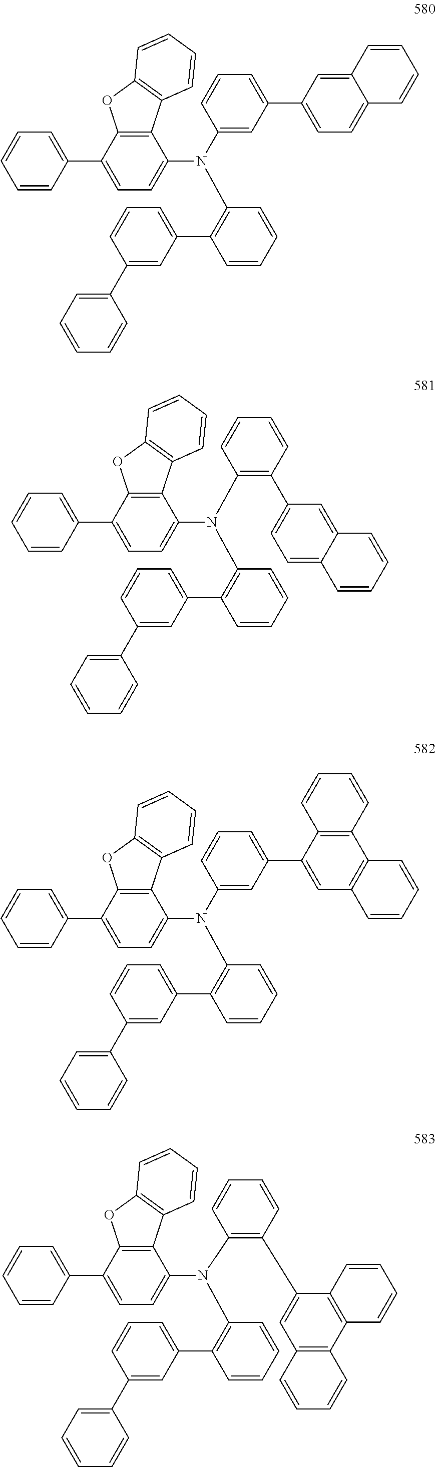 [SMILES: c1ccc(-c2cccc(-c3ccccc3N(c3cccc(-c4ccc5ccccc5c4)c3)c3ccc(-c4ccccc4)c4oc5ccccc5c34)c2)cc1 ; c1ccc(-c2cccc(-c3ccccc3N(c3ccccc3-c3ccc4ccccc4c3)c3ccc(-c4ccccc4)c4oc5ccccc5c34)c2)cc1 ; c1ccc(-c2cccc(-c3ccccc3N(c3cccc(-c4cc5ccccc5c5ccccc45)c3)c3ccc(-c4ccccc4)c4oc5ccccc5c34)c2)cc1 ; c1ccc(-c2cccc(-c3ccccc3N(c3ccccc3-c3cc4ccccc4c4ccccc34)c3ccc(-c4ccccc4)c4oc5ccccc5c34)c2)cc1]

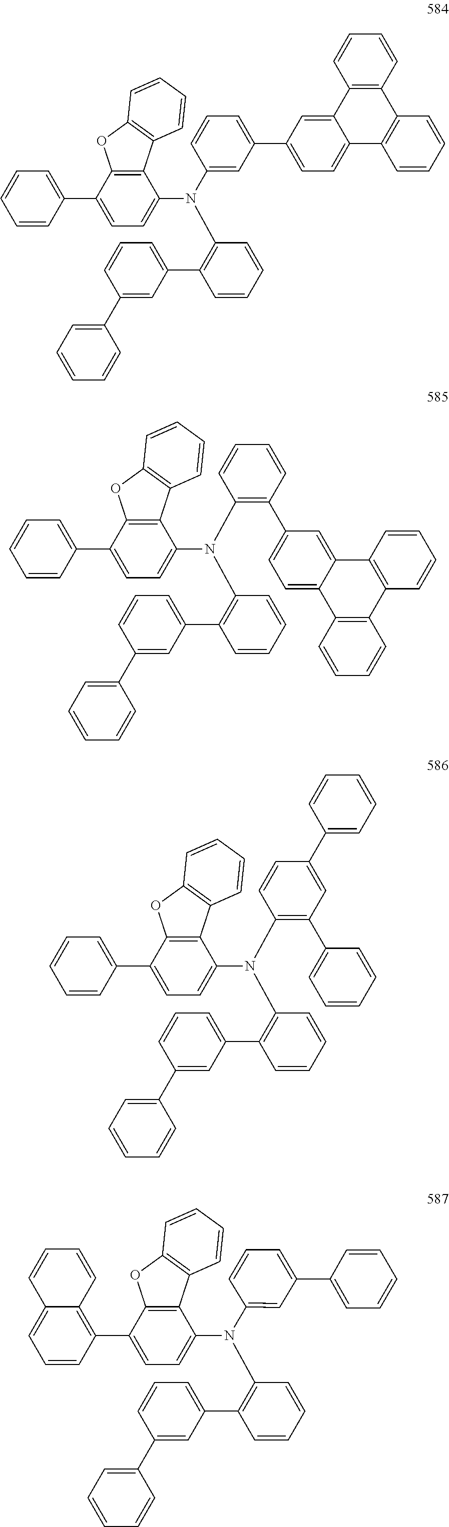 [SMILES: c1ccc(-c2cccc(-c3ccccc3N(c3cccc(-c4ccc5c6ccccc6c6ccccc6c5c4)c3)c3ccc(-c4ccccc4)c4oc5ccccc5c34)c2)cc1 ; c1ccc(-c2cccc(-c3ccccc3N(c3ccccc3-c3ccc4c5ccccc5c5ccccc5c4c3)c3ccc(-c4ccccc4)c4oc5ccccc5c34)c2)cc1 ; c1ccc(-c2cccc(-c3ccccc3N(c3ccc(-c4ccccc4)cc3-c3ccccc3)c3ccc(-c4ccccc4)c4oc5ccccc5c34)c2)cc1 ; c1ccc(-c2cccc(-c3ccccc3N(c3cccc(-c4ccccc4)c3)c3ccc(-c4cccc5ccccc45)c4oc5ccccc5c34)c2)cc1]

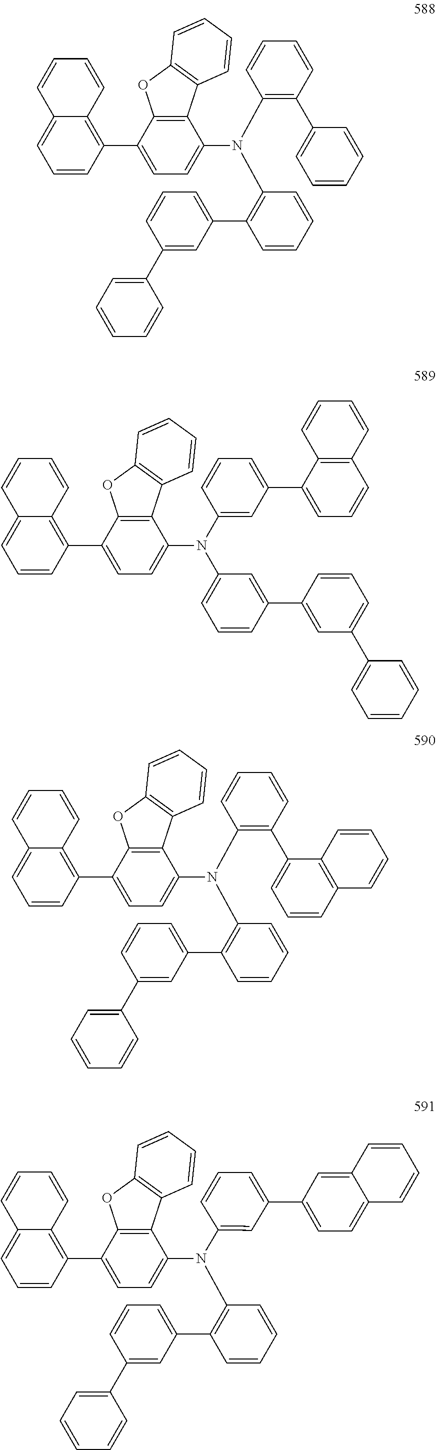 [SMILES: c1ccc(-c2cccc(-c3ccccc3N(c3ccccc3-c3ccccc3)c3ccc(-c4cccc5ccccc45)c4oc5ccccc5c34)c2)cc1 ; c1ccc(-c2cccc(-c3cccc(N(c4cccc(-c5cccc6ccccc56)c4)c4ccc(-c5cccc6ccccc56)c5oc6ccccc6c45)c3)c2)cc1 ; c1ccc(-c2cccc(-c3ccccc3N(c3ccccc3-c3cccc4ccccc34)c3ccc(-c4cccc5ccccc45)c4oc5ccccc5c34)c2)cc1 ; c1ccc(-c2cccc(-c3ccccc3N(c3cccc(-c4ccc5ccccc5c4)c3)c3ccc(-c4cccc5ccccc45)c4oc5ccccc5c34)c2)cc1]

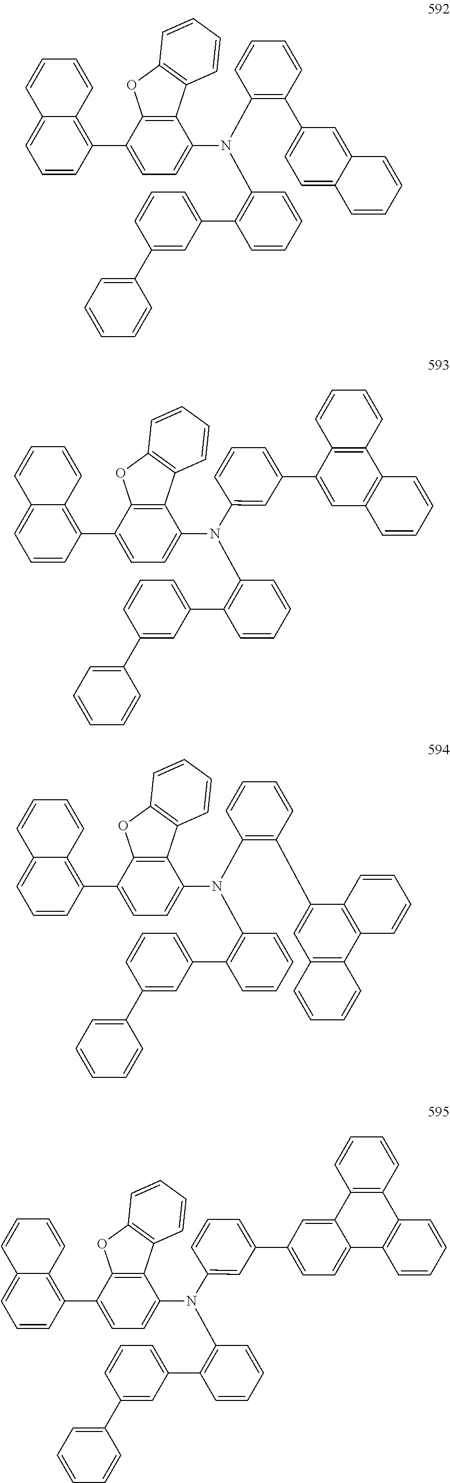 [SMILES: c1ccc(-c2cccc(-c3ccccc3N(c3ccccc3-c3ccc4ccccc4c3)c3ccc(-c4cccc5ccccc45)c4oc5ccccc5c34)c2)cc1 ; c1ccc(-c2cccc(-c3ccccc3N(c3cccc(-c4cc5ccccc5c5ccccc45)c3)c3ccc(-c4cccc5ccccc45)c4oc5ccccc5c34)c2)cc1 ; c1ccc(-c2cccc(-c3ccccc3N(c3ccccc3-c3cc4ccccc4c4ccccc34)c3ccc(-c4cccc5ccccc45)c4oc5ccccc5c34)c2)cc1 ; c1ccc(-c2cccc(-c3ccccc3N(c3cccc(-c4ccc5c6ccccc6c6ccccc6c5c4)c3)c3ccc(-c4cccc5ccccc45)c4oc5ccccc5c34)c2)cc1]

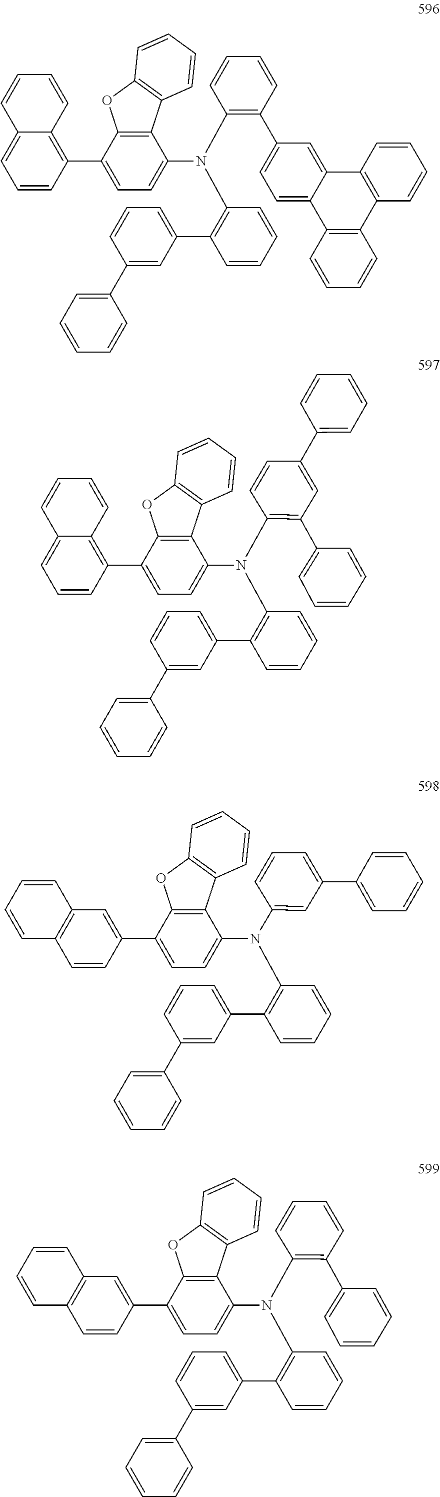 [SMILES: c1ccc(-c2cccc(-c3ccccc3N(c3ccccc3-c3ccc4c5ccccc5c5ccccc5c4c3)c3ccc(-c4cccc5ccccc45)c4oc5ccccc5c34)c2)cc1 ; c1ccc(-c2cccc(-c3ccccc3N(c3ccc(-c4ccccc4)cc3-c3ccccc3)c3ccc(-c4cccc5ccccc45)c4oc5ccccc5c34)c2)cc1 ; c1ccc(-c2cccc(-c3ccccc3N(c3cccc(-c4ccccc4)c3)c3ccc(-c4ccc5ccccc5c4)c4oc5ccccc5c34)c2)cc1 ; c1ccc(-c2cccc(-c3ccccc3N(c3ccccc3-c3ccccc3)c3ccc(-c4ccc5ccccc5c4)c4oc5ccccc5c34)c2)cc1]

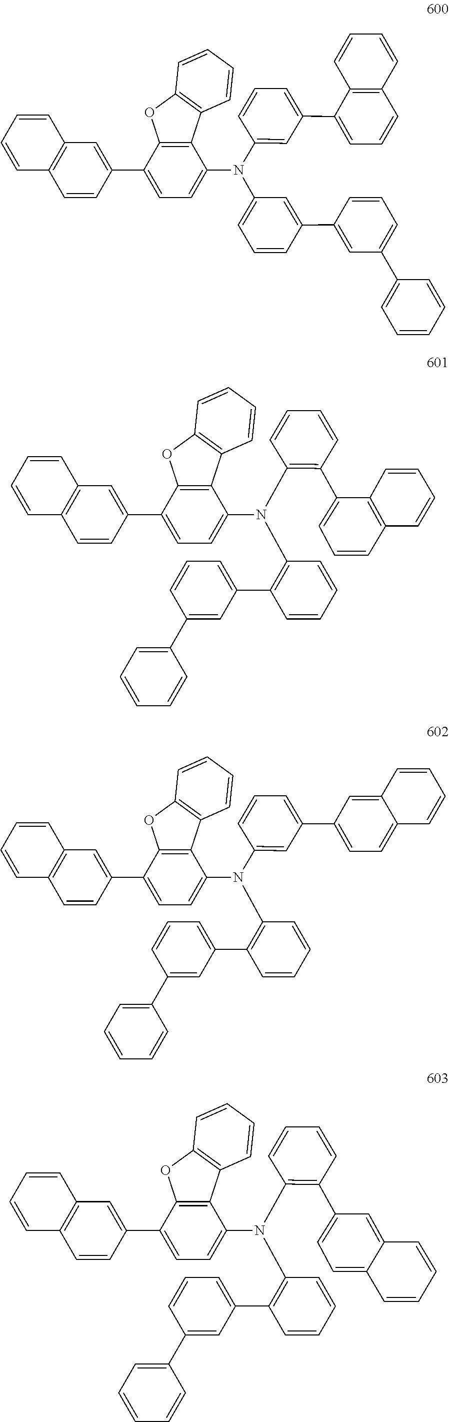 [SMILES: c1ccc(-c2cccc(-c3cccc(N(c4cccc(-c5cccc6ccccc56)c4)c4ccc(-c5ccc6ccccc6c5)c5oc6ccccc6c45)c3)c2)cc1 ; c1ccc(-c2cccc(-c3ccccc3N(c3ccccc3-c3cccc4ccccc34)c3ccc(-c4ccc5ccccc5c4)c4oc5ccccc5c34)c2)cc1 ; c1ccc(-c2cccc(-c3ccccc3N(c3cccc(-c4ccc5ccccc5c4)c3)c3ccc(-c4ccc5ccccc5c4)c4oc5ccccc5c34)c2)cc1 ; c1ccc(-c2cccc(-c3ccccc3N(c3ccccc3-c3ccc4ccccc4c3)c3ccc(-c4ccc5ccccc5c4)c4oc5ccccc5c34)c2)cc1]

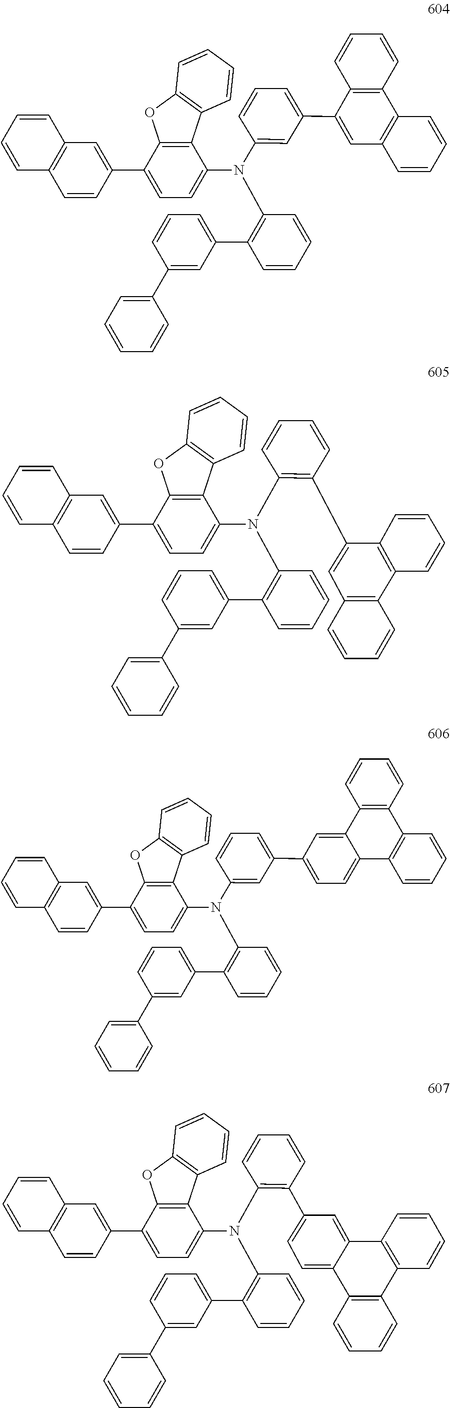 [SMILES: c1ccc(-c2cccc(-c3ccccc3N(c3cccc(-c4cc5ccccc5c5ccccc45)c3)c3ccc(-c4ccc5ccccc5c4)c4oc5ccccc5c34)c2)cc1 ; c1ccc(-c2cccc(-c3ccccc3N(c3ccccc3-c3cc4ccccc4c4ccccc34)c3ccc(-c4ccc5ccccc5c4)c4oc5ccccc5c34)c2)cc1 ; c1ccc(-c2cccc(-c3ccccc3N(c3cccc(-c4ccc5c6ccccc6c6ccccc6c5c4)c3)c3ccc(-c4ccc5ccccc5c4)c4oc5ccccc5c34)c2)cc1 ; c1ccc(-c2cccc(-c3ccccc3N(c3ccccc3-c3ccc4c5ccccc5c5ccccc5c4c3)c3ccc(-c4ccc5ccccc5c4)c4oc5ccccc5c34)c2)cc1]

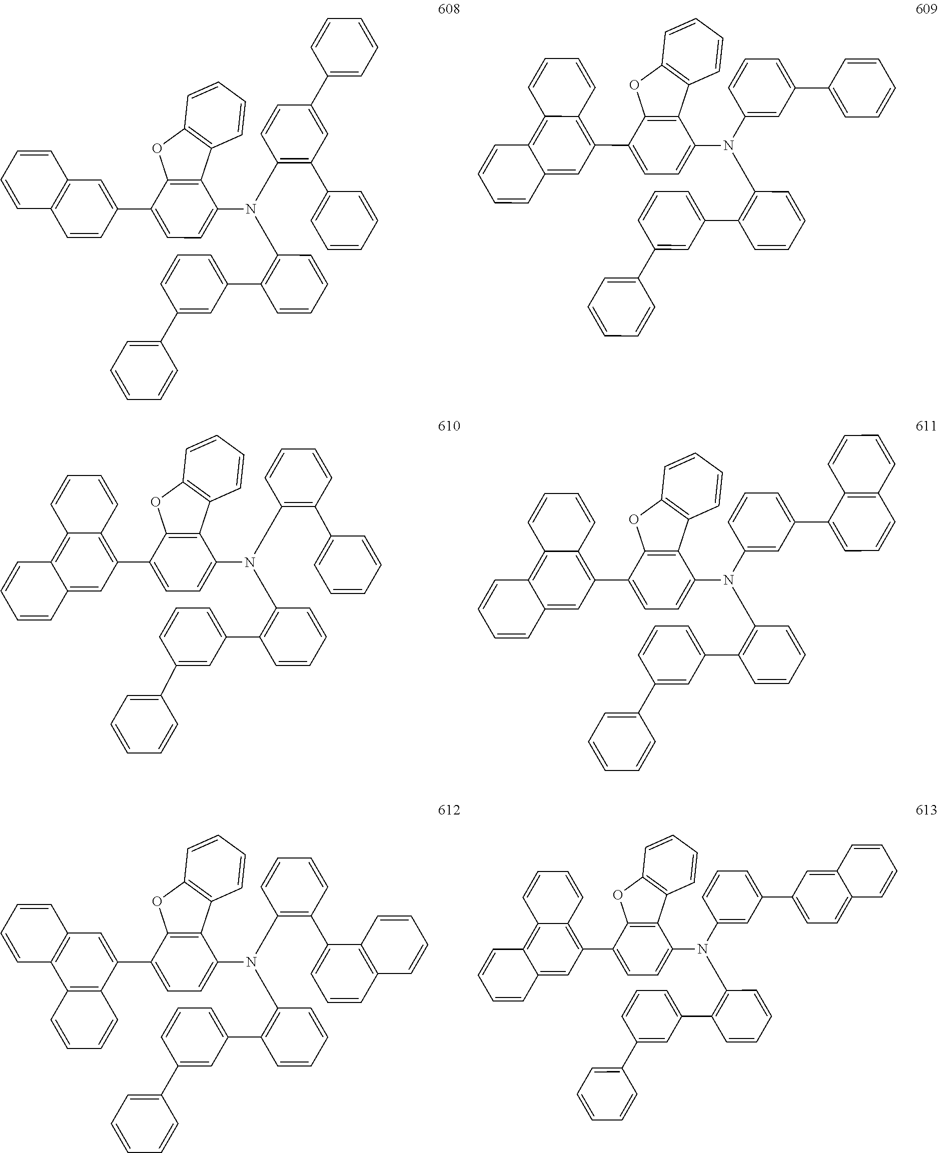

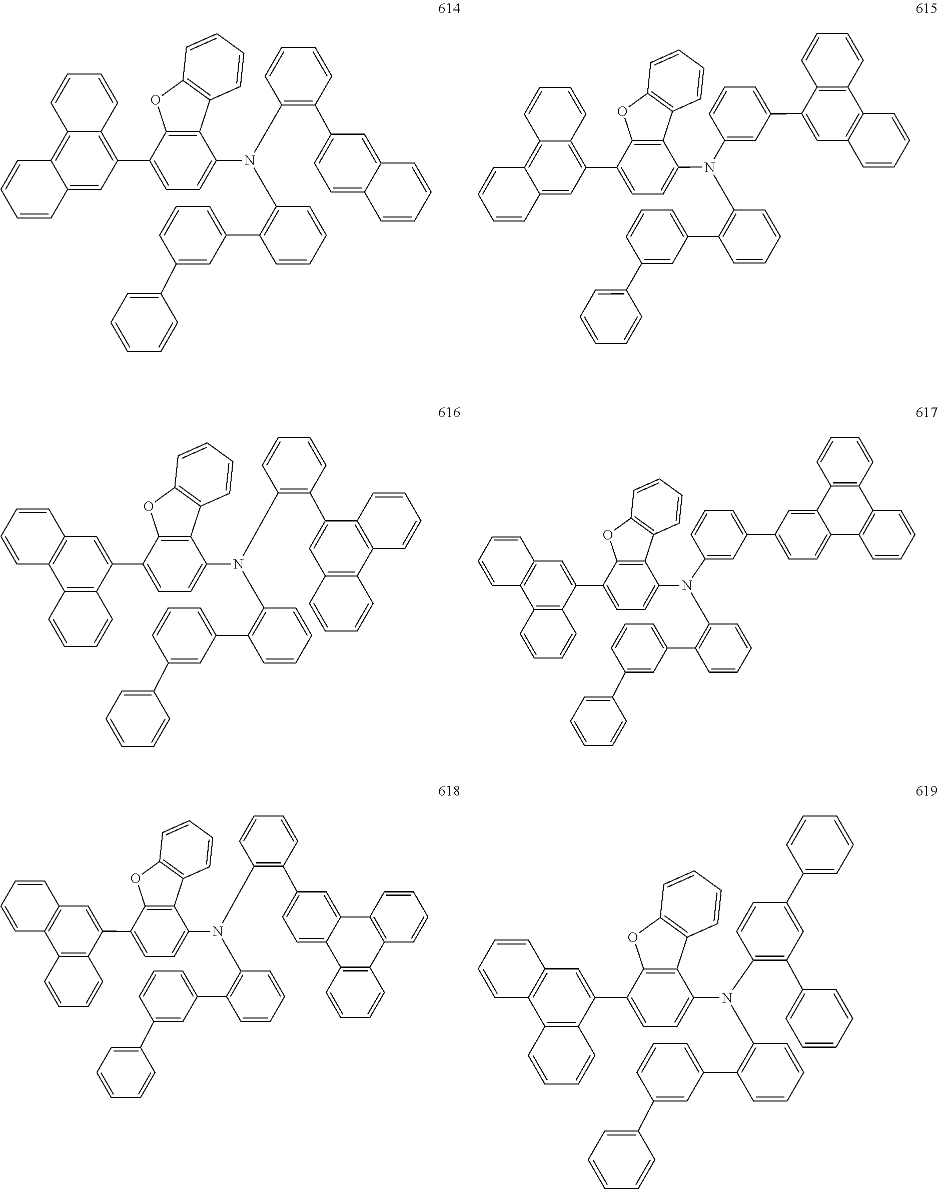

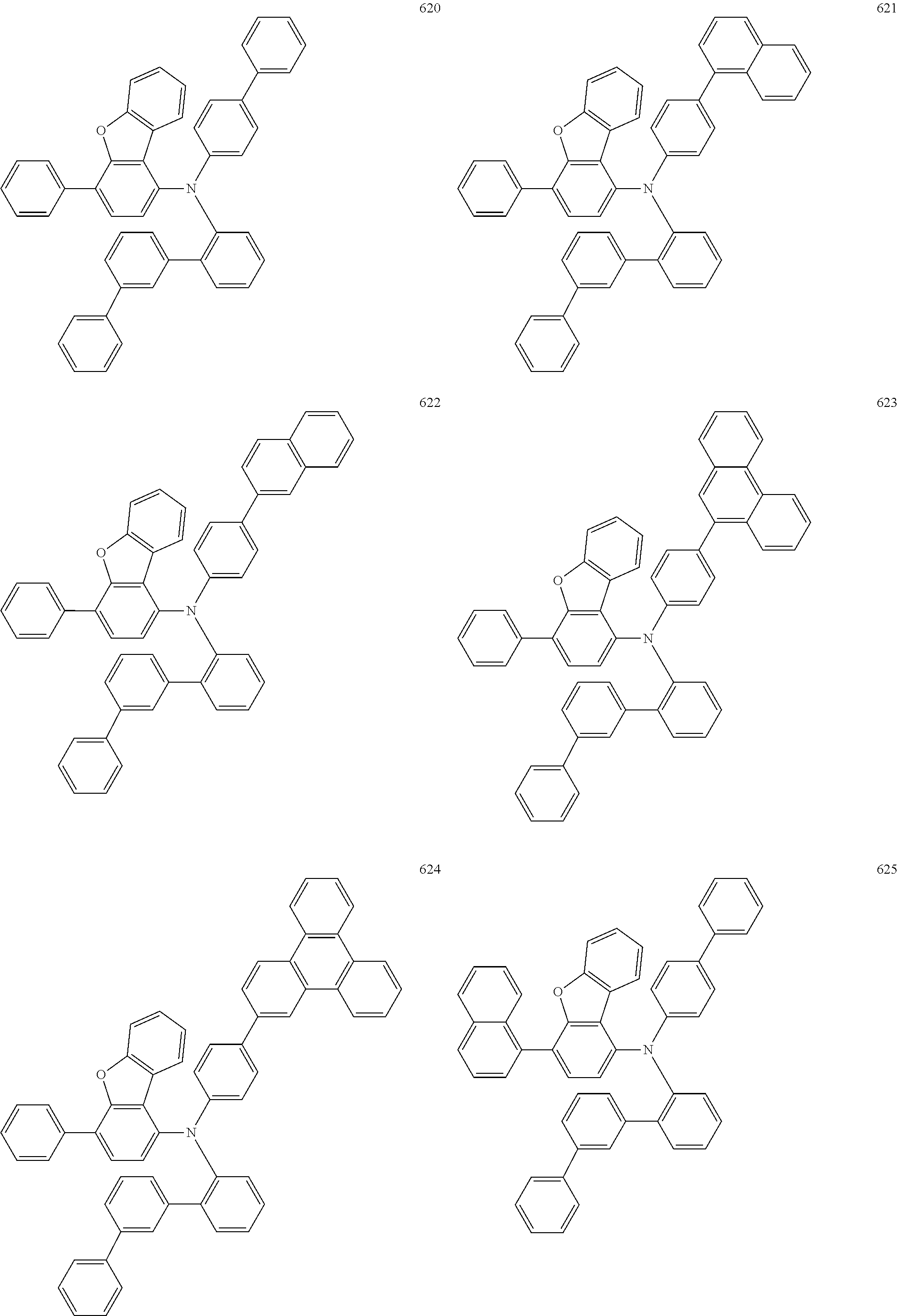

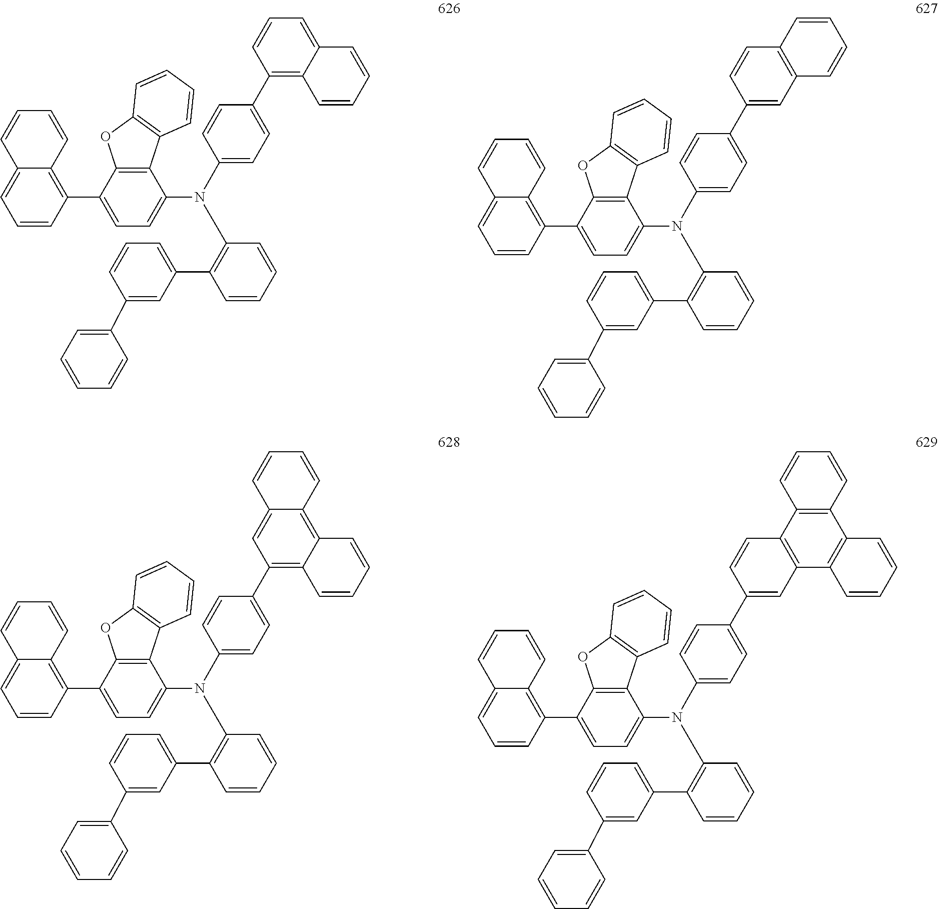 [SMILES: c1ccc(-c2cccc(-c3ccccc3N(c3ccc(-c4cccc5ccccc45)cc3)c3ccc(-c4cccc5ccccc45)c4oc5ccccc5c34)c2)cc1 ; c1ccc(-c2cccc(-c3ccccc3N(c3ccc(-c4ccc5ccccc5c4)cc3)c3ccc(-c4cccc5ccccc45)c4oc5ccccc5c34)c2)cc1 ; c1ccc(-c2cccc(-c3ccccc3N(c3ccc(-c4cc5ccccc5c5ccccc45)cc3)c3ccc(-c4cccc5ccccc45)c4oc5ccccc5c34)c2)cc1 ; c1ccc(-c2cccc(-c3ccccc3N(c3ccc(-c4ccc5c6ccccc6c6ccccc6c5c4)cc3)c3ccc(-c4cccc5ccccc45)c4oc5ccccc5c34)c2)cc1]

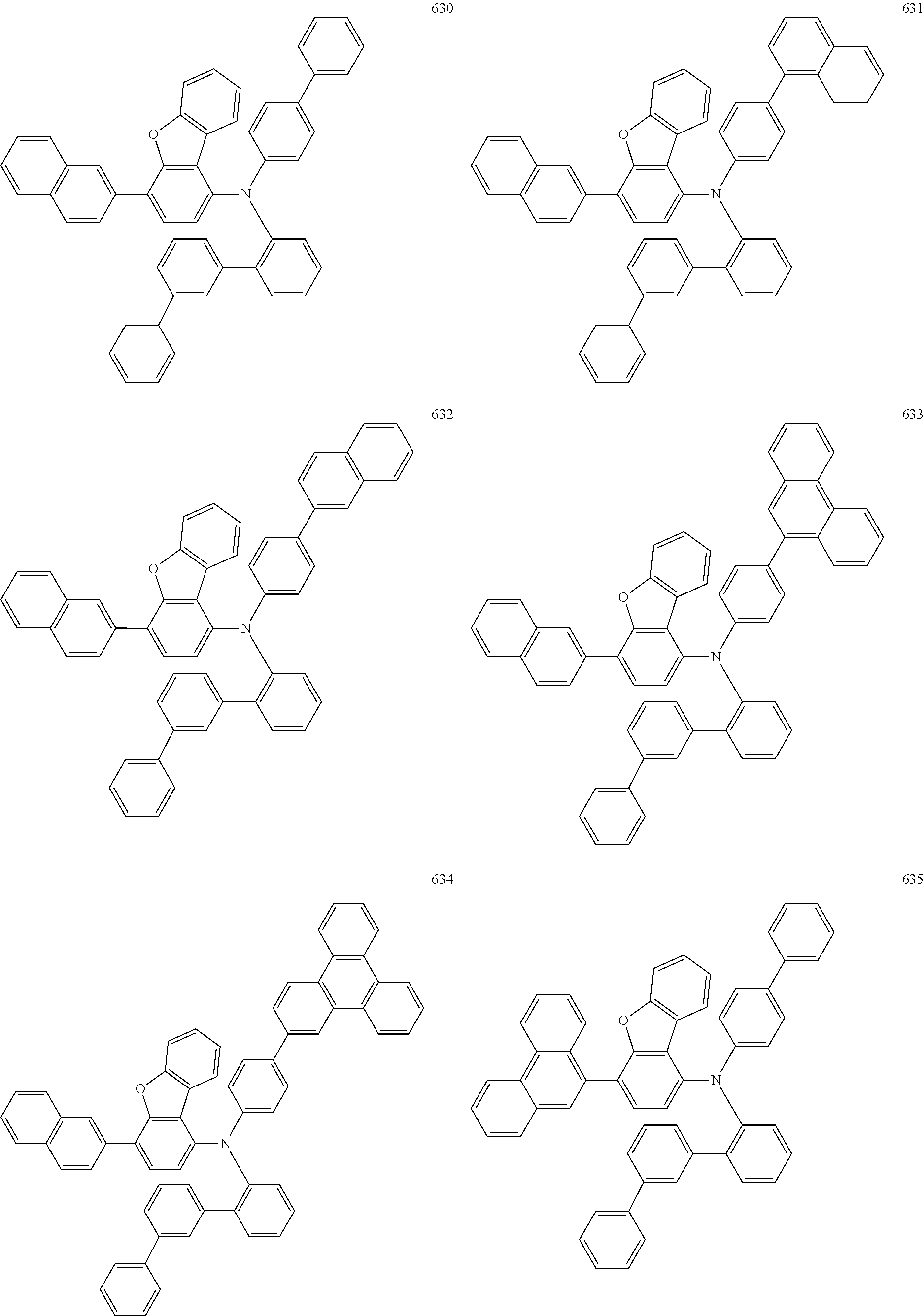

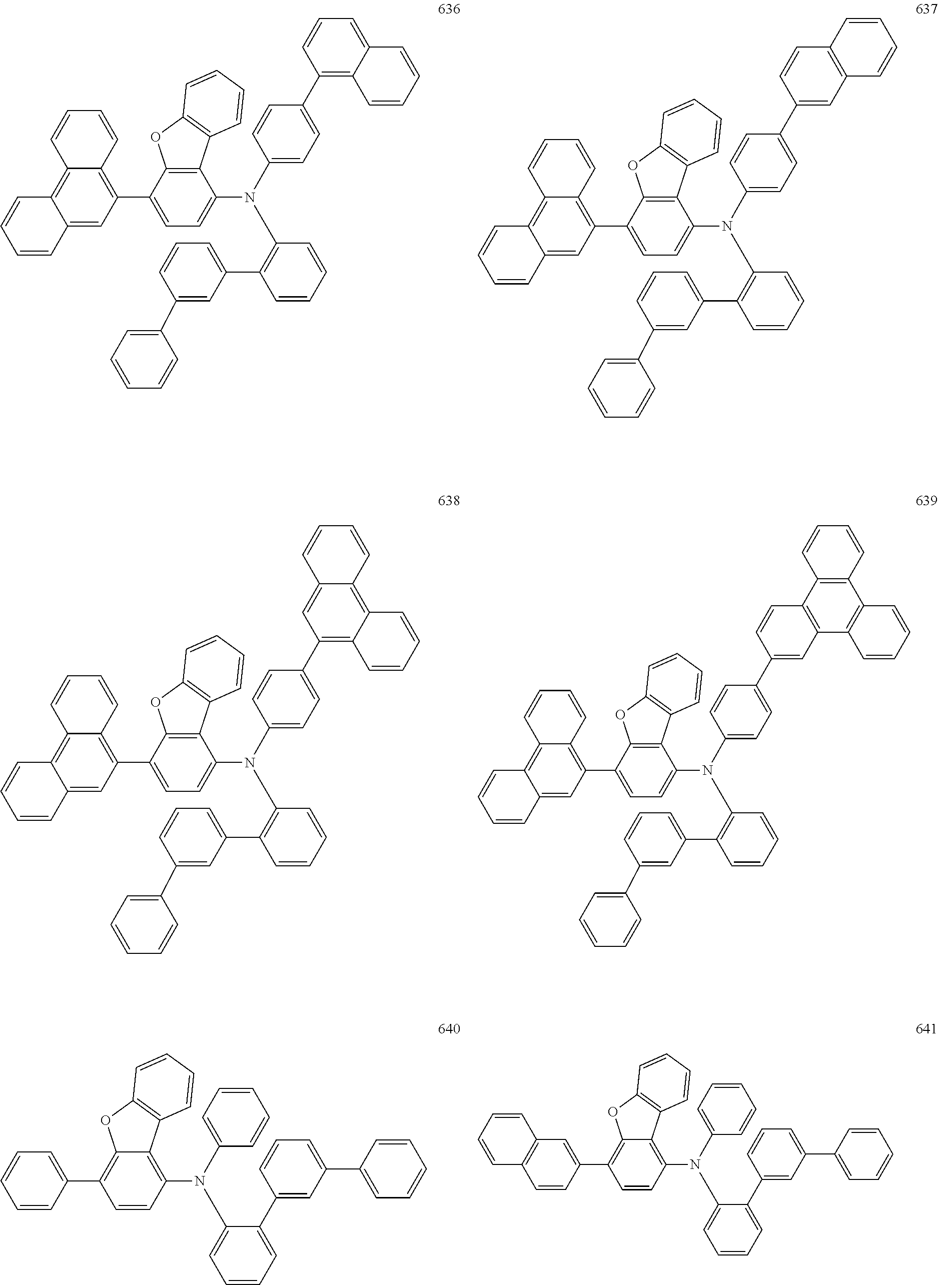

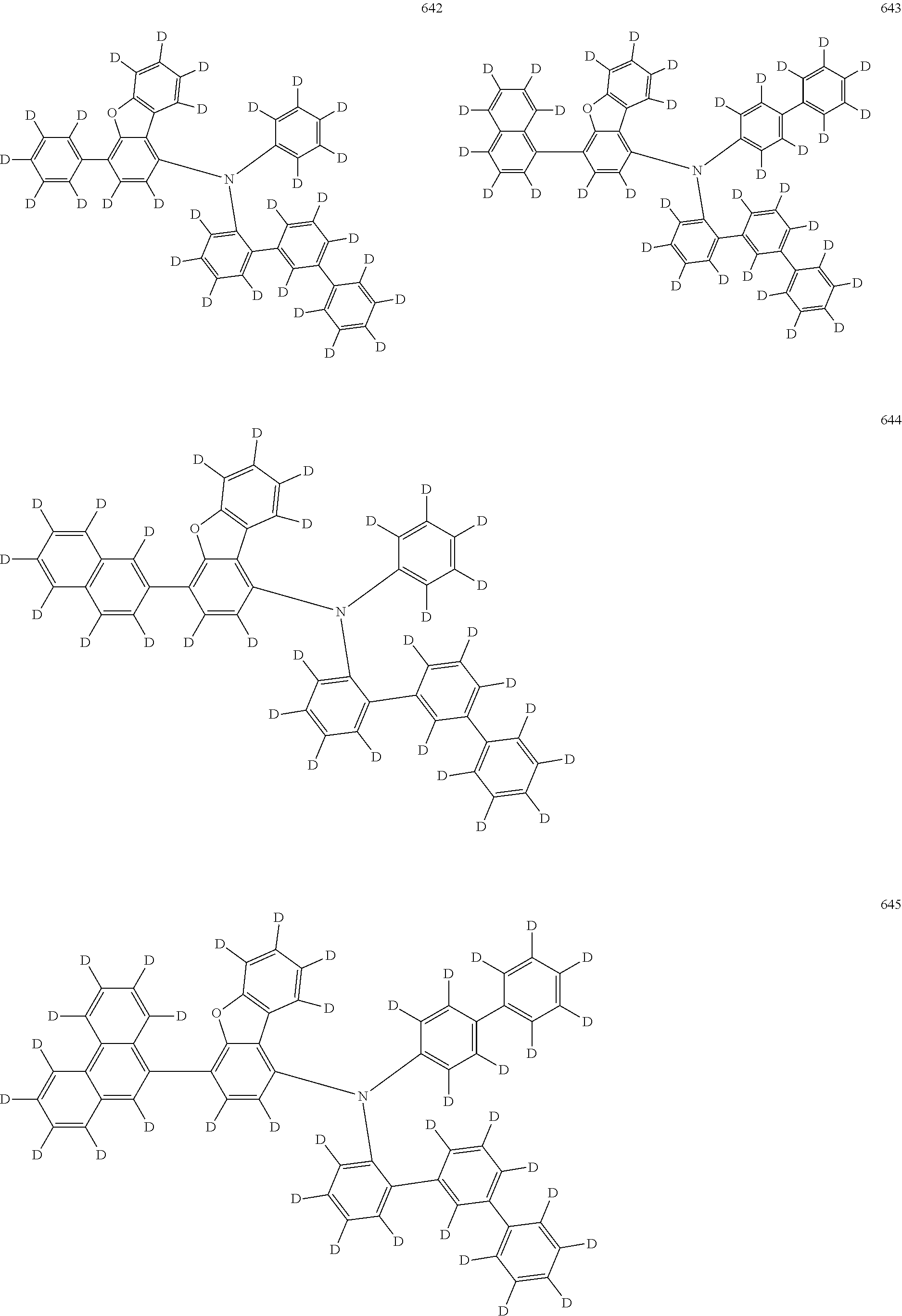

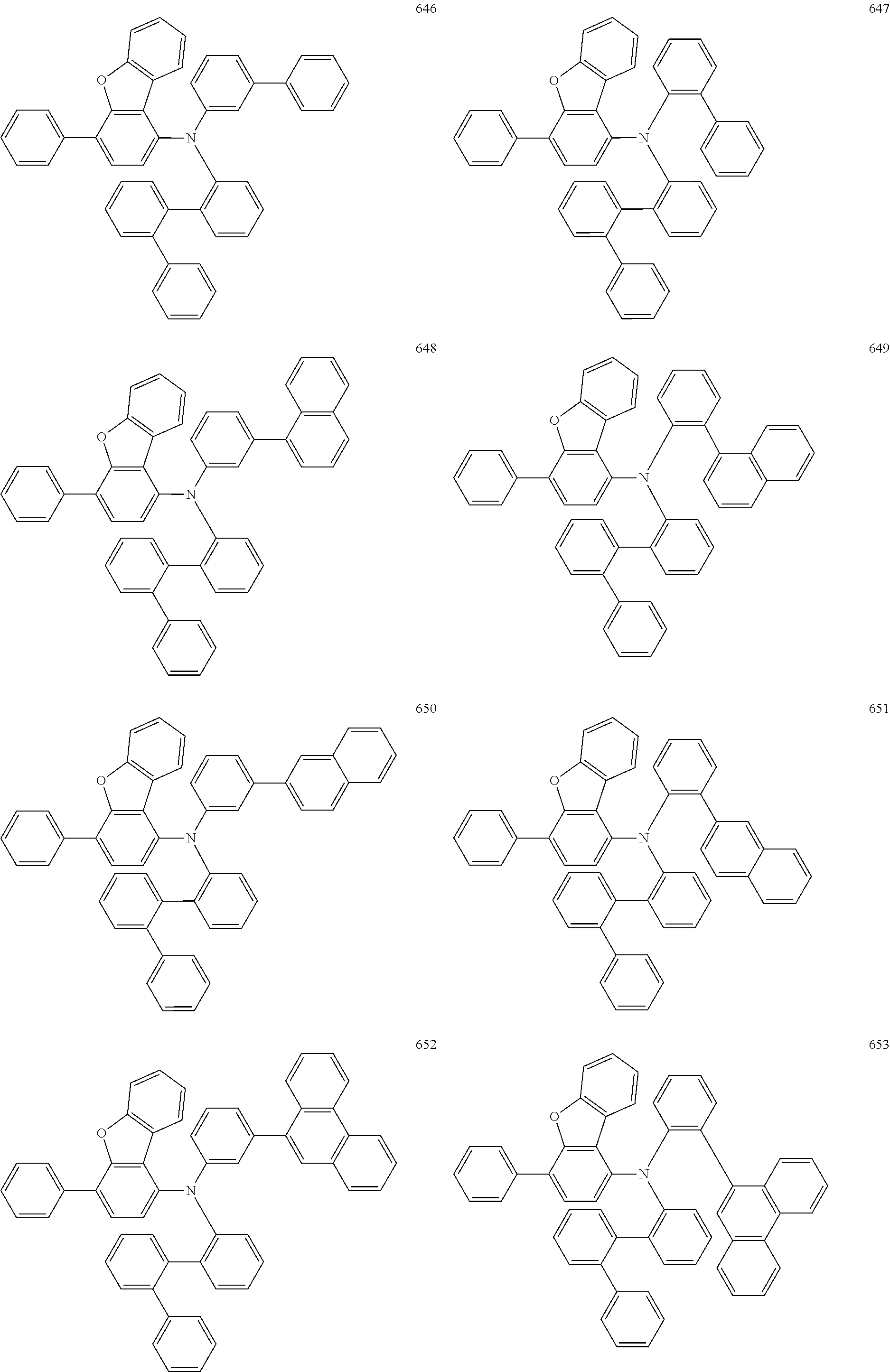

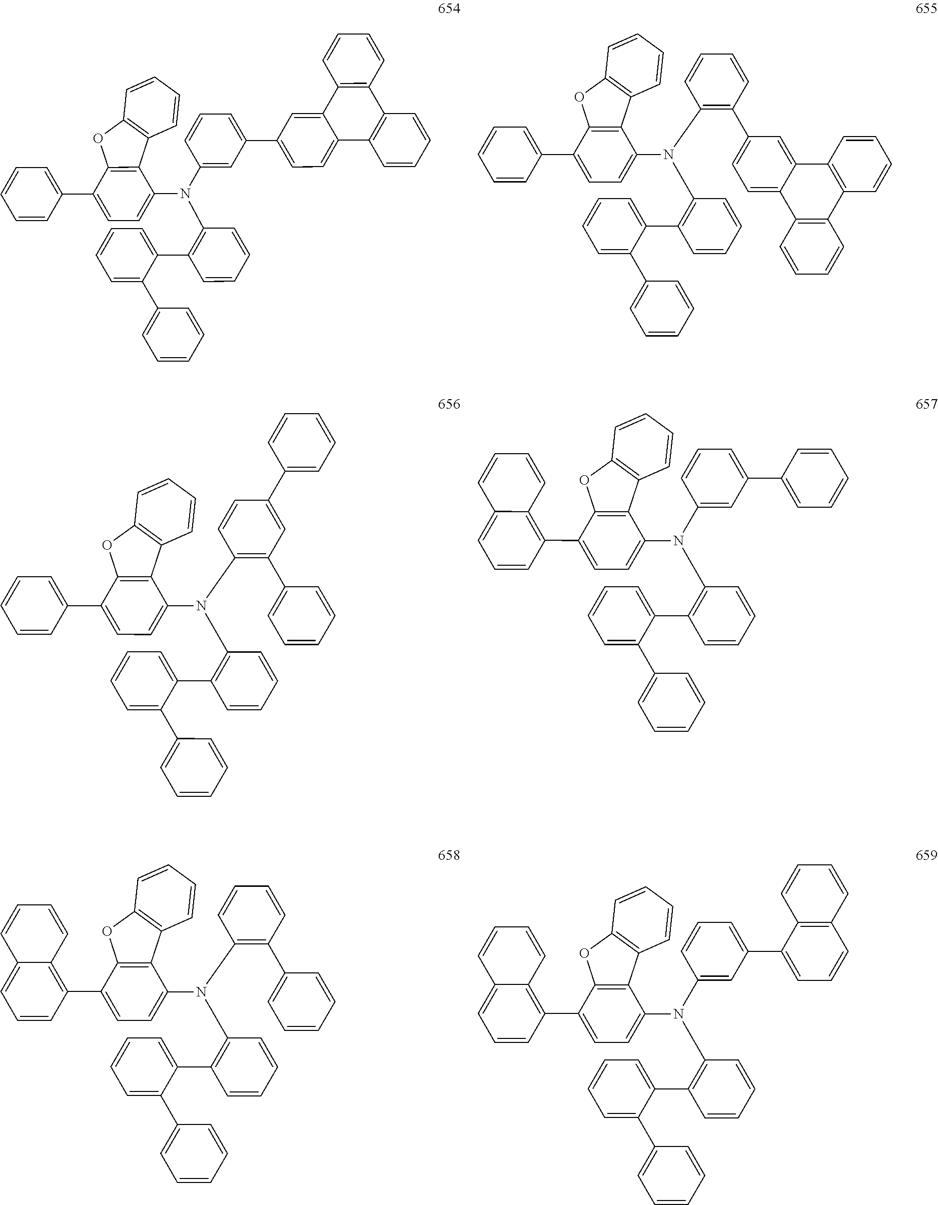

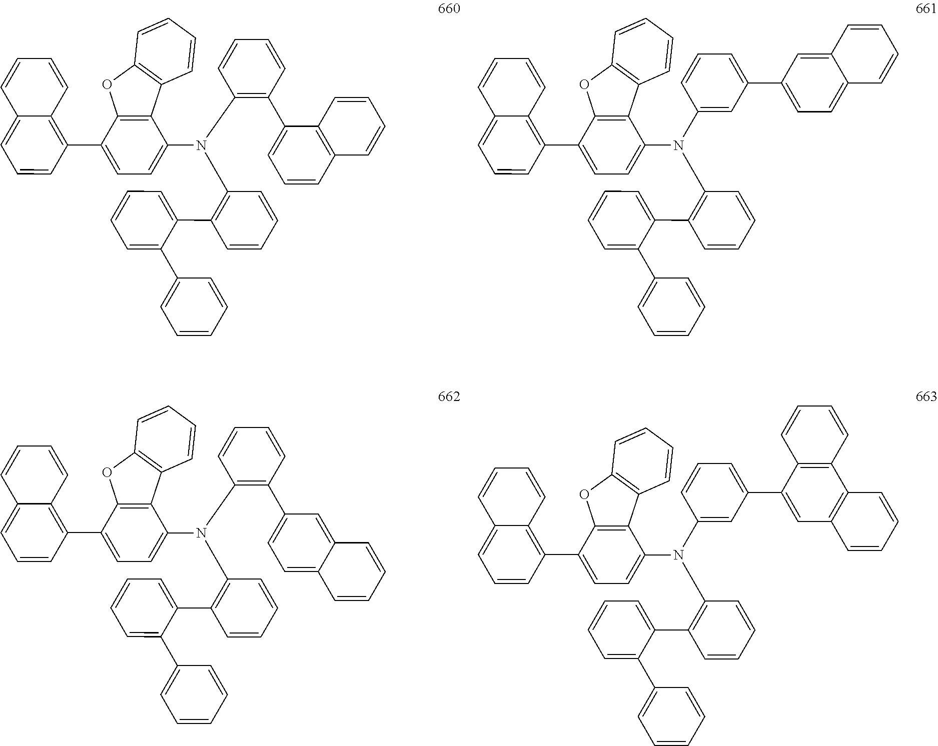 [SMILES: c1ccc(-c2ccccc2-c2ccccc2N(c2ccccc2-c2cccc3ccccc23)c2ccc(-c3cccc4ccccc34)c3oc4ccccc4c23)cc1 ; c1ccc(-c2ccccc2-c2ccccc2N(c2cccc(-c3ccc4ccccc4c3)c2)c2ccc(-c3cccc4ccccc34)c3oc4ccccc4c23)cc1 ; c1ccc(-c2ccccc2-c2ccccc2N(c2ccccc2-c2ccc3ccccc3c2)c2ccc(-c3cccc4ccccc34)c3oc4ccccc4c23)cc1 ; c1ccc(-c2ccccc2-c2ccccc2N(c2cccc(-c3cc4ccccc4c4ccccc34)c2)c2ccc(-c3cccc4ccccc34)c3oc4ccccc4c23)cc1]

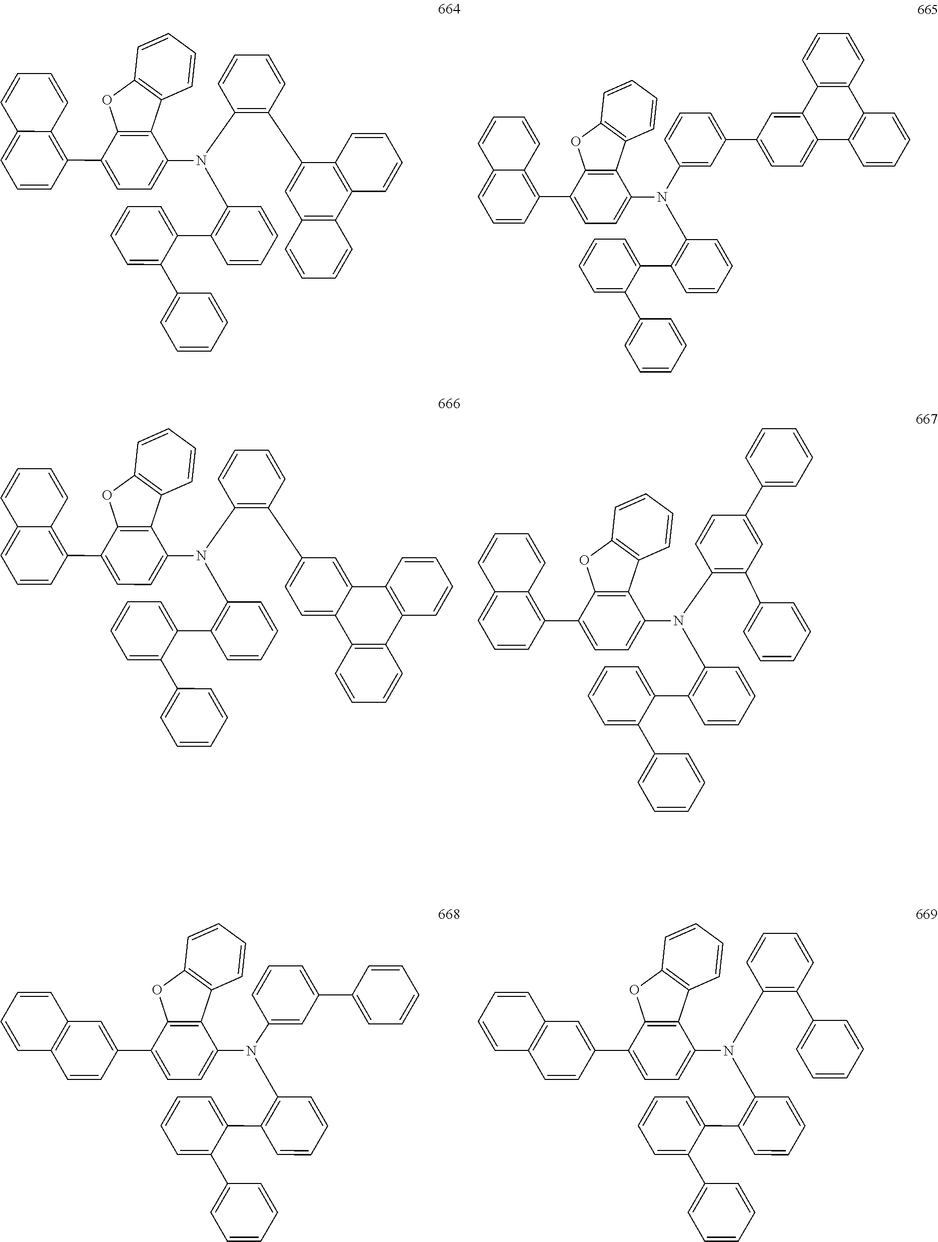

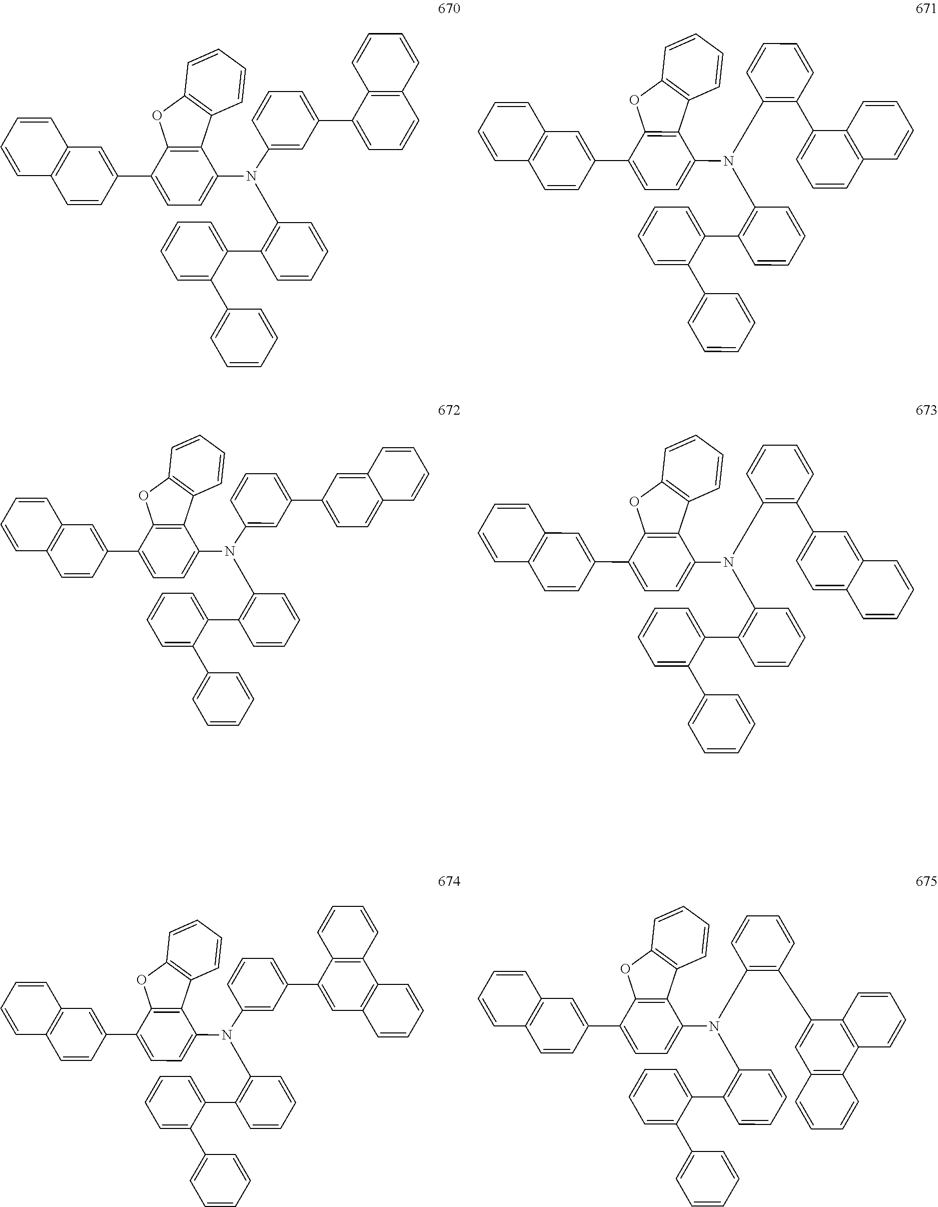

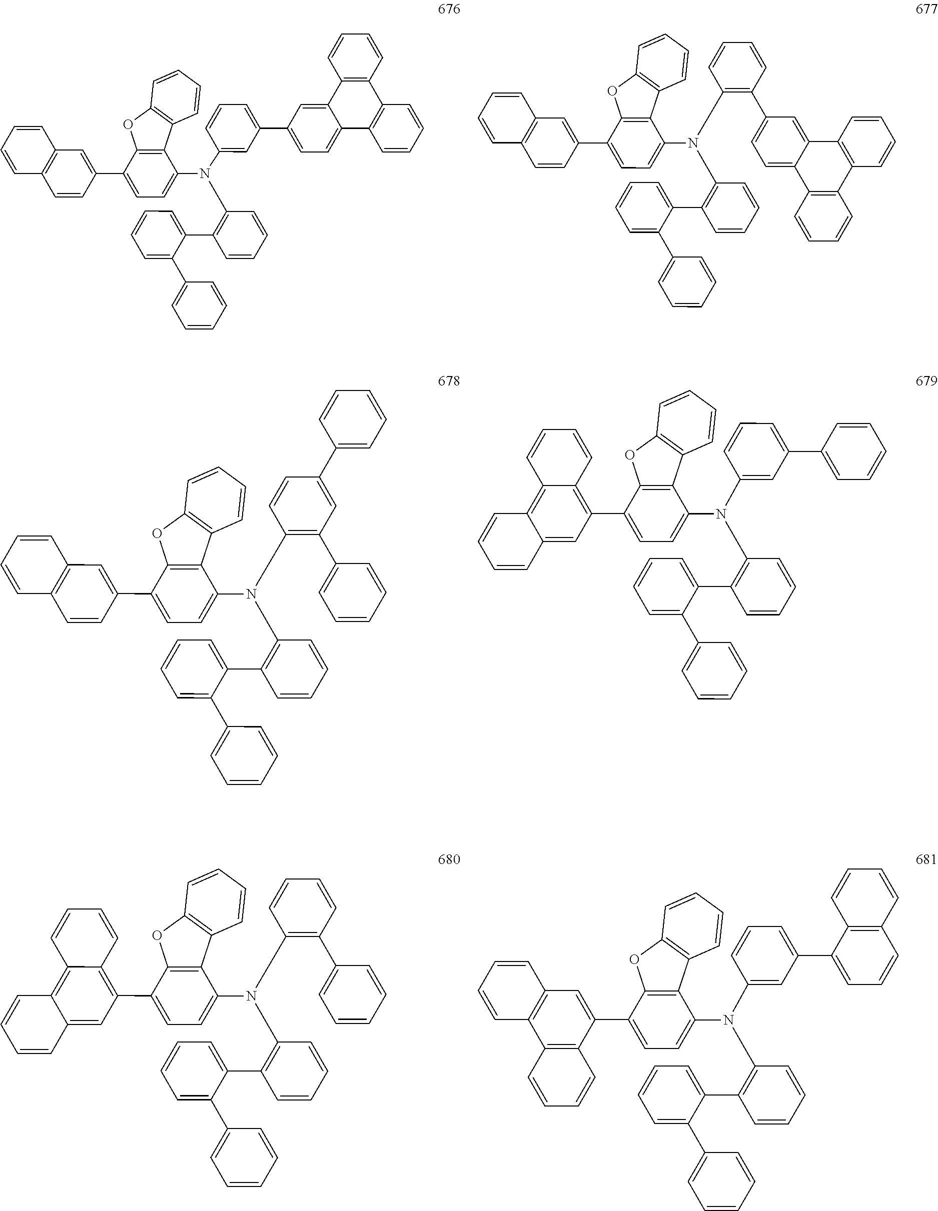

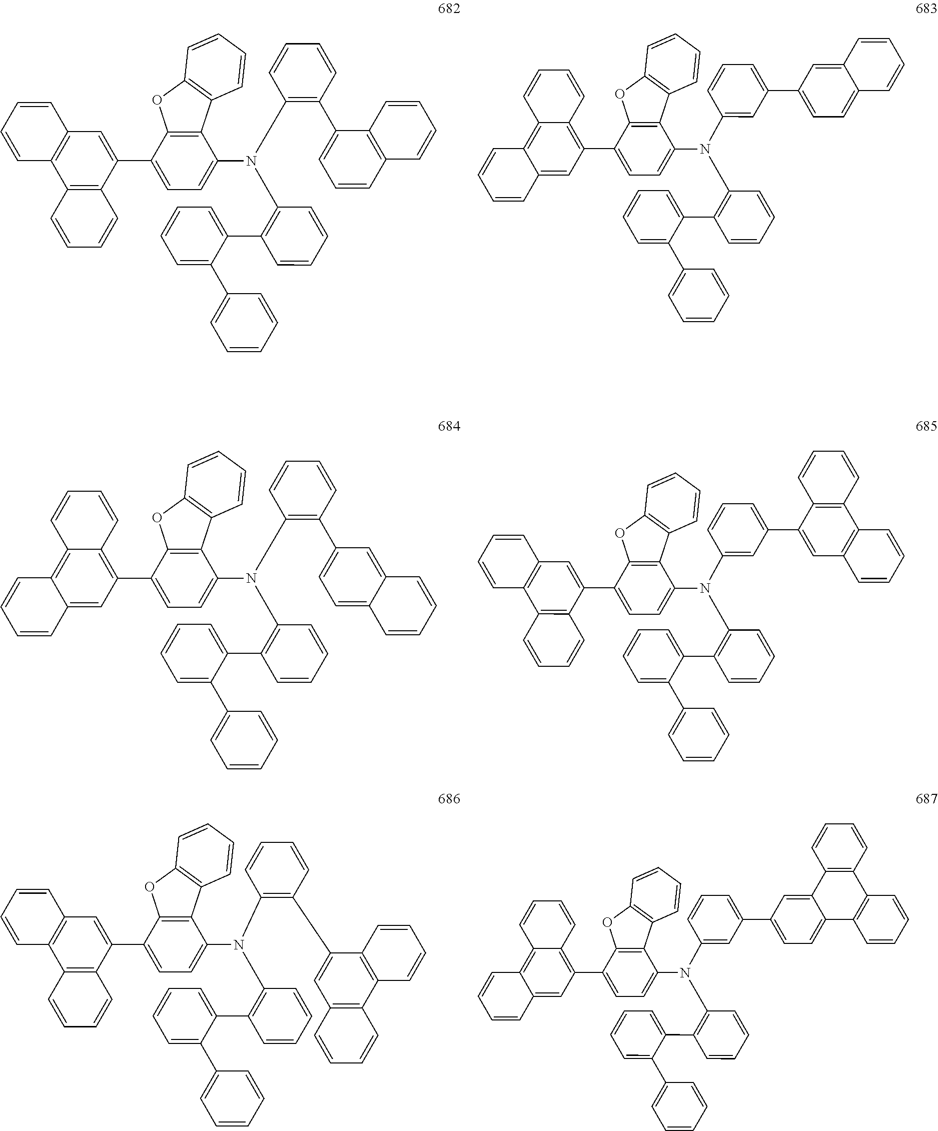

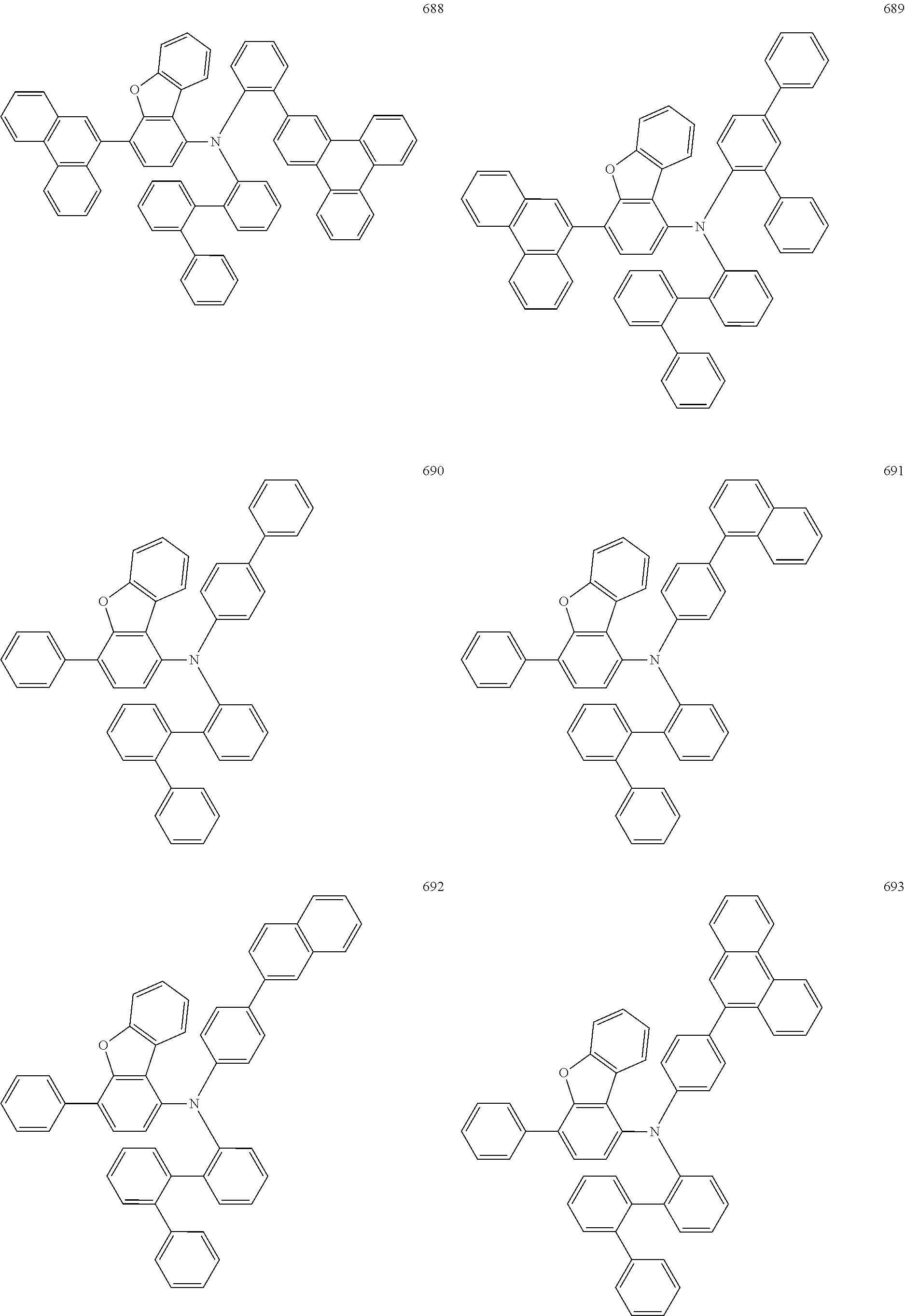

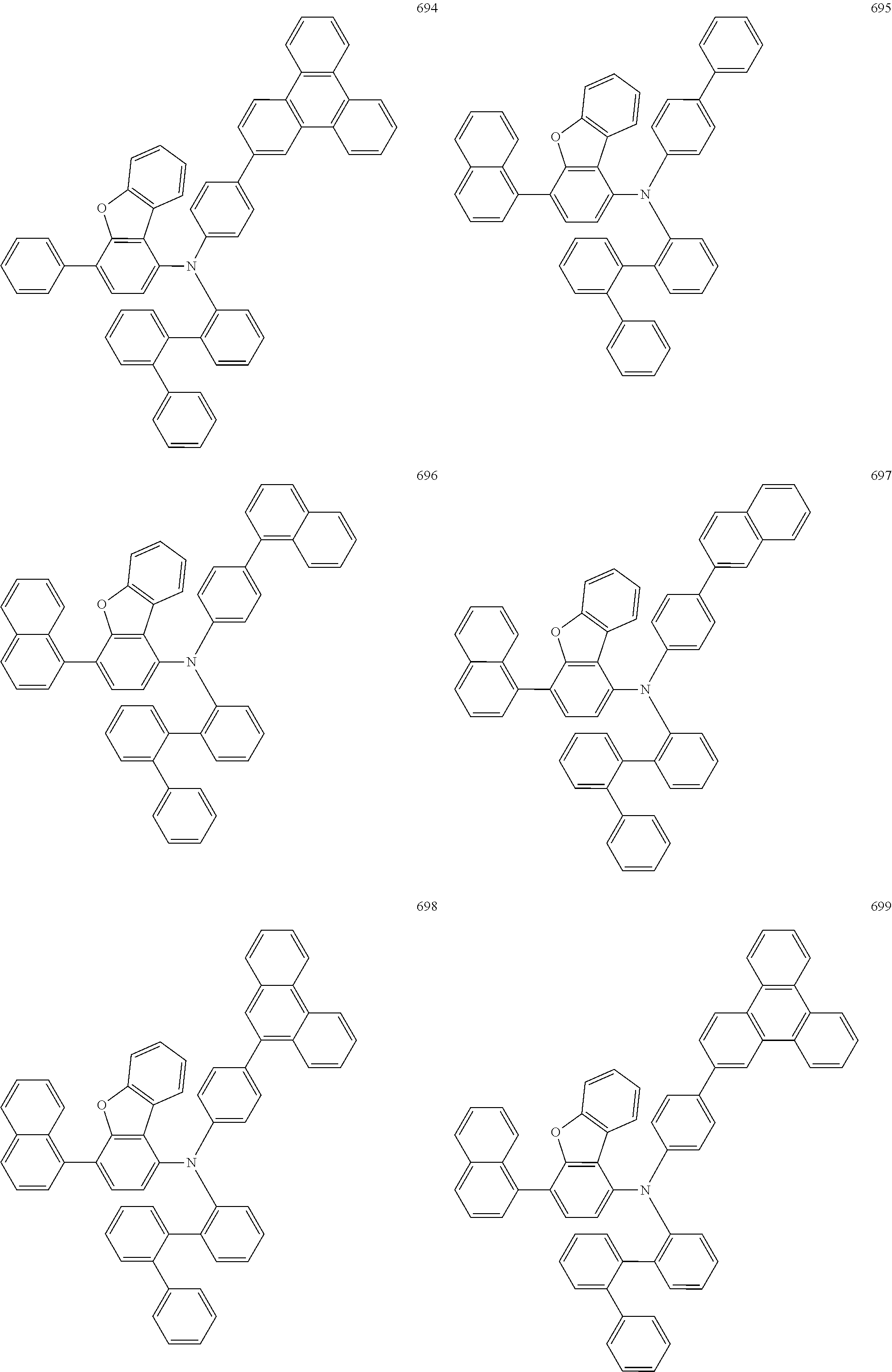

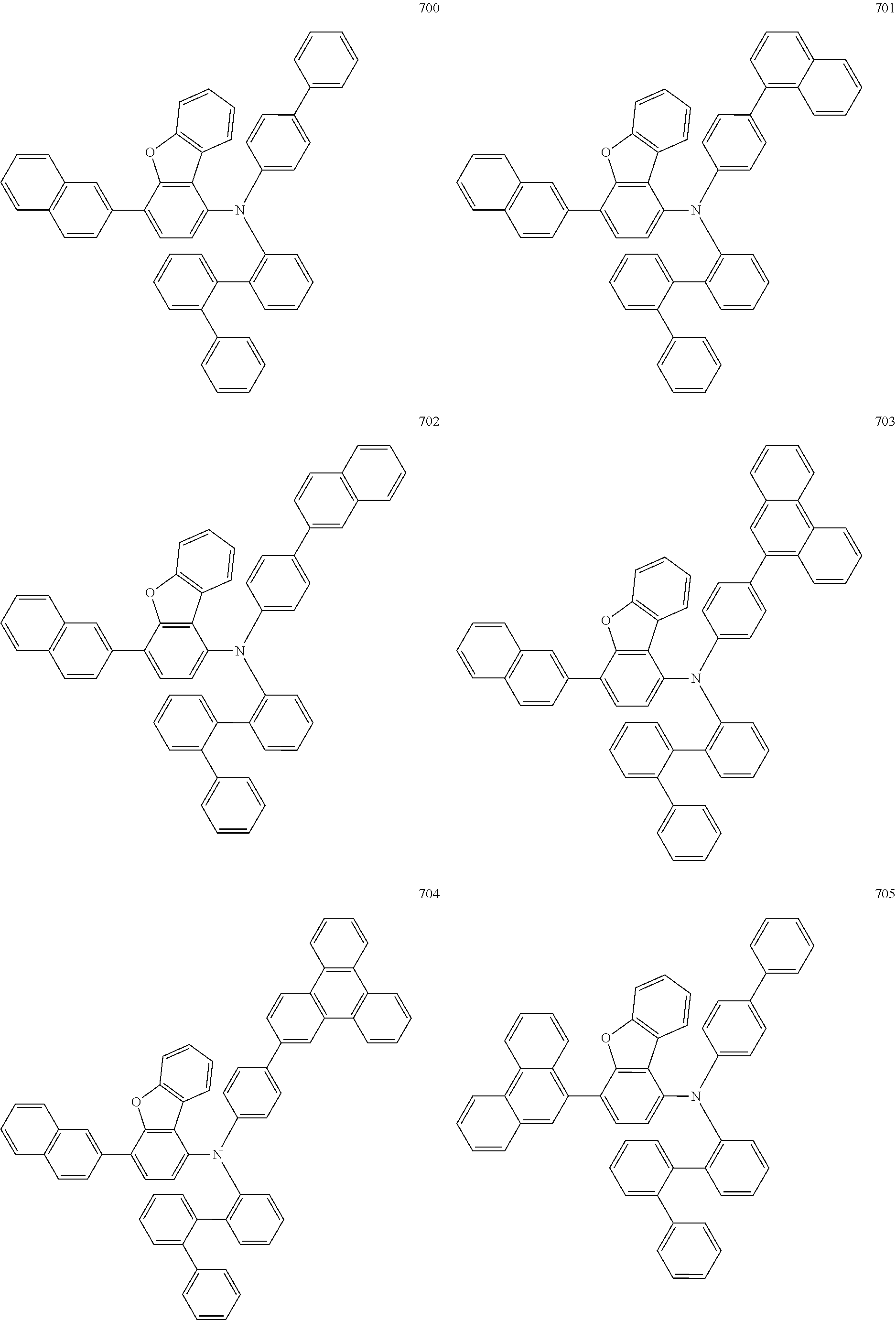

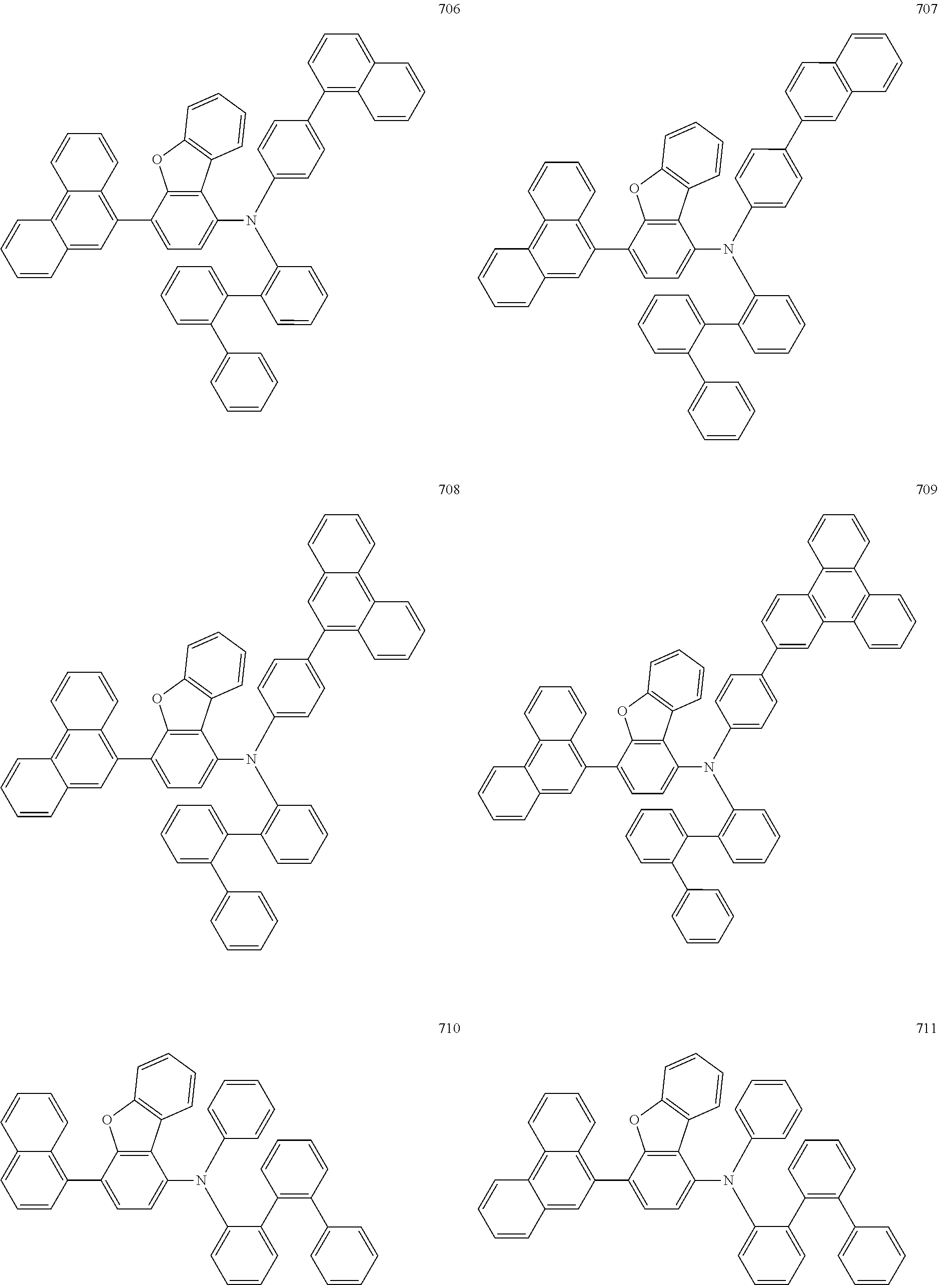

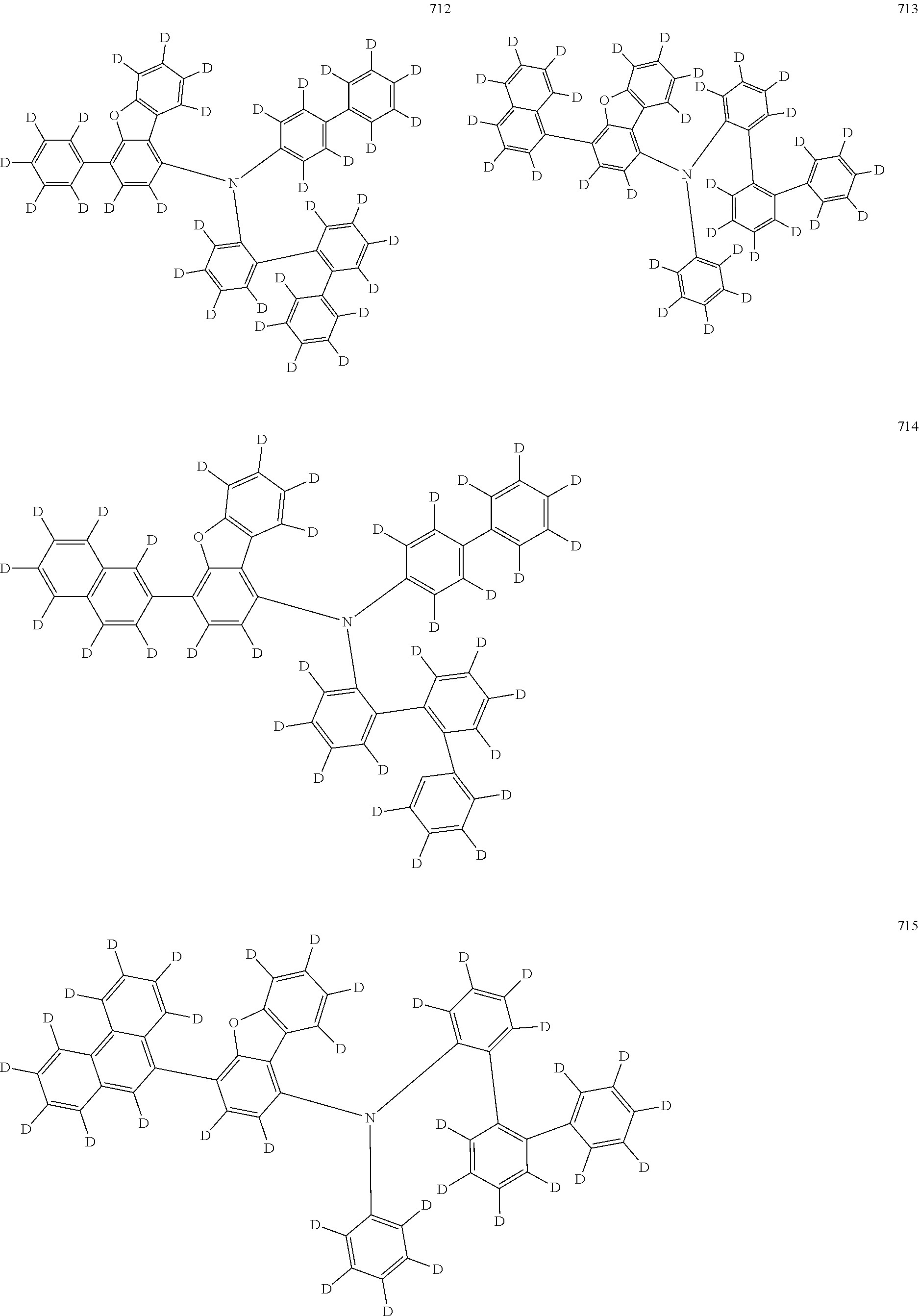

An organic light-emitting diode according to one aspect of the present disclosure may include a positive electrode and a negative electrode facing the positive electrode, and may include an organic material layer between the positive electrode and the negative electrode.

According to one embodiment of the present disclosure, at least one of the at least one organic material layer may contain the organic compound represented by the Chemical Formula 1, wherein the organic material layer containing the organic compound represented by the Chemical Formula 1 is an hole transport auxiliary layer.

According to one embodiment of the present disclosure, the at least one organic material layer may further include at least one selected from a group consisting of a hole injection layer, a hole transport layer, a light-emitting layer, an electron transport layer, and an electron injection layer.

For example, the organic light-emitting diode may have a structure in which the positive electrode, the hole injection layer (HIL), the hole transport layer (HTL), the hole transport auxiliary layer, the light-emitting layer (EML), the electron transport layer (ETL), the electron injection layer (EIL), and the negative electrode are sequentially stacked.

The organic material layer may additionally include an electron transport auxiliary layer.

The positive electrode may include a transparent and highly conductive material such as indium tin oxide (ITO), indium zinc oxide (IZO), tin oxide (SnO₂), and zinc oxide (ZnO).

The negative electrode may include a material such as lithium (Li), aluminum (Al), aluminum-lithium (Al—Li), calcium (Ca), magnesium (Mg), magnesium-indium (Mg—In), magnesium-silver (Mg—Ag). Furthermore, in a top-emission organic light-emitting diode, indium tin oxide (ITO) or indium zinc oxide (IZO) may be used to constitute a transparent negative electrode through which light may transmit.

A capping layer (CPL) may be formed on a surface of the negative electrode and may be made of a capping layer formation composition.

A hole injection layer compound or a hole transport layer compound is not specifically limited. Any compound may be used as the hole injection layer or hole transport layer compound as long as it is generally used as the hole injection layer or hole transport layer compound. Non-limiting examples of the hole injection layer or hole transport layer compound may include a phthalocyanine derivative, a porphyrin derivative, a triarylamine derivative and an indolocarbazole derivative. For example, non-limiting examples of the hole injection layer or hole transport layer compound may include 1,4,5,8,9,11-hexaazatriphenylene-hexacarbonitrile (HAT-CN), copper phthalocyanine (CuPc), 4,4′,4″-tris(3-methylphenyl)amino) triphenylamine (m-MTDATA), 4,4′,4″-tris(3-methylphenylamino)phenoxybenzene (m-MTDAPB), 4,4′,4″-tri(N-carbazolyl)triphenylamine (TCTA), 4,4′,4″-tris(N-(2-naphthyl)-N-phenylamino)-triphenylamine (2-TNATA), N4,N4,N4′,N4′-tetra([1,1′-biphenyl]-4-yl)-[1,1′-biphenyl]-4,4′-diamine, bis(N-(1-naphthyl-n-phenyl))benzidine (α-NPD), N,N′-di(naphthalen-1-yl)-N,N′-biphenyl-benzidine (NPB) or N,N′-biphenyl-N,N′-bis(3-methylphenyl)-1,1′-biphenyl-4,4′-diamine (TPD), etc.

The compound included in the light-emitting layer is not specifically limited, and any compound may be used as the compound included in the light-emitting layer as long as it is generally used as the light-emitting layer compound. A single light-emitting compound or a light-emitting host compound may be used as the light-emitting layer compound.

Examples of the light-emitting compound of the light-emitting layer may include compounds that may cause light emission via phosphorescence, fluorescence, thermally-activated delayed fluorescence, i.e., TADF (also referred to as E-type delayed fluorescence), triplet-triplet quenching, or a combination of these processes. However, the present disclosure is not limited thereto. The light-emitting compound may be selected from a variety of materials depending on a desired color to be rendered. Non-limiting examples of the light-emitting compound may include condensed cyclic derivatives such as phenanthrene, anthracene, pyrene, tetracene, pentacene, perylene, naphthopyrene, dibenzopyrene, rubrene, and chrysene, a benzoxazole derivative, a benzothiazole derivative, a benzoimidazole derivative, a benzotriazole derivative, an oxazole derivative, a oxadiazole derivative, a thiazole derivative, a imidazole derivative, a thiadiazole derivative, a triazole derivative, a pyrazoline derivative, a stilbene derivative, a thiophene derivative, a tetraphenylbutadiene derivative, a cyclopentadiene derivative, abisstyryl derivative, abisstyryl arylene derivative, a diazindacene derivative, a furan derivative, a benzofuran derivative, a isobenzofuran derivative, a dibenzofuran derivative, a coumarin derivative, a dicyanomethylenepyran derivative, a dicyanomethylenethiopyran derivative, a polymethine derivative, a cyanine derivative, a oxobenzoanthracene derivative, an xanthene derivative, a rhodamine derivative, a fluorescein derivative, a pyrylium derivative, a carbostyryl derivative, a acridine derivative, a oxazine derivative, a phenylene oxide derivative, a quinacridone derivative, a quinazoline derivative, a pyrrolopyridine derivative, a furopyridine derivative, a 1,2,5-thiadiazolopyrene derivative, a pyromethene derivative, a perinone derivative, a pyrrolopyrrole derivative, a squaryllium derivative, a biolanthrone derivative, a phenazine derivative, a acridone derivative, a deazaflavin derivative, a fluorene derivative, a benzofluorene derivative, an aromatic boron derivative, an aromatic nitrogen boron derivative, and a metal complex (complex in which a metal such as Ir, Pt, Au, Eu, Ru, Re, Ag, and Cu binds to a heteroaromatic ring ligand). For example, non-limiting examples of the light-emitting compound may include N1,N1,N6,N6-tetrakis(4-(1-silyl)phenyl)pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB), Platinum octaethylporphyrin (PtOEP), Ir(ppy)₃, Ir(ppy)₂(acac), Ir(mppy)₃, Ir(PPy)₂(m-bppy), Btplr(acac), Ir(btp)₂(acac), Ir(2-phq)₃, Hex-Ir(phq)₃, Ir(fbi)₂(acac), fac-Tris(2-(3-p-xylyl)phenyl)pyridine iridium(III), Eu(dbm)₃(Phen), Ir(piq)₃, Ir(piq)₂(acac), Ir(Fliq)₂(acac), Ir(Flq)₂(acac), Ru(dtb-bpy)₃-2(PF6), Ir(BT)₂(acac), Ir(DMP)₃, Ir(Mphq)₃, Ir(phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, etc.

As a host compound of the light-emitting layer, a light-emitting host, a hole-transporting host, an electron-transporting host, or a combination thereof may be used. Non-limiting examples of a light-emitting host compound may include condensed cyclic derivatives such as anthracene and pyrene, bisstyryl derivatives such as a bisstyryl anthracene derivative and a distyrylbenzene derivative, a tetraphenylbutadiene derivative, a cyclopentadiene derivative, a fluorene derivative, a benzofluorene derivative, a N-phenylcarbazole derivative, and a carbazonitrile derivative. Non-limiting examples of the hole-transporting host material may include a carbazole derivative, a dibenzofuran derivative, a dibenzothiophene derivative, a triarylamine derivative, an indolocarbazole derivative, and a benzoxazinophenoxazine derivative. Non-limiting examples of the electron-transporting host material may include a pyridine derivative, a triazine derivative, a phosphorus oxide derivative, a benzofuropyridine derivative, and a dibenzoxacillin derivative. For example, the non-limiting examples of the electron-transporting host material may include 9,10-bis(2-naphthyl)anthracene (ADN), tris(8-hydroxyquinolinolato)aluminum (Alq₃), Balq (8-hydroxyquinoline beryllium salt), DPVBi (4,4′-bis(2,2-biphenylethenyl)-1,1′-biphenyl), spiro-DPVBi (spiro-4,4′-bis(2,2-biphenylethenyl)-1,1′-biphenyl), LiPBO (2-(2-benzooxazolyl)-phenol lithium salt), bis(biphenylvinyl)benzene, an aluminum-quinoline metal complex, and metal complexes of imidazole, thiazole and oxazole, etc.

The electron injection layer or electron transport layer compound is not specifically limited, and any compound may be used as the electron injection layer or electron transport layer compound as long as it is generally used as the electron injection layer or electron transport layer compound. Non-limiting examples of the electron injection layer or electron transport layer compounds may include a pyridine derivative, a naphthalene derivative, a anthracene derivative, a phenanthroline derivative, a perinone derivative, a coumarin derivative, a naphthalimide derivative, a anthraquinone derivative, a diphenoquinone derivative, a diphenylquinone derivative, a perylene derivative, a oxadiazole derivative, a thiophene derivative, a triazole derivative, a thiadiazole derivative, a metal complex of an oxine derivative, a quinolinol-based metal complex, a quinoxaline derivative, a polymer of the quinoxaline derivative, a benzazole compound, a gallium complex, a pyrazole derivative, a perfluorinated phenylene derivative, a triazine derivative, a pyrazine derivative, a benzoquinoline derivative, a imidazopyridine derivative, a borane derivative, a benzoimidazole derivative, a benzoxazole derivative, a benzothiazole derivative, a quinoline derivative, an oligopyridine derivative such as terpyridine, a bipyridine derivative, a terpyridine derivative, a naphthyridine derivative, a aldazine derivative, a carbazole derivative, an indole derivative, a phosphorus oxide derivative, a bisstyryl derivative, a quinolinol-based metal complex, a hydroxyazole-based metal complex, an azomethine-based metal complex, a tropolone-based metal complex, a flavonol-based metal complex, a benzoquinoline-based metal complex, metal salts, etc. The materials as described above may be used singly, or may also be used as mixtures with other materials. For example, non-limiting examples of the electron injection layer or electron transport layer compounds may include 2-(4-(9,10-di(naphthalen-2-yl)anthracen-2-yl)phenyl)-1-phenyl-1H-benzo[d]imidazole, tris(8-hydroxyquinolinolato)aluminum (Alq₃), LiF, Liq, Li₂O, BaO, NaCl, and CsF.

The electron transport auxiliary layer may be formed between the electron transport layer and the light-emitting layer. An electron transport auxiliary layer compound is not particularly limited. Any compound may be used as the electron transport auxiliary layer compound as long as it is commonly used as the electron transport auxiliary layer compound. For example, the electron transport auxiliary layer may include pyrimidine derivatives, etc.

The organic light-emitting diode according to one embodiment of the present disclosure may be embodied as a top emission or bottom emission type light-emitting diode.

The organic light-emitting diode according to one embodiment of the present disclosure may be used as a light-emitting element in a display device.

The organic light-emitting diode according to one embodiment of the present disclosure may be applied, as a light-emitting element, to a transparent display device, a mobile display device, a flexible display device, etc. However, the present disclosure is not limited thereto.

Hereinafter, a method for synthesizing the above compounds will be described based on representative examples. However, the method of synthesis of the compounds of the present disclosure is not limited to the following examples. Further, the present disclosure is not limited to examples as set forth below.

### Synthesis Example

A final product of the present disclosure may be synthesized as shown in Reaction Formula 1 (Buchwald-Hartwig Cross Coupling Reaction) as set forth below. However, the present disclosure is not limited thereto.

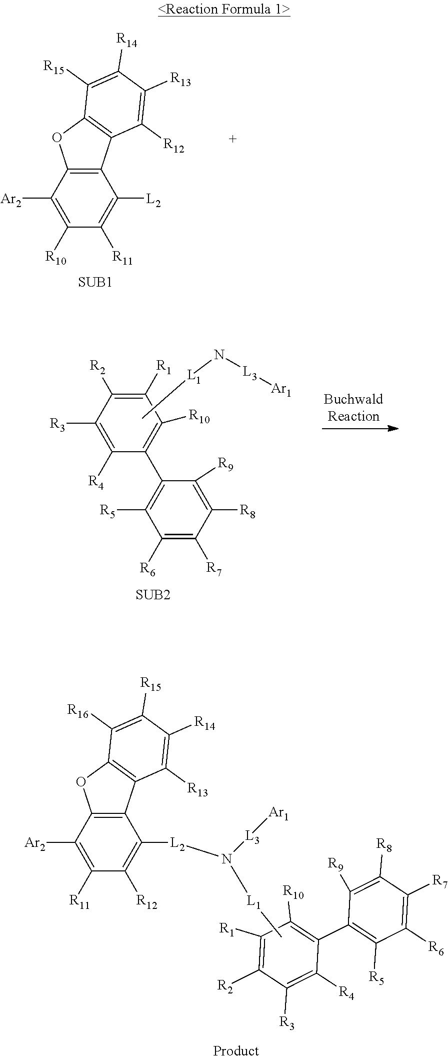

SUB 1 (reactant 1) (53.95 mmol), SUB 2 (reactant 2) (51.38 mmol), t-BuONa (102.76 mmol), Pd₂(dba)₃ (1.03 mmol), Sphos (2.06 mmol) and toluene were added to a 500 mL flask under nitrogen flow and reacted with each other under stirring and refluxing. After completion of the reaction, an organic layer was extracted using toluene and water. The extracted solution was treated with MgSO₄ to remove remaining moisture therefrom, concentrated under a reduced pressure, purified using column chromatography, and then recrystallized to obtain a product.

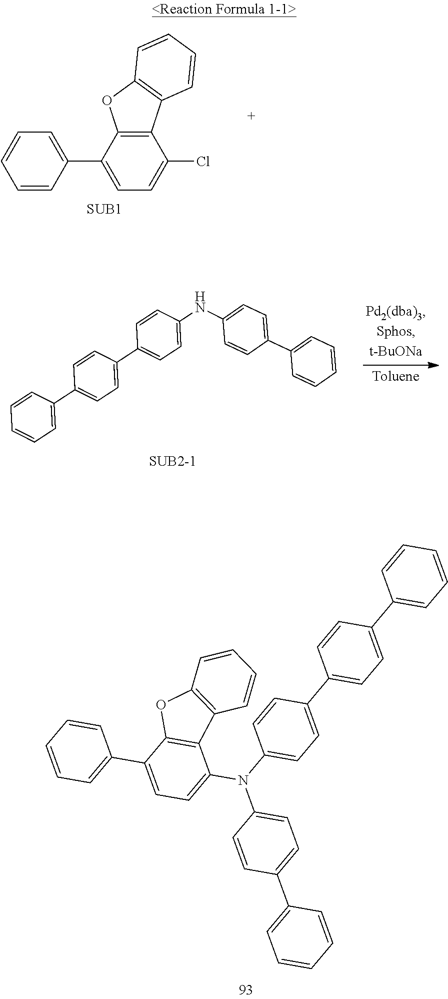

SUB1 and SUB2-1 as reactants 1 and 2, respectively were subjected to the synthesis and purification in the preparation method in <Reaction Formula 1> as set forth above to obtain 23.7 g of ‘Compound 93’ (yield 72%) as a product. m/z=639.26 (C48H33NO=639.80)

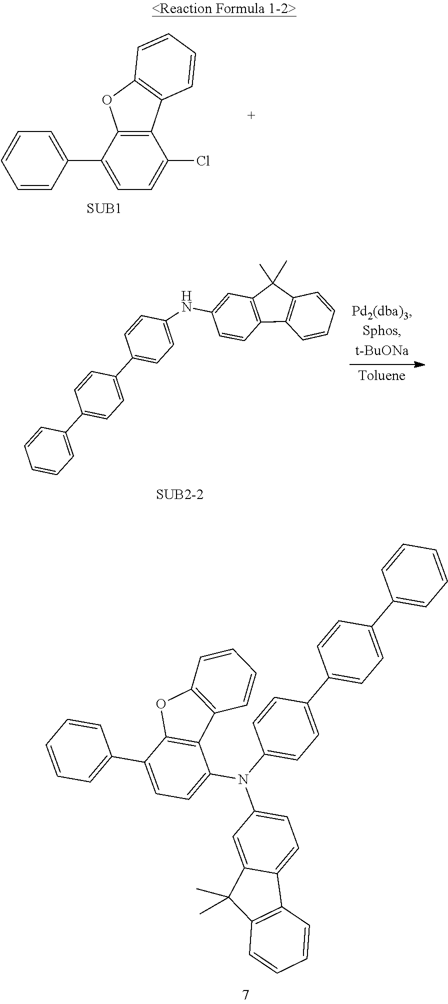

SUB1 and SUB2-2 as reactants 1 and 2, respectively were subjected to the synthesis and purification in the preparation method in <Reaction Formula 1> as set forth above to obtain 25.8 g of ‘Compound 7’ (yield 74%) as a product. m/z=679.29 (C51H37NO=679.86)

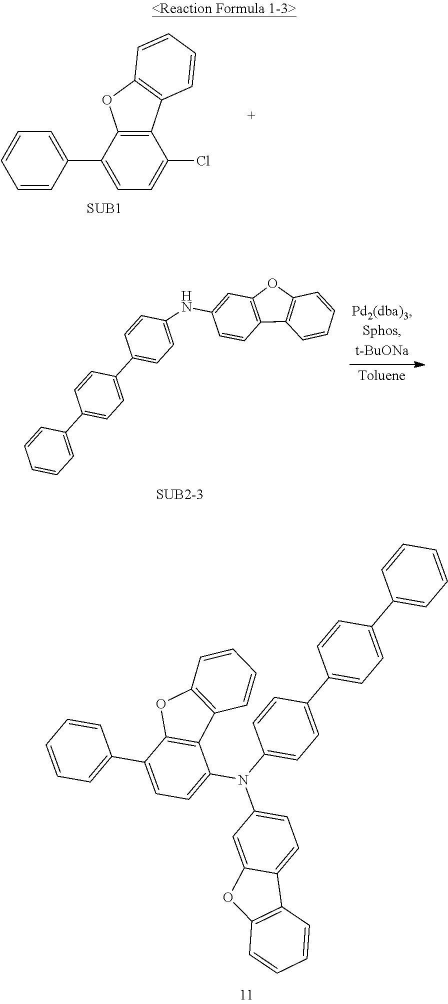

SUB1 and SUB2-3 as reactants 1 and 2, respectively were subjected to the synthesis and purification in the preparation method in <Reaction Formula 1> as set forth above to obtain 23.5 g (yield 70%) of ‘Compound 11’ as a product. m/z=653.24 (C48H32NO2=653.78)

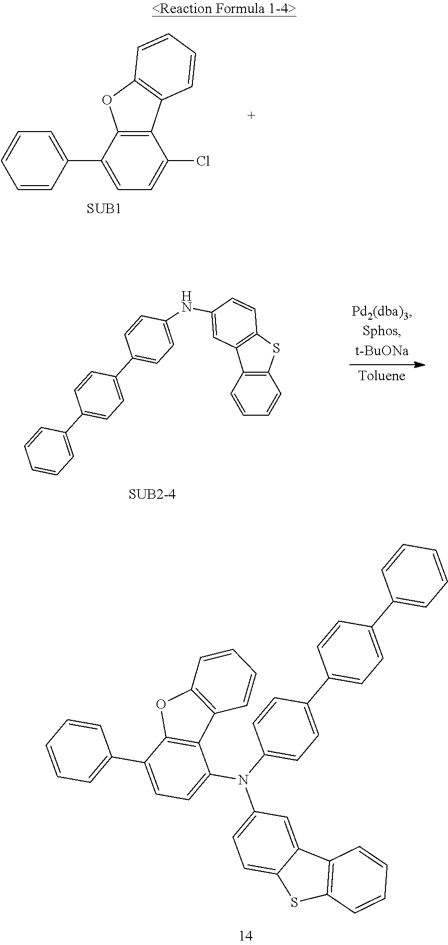

SUB1 and SUB2-4 as reactants 1 and 2, respectively were subjected to the synthesis and purification in the preparation method in <Reaction Formula 1> as set forth above to obtain 23.1 g of ‘Compound 14’ (yield 67%) as a product. m/z=669.21 (C48H31NOS=669.84)

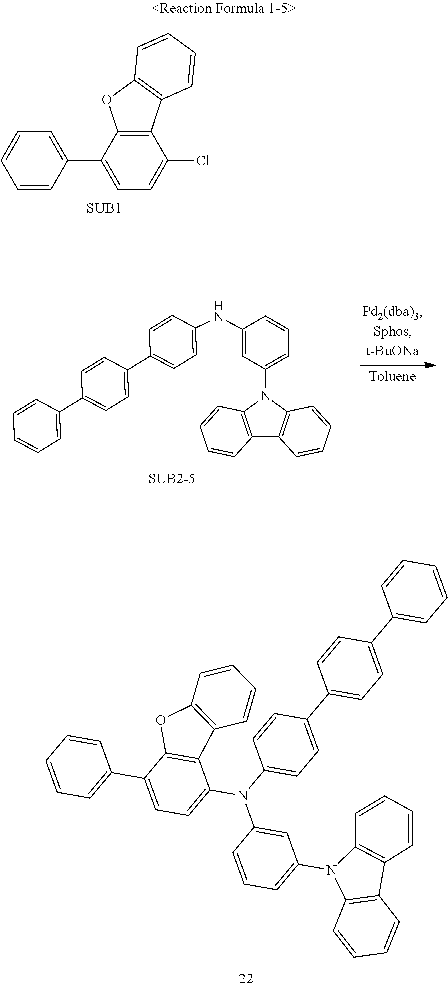

SUB1 and SUB2-5 as reactants 1 and 2, respectively were subjected to the synthesis and purification in the preparation method in <Reaction Formula 1> as set forth above to obtain 24.3 g of ‘Compound 22’ (yield 65%) as a product. m/z=728.28 (C54H36N2O=728.90)

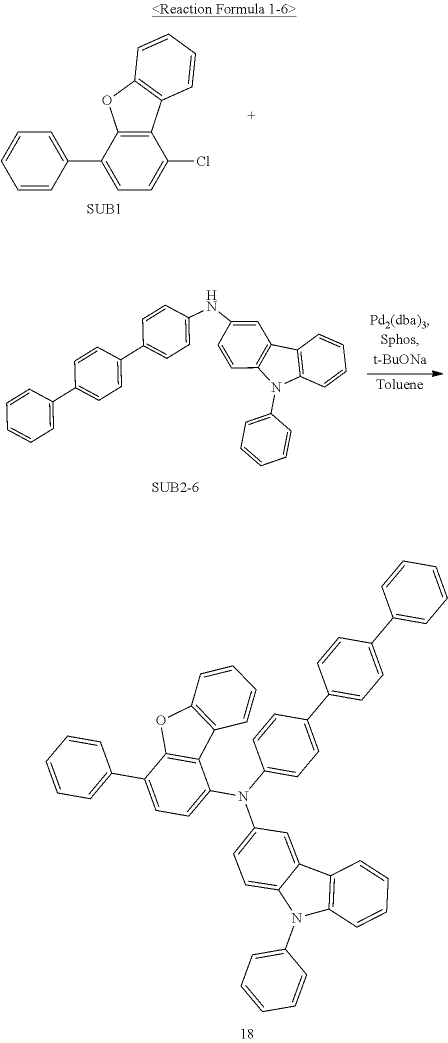

SUB1 and SUB2-6 as reactants 1 and 2, respectively were subjected to the synthesis and purification in the preparation method in <Reaction Formula 1> as set forth above to obtain 26.6 g of ‘Compound 18’ (yield 71%) as a product. m/z=728.28 (C54H36N2O=728.90)

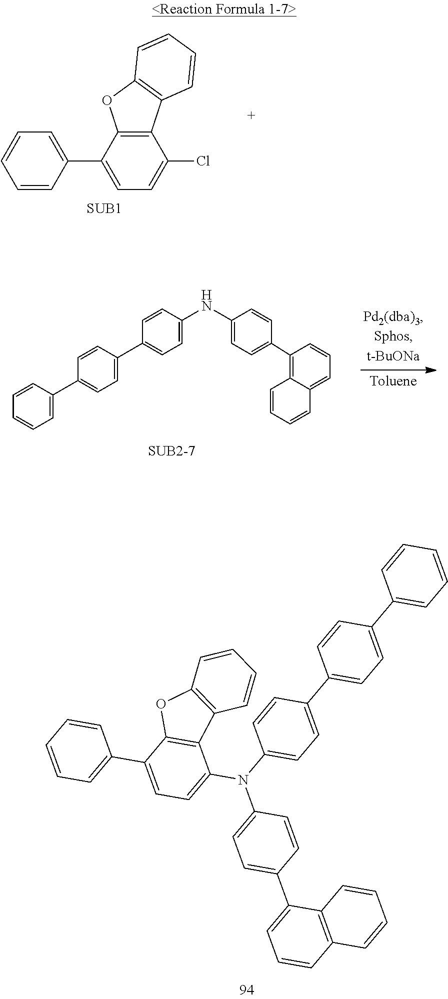

SUB1 and SUB2-7 as reactants 1 and 2, respectively were subjected to the synthesis and purification in the preparation method in <Reaction Formula 1> as set forth above to obtain 23.7 g of ‘Compound 94’ (yield 67%) as a product. m/z=689.27 (C52H35NO=689.86)

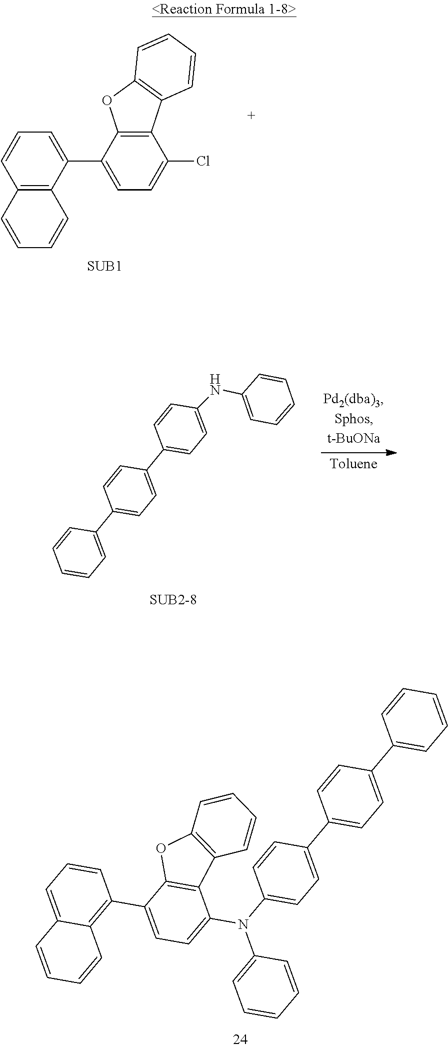

SUB1 and SUB2-8 as reactants 1 and 2, respectively were subjected to the synthesis and purification in the preparation method in <Reaction Formula 1> as set forth above to obtain 23.0 g (yield 73%) of ‘Compound 24’ as a product. m/z=613.24 (C46H31NO=613.76)

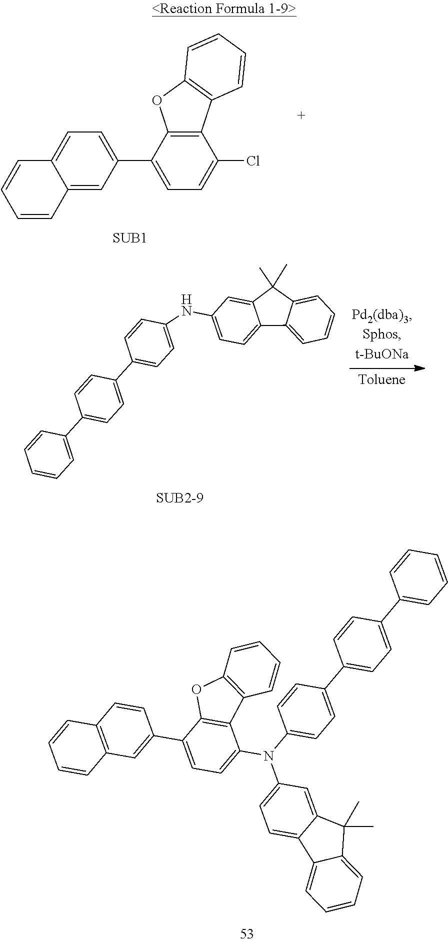

SUB1 and SUB2-9 as reactants 1 and 2, respectively were subjected to the synthesis and purification in the preparation method in <Reaction Formula 1> to obtain 25.6 g of ‘Compound 53’ (yield 68%) as a product. m/z=731.32 (C55H41NO=731.94)

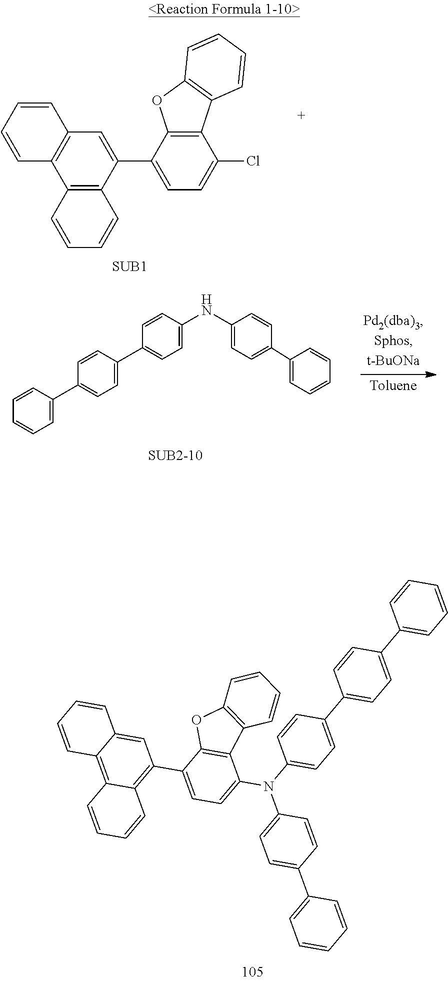

SUB1 and SUB2-10 as reactants 1 and 2, respectively were subjected to the synthesis and purification in the preparation method in <Reaction Formula 1> to obtain 28.5 g of ‘Compound 105’ (yield 75%) as a product. m/z=739.29 (C56H37NO=739.92)

Each of products were synthesized using the preparation method of <Reaction Formula 1> as set forth above, based on the reactants 1 and 2 as shown in Tables 1 to 8 as set forth below.

TABLE 1

| Item | Reactant 1 | | | | |
|---|---|---|---|---|---|
| P1 | 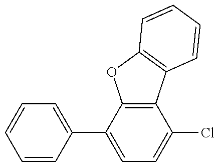 [SMILES: Clc1ccc(-c2ccccc2)c2oc3ccccc3c12] | | | | |
| P2 | 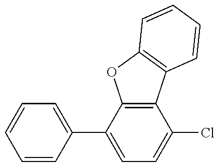 [SMILES: Clc1ccc(-c2ccccc2)c2oc3ccccc3c12] | | | | |
| P3 | 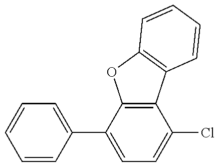 [SMILES: Clc1ccc(-c2ccccc2)c2oc3ccccc3c12] | | | | |
| P4 | 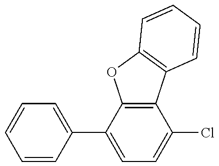 [SMILES: Clc1ccc(-c2ccccc2)c2oc3ccccc3c12] | | | | |
| P5 | 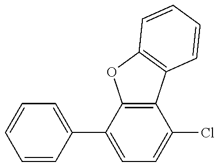 [SMILES: Clc1ccc(-c2ccccc2)c2oc3ccccc3c12] | | | | |
| P6 | 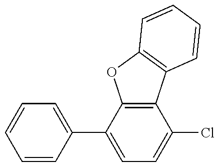 [SMILES: Clc1ccc(-c2ccccc2)c2oc3ccccc3c12] | | | | |
| P7 | 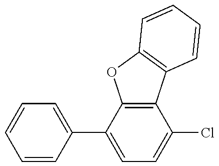 [SMILES: Clc1ccc(-c2ccccc2)c2oc3ccccc3c12] | | | | |
| P8 | 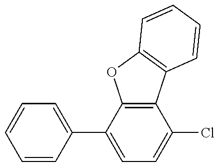 [SMILES: Clc1ccc(-c2ccccc2)c2oc3ccccc3c12] | | | | |
| P9 | 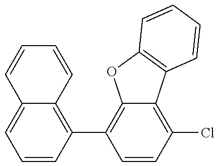 [SMILES: Clc1ccc(-c2cccc3ccccc23)c2oc3ccccc3c12] | | | | |
| P10 | 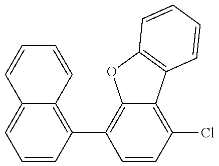 [SMILES: Clc1ccc(-c2cccc3ccccc23)c2oc3ccccc3c12] | | | | |
| P11 | 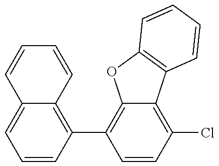 [SMILES: Clc1ccc(-c2cccc3ccccc23)c2oc3ccccc3c12] | | | | |
| P12 | 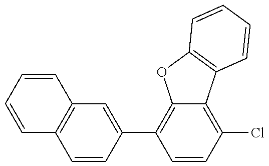 [SMILES: Clc1ccc(-c2ccc3ccccc3c2)c2oc3ccccc3c12] | | | | |
| P13 | 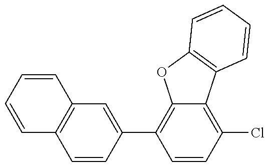 [SMILES: Clc1ccc(-c2ccc3ccccc3c2)c2oc3ccccc3c12] | | | | |
| P14 | 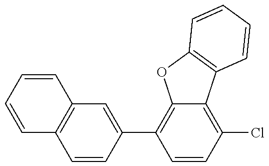 [SMILES: Clc1ccc(-c2ccc3ccccc3c2)c2oc3ccccc3c12] | | | | |
| P15 | 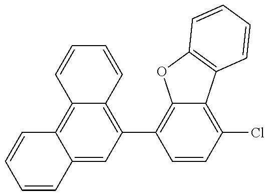 [SMILES: Clc1ccc(-c2cc3ccccc3c3ccccc23)c2oc3ccccc3c12] | | | | |
| P16 | 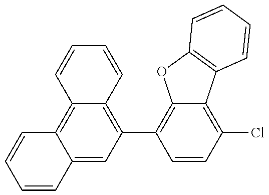 [SMILES: Clc1ccc(-c2cc3ccccc3c3ccccc23)c2oc3ccccc3c12] | | | | |
| P17 | 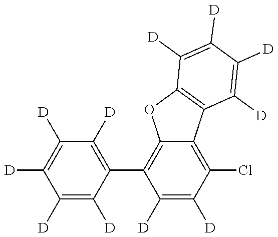 [SMILES: [2H]c1c([2H])c([2H])c(-c2c([2H])c([2H])c(Cl)c3c2oc2c([2H])c([2H])c([2H])c([2H])c23)c([2H])c1[2H]] | | | | |
| P18 | 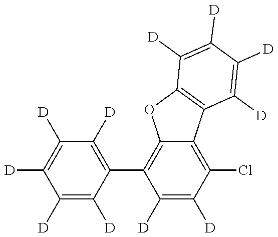 [SMILES: [2H]c1c([2H])c([2H])c(-c2c([2H])c([2H])c(Cl)c3c2oc2c([2H])c([2H])c([2H])c([2H])c23)c([2H])c1[2H]] | | | | |
| P19 | 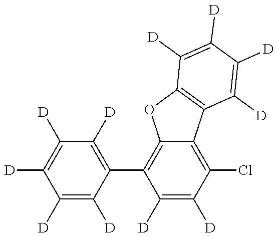 [SMILES: [2H]c1c([2H])c([2H])c(-c2c([2H])c([2H])c(Cl)c3c2oc2c([2H])c([2H])c([2H])c([2H])c23)c([2H])c1[2H]] | | | | |
| P20 | 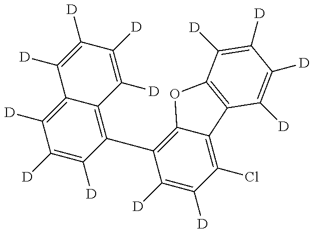 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | | | | |
| P21 | 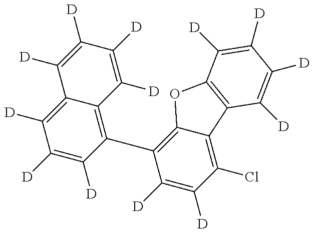 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | | | | |
| P22 | 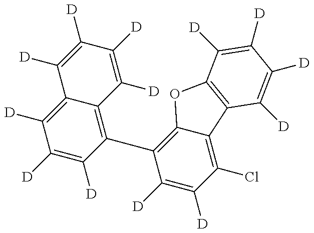 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | | | | |
| P23 | 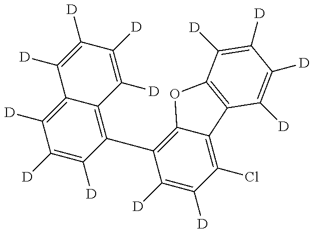 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | | | | |
| P24 | 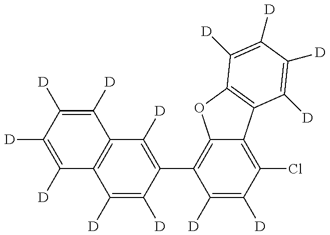 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | | | | |
| P25 | 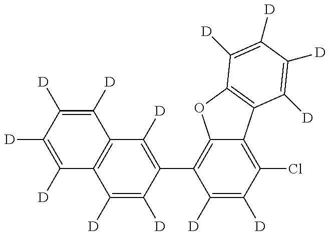 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | | | | |
| P26 | 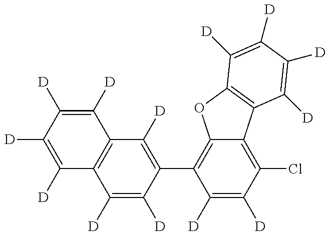 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | | | | |
| P27 | 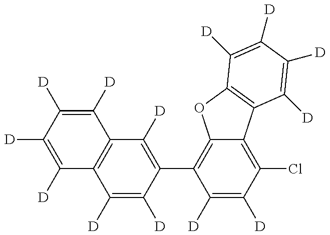 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | | | | |
| P28 | 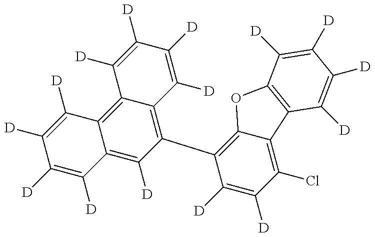 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | | | | |
| P29 | 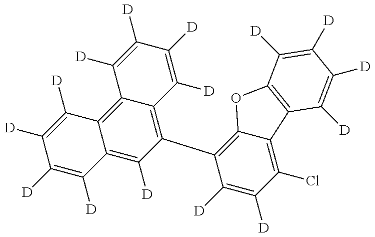 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | | | | |
| P30 | 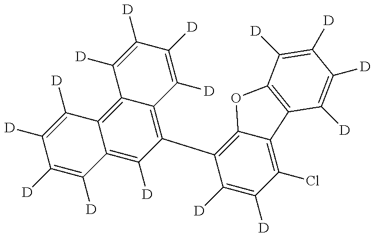 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | | | | |
| P31 | 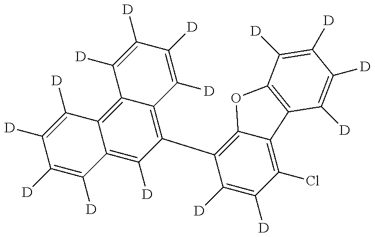 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | | | | |
| | | | | Obtained | |
| | | | | amount | |
| | Item | Reactant 2 | Product | (Yield) | [M + H]⁺ |
| | P1 | 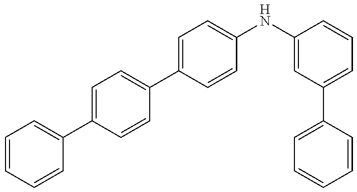 [SMILES: c1ccc(-c2ccc(-c3ccc(Nc4cccc(-c5ccccc5)c4)cc3)cc2)cc1] | Compound 115 | 16.2 g (72%) | 639.26 |
| | P2 | 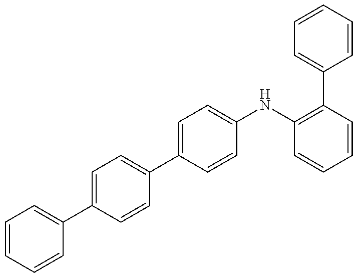 [SMILES: c1ccc(-c2ccc(-c3ccc(Nc4ccccc4-c4ccccc4)cc3)cc2)cc1] | Compound 116 | 16.7 g (74%) | 639.26 |
| | P3 | 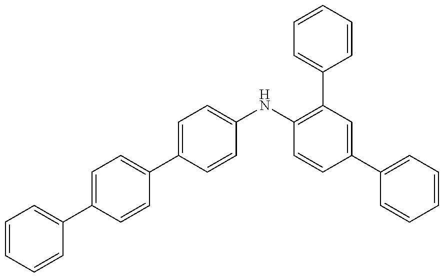 [SMILES: c1ccc(-c2ccc(-c3ccc(Nc4ccc(-c5ccccc5)cc4-c4ccccc4)cc3)cc2)cc1] | Compound 125 | 17.6 g (70%) | 715.29 |
| | P4 | 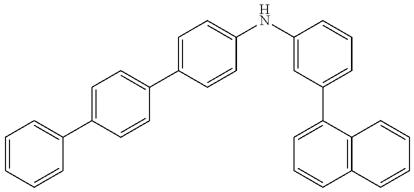 [SMILES: c1ccc(-c2ccc(-c3ccc(Nc4cccc(-c5cccc6ccccc56)c4)cc3)cc2)cc1] | Compound 118 | 16.3 g (67%) | 689.27 |
| | P5 | 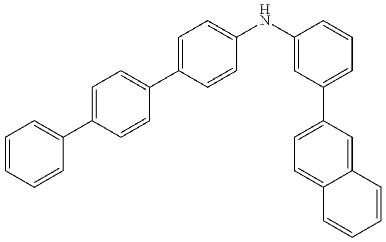 [SMILES: c1ccc(-c2ccc(-c3ccc(Nc4cccc(-c5ccc6ccccc6c5)c4)cc3)cc2)cc1] | Compound 119 | 15.8 g (65%) | 689.27 |
| | P6 | 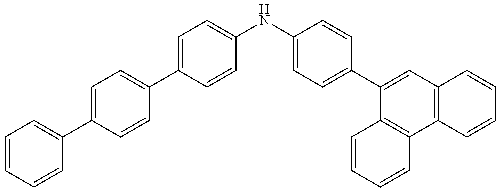 [SMILES: c1ccc(-c2ccc(-c3ccc(Nc4ccc(-c5cc6ccccc6c6ccccc56)cc4)cc3)cc2)cc1] | Compound 96 | 18.5 g (71%) | 739.29 |
| | P7 | 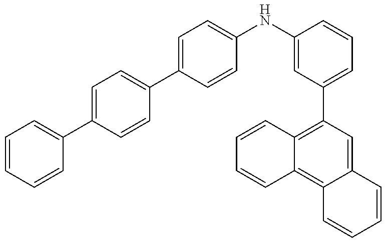 [SMILES: c1ccc(-c2ccc(-c3ccc(Nc4cccc(-c5cc6ccccc6c6ccccc56)c4)cc3)cc2)cc1] | Compound 121 | 19.0 g (73%) | 739.29 |
| | P8 | 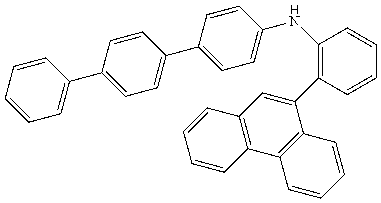 [SMILES: c1ccc(-c2ccc(-c3ccc(Nc4ccccc4-c4cc5ccccc5c5ccccc45)cc3)cc2)cc1] | Compound 122 | 17.7 g (68%) | 739.29 |
| | P9 | 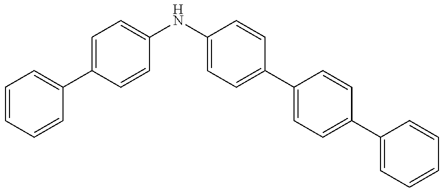 [SMILES: c1ccc(-c2ccc(Nc3ccc(-c4ccc(-c5ccccc5)cc4)cc3)cc2)cc1] | Compound 97 | 18.4 g (76%) | 689.27 |
| | P10 | 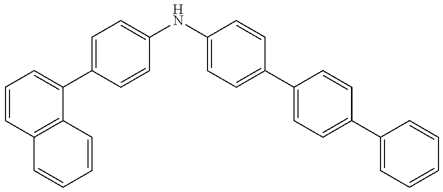 [SMILES: c1ccc(-c2ccc(-c3ccc(Nc4ccc(-c5cccc6ccccc56)cc4)cc3)cc2)cc1] | Compound 98 | 18.5 g (71%) | 739.29 |
| | P11 | 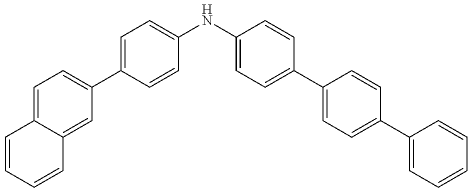 [SMILES: c1ccc(-c2ccc(-c3ccc(Nc4ccc(-c5ccc6ccccc6c5)cc4)cc3)cc2)cc1] | Compound 99 | 19.5 g (75%) | 739.29 |
| | P12 | 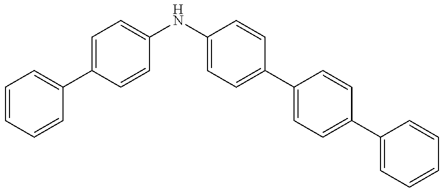 [SMILES: c1ccc(-c2ccc(Nc3ccc(-c4ccc(-c5ccccc5)cc4)cc3)cc2)cc1] | Compound 101 | 17.5 g (72%) | 689.27 |
| | P13 | 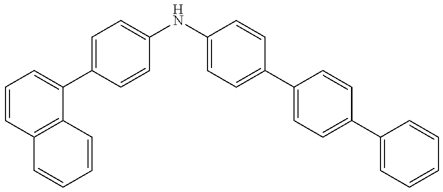 [SMILES: c1ccc(-c2ccc(-c3ccc(Nc4ccc(-c5cccc6ccccc56)cc4)cc3)cc2)cc1] | Compound 102 | 20.3 g (78%) | 739.29 |
| | P14 | 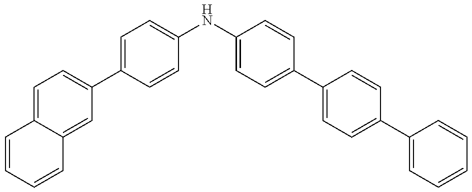 [SMILES: c1ccc(-c2ccc(-c3ccc(Nc4ccc(-c5ccc6ccccc6c5)cc4)cc3)cc2)cc1] | Compound 103 | 19.3 g (74%) | 739.29 |
| | P15 | 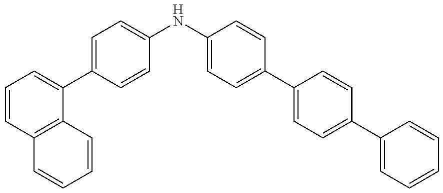 [SMILES: c1ccc(-c2ccc(-c3ccc(Nc4ccc(-c5cccc6ccccc56)cc4)cc3)cc2)cc1] | Compound 106 | 20.8 g (75%) | 789.30 |
| | P16 | 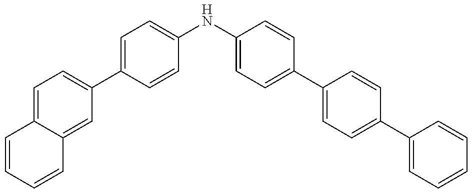 [SMILES: c1ccc(-c2ccc(-c3ccc(Nc4ccc(-c5ccc6ccccc6c5)cc4)cc3)cc2)cc1] | Compound 107 | 20.6 g (74%) | 789.30 |
| | P17 | 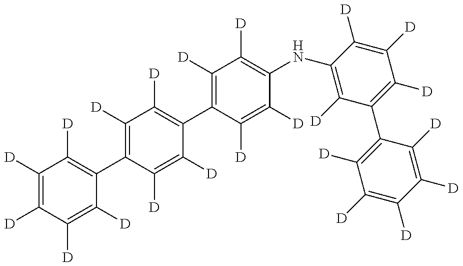 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c([2H])c5[2H])c4[2H])c([2H])c3[2H])c([2H])c2[2H])c([2H])c1[2H]] | Compound 126 | 17.3 g (73%) | 672.46 |
| | P18 | 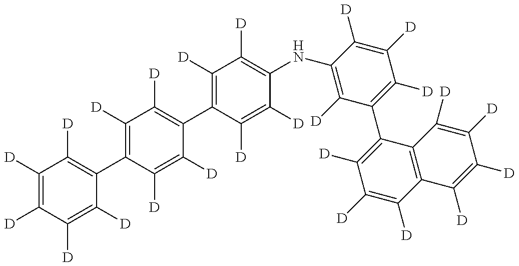 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | Compound 127 | 18.9 g (74%) | 724.49 |
| | P19 | 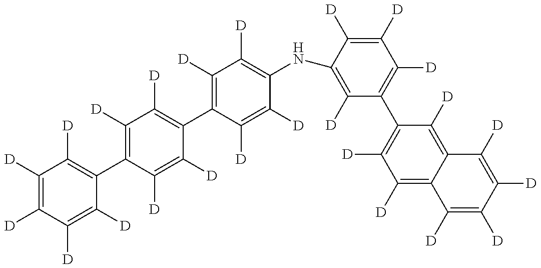 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | Compound 128 | 19.4 g (76%) | 724.49 |
| | P20 | 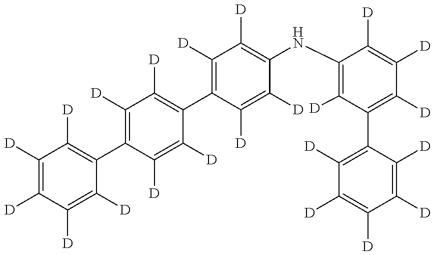 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c([2H])c5[2H])c4[2H])c([2H])c3[2H])c([2H])c2[2H])c([2H])c1[2H]] | Compound 141 | 17.9 g (70%) | 724.49 |
| | P21 | 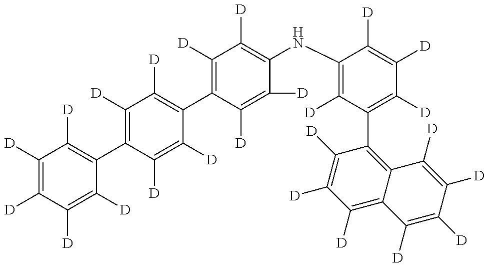 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | Compound 142 | 18.9 g (69%) | 776.52 |
| | P22 | 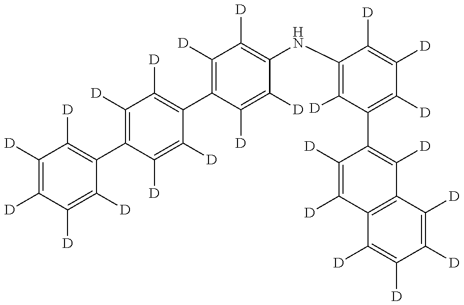 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | Compound 143 | 20.0 g (73%) | 776.52 |
| | P23 | 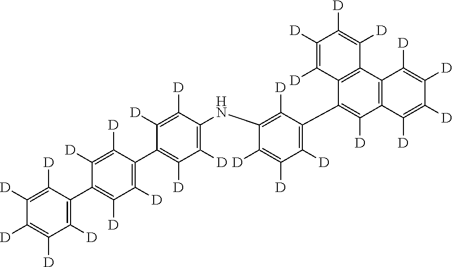 [SMILES: [2H]c1c([2H])c([2H])c(-c2c([2H])c([2H])c(-c3c([2H])c([2H])c(Nc4c([2H])c([2H])c([2H])c(-c5c([2H])c6c([2H])c([2H])c([2H])c([2H])c6c6c([2H])c([2H])c([2H])c([2H])c56)c4[2H])c([2H])c3[2H])c([2H])c2[2H])c([2H])c1[2H]] | Compound 144 | 20.7 g (71%) | 828.55 |
| | P24 | 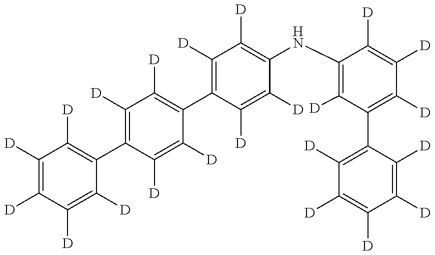 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c([2H])c5[2H])c4[2H])c([2H])c3[2H])c([2H])c2[2H])c([2H])c1[2H]] | Compound 157 | 18.4 g (72%) | 724.49 |
| | P25 | 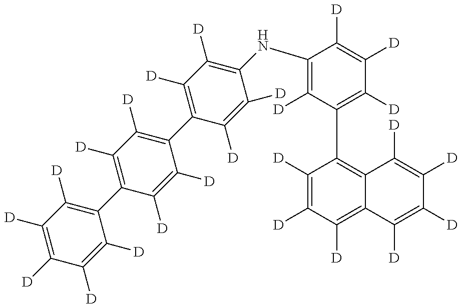 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | Compound 158 | 19.1 g (70%) | 776.52 |
| | P26 | 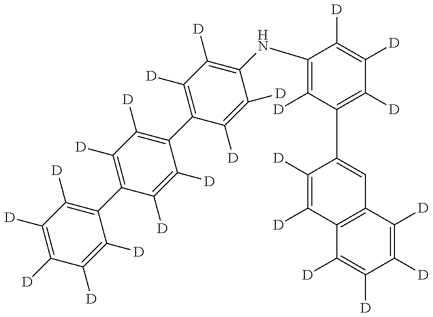 [SMILES: [2H]c1c([2H])c([2H])c(-c2c([2H])c([2H])c(-c3c([2H])c([2H])c(Nc4c([2H])c([2H])c([2H])c(-c5cc6c([2H])c([2H])c([2H])c([2H])c6c([2H])c5[2H])c4[2H])c([2H])c3[2H])c([2H])c2[2H])c([2H])c1[2H]] | Compound 159 | 20.0 g (73%) | 776.52 |
| | P27 | 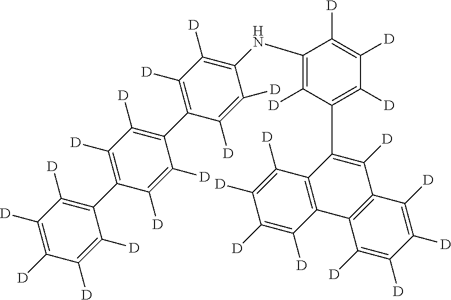 [SMILES: [2H]c1c([2H])c([2H])c(-c2c([2H])c([2H])c(-c3c([2H])c([2H])c(Nc4c([2H])c([2H])c([2H])c(-c5c([2H])c6c([2H])c([2H])c([2H])c([2H])c6c6c([2H])c([2H])c([2H])c([2H])c56)c4[2H])c([2H])c3[2H])c([2H])c2[2H])c([2H])c1[2H]] | Compound 160 | 22.2 g (76%) | 828.55 |
| | P28 | 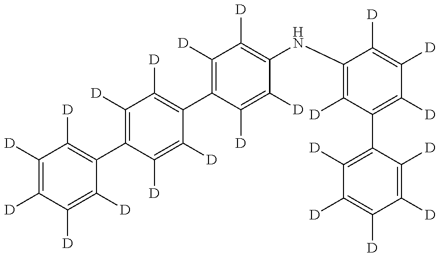 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c([2H])c5[2H])c4[2H])c([2H])c3[2H])c([2H])c2[2H])c([2H])c1[2H]] | Compound 173 | 19.4 g (71%) | 776.52 |
| | P29 | 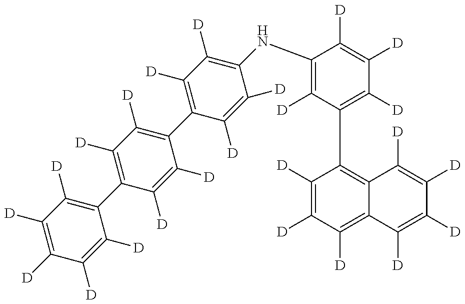 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c([2H])c6c([2H])c([2H])c([2H])c([2H])c56)c4[2H])c([2H])c3[2H])c([2H])c2[2H])c([2H])c1[2H]] | Compound 174 | 21.0 g (72%) | 828.55 |
| | P30 | 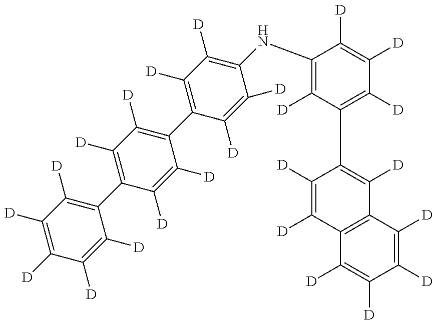 [SMILES: [2H]c1c([2H])c([2H])c(-c2c([2H])c([2H])c(-c3c([2H])c([2H])c(Nc4c([2H])c([2H])c([2H])c(-c5c([2H])c([2H])c6c([2H])c([2H])c([2H])c([2H])c6c5[2H])c4[2H])c([2H])c3[2H])c([2H])c2[2H])c([2H])c1[2H]] | Compound 175 | 21.3 g (73%) | 828.55 |
| | P31 | 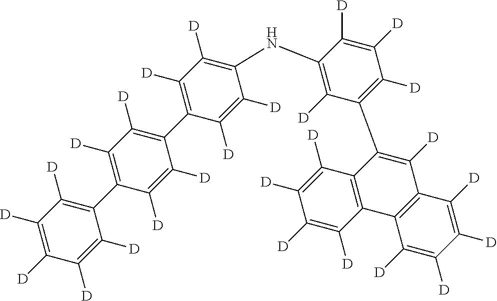 [SMILES: [2H]c1c([2H])c([2H])c(-c2c([2H])c([2H])c(-c3c([2H])c([2H])c(Nc4c([2H])c([2H])c([2H])c(-c5c([2H])c6c([2H])c([2H])c([2H])c([2H])c6c6c([2H])c([2H])c([2H])c([2H])c56)c4[2H])c([2H])c3[2H])c([2H])c2[2H])c([2H])c1[2H]] | Compound 176 | 21.7 g (70%) | 880.58 |

TABLE 2

| Item | Reactant 1 | | | | |
|---|---|---|---|---|---|
| P32 | 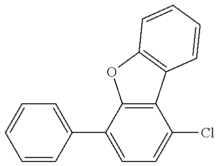 [SMILES: Clc1ccc(-c2ccccc2)c2oc3ccccc3c12] | | | | |
| P33 | 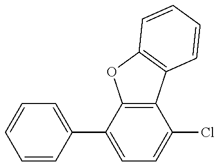 [SMILES: Clc1ccc(-c2ccccc2)c2oc3ccccc3c12] | | | | |
| P34 | 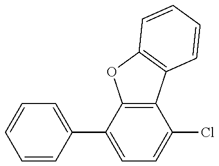 [SMILES: Clc1ccc(-c2ccccc2)c2oc3ccccc3c12] | | | | |
| P35 | 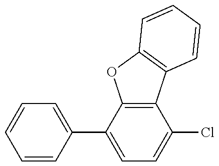 [SMILES: Clc1ccc(-c2ccccc2)c2oc3ccccc3c12] | | | | |
| P36 | 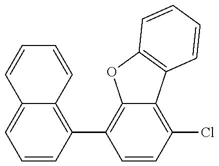 [SMILES: Clc1ccc(-c2cccc3ccccc23)c2oc3ccccc3c12] | | | | |
| P37 | 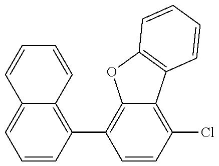 [SMILES: Clc1ccc(-c2cccc3ccccc23)c2oc3ccccc3c12] | | | | |
| P38 | 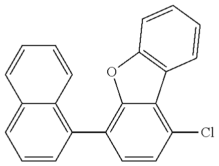 [SMILES: Clc1ccc(-c2cccc3ccccc23)c2oc3ccccc3c12] | | | | |
| P39 | 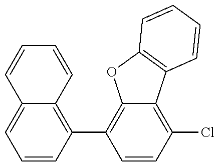 [SMILES: Clc1ccc(-c2cccc3ccccc23)c2oc3ccccc3c12] | | | | |
| P40 | 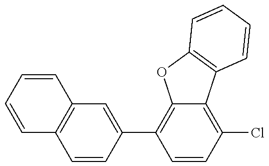 [SMILES: Clc1ccc(-c2ccc3ccccc3c2)c2oc3ccccc3c12] | | | | |
| P41 | 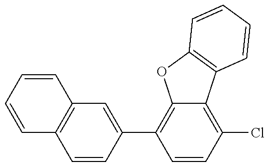 [SMILES: Clc1ccc(-c2ccc3ccccc3c2)c2oc3ccccc3c12] | | | | |
| P42 | 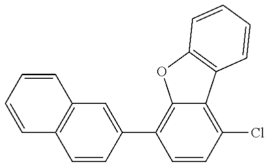 [SMILES: Clc1ccc(-c2ccc3ccccc3c2)c2oc3ccccc3c12] | | | | |
| P43 | 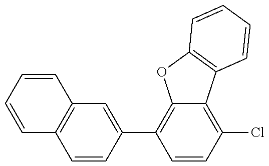 [SMILES: Clc1ccc(-c2ccc3ccccc3c2)c2oc3ccccc3c12] | | | | |
| P44 | 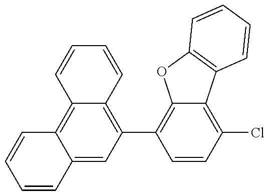 [SMILES: Clc1ccc(-c2cc3ccccc3c3ccccc23)c2oc3ccccc3c12] | | | | |
| P45 | 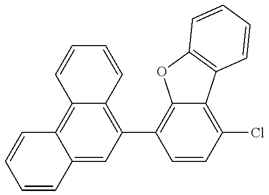 [SMILES: Clc1ccc(-c2cc3ccccc3c3ccccc23)c2oc3ccccc3c12] | | | | |
| P46 | 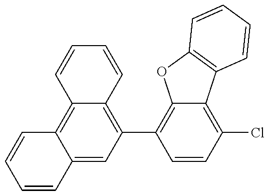 [SMILES: Clc1ccc(-c2cc3ccccc3c3ccccc23)c2oc3ccccc3c12] | | | | |
| P47 | 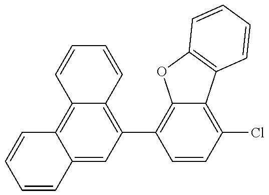 [SMILES: Clc1ccc(-c2cc3ccccc3c3ccccc23)c2oc3ccccc3c12] | | | | |
| P48 | 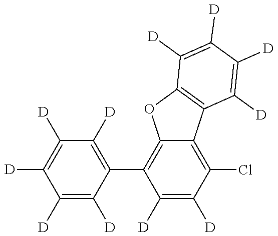 [SMILES: [2H]c1c([2H])c([2H])c(-c2c([2H])c([2H])c(Cl)c3c2oc2c([2H])c([2H])c([2H])c([2H])c23)c([2H])c1[2H]] | | | | |
| P49 | 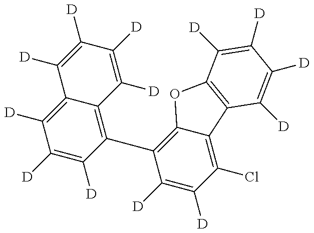 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | | | | |
| P50 | 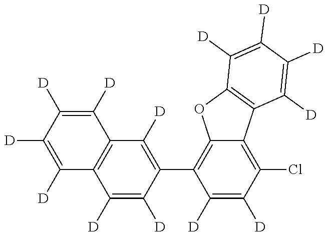 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | | | | |
| P51 | 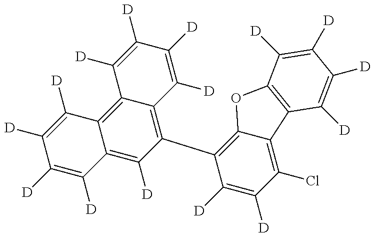 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | | | | |
| | | | | Obtained | |
| | | | | amount | |
| | Item | Reactant 2 | Product | (Yield) | [M + H]⁺ |
| | P32 | 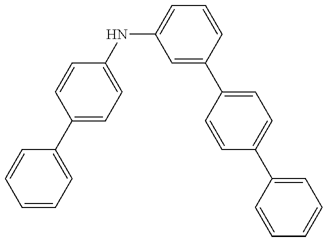 [SMILES: c1ccc(-c2ccc(Nc3cccc(-c4ccc(-c5ccccc5)cc4)c3)cc2)cc1] | Compound 225 | 16.2 g (72%) | 639.26 |
| | P33 | 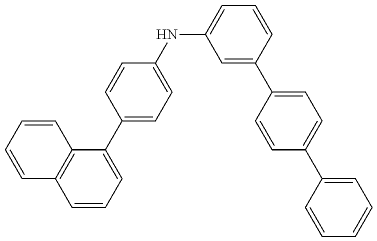 [SMILES: c1ccc(-c2ccc(-c3cccc(Nc4ccc(-c5cccc6ccccc56)cc4)c3)cc2)cc1] | Compound 226 | 18.0 g (74%) | 689.27 |
| | P34 | 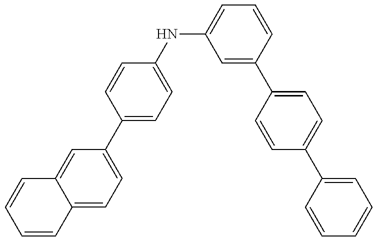 [SMILES: c1ccc(-c2ccc(-c3cccc(Nc4ccc(-c5ccc6ccccc6c5)cc4)c3)cc2)cc1] | Compound 227 | 17.0 g (70%) | 689.27 |
| | P35 | 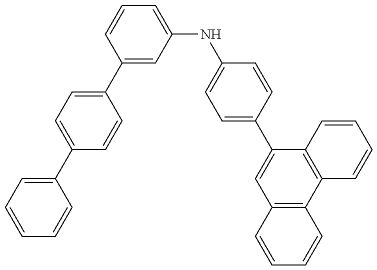 [SMILES: c1ccc(-c2ccc(-c3cccc(Nc4ccc(-c5cc6ccccc6c6ccccc56)cc4)c3)cc2)cc1] | Compound 228 | 19.5 g (75%) | 739.29 |
| | P36 | 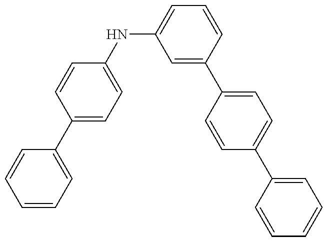 [SMILES: c1ccc(-c2ccc(Nc3cccc(-c4ccc(-c5ccccc5)cc4)c3)cc2)cc1] | Compound 230 | 15.8 g (65%) | 689.27 |
| | P37 | 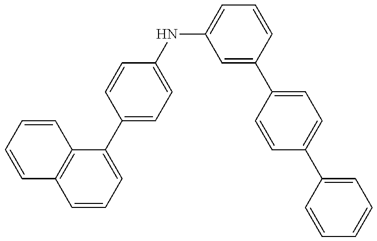 [SMILES: c1ccc(-c2ccc(-c3cccc(Nc4ccc(-c5cccc6ccccc56)cc4)c3)cc2)cc1] | Compound 231 | 18.5 g (71%) | 739.29 |
| | P38 | 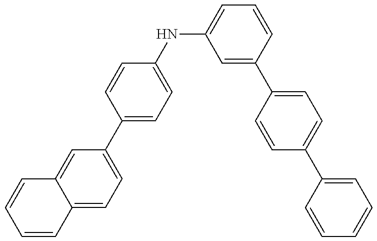 [SMILES: c1ccc(-c2ccc(-c3cccc(Nc4ccc(-c5ccc6ccccc6c5)cc4)c3)cc2)cc1] | Compound 232 | 18.5 g (71%) | 739.29 |
| | P39 | 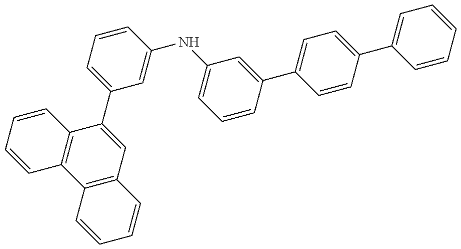 [SMILES: c1ccc(-c2ccc(-c3cccc(Nc4cccc(-c5cc6ccccc6c6ccccc56)c4)c3)cc2)cc1] | Compound 198 | 20.3 g (73%) | 789.30 |
| | P40 | 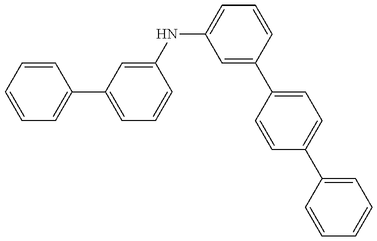 [SMILES: c1ccc(-c2ccc(-c3cccc(Nc4cccc(-c5ccccc5)c4)c3)cc2)cc1] | Compound 203 | 16.5 g (68%) | 689.27 |
| | P41 | 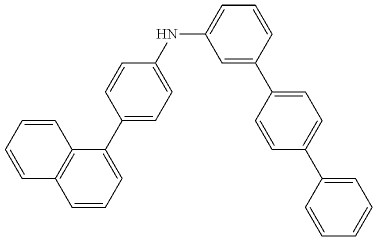 [SMILES: c1ccc(-c2ccc(-c3cccc(Nc4ccc(-c5cccc6ccccc56)cc4)c3)cc2)cc1] | Compound 236 | 19.5 g (75%) | 739.29 |
| | P42 | 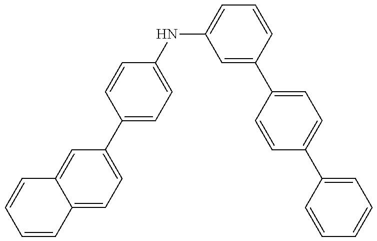 [SMILES: c1ccc(-c2ccc(-c3cccc(Nc4ccc(-c5ccc6ccccc6c5)cc4)c3)cc2)cc1] | Compound 237 | 17.4 g (67%) | 739.29 |
| | P43 | 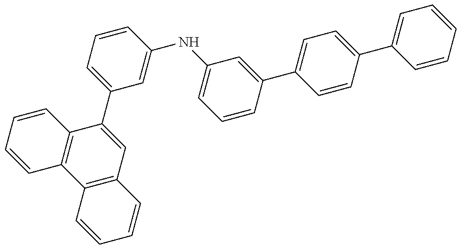 [SMILES: c1ccc(-c2ccc(-c3cccc(Nc4cccc(-c5cc6ccccc6c6ccccc56)c4)c3)cc2)cc1] | Compound 209 | 17.2 g (62%) | 789.30 |
| | P44 | 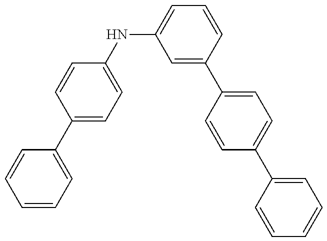 [SMILES: c1ccc(-c2ccc(Nc3cccc(-c4ccc(-c5ccccc5)cc4)c3)cc2)cc1] | Compound 240 | 15.9 g (61%) | 739.29 |
| | P45 | 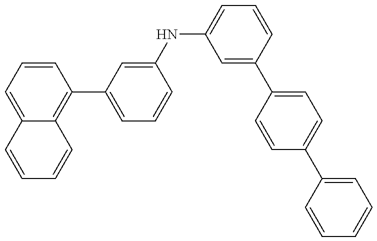 [SMILES: c1ccc(-c2ccc(-c3cccc(Nc4cccc(-c5cccc6ccccc56)c4)c3)cc2)cc1] | Compound 241 | 17.5 g (63%) | 789.30 |
| | P46 | 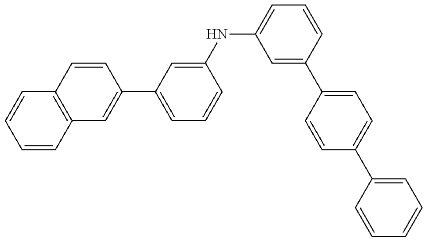 [SMILES: c1ccc(-c2ccc(-c3cccc(Nc4cccc(-c5ccc6ccccc6c5)c4)c3)cc2)cc1] | Compound 218 | 18.1 g (65%) | 789.30 |
| | P47 | 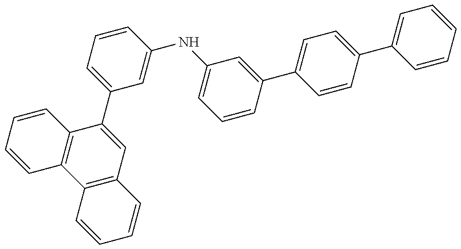 [SMILES: c1ccc(-c2ccc(-c3cccc(Nc4cccc(-c5cc6ccccc6c6ccccc56)c4)c3)cc2)cc1] | Compound 220 | 20.7 g (70%) | 839.32 |
| | P48 | 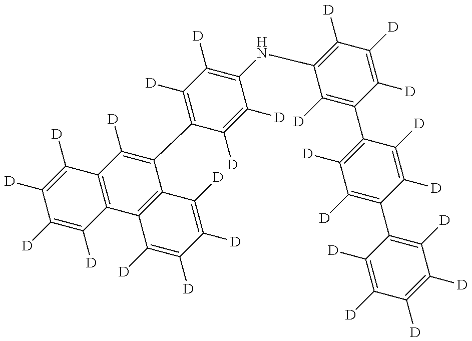 [SMILES: [2H]c1c([2H])c([2H])c(-c2c([2H])c([2H])c(-c3c([2H])c([2H])c([2H])c(Nc4c([2H])c([2H])c(-c5c([2H])c6c([2H])c([2H])c([2H])c([2H])c6c6c([2H])c([2H])c([2H])c([2H])c56)c([2H])c4[2H])c3[2H])c([2H])c2[2H])c([2H])c1[2H]] | Compound 265 | 17.2 g (63%) | 776.52 |
| | P49 | 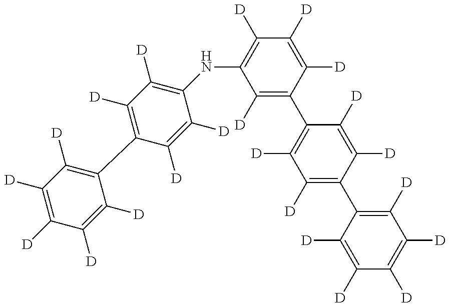 [SMILES: [2H]c1c([2H])c([2H])c(-c2c([2H])c([2H])c(Nc3c([2H])c([2H])c([2H])c(-c4c([2H])c([2H])c(-c5c([2H])c([2H])c([2H])c([2H])c5[2H])c([2H])c4[2H])c3[2H])c([2H])c2[2H])c([2H])c1[2H]] | Compound 266 | 18.6 g (73%) | 724.49 |
| | P50 | 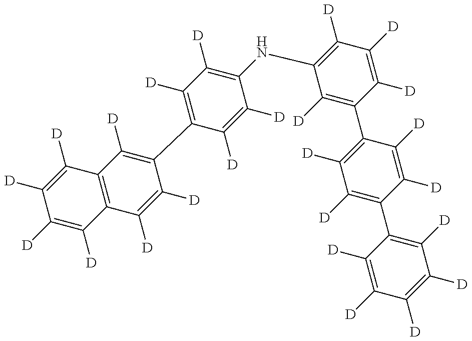 [SMILES: [2H]c1c([2H])c([2H])c(-c2c([2H])c([2H])c(-c3c([2H])c([2H])c([2H])c(Nc4c([2H])c([2H])c(-c5c([2H])c([2H])c6c([2H])c([2H])c([2H])c([2H])c6c5[2H])c([2H])c4[2H])c3[2H])c([2H])c2[2H])c([2H])c1[2H]] | Compound 267 | 16.9 g (62%) | 776.52 |
| | P51 | 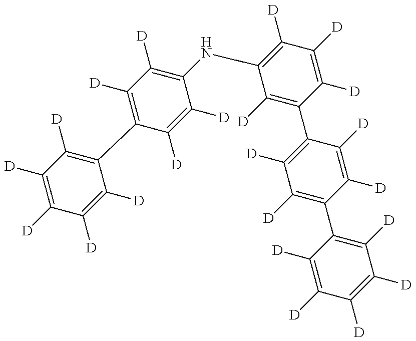 [SMILES: [2H]c1c([2H])c([2H])c(-c2c([2H])c([2H])c(Nc3c([2H])c([2H])c([2H])c(-c4c([2H])c([2H])c(-c5c([2H])c([2H])c([2H])c([2H])c5[2H])c([2H])c4[2H])c3[2H])c([2H])c2[2H])c([2H])c1[2H]] | Compound 268 | 19.7 g (72%) | 776.52 |

TABLE 3

| | | | | Obtained | |
|---|---|---|---|---|---|
| | | | | amount | [M + |
| Item | Reactant 1 | Reactant 2 | Product | (Yield) | H]⁺ |
| P52 | 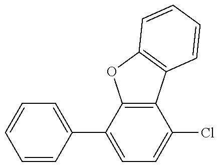 [SMILES: Clc1ccc(-c2ccccc2)c2oc3ccccc3c12] | 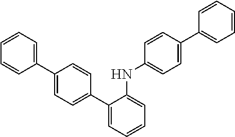 [SMILES: c1ccc(-c2ccc(Nc3ccccc3-c3ccc(-c4ccccc4)cc3)cc2)cc1] | Compound 313 | 16.0 g (71%) | 639.26 |
| P53 | 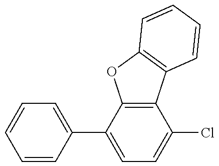 [SMILES: Clc1ccc(-c2ccccc2)c2oc3ccccc3c12] | 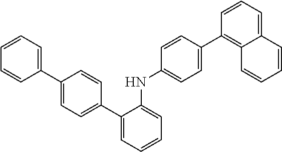 [SMILES: c1ccc(-c2ccc(-c3ccccc3Nc3ccc(-c4cccc5ccccc45)cc3)cc2)cc1] | Compound 314 | 17.5 g (72%) | 389.27 |
| P54 | 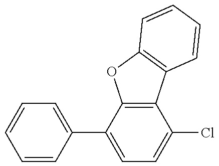 [SMILES: Clc1ccc(-c2ccccc2)c2oc3ccccc3c12] | 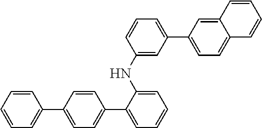 [SMILES: c1ccc(-c2ccc(-c3ccccc3Nc3cccc(-c4ccc5ccccc5c4)c3)cc2)cc1] | Compound 273 | 16.5 g (68%) | 689.28 |
| P55 | 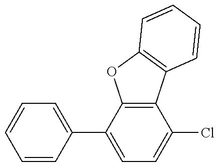 [SMILES: Clc1ccc(-c2ccccc2)c2oc3ccccc3c12] | 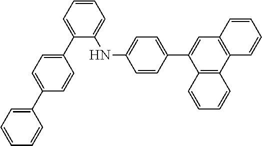 [SMILES: c1ccc(-c2ccc(-c3ccccc3Nc3ccc(-c4cc5ccccc5c5ccccc45)cc3)cc2)cc1] | Compound 316 | 17.4g (67%) | 739.29 |
| P56 | 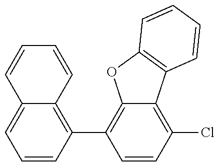 [SMILES: Clc1ccc(-c2cccc3ccccc23)c2oc3ccccc3c12] | 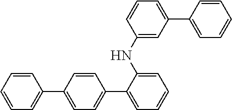 [SMILES: c1ccc(-c2ccc(-c3ccccc3Nc3cccc(-c4ccccc4)c3)cc2)cc1] | Compound 280 | 16.5 g (68%) | 689.27 |
| P57 | 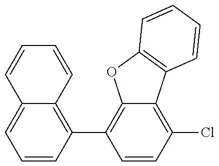 [SMILES: Clc1ccc(-c2cccc3ccccc23)c2oc3ccccc3c12] | 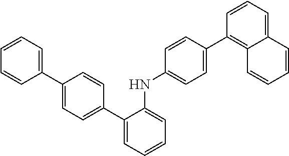 [SMILES: c1ccc(-c2ccc(-c3ccccc3Nc3ccc(-c4cccc5ccccc45)cc3)cc2)cc1] | Compound 314 | 18.5 g (71%) | 739.29 |
| P58 | 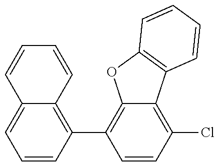 [SMILES: Clc1ccc(-c2cccc3ccccc23)c2oc3ccccc3c12] | 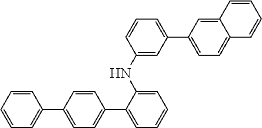 [SMILES: c1ccc(-c2ccc(-c3ccccc3Nc3cccc(-c4ccc5ccccc5c4)c3)cc2)cc1] | Compound 284 | 18.2 g (70%) | 739.29 |
| P59 | 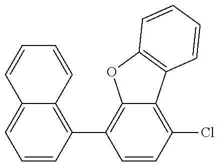 [SMILES: Clc1ccc(-c2cccc3ccccc23)c2oc3ccccc3c12] | 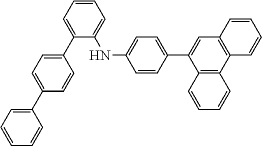 [SMILES: c1ccc(-c2ccc(-c3ccccc3Nc3ccc(-c4cc5ccccc5c5ccccc45)cc3)cc2)cc1] | Compound 321 | 20.3 g (73%) | 789.30 |
| P60 | 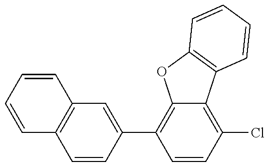 [SMILES: Clc1ccc(-c2ccc3ccccc3c2)c2oc3ccccc3c12] | 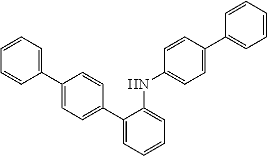 [SMILES: c1ccc(-c2ccc(Nc3ccccc3-c3ccc(-c4ccccc4)cc3)cc2)cc1] | Compound 323 | 16.3 g (67%) | 689.27 |
| P61 | 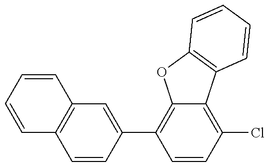 [SMILES: Clc1ccc(-c2ccc3ccccc3c2)c2oc3ccccc3c12] | 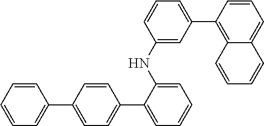 [SMILES: c1ccc(-c2ccc(-c3ccccc3Nc3cccc(-c4cccc5ccccc45)c3)cc2)cc1] | Compound 293 | 17.7 g (68%) | 739.29 |
| P62 | 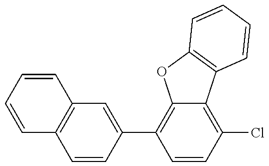 [SMILES: Clc1ccc(-c2ccc3ccccc3c2)c2oc3ccccc3c12] | 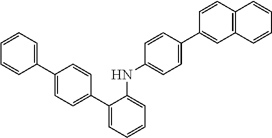 [SMILES: c1ccc(-c2ccc(-c3ccccc3Nc3ccc(-c4ccc5ccccc5c4)cc3)cc2)cc1] | Compound 325 | 17.7 g (68%) | 739.29 |
| P63 | 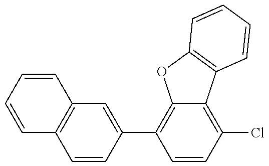 [SMILES: Clc1ccc(-c2ccc3ccccc3c2)c2oc3ccccc3c12] | 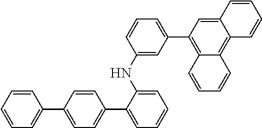 [SMILES: c1ccc(-c2ccc(-c3ccccc3Nc3cccc(-c4cc5ccccc5c5ccccc45)c3)cc2)cc1] | Compound 297 | 20.3 g (73%) | 789.30 |
| P64 | 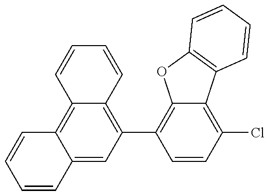 [SMILES: Clc1ccc(-c2cc3ccccc3c3ccccc23)c2oc3ccccc3c12] | 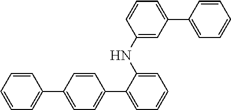 [SMILES: c1ccc(-c2ccc(-c3ccccc3Nc3cccc(-c4ccccc4)c3)cc2)cc1] | Compound 302 | 19.5 g (75%) | 739.29 |
| P65 | 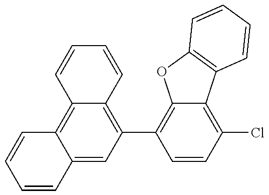 [SMILES: Clc1ccc(-c2cc3ccccc3c3ccccc23)c2oc3ccccc3c12] | 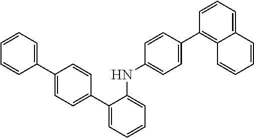 [SMILES: c1ccc(-c2ccc(-c3ccccc3Nc3ccc(-c4cccc5ccccc45)cc3)cc2)cc1] | Compound 329 | 20.6 g (74%) | 789.30 |
| P66 | 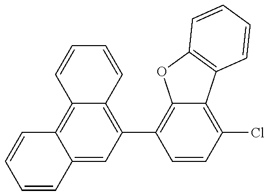 [SMILES: Clc1ccc(-c2cc3ccccc3c3ccccc23)c2oc3ccccc3c12] | 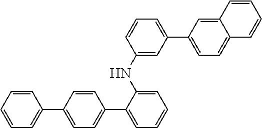 [SMILES: c1ccc(-c2ccc(-c3ccccc3Nc3cccc(-c4ccc5ccccc5c4)c3)cc2)cc1] | Compound 306 | 19.4 g (70%) | 789.30 |
| P67 | 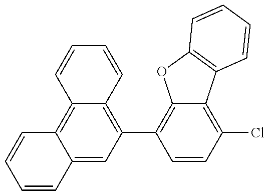 [SMILES: Clc1ccc(-c2cc3ccccc3c3ccccc23)c2oc3ccccc3c12] | 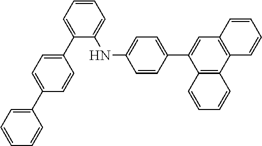 [SMILES: c1ccc(-c2ccc(-c3ccccc3Nc3ccc(-c4cc5ccccc5c5ccccc45)cc3)cc2)cc1] | Compound 331 | 20.7 g (70%) | 839.32 |
| P68 | 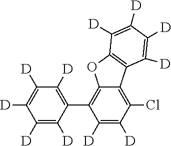 [SMILES: [2H]c1c([2H])c([2H])c(-c2c([2H])c([2H])c(Cl)c3c2oc2c([2H])c([2H])c([2H])c([2H])c23)c([2H])c1[2H]] | 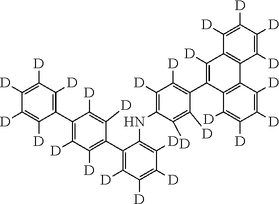 [SMILES: [2H]c1c([2H])c([2H])c(-c2c([2H])c([2H])c(-c3c([2H])c([2H])c([2H])c([2H])c3Nc3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | Compound 353 | 16.7 g (61%) | 776.52 |
| P69 | 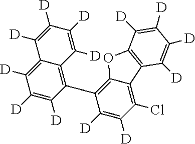 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 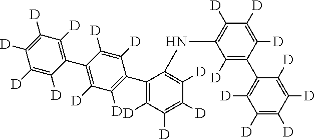 [SMILES: [2H]c1c([2H])c([2H])c(-c2c([2H])c([2H])c(-c3c([2H])c([2H])c([2H])c([2H])c3Nc3c([2H])c([2H])c([2H])c(-c4c([2H])c([2H])c([2H])c([2H])c4[2H])c3[2H])c([2H])c2[2H])c([2H])c1[2H]] | Compound 354 | 17.3 g (68%) | 724.49 |
| P70 | 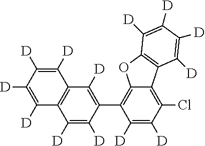 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 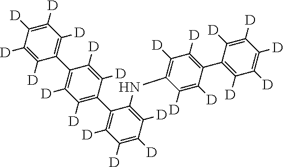 [SMILES: [2H]c1c([2H])c([2H])c(-c2c([2H])c([2H])c(Nc3c([2H])c([2H])c([2H])c([2H])c3-c3c([2H])c([2H])c(-c4c([2H])c([2H])c([2H])c([2H])c4[2H])c([2H])c3[2H])c([2H])c2[2H])c([2H])c1[2H]] | Compound 355 | 17.1 g (67%) | 724.49 |
| P71 | 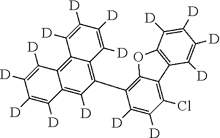 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 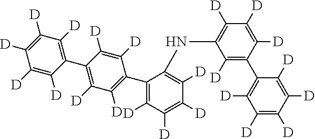 [SMILES: [2H]c1c([2H])c([2H])c(-c2c([2H])c([2H])c(-c3c([2H])c([2H])c([2H])c([2H])c3Nc3c([2H])c([2H])c([2H])c(-c4c([2H])c([2H])c([2H])c([2H])c4[2H])c3[2H])c([2H])c2[2H])c([2H])c1[2H]] | Compound 356 | 20.0 g (73%) | 776.52 |

TABLE 4

| | | | | Obtained | |
|---|---|---|---|---|---|
| | | | | amount | |
| Item | Reactant 1 | Reactant 2 | Product | (Yield) | [M + H]⁺ |
| P72 | 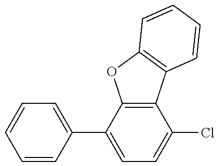 [SMILES: Clc1ccc(-c2ccccc2)c2oc3ccccc3c12] | 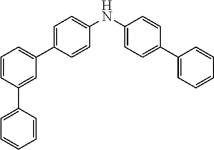 [SMILES: c1ccc(-c2ccc(Nc3ccc(-c4cccc(-c5ccccc5)c4)cc3)cc2)cc1] | Compound 403 | 17.1 g (76%) | 639.26 |
| P73 | 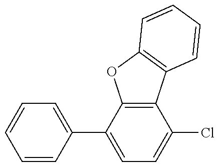 [SMILES: Clc1ccc(-c2ccccc2)c2oc3ccccc3c12] | 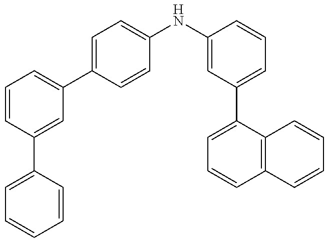 [SMILES: c1ccc(-c2cccc(-c3ccc(Nc4cccc(-c5cccc6ccccc56)c4)cc3)c2)cc1] | Compound 359 | 17.2 g (71%) | 689.27 |
| P74 | 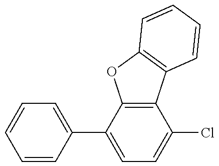 [SMILES: Clc1ccc(-c2ccccc2)c2oc3ccccc3c12] | 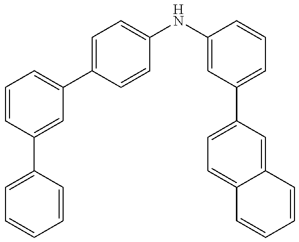 [SMILES: c1ccc(-c2cccc(-c3ccc(Nc4cccc(-c5ccc6ccccc6c5)c4)cc3)c2)cc1] | Compound 361 | 16.7 g (69%) | 689.27 |
| P75 | 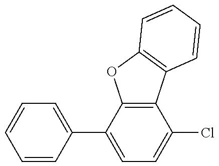 [SMILES: Clc1ccc(-c2ccccc2)c2oc3ccccc3c12] | 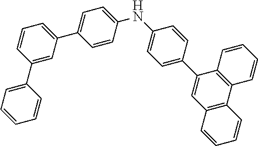 [SMILES: c1ccc(-c2cccc(-c3ccc(Nc4ccc(-c5cc6ccccc6c6ccccc56)cc4)cc3)c2)cc1] | Compound 406 | 16.1g (62%) | 739.29 |
| P76 | 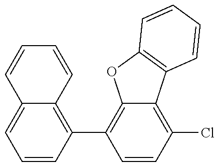 [SMILES: Clc1ccc(-c2cccc3ccccc23)c2oc3ccccc3c12] | 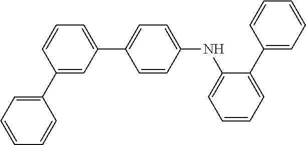 [SMILES: c1ccc(-c2cccc(-c3ccc(Nc4ccccc4-c4ccccc4)cc3)c2)cc1] | Compound 371 | 16.7 g (69%) | 689.27 |
| P77 | 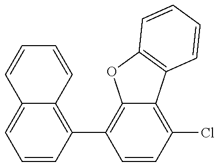 [SMILES: Clc1ccc(-c2cccc3ccccc23)c2oc3ccccc3c12] | 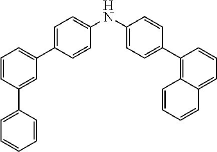 [SMILES: c1ccc(-c2cccc(-c3ccc(Nc4ccc(-c5cccc6ccccc56)cc4)cc3)c2)cc1] | Compound 409 | 17.4 g (67%) | 739.29 |
| P78 | 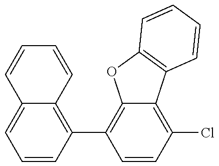 [SMILES: Clc1ccc(-c2cccc3ccccc23)c2oc3ccccc3c12] | 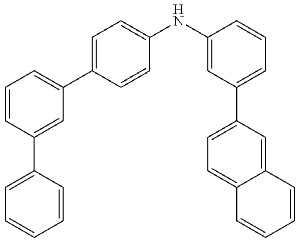 [SMILES: c1ccc(-c2cccc(-c3ccc(Nc4cccc(-c5ccc6ccccc6c5)c4)cc3)c2)cc1] | Compound 374 | 18.7 g (72%) | 739.29 |
| P79 | 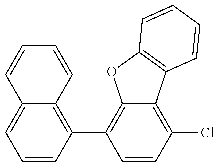 [SMILES: Clc1ccc(-c2cccc3ccccc23)c2oc3ccccc3c12] | 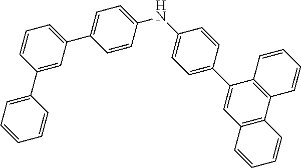 [SMILES: c1ccc(-c2cccc(-c3ccc(Nc4ccc(-c5cc6ccccc6c6ccccc56)cc4)cc3)c2)cc1] | Compound 411 | 20.3 g (73%) | 789.30 |
| P80 | 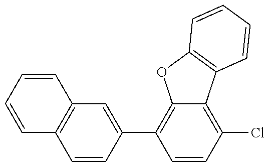 [SMILES: Clc1ccc(-c2ccc3ccccc3c2)c2oc3ccccc3c12] | 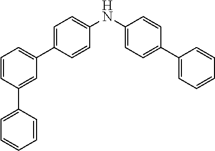 [SMILES: c1ccc(-c2ccc(Nc3ccc(-c4cccc(-c5ccccc5)c4)cc3)cc2)cc1] | Compound 413 | 16.3 g (67%) | 689.27 |
| P81 | 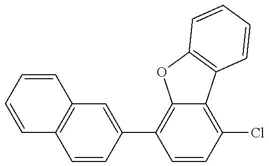 [SMILES: Clc1ccc(-c2ccc3ccccc3c2)c2oc3ccccc3c12] | 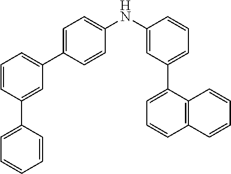 [SMILES: c1ccc(-c2cccc(-c3ccc(Nc4cccc(-c5cccc6ccccc56)c4)cc3)c2)cc1] | Compound 383 | 17.7 g (66%) | 739.29 |
| P82 | 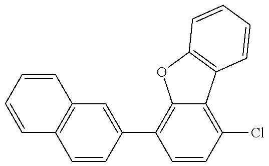 [SMILES: Clc1ccc(-c2ccc3ccccc3c2)c2oc3ccccc3c12] | 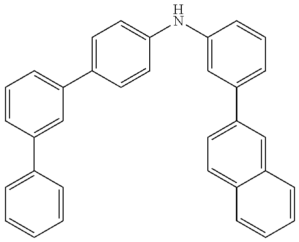 [SMILES: c1ccc(-c2cccc(-c3ccc(Nc4cccc(-c5ccc6ccccc6c5)c4)cc3)c2)cc1] | Compound 385 | 17.4 g (70%) | 739.29 |
| P83 | 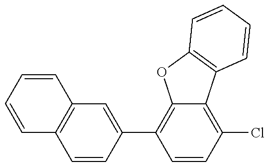 [SMILES: Clc1ccc(-c2ccc3ccccc3c2)c2oc3ccccc3c12] | 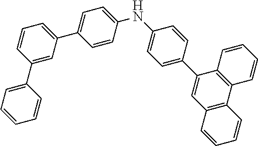 [SMILES: c1ccc(-c2cccc(-c3ccc(Nc4ccc(-c5cc6ccccc6c6ccccc56)cc4)cc3)c2)cc1] | Compound 416 | 18.3 g (76%) | 789.30 |
| P84 | 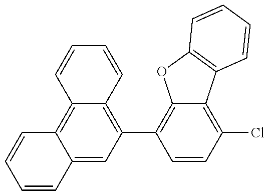 [SMILES: Clc1ccc(-c2cc3ccccc3c3ccccc23)c2oc3ccccc3c12] | 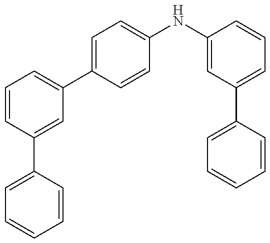 [SMILES: c1ccc(-c2cccc(Nc3ccc(-c4cccc(-c5ccccc5)c4)cc3)c2)cc1] | Compound 392 | 18.2 g (70%) | 739.29 |
| P85 | 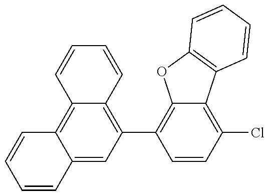 [SMILES: Clc1ccc(-c2cc3ccccc3c3ccccc23)c2oc3ccccc3c12] | 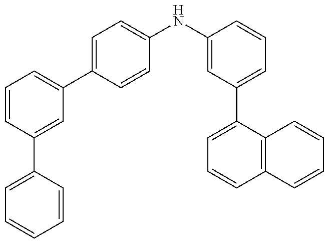 [SMILES: c1ccc(-c2cccc(-c3ccc(Nc4cccc(-c5cccc6ccccc56)c4)cc3)c2)cc1] | Compound 394 | 21.1 g (76%) | 789.30 |
| P86 | 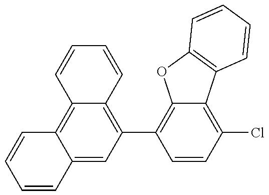 [SMILES: Clc1ccc(-c2cc3ccccc3c3ccccc23)c2oc3ccccc3c12] | 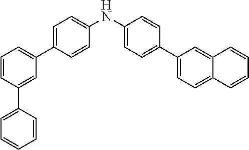 [SMILES: c1ccc(-c2cccc(-c3ccc(Nc4ccc(-c5ccc6ccccc6c5)cc4)cc3)c2)cc1] | Compound 420 | 19.4 g (70%) | 789.30 |
| P87 | 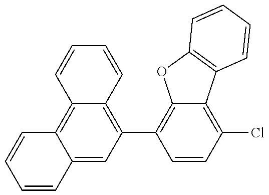 [SMILES: Clc1ccc(-c2cc3ccccc3c3ccccc23)c2oc3ccccc3c12] | 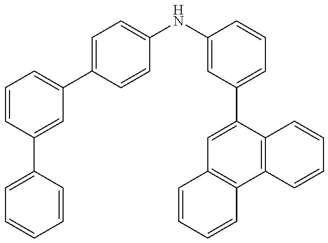 [SMILES: c1ccc(-c2cccc(-c3ccc(Nc4cccc(-c5cc6ccccc6c6ccccc56)c4)cc3)c2)cc1] | Compound 398 | 21.0 g (71%) | 839.32 |
| P88 | 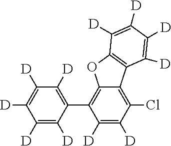 [SMILES: [2H]c1c([2H])c([2H])c(-c2c([2H])c([2H])c(Cl)c3c2oc2c([2H])c([2H])c([2H])c([2H])c23)c([2H])c1[2H]] | 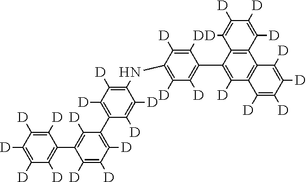 [SMILES: [2H]c1c([2H])c([2H])c(-c2c([2H])c([2H])c([2H])c(-c3c([2H])c([2H])c(Nc4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c2[2H])c([2H])c1[2H]] | Compound 423 | 19.7 g (72%) | 776.52 |
| P89 | 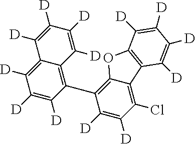 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 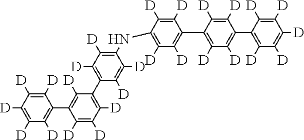 [SMILES: [2H]c1c([2H])c([2H])c(-c2c([2H])c([2H])c(-c3c([2H])c([2H])c(Nc4c([2H])c([2H])c(-c5c([2H])c([2H])c([2H])c(-c6c([2H])c([2H])c([2H])c([2H])c6[2H])c5[2H])c([2H])c4[2H])c([2H])c3[2H])c([2H])c2[2H])c([2H])c1[2H]] | Compound 424 | 18.6 g (73%) | 724.49 |
| P90 | 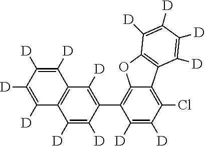 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 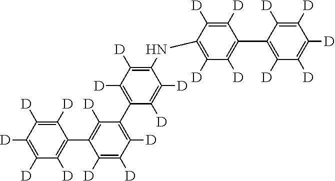 [SMILES: [2H]c1c([2H])c([2H])c(-c2c([2H])c([2H])c(Nc3c([2H])c([2H])c(-c4c([2H])c([2H])c([2H])c(-c5c([2H])c([2H])c([2H])c([2H])c5[2H])c4[2H])c([2H])c3[2H])c([2H])c2[2H])c([2H])c1[2H]] | Compound 425 | 17.3 g (68%) | 724.49 |
| P91 | 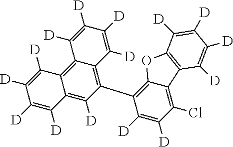 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 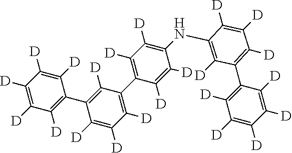 [SMILES: [2H]c1c([2H])c([2H])c(-c2c([2H])c([2H])c([2H])c(Nc3c([2H])c([2H])c(-c4c([2H])c([2H])c([2H])c(-c5c([2H])c([2H])c([2H])c([2H])c5[2H])c4[2H])c([2H])c3[2H])c2[2H])c([2H])c1[2H]] | Compound 426 | 18.3 g (67%) | 776.52 |

TABLE 5

| | | | | Obtained | |
|---|---|---|---|---|---|
| | | | | amount | |
| Item | Reactant 1 | Reactant 2 | Product | (Yield) | [M + H]⁺ |
| P92 | 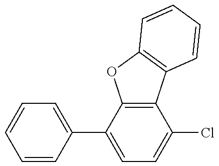 [SMILES: Clc1ccc(-c2ccccc2)c2oc3ccccc3c12] | 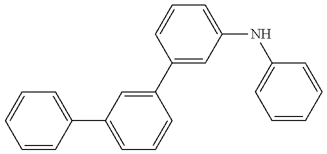 [SMILES: c1ccc(Nc2cccc(-c3cccc(-c4ccccc4)c3)c2)cc1] | Compound 494 | 13.1 g (66%) | 563.22 |
| P93 | 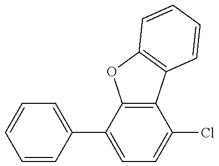 [SMILES: Clc1ccc(-c2ccccc2)c2oc3ccccc3c12] | 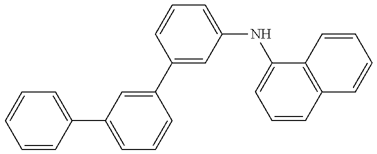 [SMILES: c1ccc(-c2cccc(-c3cccc(Nc4cccc5ccccc45)c3)c2)cc1] | Compound 495 | 13.6 g (63%) | 613.24 |
| P94 | 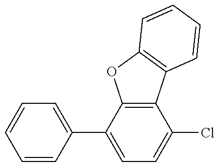 [SMILES: Clc1ccc(-c2ccccc2)c2oc3ccccc3c12] | 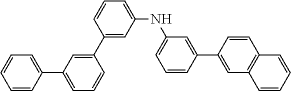 [SMILES: c1ccc(-c2cccc(-c3cccc(Nc4cccc(-c5ccc6ccccc6c5)c4)c3)c2)cc1] | Compound 431 | 15.0 g (62%) | 689.27 |
| P95 | 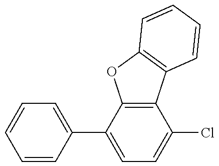 [SMILES: Clc1ccc(-c2ccccc2)c2oc3ccccc3c12] | 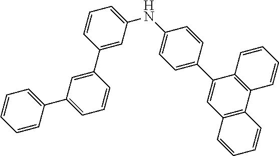 [SMILES: c1ccc(-c2cccc(-c3cccc(Nc4ccc(-c5cc6ccccc6c6ccccc56)cc4)c3)c2)cc1] | Compound 474 | 16.7 g (64%) | 739.29 |
| P96 | 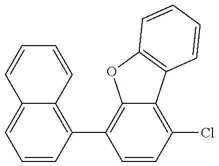 [SMILES: Clc1ccc(-c2cccc3ccccc23)c2oc3ccccc3c12] | 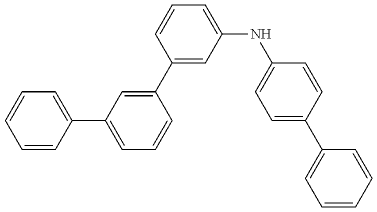 [SMILES: c1ccc(-c2ccc(Nc3cccc(-c4cccc(-c5ccccc5)c4)c3)cc2)cc1] | Compound 479 | 16.0 g (66%) | 689.27 |
| P97 | 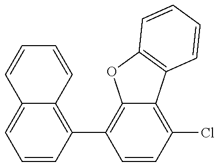 [SMILES: Clc1ccc(-c2cccc3ccccc23)c2oc3ccccc3c12] | 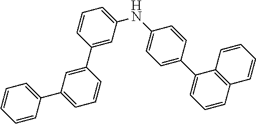 [SMILES: c1ccc(-c2cccc(-c3cccc(Nc4ccc(-c5cccc6ccccc56)cc4)c3)c2)cc1] | Compound 480 | 18.5 g (71%) | 739.29 |
| P98 | 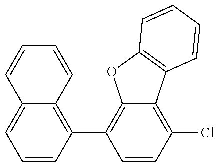 [SMILES: Clc1ccc(-c2cccc3ccccc23)c2oc3ccccc3c12] | 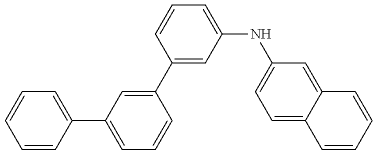 [SMILES: c1ccc(-c2cccc(-c3cccc(Nc4ccc5ccccc5c4)c3)c2)cc1] | Compound 496 | 14.9 g (64%) | 663.26 |
| P99 | 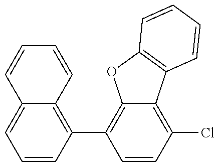 [SMILES: Clc1ccc(-c2cccc3ccccc23)c2oc3ccccc3c12] | 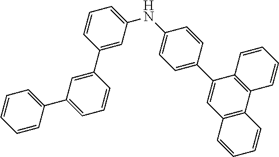 [SMILES: c1ccc(-c2cccc(-c3cccc(Nc4ccc(-c5cc6ccccc6c6ccccc56)cc4)c3)c2)cc1] | Compound 482 | 20.0 g (72%) | 789.30 |
| P100 | 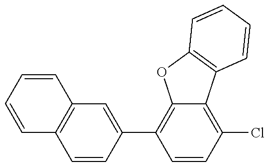 [SMILES: Clc1ccc(-c2ccc3ccccc3c2)c2oc3ccccc3c12] | 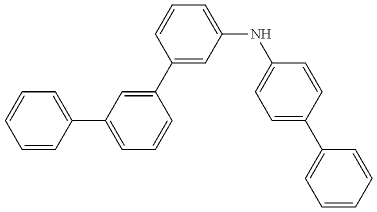 [SMILES: c1ccc(-c2ccc(Nc3cccc(-c4cccc(-c5ccccc5)c4)c3)cc2)cc1] | Compound 484 | 15.8 g (65%) | 689.27 |
| P101 | 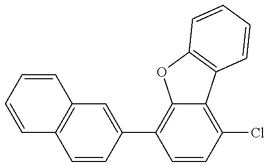 [SMILES: Clc1ccc(-c2ccc3ccccc3c2)c2oc3ccccc3c12] | 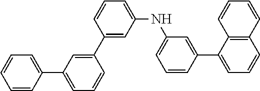 [SMILES: c1ccc(-c2cccc(-c3cccc(Nc4cccc(-c5cccc6ccccc56)c4)c3)c2)cc1] | Compound 451 | 19.0 g (73%) | 739.29 |
| P102 | 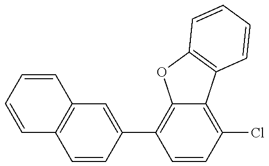 [SMILES: Clc1ccc(-c2ccc3ccccc3c2)c2oc3ccccc3c12] | 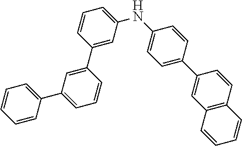 [SMILES: c1ccc(-c2cccc(-c3cccc(Nc4ccc(-c5ccc6ccccc6c5)cc4)c3)c2)cc1] | Compound 486 | 19.5 g (75%) | 739.29 |
| P103 | 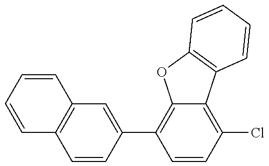 [SMILES: Clc1ccc(-c2ccc3ccccc3c2)c2oc3ccccc3c12] | 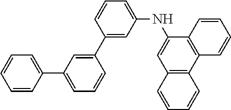 [SMILES: c1ccc(-c2cccc(-c3cccc(Nc4cc5ccccc5c5ccccc45)c3)c2)cc1] | Compound 498 | 17.8 g (71%) | 713.27 |
| P104 | 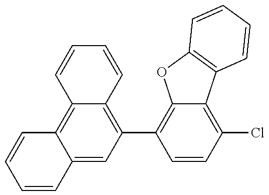 [SMILES: Clc1ccc(-c2cc3ccccc3c3ccccc23)c2oc3ccccc3c12] | 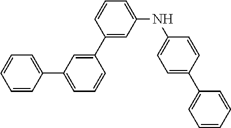 [SMILES: c1ccc(-c2ccc(Nc3cccc(-c4cccc(-c5ccccc5)c4)c3)cc2)cc1] | Compound 489 | 19.0 g (73%) | 739.29 |
| P105 | 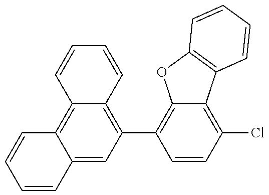 [SMILES: Clc1ccc(-c2cc3ccccc3c3ccccc23)c2oc3ccccc3c12] | 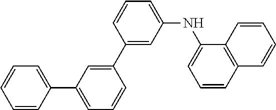 [SMILES: c1ccc(-c2cccc(-c3cccc(Nc4cccc5ccccc45)c3)c2)cc1] | Compound 499 | 18.3 g (73%) | 713.27 |
| P106 | 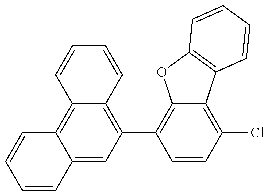 [SMILES: Clc1ccc(-c2cc3ccccc3c3ccccc23)c2oc3ccccc3c12] | 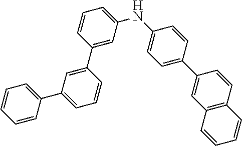 [SMILES: c1ccc(-c2cccc(-c3cccc(Nc4ccc(-c5ccc6ccccc6c5)cc4)c3)c2)cc1] | Compound 491 | 19.2 g (69%) | 789.30 |
| P107 | 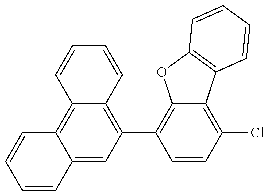 [SMILES: Clc1ccc(-c2cc3ccccc3c3ccccc23)c2oc3ccccc3c12] | 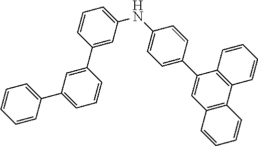 [SMILES: c1ccc(-c2cccc(-c3cccc(Nc4ccc(-c5cc6ccccc6c6ccccc56)cc4)c3)c2)cc1] | Compound 492 | 20.7 g (70%) | 839.32 |
| P108 | 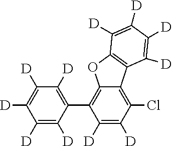 [SMILES: [2H]c1c([2H])c([2H])c(-c2c([2H])c([2H])c(Cl)c3c2oc2c([2H])c([2H])c([2H])c([2H])c23)c([2H])c1[2H]] | 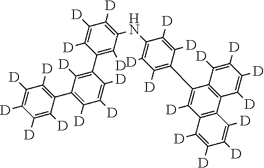 [SMILES: [2H]c1c([2H])c([2H])c(-c2c([2H])c([2H])c([2H])c(-c3c([2H])c([2H])c([2H])c(Nc4c([2H])c([2H])c(-c5c([2H])c6c([2H])c([2H])c([2H])c([2H])c6c6c([2H])c([2H])c([2H])c([2H])c56)c([2H])c4[2H])c3[2H])c2[2H])c([2H])c1[2H]] | Compound 500 | 17.2 g (63%) | 776.52 |
| P109 | 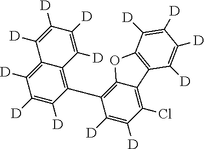 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 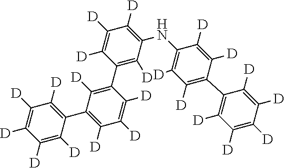 [SMILES: [2H]c1c([2H])c([2H])c(-c2c([2H])c([2H])c(Nc3c([2H])c([2H])c([2H])c(-c4c([2H])c([2H])c([2H])c(-c5c([2H])c([2H])c([2H])c([2H])c5[2H])c4[2H])c3[2H])c([2H])c2[2H])c([2H])c1[2H]] | Compound 501 | 17.3 g (68%) | 724.49 |
| P110 | 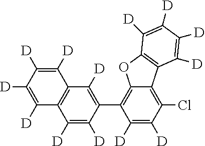 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 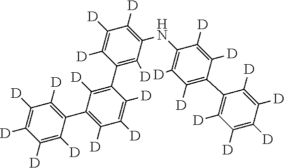 [SMILES: [2H]c1c([2H])c([2H])c(-c2c([2H])c([2H])c(Nc3c([2H])c([2H])c([2H])c(-c4c([2H])c([2H])c([2H])c(-c5c([2H])c([2H])c([2H])c([2H])c5[2H])c4[2H])c3[2H])c([2H])c2[2H])c([2H])c1[2H]] | Compound 502 | 17.6 g (69%) | 724.49 |
| P111 | 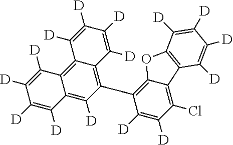 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 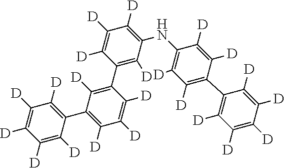 [SMILES: [2H]c1c([2H])c([2H])c(-c2c([2H])c([2H])c(Nc3c([2H])c([2H])c([2H])c(-c4c([2H])c([2H])c([2H])c(-c5c([2H])c([2H])c([2H])c([2H])c5[2H])c4[2H])c3[2H])c([2H])c2[2H])c([2H])c1[2H]] | Compound 503 | 19.1 g (70%) | 776.52 |

TABLE 6

| | | | | Obtained | |
|---|---|---|---|---|---|
| | | | | amount | |
| Item | Reactant 1 | Reactant 2 | Product | (Yield) | [M + H]⁺ |
| P112 | 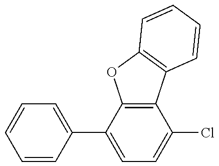 [SMILES: Clc1ccc(-c2ccccc2)c2oc3ccccc3c12] | 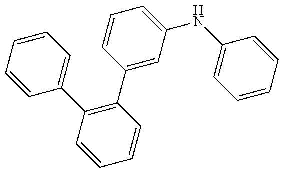 [SMILES: c1ccc(Nc2cccc(-c3ccccc3-c3ccccc3)c2)cc1] | Compound 568 | 13.5 g (68%) | 563.22 |
| P113 | 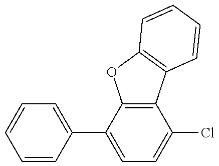 [SMILES: Clc1ccc(-c2ccccc2)c2oc3ccccc3c12] | 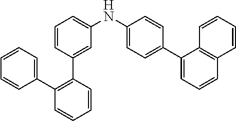 [SMILES: c1ccc(-c2ccccc2-c2cccc(Nc3ccc(-c4cccc5ccccc45)cc3)c2)cc1] | Compound 549 | 15.0 g (62%) | 689.27 |
| P114 | 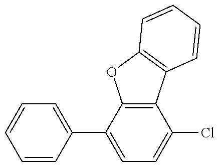 [SMILES: Clc1ccc(-c2ccccc2)c2oc3ccccc3c12] | 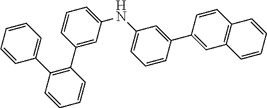 [SMILES: c1ccc(-c2ccccc2-c2cccc(Nc3cccc(-c4ccc5ccccc5c4)c3)c2)cc1] | Compound 508 | 15.3 g (63%) | 689.27 |
| P115 | 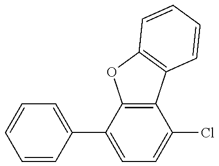 [SMILES: Clc1ccc(-c2ccccc2)c2oc3ccccc3c12] | 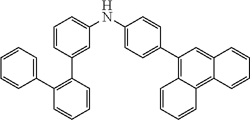 [SMILES: c1ccc(-c2ccccc2-c2cccc(Nc3ccc(-c4cc5ccccc5c5ccccc45)cc3)c2)cc1] | Compound 551 | 16.9 g (65%) | 739.29 |
| P116 | 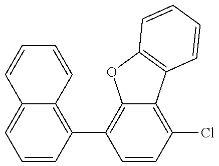 [SMILES: Clc1ccc(-c2cccc3ccccc23)c2oc3ccccc3c12] | 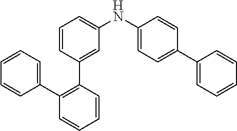 [SMILES: c1ccc(-c2ccc(Nc3cccc(-c4ccccc4-c4ccccc4)c3)cc2)cc1] | Compound 553 | 15.8 g (65%) | 689.86 |
| P117 | 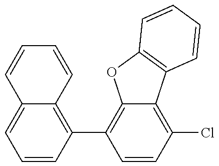 [SMILES: Clc1ccc(-c2cccc3ccccc23)c2oc3ccccc3c12] | 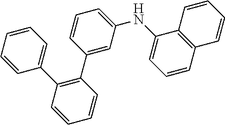 [SMILES: c1ccc(-c2ccccc2-c2cccc(Nc3cccc4ccccc34)c2)cc1] | Compound 569 | 16.3 g (70%) | 663.26 |
| P118 | 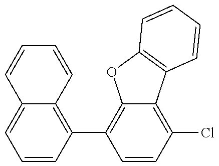 [SMILES: Clc1ccc(-c2cccc3ccccc23)c2oc3ccccc3c12] | 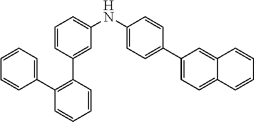 [SMILES: c1ccc(-c2ccccc2-c2cccc(Nc3ccc(-c4ccc5ccccc5c4)cc3)c2)cc1] | Compound 555 | 16.4 g (63%) | 739.29 |
| P119 | 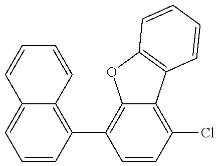 [SMILES: Clc1ccc(-c2cccc3ccccc23)c2oc3ccccc3c12] | 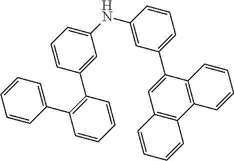 [SMILES: c1ccc(-c2ccccc2-c2cccc(Nc3cccc(-c4cc5ccccc5c5ccccc45)c3)c2)cc1] | Compound 521 | 20.3 g (73%) | 789.30 |
| P120 | 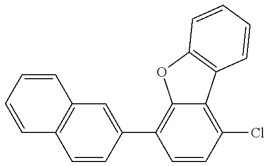 [SMILES: Clc1ccc(-c2ccc3ccccc3c2)c2oc3ccccc3c12] | 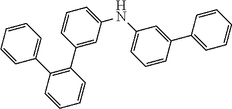 [SMILES: c1ccc(-c2cccc(Nc3cccc(-c4ccccc4-c4ccccc4)c3)c2)cc1] | Compound 526 | 16.0 g (66%) | 689.27 |
| P121 | 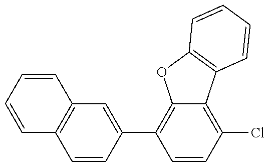 [SMILES: Clc1ccc(-c2ccc3ccccc3c2)c2oc3ccccc3c12] | 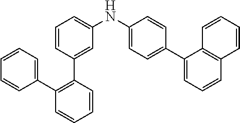 [SMILES: c1ccc(-c2ccccc2-c2cccc(Nc3ccc(-c4cccc5ccccc45)cc3)c2)cc1] | Compound 559 | 19.3 g (74%) | 739.29 |
| P122 | 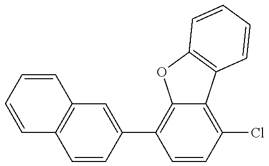 [SMILES: Clc1ccc(-c2ccc3ccccc3c2)c2oc3ccccc3c12] | 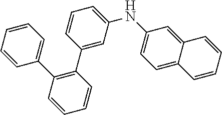 [SMILES: c1ccc(-c2ccccc2-c2cccc(Nc3ccc4ccccc4c3)c2)cc1] | Compound 570 | 17.5 g (75%) | 663.26 |
| P123 | 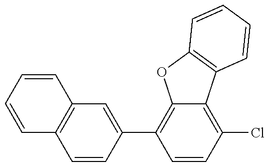 [SMILES: Clc1ccc(-c2ccc3ccccc3c2)c2oc3ccccc3c12] | 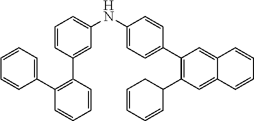 [SMILES: C1=CCC(c2cc3ccccc3cc2-c2ccc(Nc3cccc(-c4ccccc4-c4ccccc4)c3)cc2)C=C1] | Compound 561 | 21.1 g (76%) | 789.30 |
| P124 | 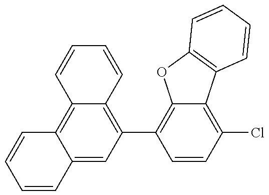 [SMILES: Clc1ccc(-c2cc3ccccc3c3ccccc23)c2oc3ccccc3c12] | 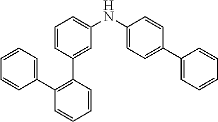 [SMILES: c1ccc(-c2ccc(Nc3cccc(-c4ccccc4-c4ccccc4)c3)cc2)cc1] | Compound 563 | 18.5 g (71%) | 739.29 |
| P125 | 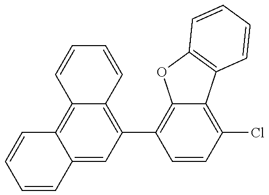 [SMILES: Clc1ccc(-c2cc3ccccc3c3ccccc23)c2oc3ccccc3c12] | 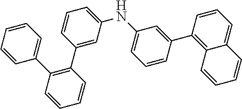 [SMILES: c1ccc(-c2ccccc2-c2cccc(Nc3cccc(-c4cccc5ccccc45)c3)c2)cc1] | Compound 539 | 20.6 g (74%) | 789.30 |
| P126 | 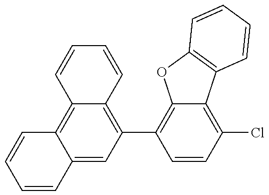 [SMILES: Clc1ccc(-c2cc3ccccc3c3ccccc23)c2oc3ccccc3c12] | 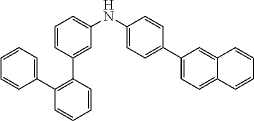 [SMILES: c1ccc(-c2ccccc2-c2cccc(Nc3ccc(-c4ccc5ccccc5c4)cc3)c2)cc1] | Compound 565 | 19.7 g (71%) | 789.30 |
| P127 | 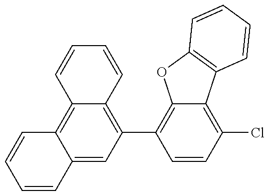 [SMILES: Clc1ccc(-c2cc3ccccc3c3ccccc23)c2oc3ccccc3c12] | 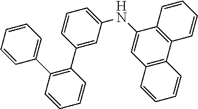 [SMILES: c1ccc(-c2ccccc2-c2cccc(Nc3cc4ccccc4c4ccccc34)c2)cc1] | Compound 571 | 17.2 g (64%) | 763.29 |
| P128 | 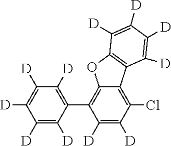 [SMILES: [2H]c1c([2H])c([2H])c(-c2c([2H])c([2H])c(Cl)c3c2oc2c([2H])c([2H])c([2H])c([2H])c23)c([2H])c1[2H]] | 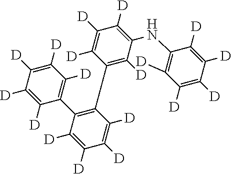 [SMILES: [2H]c1c([2H])c([2H])c(Nc2c([2H])c([2H])c([2H])c(-c3c([2H])c([2H])c([2H])c([2H])c3-c3c([2H])c([2H])c([2H])c([2H])c3[2H])c2[2H])c([2H])c1[2H]] | Compound 572 | 14.2 g (68%) | 592.41 |
| P129 | 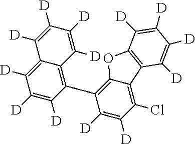 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 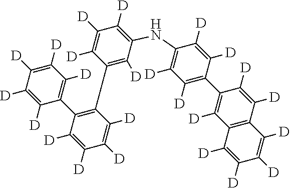 [SMILES: [2H]c1c([2H])c([2H])c(-c2c([2H])c([2H])c([2H])c([2H])c2-c2c([2H])c([2H])c([2H])c(Nc3c([2H])c([2H])c(-c4c([2H])c([2H])c5c([2H])c([2H])c([2H])c([2H])c5c4[2H])c([2H])c3[2H])c2[2H])c([2H])c1[2H]] | Compound 573 | 18.3 g (67%) | 776.52 |
| P130 | 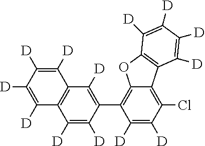 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 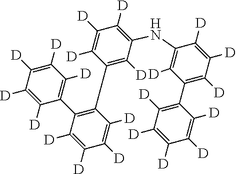 [SMILES: [2H]c1c([2H])c([2H])c(-c2c([2H])c([2H])c([2H])c(Nc3c([2H])c([2H])c([2H])c(-c4c([2H])c([2H])c([2H])c([2H])c4-c4c([2H])c([2H])c([2H])c([2H])c4[2H])c3[2H])c2[2H])c([2H])c1[2H]] | Compound 574 | 17.3 g (68%) | 724.49 |
| P131 | 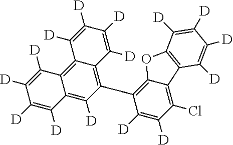 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 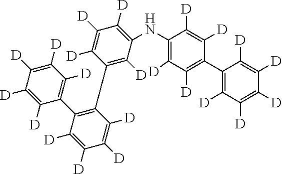 [SMILES: [2H]c1c([2H])c([2H])c(-c2c([2H])c([2H])c(Nc3c([2H])c([2H])c([2H])c(-c4c([2H])c([2H])c([2H])c([2H])c4-c4c([2H])c([2H])c([2H])c([2H])c4[2H])c3[2H])c([2H])c2[2H])c([2H])c1[2H]] | Compound 575 | 19.4 g (71%) | 776.52 |

TABLE 7

| | | | | Obtained | |
|---|---|---|---|---|---|
| | | | | amount | |
| Item | Reactant 1 | Reactant 2 | Product | (Yield) | [M + H]⁺ |
| P132 | 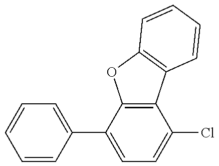 [SMILES: Clc1ccc(-c2ccccc2)c2oc3ccccc3c12] | 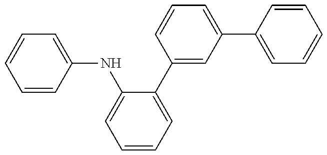 [SMILES: c1ccc(Nc2ccccc2-c2cccc(-c3ccccc3)c2)cc1] | Compound 576 | 13.9 g (70%) | 563.22 |
| P133 | 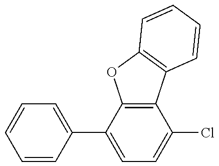 [SMILES: Clc1ccc(-c2ccccc2)c2oc3ccccc3c12] | 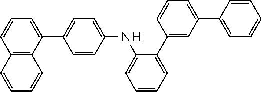 [SMILES: c1ccc(-c2cccc(-c3ccccc3Nc3ccc(-c4cccc5ccccc45)cc3)c2)cc1] | Compound 621 | 15.5 g (64%) | 689.27 |
| P134 | 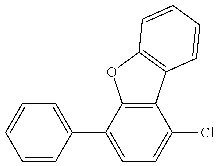 [SMILES: Clc1ccc(-c2ccccc2)c2oc3ccccc3c12] | 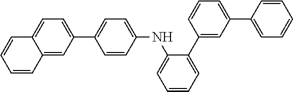 [SMILES: c1ccc(-c2cccc(-c3ccccc3Nc3ccc(-c4ccc5ccccc5c4)cc3)c2)cc1] | Compound 622 | 16.0 g (66%) | 689.27 |
| P135 | 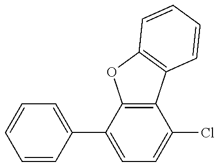 [SMILES: Clc1ccc(-c2ccccc2)c2oc3ccccc3c12] | 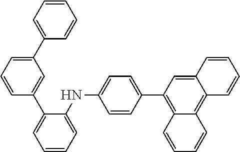 [SMILES: c1ccc(-c2cccc(-c3ccccc3Nc3ccc(-c4cc5ccccc5c5ccccc45)cc3)c2)cc1] | Compound 623 | 16.7 g (64%) | 739.29 |
| P136 | 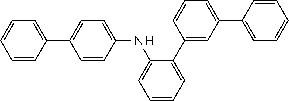 [SMILES: c1ccc(-c2ccc(Nc3ccccc3-c3cccc(-c4ccccc4)c3)cc2)cc1] | 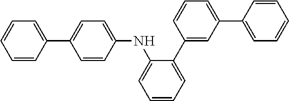 [SMILES: c1ccc(-c2ccc(Nc3ccccc3-c3cccc(-c4ccccc4)c3)cc2)cc1] | Compound 625 | 16.0 g (66%) | 689.27 |
| P137 | 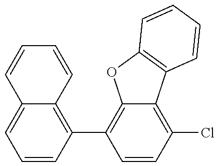 [SMILES: Clc1ccc(-c2cccc3ccccc23)c2oc3ccccc3c12] | 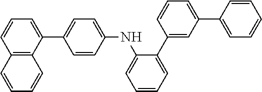 [SMILES: c1ccc(-c2cccc(-c3ccccc3Nc3ccc(-c4cccc5ccccc45)cc3)c2)cc1] | Compound 626 | 18.5 g (71%) | 739.29 |
| P138 | 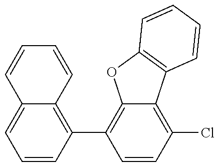 [SMILES: Clc1ccc(-c2cccc3ccccc23)c2oc3ccccc3c12] | 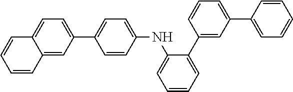 [SMILES: c1ccc(-c2cccc(-c3ccccc3Nc3ccc(-c4ccc5ccccc5c4)cc3)c2)cc1] | Compound 627 | 18.2 g (70%) | 739.29 |
| P139 | 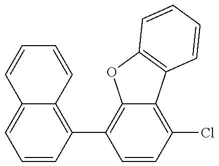 [SMILES: Clc1ccc(-c2cccc3ccccc23)c2oc3ccccc3c12] | 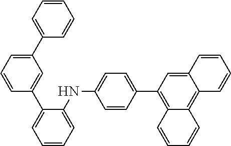 [SMILES: c1ccc(-c2cccc(-c3ccccc3Nc3ccc(-c4cc5ccccc5c5ccccc45)cc3)c2)cc1] | Compound 628 | 20.6 g (74%) | 789.30 |
| P140 | 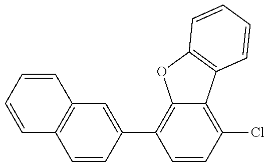 [SMILES: Clc1ccc(-c2ccc3ccccc3c2)c2oc3ccccc3c12] | 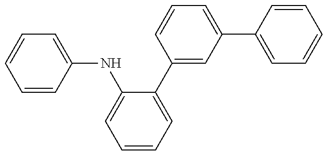 [SMILES: c1ccc(Nc2ccccc2-c2cccc(-c3ccccc3)c2)cc1] | Compound 641 | 14.5 g (67%) | 613.24 |
| P141 | 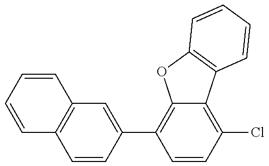 [SMILES: Clc1ccc(-c2ccc3ccccc3c2)c2oc3ccccc3c12] | 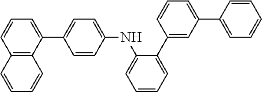 [SMILES: c1ccc(-c2cccc(-c3ccccc3Nc3ccc(-c4cccc5ccccc45)cc3)c2)cc1] | Compound 631 | 18.5 g (71%) | 739.29 |
| P142 | 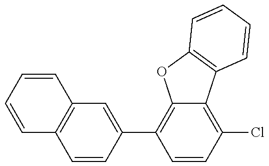 [SMILES: Clc1ccc(-c2ccc3ccccc3c2)c2oc3ccccc3c12] | 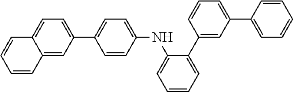 [SMILES: c1ccc(-c2cccc(-c3ccccc3Nc3ccc(-c4ccc5ccccc5c4)cc3)c2)cc1] | Compound 632 | 19.3 g (74%) | 739.29 |
| P143 | 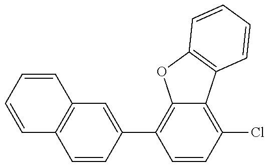 [SMILES: Clc1ccc(-c2ccc3ccccc3c2)c2oc3ccccc3c12] | 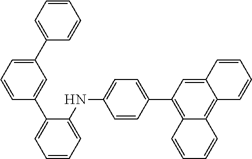 [SMILES: c1ccc(-c2cccc(-c3ccccc3Nc3ccc(-c4cc5ccccc5c5ccccc45)cc3)c2)cc1] | Compound 633 | 20.8 g (75%) | 789.30 |
| P144 | 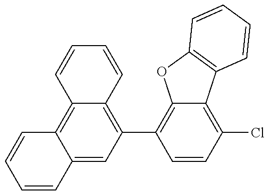 [SMILES: Clc1ccc(-c2cc3ccccc3c3ccccc23)c2oc3ccccc3c12] | 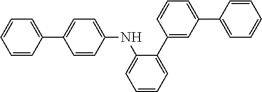 [SMILES: c1ccc(-c2ccc(Nc3ccccc3-c3cccc(-c4ccccc4)c3)cc2)cc1] | Compound 635 | 18.2 g (70%) | 739.29 |
| P145 | 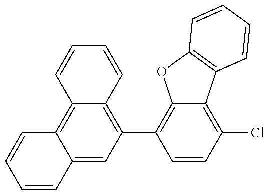 [SMILES: Clc1ccc(-c2cc3ccccc3c3ccccc23)c2oc3ccccc3c12] | 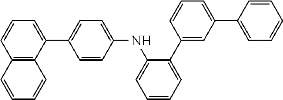 [SMILES: c1ccc(-c2cccc(-c3ccccc3Nc3ccc(-c4cccc5ccccc45)cc3)c2)cc1] | Compound 636 | 20.8 g (75%) | 789.30 |
| P146 | 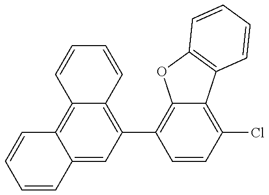 [SMILES: Clc1ccc(-c2cc3ccccc3c3ccccc23)c2oc3ccccc3c12] | 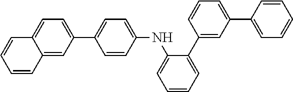 [SMILES: c1ccc(-c2cccc(-c3ccccc3Nc3ccc(-c4ccc5ccccc5c4)cc3)c2)cc1] | Compound 637 | 20.3 g (73%) | 789.30 |
| P147 | 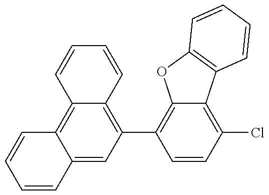 [SMILES: Clc1ccc(-c2cc3ccccc3c3ccccc23)c2oc3ccccc3c12] | 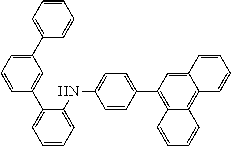 [SMILES: c1ccc(-c2cccc(-c3ccccc3Nc3ccc(-c4cc5ccccc5c5ccccc45)cc3)c2)cc1] | Compound 638 | 19.8 g (67%) | 839.32 |
| P148 | 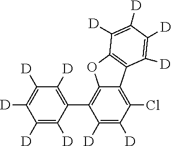 [SMILES: [2H]c1c([2H])c([2H])c(-c2c([2H])c([2H])c(Cl)c3c2oc2c([2H])c([2H])c([2H])c([2H])c23)c([2H])c1[2H]] | 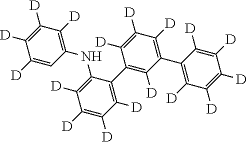 [SMILES: [2H]c1cc(Nc2c([2H])c([2H])c([2H])c([2H])c2-c2c([2H])c([2H])c([2H])c(-c3c([2H])c([2H])c([2H])c([2H])c3[2H])c2[2H])c([2H])c([2H])c1[2H]] | Compound 642 | 13.8 g (66%) | 592.41 |
| P149 | 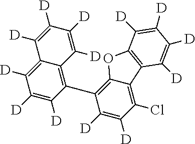 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 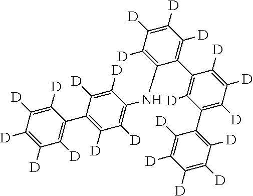 [SMILES: [2H]c1c([2H])c([2H])c(-c2c([2H])c([2H])c(Nc3c([2H])c([2H])c([2H])c([2H])c3-c3c([2H])c([2H])c([2H])c(-c4c([2H])c([2H])c([2H])c([2H])c4[2H])c3[2H])c([2H])c2[2H])c([2H])c1[2H]] | Compound 643 | 17.3 g (68%) | 724.49 |
| P150 | 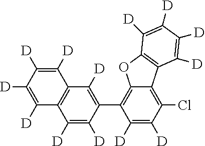 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 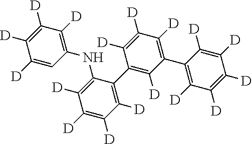 [SMILES: [2H]c1cc(Nc2c([2H])c([2H])c([2H])c([2H])c2-c2c([2H])c([2H])c([2H])c(-c3c([2H])c([2H])c([2H])c([2H])c3[2H])c2[2H])c([2H])c([2H])c1[2H]] | Compound 644 | 15.7 g (69%) | 644.44 |
| P151 | 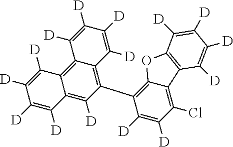 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 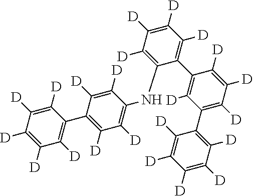 [SMILES: [2H]c1c([2H])c([2H])c(-c2c([2H])c([2H])c(Nc3c([2H])c([2H])c([2H])c([2H])c3-c3c([2H])c([2H])c([2H])c(-c4c([2H])c([2H])c([2H])c([2H])c4[2H])c3[2H])c([2H])c2[2H])c([2H])c1[2H]] | Compound 645 | 19.1 g (70%) | 776.52 |

TABLE 8

| | | | | Obtained | |
|---|---|---|---|---|---|
| | | | | amount | |
| Item | Reactant 1 | Reactant 2 | Product | (Yield) | [M + H]⁺ |
| P152 | 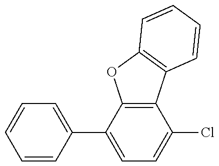 [SMILES: Clc1ccc(-c2ccccc2)c2oc3ccccc3c12] | 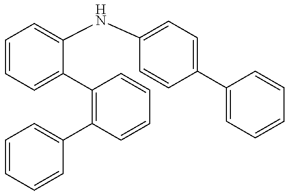 [SMILES: c1ccc(-c2ccc(Nc3ccccc3-c3ccccc3-c3ccccc3)cc2)cc1] | Compound 690 | 16.2 g (72%) | 639.80 |
| P153 | 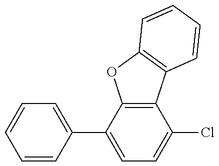 [SMILES: Clc1ccc(-c2ccccc2)c2oc3ccccc3c12] | 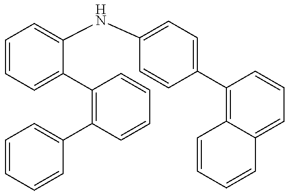 [SMILES: c1ccc(-c2ccccc2-c2ccccc2Nc2ccc(-c3cccc4ccccc34)cc2)cc1] | Compound 691 | 15.8 g (65%) | 689.86 |
| P154 | 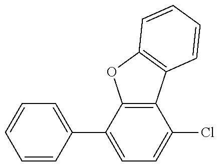 [SMILES: Clc1ccc(-c2ccccc2)c2oc3ccccc3c12] | 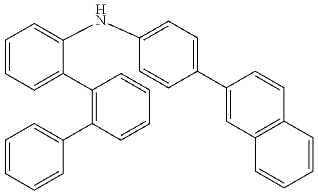 [SMILES: c1ccc(-c2ccccc2-c2ccccc2Nc2ccc(-c3ccc4ccccc4c3)cc2)cc1] | Compound 692 | 16.3 g (67%) | 689.86 |
| P155 | 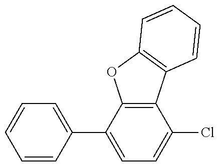 [SMILES: Clc1ccc(-c2ccccc2)c2oc3ccccc3c12] | 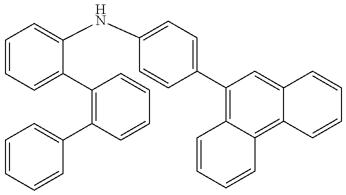 [SMILES: c1ccc(-c2ccccc2-c2ccccc2Nc2ccc(-c3cc4ccccc4c4ccccc34)cc2)cc1] | Compound 693 | 16.9 g (65%) | 739.92 |
| P156 | 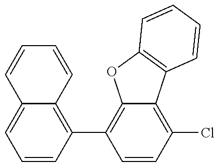 [SMILES: Clc1ccc(-c2cccc3ccccc23)c2oc3ccccc3c12] | 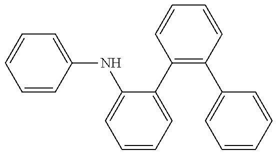 [SMILES: c1ccc(Nc2ccccc2-c2ccccc2-c2ccccc2)cc1] | Compound 710 | 14.5 g (67%) | 613.24 |
| P157 | 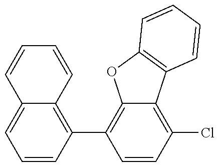 [SMILES: Clc1ccc(-c2cccc3ccccc23)c2oc3ccccc3c12] | 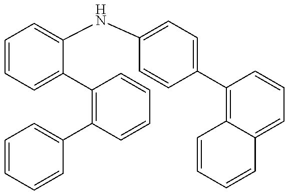 [SMILES: c1ccc(-c2ccccc2-c2ccccc2Nc2ccc(-c3cccc4ccccc34)cc2)cc1] | Compound 696 | 18.2 g (70%) | 739.29 |
| P158 | 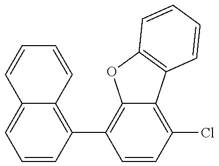 [SMILES: Clc1ccc(-c2cccc3ccccc23)c2oc3ccccc3c12] | 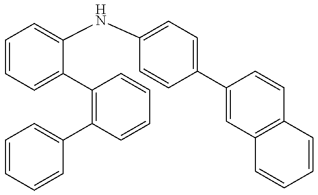 [SMILES: c1ccc(-c2ccccc2-c2ccccc2Nc2ccc(-c3ccc4ccccc4c3)cc2)cc1] | Compound 697 | 18.7 g (72%) | 739.29 |
| P159 | 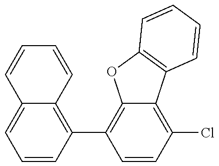 [SMILES: Clc1ccc(-c2cccc3ccccc23)c2oc3ccccc3c12] | 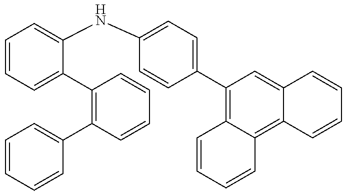 [SMILES: c1ccc(-c2ccccc2-c2ccccc2Nc2ccc(-c3cc4ccccc4c4ccccc34)cc2)cc1] | Compound 698 | 20.3 g (73%) | 789.30 |
| P160 | 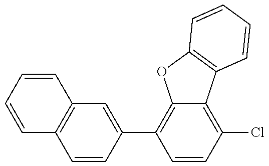 [SMILES: Clc1ccc(-c2ccc3ccccc3c2)c2oc3ccccc3c12] | 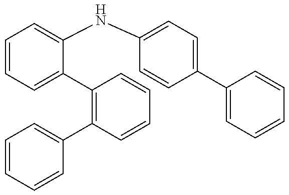 [SMILES: c1ccc(-c2ccc(Nc3ccccc3-c3ccccc3-c3ccccc3)cc2)cc1] | Compound 700 | 16.7 g (69%) | 689.27 |
| P161 | 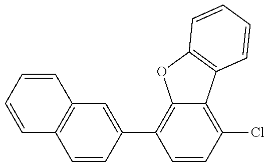 [SMILES: Clc1ccc(-c2ccc3ccccc3c2)c2oc3ccccc3c12] | 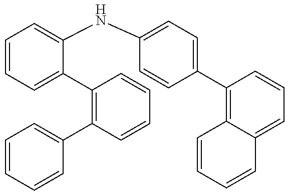 [SMILES: c1ccc(-c2ccccc2-c2ccccc2Nc2ccc(-c3cccc4ccccc34)cc2)cc1] | Compound 701 | 18.2 g (70%) | 739.29 |
| P162 | 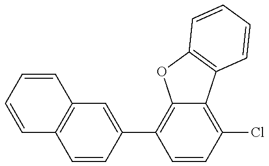 [SMILES: Clc1ccc(-c2ccc3ccccc3c2)c2oc3ccccc3c12] | 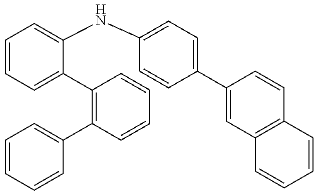 [SMILES: c1ccc(-c2ccccc2-c2ccccc2Nc2ccc(-c3ccc4ccccc4c3)cc2)cc1] | Compound 702 | 19.0 g (73%) | 739.29 |
| P163 | 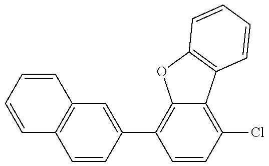 [SMILES: Clc1ccc(-c2ccc3ccccc3c2)c2oc3ccccc3c12] | 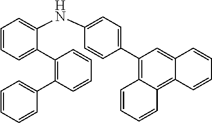 [SMILES: c1ccc(-c2ccccc2-c2ccccc2Nc2ccc(-c3cc4ccccc4c4ccccc34)cc2)cc1] | Compound 703 | 20.6 g (74%) | 789.30 |
| P164 | 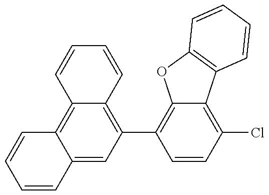 [SMILES: Clc1ccc(-c2cc3ccccc3c3ccccc23)c2oc3ccccc3c12] | 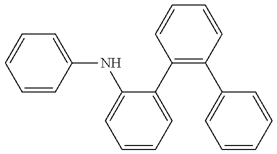 [SMILES: c1ccc(Nc2ccccc2-c2ccccc2-c2ccccc2)cc1] | Compound 711 | 16.8 g (72%) | 663.26 |
| P165 | 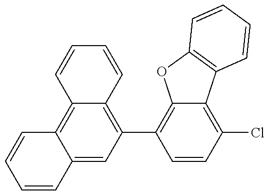 [SMILES: Clc1ccc(-c2cc3ccccc3c3ccccc23)c2oc3ccccc3c12] | 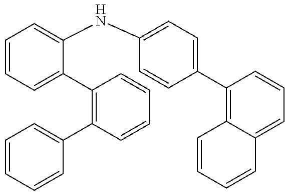 [SMILES: c1ccc(-c2ccccc2-c2ccccc2Nc2ccc(-c3cccc4ccccc34)cc2)cc1] | Compound 706 | 20.3 g (73%) | 789.30 |
| P166 | 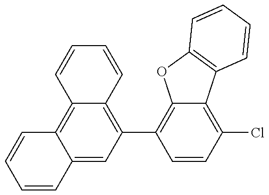 [SMILES: Clc1ccc(-c2cc3ccccc3c3ccccc23)c2oc3ccccc3c12] | 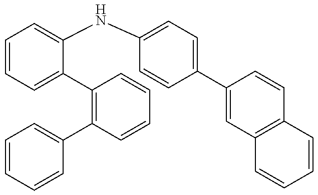 [SMILES: c1ccc(-c2ccccc2-c2ccccc2Nc2ccc(-c3ccc4ccccc4c3)cc2)cc1] | Compound 707 | 19.7g (71%) | 789.30 |
| P167 | 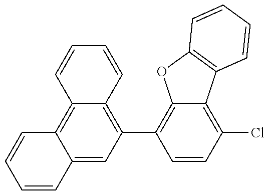 [SMILES: Clc1ccc(-c2cc3ccccc3c3ccccc23)c2oc3ccccc3c12] | 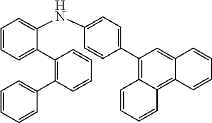 [SMILES: c1ccc(-c2ccccc2-c2ccccc2Nc2ccc(-c3cc4ccccc4c4ccccc34)cc2)cc1] | Compound 708 | 19.5 g (66%) | 839.32 |
| P168 | 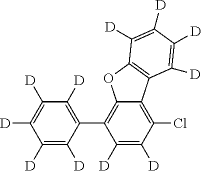 [SMILES: [2H]c1c([2H])c([2H])c(-c2c([2H])c([2H])c(Cl)c3c2oc2c([2H])c([2H])c([2H])c([2H])c23)c([2H])c1[2H]] | 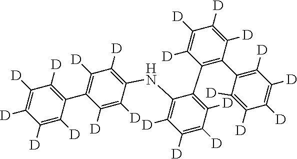 [SMILES: [2H]c1c([2H])c([2H])c(-c2c([2H])c([2H])c(Nc3c([2H])c([2H])c([2H])c([2H])c3-c3c([2H])c([2H])c([2H])c([2H])c3-c3c([2H])c([2H])c([2H])c([2H])c3[2H])c([2H])c2[2H])c([2H])c1[2H]] | Compound 712 | 16.1 g (68%) | 672.46 |
| P169 | 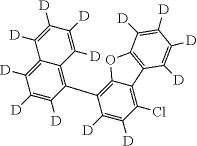 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 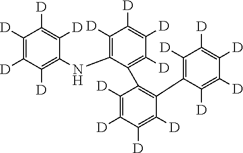 [SMILES: [2H]c1c([2H])c([2H])c(Nc2c([2H])c([2H])c([2H])c([2H])c2-c2c([2H])c([2H])c([2H])c([2H])c2-c2c([2H])c([2H])c([2H])c([2H])c2[2H])c([2H])c1[2H]] | Compound 713 | 15.9 g (70%) | 644.44 |
| P170 | 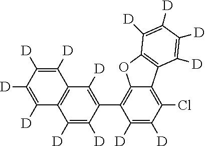 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 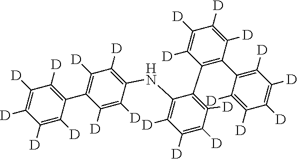 [SMILES: [2H]c1c([2H])c([2H])c(-c2c([2H])c([2H])c(Nc3c([2H])c([2H])c([2H])c([2H])c3-c3c([2H])c([2H])c([2H])c([2H])c3-c3c([2H])c([2H])c([2H])c([2H])c3[2H])c([2H])c2[2H])c([2H])c1[2H]] | Compound 714 | 18.1 g (71%) | 724.49 |
| P171 | 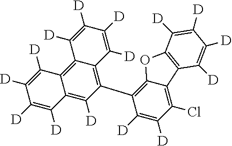 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 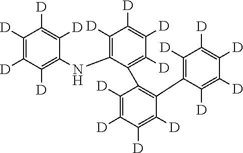 [SMILES: [2H]c1c([2H])c([2H])c(Nc2c([2H])c([2H])c([2H])c([2H])c2-c2c([2H])c([2H])c([2H])c([2H])c2-c2c([2H])c([2H])c([2H])c([2H])c2[2H])c([2H])c1[2H]] | Compound 715 | 16.7 g (68%) | 696.46 |

### [Experimental Example 1] Measurement of HOMO and LUMO

The hole transport auxiliary layer plays a role in reducing accumulation of holes at an interface between the hole transport layer and the light-emitting layer due to a difference between a HOMO level of the hole transport layer and a HOMO level of the light-emitting layer. To this end, a difference between the HOMO level of the light-emitting layer and a HOMO level of the hole transport auxiliary layer should be smaller than a difference between the HOMO level of the hole injection layer and the HOMO level of the hole transport auxiliary layer. Furthermore, the hole transport auxiliary layer should have a higher LUMO energy level than a LUMO energy level of the light-emitting layer to minimize electrons leaking from the light-emitting layer to the hole transport layer.

In order to check whether the compound represented by the Chemical Formula 1 in accordance with the present disclosure is suitable as a material of the hole transport auxiliary layer, the HOMO energy level (eV) and the LUMO energy level (eV) of the hole transport auxiliary layer containing the compound represented by the Chemical Formula 1 in accordance with the present disclosure were calculated using Spartan software (B3LYP DFT 6-31G* by spartan'16) and the calculation results are shown in Table 9 as set forth below.

TABLE 9

| | HOMO | LUMO |
|---|---|---|
| Compound | (calculation) | (calculation) |
| Compound 93 | −5.01 | −1.19 |
| Compound 97 | −5.03 | −1.18 |
| Compound 101 | −5.01 | −1.34 |
| Compound 7 | −4.89 | −1.18 |
| Compound 9 | −5.08 | −1.22 |
| Compound 10 | −4.98 | −1.18 |
| Compound 11 | −5.00 | −1.22 |
| Compound 12 | −5.03 | −1.21 |
| Compound 16 | −5.04 | −1.18 |
| Compound 15 | −5.04 | −1.23 |
| Compound 14 | −4.97 | −1.19 |
| Compound 13 | −5.11 | −1.29 |
| Compound 17 | −4.93 | −1.11 |
| Compound 18 | −4.76 | −1.12 |
| Compound 19 | −4.90 | −1.16 |
| Compound 20 | −4.86 | −1.13 |
| Compound 21 | −4.95 | −1.25 |
| Compound 22 | −5.14 | −1.23 |
| Compound 23 | −5.08 | −1.14 |

### [Present Example 1] Manufacturing of Organic Light-Emitting Diode (Blue Light-Emitting Layer)

A substrate on which ITO (100 nm) as a positive electrode of an organic light-emitting diode was deposited was patterned in a distinguishing manner of a positive electrode area, a negative electrode area, and an insulating layer area from each other in an exposure (Photo-Lithography) process. Then, for the purpose of increasing a work-function of the positive electrode and cleaning, a surface-treatment was performed thereon using UV-ozone and O₂:N₂ plasma.

Next, NDP-9 (2-(7-Dicyanomethylene-1,3,4,5,6,8,9,10-octafluoro-7H-pyren-2-ylidene)-malononitrile) and N4,N4,N4′,N4′-Tetra([1,1′-biphenyl]-4-yl)-[1,1′-biphenyl]-4,4′-diamine were mixed with each other in a ratio of 3:97 to produce a mixture which in turn was deposited on the positive electrode to form the hole injection layer (HIL) of a thickness of 10 nm.

Then, on the hole injection layer, N4,N4,N4′,N4′-tetra([1,1′-biphenyl]-4-yl)-[1,1′-biphenyl]-4,4′-diamine was vacuum-deposited to form the hole transport layer of a thickness of 100 nm. Then, the Compound 93 was deposited on the hole transport layer (HTL) to form the hole transport auxiliary layer of a thickness of 15 nm.

On the hole transport auxiliary layer, a blue light-emitting layer of 25 nm was deposited using 9,10-bis(2-naphthyl)anthracene (ADN) as a host and 2,12-di-tert-butyl-5,9-bis(4-(tert-butyl)phenyl)-7-(3,5-di-tert-butylphenyl)-5,9-dihydro5,9-diaza-13b-boranaphtho[3,2,1-de]anthracene (t-DABNA-dtB) as a dopant, wherein a mixing ratio of host:dopant (by weight) was 97:3.

On the blue light-emitting layer, the electron transport layer (ETL) of a thickness of 25 nm was deposited using a mixture of 2-(4-(9,10-di(naphthalene-2-yl)anthracene-2-yl)phenyl)-1-phenyl-1H-benzo[d]imidazole and Liq at a weight ratio of 1:1.

On the electron transport layer (ETL), the electron injection layer (EIL) of a thickness of 1 nm was deposited using Liq. Then, the negative electrode was deposited on the electron injection layer (EIL) so as to have a thickness of 16 nm using a mixture of magnesium and silver at a weight ratio of 1:4. Then, a capping layer made of N4,N4′-bis[4-[bis(3-methylphenyl)amino]phenyl]-N4,N4′-diphenyl-[1,1′-biphenyl]-4,4′-diamine (DNTPD) was deposited so as to have a thickness of 60 nm on the negative electrode. A seal cap containing a moisture absorbent was bonded to the capping layer using a UV curable adhesive to form a protective film (encapsulation layer or protecting layer) to protect the organic light-emitting diode from atmospheric oxygen or moisture. In this way, the organic light-emitting diode was manufactured.

### Comparative Examples 1 to 10

The organic light-emitting diode of each of Comparative Examples 1 to 10 was manufactured in the same manner as in Present Example 1, except that the Compound 93 used as the hole transport auxiliary layer material in Present Example 1 was replaced with what is shown in Table 11 as set forth below. The structures of Compounds A to J which are used as the hole transport auxiliary layer materials respectively used in Comparative Examples 1 to 10, are the same as those shown in Table 10 as set forth below.

### Present Examples 2 to 181

The organic light-emitting diode of each of Present Examples 2 to 181 was manufactured in the same manner as in Present Example 1, except that the Compound 93 used as the hole transport auxiliary layer material in Present Example 1 was replaced with what is shown in Tables 12 to 19 as set forth below.

### [Experimental Example 2] Organic Light-Emitting Diode Performance Evaluation (Blue Organic Light-Emitting Diode)

A current of 10 mA/cm² was applied to each of the organic light-emitting diodes of Present Examples 1 to 181 and Comparative Examples 1 to 10 using a CS-2000 from KONICA MINOLTA. Then, the operation voltage and external quantum efficiency (EQE) (%) were measured. Furthermore, the lifetime (LT95) was measured based on a time duration for which luminance decreases from initial luminance to 95% thereof under application of a constant current of 10 mA/cm² using M6000 from McScience. The measurement results are shown in Tables 11 to 19 as set forth below.

TABLE 10

| Comparative Example 1 | |
|---|---|
| 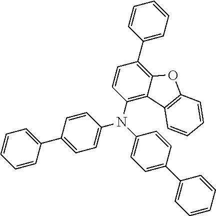 [SMILES: c1ccc(-c2ccc(N(c3ccc(-c4ccccc4)cc3)c3ccc(-c4ccccc4)c4oc5ccccc5c34)cc2)cc1] | Compound A |
| Comparative Example 2 | |
| 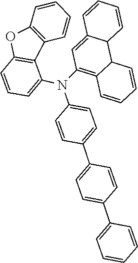 [SMILES: C1=CC2=CC(N(c3ccc(-c4ccc(-c5ccccc5)cc4)cc3)c3cccc4oc5ccccc5c34)=C3C=CC=CC3C2C=C1] | Compound B |
| Comparative Example 3 | |
| 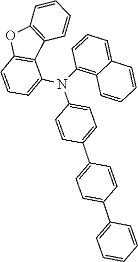 [SMILES: c1ccc(-c2ccc(-c3ccc(N(c4cccc5ccccc45)c4cccc5oc6ccccc6c45)cc3)cc2)cc1] | Compound C |
| Comparative Example 4 | |
| 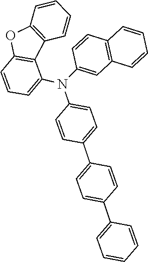 [SMILES: c1ccc(-c2ccc(-c3ccc(N(c4ccc5ccccc5c4)c4cccc5oc6ccccc6c45)cc3)cc2)cc1] | Compound D |
| Comparative Example 5 | |
| 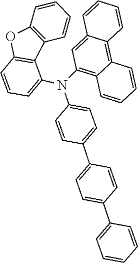 [SMILES: c1ccc(-c2ccc(-c3ccc(N(c4cc5ccccc5c5ccccc45)c4cccc5oc6ccccc6c45)cc3)cc2)cc1] | Compound E |
| Comparative Example 6 | |
| 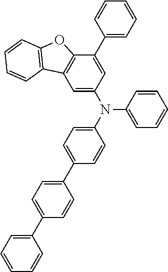 [SMILES: c1ccc(-c2ccc(-c3ccc(N(c4ccccc4)c4cc(-c5ccccc5)c5oc6ccccc6c5c4)cc3)cc2)cc1] | Compound F |
| Comparative Example 7 | |
| 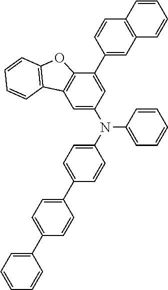 [SMILES: c1ccc(-c2ccc(-c3ccc(N(c4ccccc4)c4cc(-c5ccc6ccccc6c5)c5oc6ccccc6c5c4)cc3)cc2)cc1] | Compound G |
| Comparative Example 8 | |
| 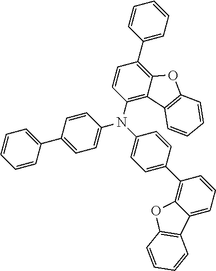 [SMILES: c1ccc(-c2ccc(N(c3ccc(-c4cccc5c4oc4ccccc45)cc3)c3ccc(-c4ccccc4)c4oc5ccccc5c34)cc2)cc1] | Compound H |
| Comparative Example 9 | |
| 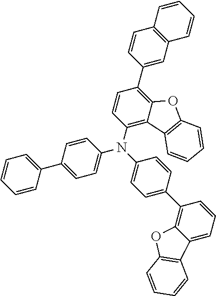 [SMILES: c1ccc(-c2ccc(N(c3ccc(-c4cccc5c4oc4ccccc45)cc3)c3ccc(-c4ccc5ccccc5c4)c4oc5ccccc5c34)cc2)cc1] | Compound I |
| Comparative Example 10 | |
| 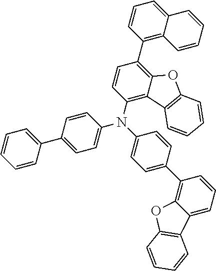 [SMILES: c1ccc(-c2ccc(N(c3ccc(-c4cccc5c4oc4ccccc45)cc3)c3ccc(-c4cccc5ccccc45)c4oc5ccccc5c34)cc2)cc1] | Compound J |

TABLE 11

| | Hole transport | | | Lifetime |
|---|---|---|---|---|
| Comparative | auxiliary layer | Operation | | (LT95) |
| Example | material | voltage (V) | EQE (%) | (hrs) |
| Comparative | Compound A | 3.80 | 12.9 | 252 |
| Example 1 | | | | |
| Comparative | Compound B | 3.80 | 12.5 | 245 |
| Example 2 | | | | |
| Comparative | Compound C | 3.81 | 12.3 | 230 |
| Example 3 | | | | |
| Comparative | Compound D | 3.81 | 12.9 | 226 |
| Example 4 | | | | |
| Comparative | Compound E | 3.82 | 13.0 | 225 |
| Example 5 | | | | |
| Comparative | Compound F | 3.82 | 13.0 | 220 |
| Example 6 | | | | |
| Comparative | Compound G | 3.83 | 12.8 | 220 |
| Example 7 | | | | |
| Comparative | Compound H | 3.83 | 12.6 | 214 |
| Example 8 | | | | |
| Comparative | Compound I | 3.85 | 12.6 | 210 |
| Example 9 | | | | |
| Comparative | Compound J | 3.85 | 12.8 | 210 |
| Example 10 | | | | |

TABLE 12

| | Hole transport | | | Lifetime |
|---|---|---|---|---|
| Present | auxiliary layer | Operation | | (LT95) |
| Example | material | voltage (V) | EQE (%) | (hrs) |
| Present | Compound 93 | 3.73 | 17.2 | 370 |
| Example 1 | | | | |
| Present | Compound 94 | 3.72 | 17.3 | 371 |
| Example 2 | | | | |
| Present | Compound 115 | 3.73 | 17.2 | 370 |
| Example 3 | | | | |
| Present | Compound 116 | 3.73 | 17.2 | 370 |
| Example 4 | | | | |
| Present | Compound 2 | 3.72 | 17.4 | 369 |
| Example 5 | | | | |
| Present | Compound 3 | 3.72 | 17.4 | 369 |
| Example 6 | | | | |
| Present | Compound 4 | 3.70 | 17.5 | 369 |
| Example 7 | | | | |
| Present | Compound 7 | 3.73 | 17.5 | 370 |
| Example 8 | | | | |
| Present | Compound 125 | 3.73 | 17.2 | 371 |
| Example 9 | | | | |
| Present | Compound 95 | 3.71 | 17.3 | 374 |
| Example 10 | | | | |
| Present | Compound 118 | 3.71 | 17.3 | 373 |
| Example 11 | | | | |
| Present | Compound 119 | 3.71 | 17.4 | 373 |
| Example 12 | | | | |
| Present | Compound 96 | 3.70 | 17.5 | 375 |
| Example 13 | | | | |
| Present | Compound 121 | 3.70 | 17.5 | 374 |
| Example 14 | | | | |
| Present | Compound 122 | 3.70 | 17.4 | 374 |
| Example 15 | | | | |
| Present | Compound 24 | 3.75 | 17.1 | 365 |
| Example 16 | | | | |
| Present | Compound 97 | 3.75 | 17.1 | 363 |
| Example 17 | | | | |
| Present | Compound 98 | 3.74 | 17.2 | 368 |
| Example 18 | | | | |
| Present | Compound 99 | 3.74 | 17.2 | 367 |
| Example 19 | | | | |
| Present | Compound 53 | 3.75 | 17.1 | 366 |
| Example 20 | | | | |
| Present | Compound 101 | 3.75 | 17.1 | 364 |
| Example 21 | | | | |
| Present | Compound 102 | 3.74 | 17.1 | 365 |
| Example 22 | | | | |
| Present | Compound 103 | 3.75 | 17.2 | 367 |
| Example 23 | | | | |
| Present | Compound 105 | 3.77 | 16.9 | 359 |
| Example 24 | | | | |
| Present | Compound 106 | 3.76 | 17.0 | 361 |
| Example 25 | | | | |
| Present | Compound 107 | 3.76 | 17.1 | 360 |
| Example 26 | | | | |
| Present | Compound 126 | 3.73 | 17.2 | 444 |
| Example 27 | | | | |
| Present | Compound 127 | 3.71 | 17.4 | 448 |
| Example 28 | | | | |
| Present | Compound 128 | 3.71 | 17.4 | 447 |
| Example 29 | | | | |
| Present | Compound 141 | 3.74 | 17.2 | 435 |
| Example 30 | | | | |
| Present | Compound 142 | 3.75 | 17.1 | 438 |
| Example 31 | | | | |
| Present | Compound 143 | 3.75 | 17.2 | 439 |
| Example 32 | | | | |
| Present | Compound 144 | 3.74 | 17.2 | 441 |
| Example 33 | | | | |
| Present | Compound 157 | 3.75 | 17.1 | 436 |
| Example 34 | | | | |
| Present | Compound 158 | 3.74 | 17.1 | 438 |
| Example 35 | | | | |
| Present | Compound 159 | 3.74 | 17.1 | 440 |
| Example 36 | | | | |
| Present | Compound 160 | 3.74 | 17.2 | 441 |
| Example 37 | | | | |
| Present | Compound 173 | 3.77 | 16.9 | 430 |
| Example 38 | | | | |
| Present | Compound 174 | 3.76 | 17.0 | 432 |
| Example 39 | | | | |
| Present | Compound 175 | 3.76 | 17.0 | 433 |
| Example 40 | | | | |
| Present | Compound 176 | 3.76 | 17.0 | 433 |
| Example 41 | | | | |

TABLE 13

| | Hole transport | | | Lifetime |
|---|---|---|---|---|
| Present | auxiliary layer | Operation | | (LT95) |
| Example | material | voltage(V) | EQE (%) | (hrs) |
| Present | Compound 225 | 3.42 | 21.5 | 447 |
| Example 42 | | | | |
| Present | Compound 226 | 3.41 | 21.7 | 448 |
| Example 43 | | | | |
| Present | Compound 227 | 3.41 | 21.6 | 448 |
| Example 44 | | | | |
| Present | Compound 228 | 3.38 | 21.9 | 450 |
| Example 45 | | | | |
| Present | Compound 230 | 3.47 | 21.0 | 442 |
| Example 46 | | | | |
| Present | Compound 231 | 3.45 | 21.3 | 444 |
| Example 47 | | | | |
| Present | Compound 232 | 3.46 | 21.2 | 444 |
| Example 48 | | | | |
| Present | Compound 198 | 3.42 | 21.4 | 446 |
| Example 49 | | | | |
| Present | Compound 203 | 3.46 | 21.1 | 443 |
| Example 50 | | | | |
| Present | Compound 236 | 3.44 | 21.2 | 444 |
| Example 51 | | | | |
| Present | Compound 237 | 3.44 | 21.2 | 445 |
| Example 52 | | | | |
| Present | Compound 209 | 3.42 | 21.3 | 445 |
| Example 53 | | | | |
| Present | Compound 240 | 3.56 | 20.1 | 435 |
| Example 54 | | | | |
| Present | Compound 241 | 3.50 | 20.5 | 437 |
| Example 55 | | | | |
| Present | Compound 218 | 3.44 | 20.6 | 440 |
| Example 56 | | | | |
| Present | Compound 220 | 3.48 | 20.9 | 441 |
| Example 57 | | | | |
| Present | Compound 265 | 3.38 | 21.9 | 540 |
| Example 58 | | | | |
| Present | Compound 266 | 3.47 | 21.2 | 530 |
| Example 59 | | | | |
| Present | Compound 267 | 3.44 | 21.2 | 534 |
| Example 60 | | | | |
| Present | Compound 268 | 3.48 | 20.9 | 529 |
| Example 61 | | | | |

TABLE 14

| | Hole transport | | | Lifetime |
|---|---|---|---|---|
| Present | auxiliary layer | Operation | | (LT95) |
| Example | material | voltage(V) | EQE(%) | (hrs) |
| Present | Compound 313 | 3.55 | 18.8 | 412 |
| Example 62 | | | | |
| Present | Compound 314 | 3.55 | 18.9 | 414 |
| Example 63 | | | | |
| Present | Compound 273 | 3.54 | 19.0 | 414 |
| Example 64 | | | | |
| Present | Compound 316 | 3.54 | 19.0 | 415 |
| Example 65 | | | | |
| Present | Compound 280 | 3.57 | 18.6 | 407 |
| Example 66 | | | | |
| Present | Compound 319 | 3.56 | 18.6 | 409 |
| Example 67 | | | | |
| Present | Compound 284 | 3.57 | 18.7 | 409 |
| Example 68 | | | | |
| Present | Compound 321 | 3.56 | 18.8 | 411 |
| Example 69 | | | | |
| Present | Compound 323 | 3.56 | 18.6 | 407 |
| Example 70 | | | | |
| Present | Compound 293 | 3.57 | 18.7 | 409 |
| Example 71 | | | | |
| Present | Compound 325 | 3.56 | 18.6 | 408 |
| Example 72 | | | | |
| Present | Compound 297 | 3.56 | 18.7 | 410 |
| Example 73 | | | | |
| Present | Compound 302 | 3.60 | 18.3 | 400 |
| Example 74 | | | | |
| Present | Compound 329 | 3.60 | 18.4 | 403 |
| Example 75 | | | | |
| Present | Compound 306 | 3.59 | 18.3 | 405 |
| Example 76 | | | | |
| Present | Compound 331 | 3.58 | 18.5 | 406 |
| Example 77 | | | | |
| Present | Compound 353 | 3.55 | 18.9 | 498 |
| Example 78 | | | | |
| Present | Compound 354 | 3.57 | 18.7 | 488 |
| Example 79 | | | | |
| Present | Compound 355 | 3.56 | 18.7 | 487 |
| Example 80 | | | | |
| Present | Compound 356 | 3.60 | 18.4 | 480 |
| Example 81 | | | | |

TABLE 15

| | Hole transport | | | Lifetime |
|---|---|---|---|---|
| Present | auxiliary layer | Operation | | (LT95) |
| Example | material | voltage(V) | EQE(%) | (hrs) |
| Present | Compound 403 | 3.64 | 17.8 | 387 |
| Example 82 | | | | |
| Present | Compound 359 | 3.64 | 17.8 | 389 |
| Example 83 | | | | |
| Present | Compound 361 | 3.64 | 17.9 | 388 |
| Example 84 | | | | |
| Present | Compound 406 | 3.63 | 17.9 | 390 |
| Example 85 | | | | |
| Present | Compound 371 | 3.65 | 17.7 | 382 |
| Example 86 | | | | |
| Present | Compound 409 | 3.64 | 17.7 | 383 |
| Example 87 | | | | |
| Present | Compound 374 | 3.64 | 17.8 | 385 |
| Example 88 | | | | |
| Present | Compound 411 | 3.64 | 17.8 | 386 |
| Example 89 | | | | |
| Present | Compound 413 | 3.65 | 17.7 | 383 |
| Example 90 | | | | |
| Present | Compound 383 | 3.64 | 17.8 | 384 |
| Example 91 | | | | |
| Present | Compound 385 | 3.65 | 17.9 | 384 |
| Example 92 | | | | |
| Present | Compound 416 | 3.64 | 17.9 | 385 |
| Example 93 | | | | |
| Present | Compound 392 | 3.66 | 17.5 | 378 |
| Example 94 | | | | |
| Present | Compound 394 | 3.66 | 17.5 | 380 |
| Example 95 | | | | |
| Present | Compound 420 | 3.65 | 17.6 | 380 |
| Example 96 | | | | |
| Present | Compound 398 | 3.67 | 17.7 | 381 |
| Example 97 | | | | |
| Present | Compound 423 | 3.66 | 17.9 | 468 |
| Example 98 | | | | |
| Present | Compound 424 | 3.65 | 17.8 | 458 |
| Example 99 | | | | |
| Present | Compound 425 | 3.65 | 17.7 | 459 |
| Example 100 | | | | |
| Present | Compound 426 | 3.67 | 17.6 | 453 |
| Example 101 | | | | |

TABLE 16

| | Hole transport | | | Lifetime |
|---|---|---|---|---|
| Present | auxiliary layer | Operation | | (LT95) |
| Example | material | voltage (V) | EQE (%) | (hrs) |
| Present | Compound 494 | 3.50 | 20.5 | 433 |
| Example 102 | | | | |
| Present | Compound 495 | 3.47 | 20.7 | 435 |
| Example 103 | | | | |
| Present | Compound 431 | 3.47 | 20.8 | 437 |
| Example 104 | | | | |
| Present | Compound 474 | 3.45 | 21.0 | 440 |
| Example 105 | | | | |
| Present | Compound 479 | 3.52 | 19.9 | 425 |
| Example 106 | | | | |
| Present | Compound 480 | 3.52 | 20.1 | 427 |
| Example 107 | | | | |
| Present | Compound 496 | 3.51 | 20.2 | 427 |
| Example 108 | | | | |
| Present | Compound 482 | 3.50 | 20.4 | 429 |
| Example 109 | | | | |
| Present | Compound 484 | 3.52 | 20.1 | 426 |
| Example 110 | | | | |
| Present | Compound 451 | 3.52 | 20.3 | 428 |
| Example 111 | | | | |
| Present | Compound 486 | 3.51 | 20.3 | 427 |
| Example 112 | | | | |
| Present | Compound 498 | 3.51 | 20.4 | 429 |
| Example 113 | | | | |
| Present | Compound 489 | 3.56 | 19.3 | 420 |
| Example 114 | | | | |
| Present | Compound 499 | 3.54 | 19.6 | 422 |
| Example 115 | | | | |
| Present | Compound 491 | 3.54 | 19.5 | 423 |
| Example 116 | | | | |
| Present | Compound 492 | 3.53 | 19.8 | 424 |
| Example 117 | | | | |
| Present | Compound 500 | 3.46 | 20.9 | 516 |
| Example 118 | | | | |
| Present | Compound 501 | 3.52 | 20.1 | 510 |
| Example 119 | | | | |
| Present | Compound 502 | 3.52 | 20.3 | 511 |
| Example 120 | | | | |
| Present | Compound 503 | 3.54 | 19.5 | 504 |
| Example 121 | | | | |

TABLE 17

| | Hole transport | | | Lifetime |
|---|---|---|---|---|
| Present | auxiliary layer | Operation | | (LT95) |
| Example | material | voltage (V) | EQE (%) | (hrs) |
| Present | Compound 568 | 3.75 | 16.6 | 350 |
| Example 122 | | | | |
| Present | Compound 549 | 3.75 | 16.8 | 354 |
| Example 123 | | | | |
| Present | Compound 508 | 3.76 | 16.8 | 355 |
| Example 124 | | | | |
| Present | Compound 551 | 3.75 | 17.0 | 358 |
| Example 125 | | | | |
| Present | Compound 553 | 3.76 | 17.3 | 355 |
| Example 126 | | | | |
| Present | Compound 569 | 3.77 | 16.3 | 346 |
| Example 127 | | | | |
| Present | Compound 555 | 3.77 | 16.5 | 346 |
| Example 128 | | | | |
| Present | Compound 521 | 3.76 | 16.5 | 348 |
| Example 129 | | | | |
| Present | Compound 526 | 3.77 | 16.3 | 346 |
| Example 130 | | | | |
| Present | Compound 559 | 3.76 | 16.4 | 348 |
| Example 131 | | | | |
| Present | Compound 570 | 3.77 | 16.4 | 347 |
| Example 132 | | | | |
| Present | Compound 561 | 3.76 | 16.4 | 349 |
| Example 133 | | | | |
| Present | Compound 563 | 3.79 | 16.1 | 342 |
| Example 134 | | | | |
| Present | Compound 539 | 3.78 | 16.2 | 343 |
| Example 135 | | | | |
| Present | Compound 565 | 3.78 | 16.2 | 345 |
| Example 136 | | | | |
| Present | Compound 571 | 3.77 | 16.3 | 345 |
| Example 137 | | | | |
| Present | Compound 572 | 3.76 | 16.7 | 420 |
| Example 138 | | | | |
| Present | Compound 573 | 3.75 | 16.3 | 415 |
| Example 139 | | | | |
| Present | Compound 574 | 3.77 | 16.4 | 415 |
| Example 140 | | | | |
| Present | Compound 575 | 3.78 | 16.2 | 410 |
| Example 141 | | | | |

TABLE 18

| | Hole transport | | | Lifetime |
|---|---|---|---|---|
| Present | auxiliary layer | Operation | | (LT95) |
| Example | material | voltage (V) | EQE (%) | (hrs) |
| Present | Compound 576 | 3.56 | 18.5 | 408 |
| Example 142 | | | | |
| Present | Compound 621 | 3.55 | 18.6 | 410 |
| Example 143 | | | | |
| Present | Compound 622 | 3.55 | 18.7 | 410 |
| Example 144 | | | | |
| Present | Compound 623 | 3.54 | 18.7 | 411 |
| Example 145 | | | | |
| Present | Compound 625 | 3.60 | 18.2 | 402 |
| Example 146 | | | | |
| Present | Compound 626 | 3.59 | 18.4 | 406 |
| Example 147 | | | | |
| Present | Compound 627 | 3.58 | 18.3 | 405 |
| Example 148 | | | | |
| Present | Compound 628 | 3.58 | 18.5 | 407 |
| Example 149 | | | | |
| Present | Compound 641 | 3.59 | 18.2 | 403 |
| Example 150 | | | | |
| Present | Compound 631 | 3.59 | 18.3 | 405 |
| Example 151 | | | | |
| Present | Compound 632 | 3.58 | 18.5 | 406 |
| Example 152 | | | | |
| Present | Compound 633 | 3.57 | 18.5 | 408 |
| Example 153 | | | | |
| Present | Compound 635 | 3.63 | 17.9 | 395 |
| Example 154 | | | | |
| Present | Compound 636 | 3.62 | 17.9 | 398 |
| Example 155 | | | | |
| Present | Compound 637 | 3.62 | 18.0 | 397 |
| Example 156 | | | | |
| Present | Compound 638 | 3.60 | 18.1 | 401 |
| Example 157 | | | | |
| Present | Compound 642 | 3.55 | 18.6 | 489 |
| Example 158 | | | | |
| Present | Compound 643 | 3.57 | 18.3 | 482 |
| Example 159 | | | | |
| Present | Compound 644 | 3.59 | 18.2 | 483 |
| Example 160 | | | | |
| Present | Compound 645 | 3.63 | 18.0 | 474 |
| Example 161 | | | | |

TABLE 19

| | Hole transport | | | Lifetime |
|---|---|---|---|---|
| Present | auxiliary layer | Operation | | (LT95) |
| Example | material | voltage (V) | EQE (%) | (hrs) |
| Present | Compound 690 | 3.77 | 16.6 | 351 |
| Example 162 | | | | |
| Present | Compound 691 | 3.76 | 16.8 | 356 |
| Example 163 | | | | |
| Present | Compound 692 | 3.76 | 16.8 | 355 |
| Example 164 | | | | |
| Present | Compound 693 | 3.75 | 16.8 | 357 |
| Example 165 | | | | |
| Present | Compound 710 | 3.77 | 16.4 | 346 |
| Example 166 | | | | |
| Present | Compound 696 | 3.76 | 16.4 | 347 |
| Example 167 | | | | |
| Present | Compound 697 | 3.77 | 16.4 | 349 |
| Example 168 | | | | |
| Present | Compound 698 | 3.76 | 16.5 | 349 |
| Example 169 | | | | |
| Present | Compound 700 | 3.77 | 16.3 | 347 |
| Example 170 | | | | |
| Present | Compound 701 | 3.76 | 16.4 | 349 |
| Example 171 | | | | |
| Present | Compound 702 | 3.76 | 16.3 | 348 |
| Example 172 | | | | |
| Present | Compound 703 | 3.76 | 16.4 | 350 |
| Example 173 | | | | |
| Present | Compound 711 | 3.79 | 16.0 | 344 |
| Example 174 | | | | |
| Present | Compound 706 | 3.78 | 16.2 | 347 |
| Example 175 | | | | |
| Present | Compound 707 | 3.79 | 16.2 | 347 |
| Example 176 | | | | |
| Present | Compound 708 | 3.78 | 16.2 | 348 |
| Example 177 | | | | |
| Present | Compound 712 | 3.76 | 16.8 | 421 |
| Example 178 | | | | |
| Present | Compound 713 | 3.76 | 16.5 | 415 |
| Example 179 | | | | |
| Present | Compound 714 | 3.77 | 16.3 | 416 |
| Example 180 | | | | |
| Present | Compound 715 | 3.78 | 16.2 | 412 |
| Example 181 | | | | |

### [Present Example 182] Manufacturing of Organic Light-Emitting Diode (Green Light-Emitting Layer)

A substrate on which ITO (100 nm) as a positive electrode of an organic light-emitting device was deposited was patterned in a distinguishing manner of a positive electrode area, a negative electrode area, and an insulating layer area from each other in an exposure (Photo-Lithography) process. Then, for the purpose of increasing a work-function of the positive electrode and cleaning, a surface-treatment was performed thereon using UV-ozone and O₂:N₂ plasma.

Next, NDP-9 (2-(7-Dicyanomethylene-1,3,4,5,6,8,9,10-octafluoro-7H-pyren-2-ylidene)-malononitrile) and N4,N4,N4′,N4′-Tetra([1,1′-biphenyl]-4-yl)-[1,1′-biphenyl]-4,4′-diamine were mixed with each other in a ratio of 3:97 to produce a mixture which in turn was deposited on the positive electrode to form the hole injection layer (HIL) of a thickness of 10 nm.

Then, on the hole injection layer, N4,N4,N4′,N4′-tetra([1,1′-biphenyl]-4-yl)-[1,1′-biphenyl]-4,4′-diamine was vacuum-deposited to form the hole transport layer of a thickness of 100 nm. Then, the Compound 93 was deposited on the hole transport layer (HTL) to form the hole transport auxiliary layer of a thickness of 15 nm.

On the hole transport auxiliary layer, a green light-emitting layer of 35 nm was deposited using 4,4′-N,N′-dicarbazole-biphenyl (CBP) as a host and Ir(ppy)₃ [tris(2-phenylpyridine)-iridium] as a dopant, wherein a mixing ratio of host:dopant (by weight) was 95:5.

On the green light-emitting layer, the electron transport layer (ETL) of a thickness of 25 nm was deposited using a mixture of 2-(4-(9,10-di(naphthalene-2-yl)anthracene-2-yl)phenyl)-1-phenyl-TH-benzo[d]imidazole and Liq at a weight ratio of 1:1.

On the electron transport layer (ETL), the electron injection layer (EIL) of a thickness of 1 nm was deposited using Liq. Then, the negative electrode was deposited on the electron injection layer (EIL) so as to have a thickness of 16 nm using a mixture of magnesium and silver at a weight ratio of 1:4. Then, a capping layer made of N4,N4′-bis[4-[bis(3-methylphenyl)amino]phenyl]-N4,N4′-diphenyl-[1,1′-biphenyl]-4,4′-diamine (DNTPD) was deposited so as to have a thickness of 60 nm on the negative electrode. A seal cap containing a moisture absorbent was bonded to the capping layer using a UV curable adhesive to form a protective film (encapsulation layer or protecting layer) to protect the organic light-emitting diode from atmospheric oxygen or moisture. In this way, the light-emitting diode was manufactured.

### Comparative Examples 11 to 20

The organic light-emitting diode of each of Comparative Examples 11 to 20 was manufactured in the same manner as in Present Example 182, except that the Compound 93 used as the hole transport auxiliary layer material in Present Example 182 was replaced with what is shown in Table 20 as set forth below. The structures of Compounds A to J which are used as the hole transport auxiliary layer materials respectively used in Comparative Examples 11 to 20, are the same as those shown in Table 10 as set forth above.

### Present Examples 183 to 205

The organic light-emitting diode of each of Present Examples 183 to 205 was manufactured in the same manner as in Present Example 182, except that the Compound 93 used as the hole transport auxiliary layer material in Present Example 182 was replaced with what is shown in Table 21 as set forth below.

### [Experimental Example 3] Organic Light-Emitting Diode Performance Evaluation (Green Organic Light-Emitting Diode)

A current of 10 mA/cm² was applied to each of the organic light-emitting diodes of Present Examples 182 to 205 and Comparative Examples 11 to 20 using a CS-2000 from KONICA MINOLTA. Then, the operation voltage and external quantum efficiency (EQE) (%) were measured. Furthermore, the lifetime (LT95) was measured based on a time duration for which luminance decreases from initial luminance to 95% thereof under application of a constant current of 10 mA/cm² using M6000 from McScience. The measurement results are

TABLE 20

| | Hole transport | | | Lifetime |
|---|---|---|---|---|
| Comparative | auxiliary layer | Operation | | (LT95) |
| Example | material | voltage (V) | EQE (%) | (hrs) |
| Comparative | Compound A | 3.70 | 40.0 | 650 |
| Example 11 | | | | |
| Comparative | Compound B | 3.75 | 40.2 | 680 |
| Example 12 | | | | |
| Comparative | Compound C | 3.72 | 39.5 | 700 |
| Example 13 | | | | |
| Comparative | Compound D | 3.71 | 38.5 | 685 |
| Example 14 | | | | |
| Comparative | Compound E | 3.73 | 40.3 | 670 |
| Example 15 | | | | |
| Comparative | Compound F | 3.76 | 39.4 | 673 |
| Example 16 | | | | |
| Comparative | Compound G | 3.74 | 40.6 | 690 |
| Example 17 | | | | |
| Comparative | Compound H | 3.77 | 39.2 | 660 |
| Example 18 | | | | |
| Comparative | Compound I | 3.76 | 40.9 | 645 |
| Example 19 | | | | |
| Comparative | Compound J | 3.78 | 40.1 | 640 |
| Example 20 | | | | |

TABLE 21

| | Hole transport | | | Lifetime |
|---|---|---|---|---|
| Present | auxiliary layer | Operation | | (LT95) |
| Example | material | voltage (V) | EQE (%) | (hrs) |
| Present | Compound 93 | 3.52 | 45.3 | 910 |
| Example 182 | | | | |
| Present | Compound 98 | 3.53 | 45.2 | 910 |
| Example 183 | | | | |
| Present | Compound 105 | 3.54 | 45.2 | 900 |
| Example 184 | | | | |
| Present | Compound 228 | 3.46 | 46.6 | 950 |
| Example 185 | | | | |
| Present | Compound 230 | 3.46 | 46.3 | 945 |
| Example 186 | | | | |
| Present | Compound 218 | 3.45 | 46.5 | 930 |
| Example 187 | | | | |
| Present | Compound 316 | 3.48 | 46.2 | 930 |
| Example 188 | | | | |
| Present | Compound 280 | 3.49 | 46.1 | 920 |
| Example 189 | | | | |
| Present | Compound 329 | 3.48 | 46.1 | 915 |
| Example 190 | | | | |
| Present | Compound 406 | 3.52 | 45.8 | 915 |
| Example 191 | | | | |
| Present | Compound 383 | 3.52 | 45.5 | 910 |
| Example 192 | | | | |
| Present | Compound 392 | 3.53 | 45.4 | 905 |
| Example 193 | | | | |
| Present | Compound 495 | 3.45 | 46.8 | 940 |
| Example 194 | | | | |
| Present | Compound 498 | 3.46 | 46.5 | 935 |
| Example 195 | | | | |
| Present | Compound 489 | 3.45 | 46.3 | 933 |
| Example 196 | | | | |
| Present | Compound 568 | 3.54 | 43.5 | 865 |
| Example 197 | | | | |
| Present | Compound 555 | 3.58 | 43.2 | 863 |
| Example 198 | | | | |
| Present | Compound 563 | 3.57 | 43.1 | 860 |
| Example 199 | | | | |
| Present | Compound 621 | 3.49 | 46.1 | 925 |
| Example 200 | | | | |
| Present | Compound 641 | 3.50 | 46.0 | 920 |
| Example 201 | | | | |
| Present | Compound 635 | 3.50 | 46.0 | 915 |
| Example 202 | | | | |
| Present | Compound 690 | 3.58 | 44.0 | 865 |
| Example 203 | | | | |
| Present | Compound 710 | 3.58 | 43.4 | 860 |
| Example 204 | | | | |
| Present | Compound 706 | 3.59 | 43.3 | 825 |
| Example 205 | | | | |

Since the compound represented by the Chemical Formula 1 in accordance with the present disclosure has the characteristic structural form as described above, the hole injection characteristics may be controlled compared to the Comparative Example compounds that do not satisfy the structure of the Chemical Formula 1, thereby reducing the accumulation of holes at the interface between the hole transport auxiliary layer and the light-emitting layer. Thus, a quenching phenomenon in which excitons are annihilated by polarons at the interface between the hole transport auxiliary layer and the light-emitting layer may be reduced. As a result, it was confirmed that the deterioration phenomenon of the device could be reduced compared to the device using each of the Comparative Example compounds, thereby lowering the operation voltage and improving efficiency and lifetime of the device.

Although embodiments of the present disclosure have been described with reference to the accompanying drawings, the present disclosure is not limited to the above embodiments, but may be implemented in various different forms. A person skilled in the art may appreciate that the present disclosure may be practiced in other concrete forms without changing the technical spirit or essential characteristics of the present disclosure. Therefore, it should be appreciated that the embodiments as described above is not restrictive but illustrative in all respects.

What is claimed is:

1. An organic compound represented by a following Chemical Formula 1: 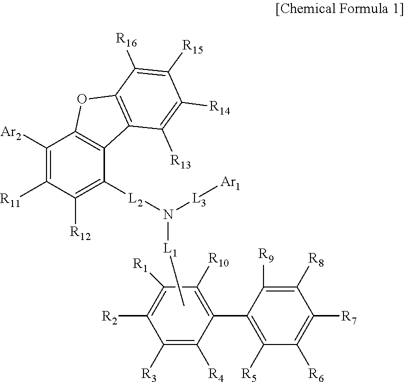
wherein in the Chemical Formula 1,
each of L₁ to L₃ independently represents one selected from a group consisting of a single bond, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, and a substituted or unsubstituted heteroarylalkyl group having 6 to 60 carbon atoms,
R₁ to R₁₆ and Ar are identical with or different from each other, wherein each of R₁ to R₁₆ and Ar₁ independently represents one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6 to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms,
Ar₂ is a substituted or unsubstituted aryl group having 6 to 30 carbon atoms,
optionally, each of L₁ to L₃, R₁ to R₁₆, and Ar₁ and Ar₂ may be substituted with at least one substituent selected from a group consisting of deuterium, a cyano group, a trifluoromethyl group, a nitro group, a halogen group, a hydroxy group, a trimethylsilylethynyl group (TMS), an alkyl group having 1 to 30 carbon atoms, a cycloalkyl group having 3 to 20 carbon atoms, an alkenyl group having 2 to 30 carbon atoms, a cycloalkenyl group having 3 to 20 carbon atoms, an alkynyl group having 2 to 30 carbon atoms, a cycloalkynyl group having 3 to 20 carbon atoms, an aryl group having 6 to 30 carbon atoms, an aralkyl group having 7 to 30 carbon atoms, a heteroaryl group having 5 to 60 carbon atoms, a heteroaralkyl group having 6 to 60 carbon atoms, an amine group, an alkylamino group having 1 to 30 carbon atoms, an aralkylamino group having 7 to 30 carbon atoms, an arylamino group having 6 to 30 carbon atoms, a heteroarylamino group having 5 to 60 carbon atoms, a silyl group, an alkylsilyl group having 1 to 30 carbon atoms, an arylsilyl group having 6 to 30 carbon atoms, an alkoxy group having 1 to 30 carbon atoms, an aryloxy group having 6 to 30 carbon atoms, an alkylthio group having 1 to 30 carbon atoms, and an arylthio group having 6 to 30 carbon atoms,
when the at least one substituent includes a plurality of substituents, the plurality of substituents are identical with or different from each other.

2. The organic compound of claim 1, wherein R₇ is a substituent represented by a following Chemical Formula 2: 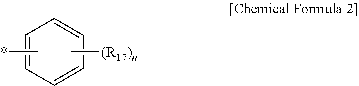
wherein in the Chemical Formula 2,
* denotes a binding site,
n is an integer of 0 to 5, and
R₁₇ independently represents one selected from a group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 5 to 60 carbon atoms.

3. The organic compound of claim 1, wherein each of Ar₁ independently represents a substituted or unsubstituted aryl group having 6 to 30 carbon atoms.

4. The organic compound of claim 1, wherein Ar₂ is one selected from substituents respectively represented by following Chemical Formula 3 to Chemical Formula 5: 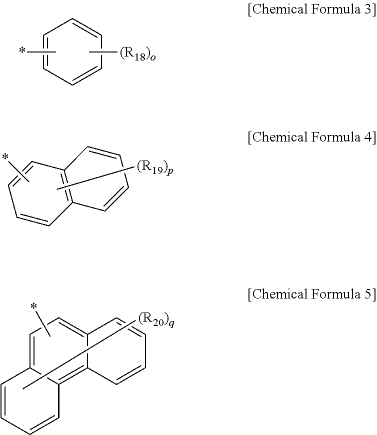
wherein in each of the Chemical Formulas 3 to 5,
* denotes a binding site,
n is an integer of 0 to 5, p is an integer from 0 to 7, and q is an integer from 0 to 9,
each of R₁₈ to R₂₀ independently represents one selected from a group consisting of hydrogen, deuterium, a substituted or unsubstituted alkyl group having 1 to 30 carbon atoms, a substituted or unsubstituted aryl group having 6 to 30 carbon atoms, and a substituted or unsubstituted heteroaryl group having 5 to 60 carbon atoms.

5. The organic compound of claim 1, wherein Ar is a substituted or unsubstituted aryl group having 6 to 18 carbon atoms.

6. The organic compound of claim 1, wherein the Chemical Formula 1 is selected from following Chemical Formula 6 to Chemical Formula 13: 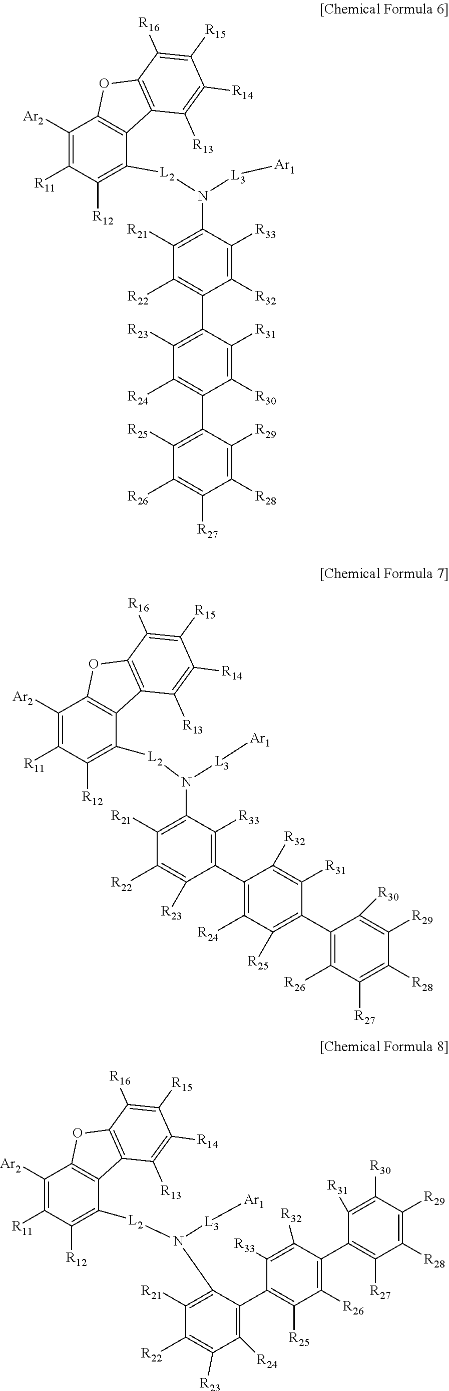 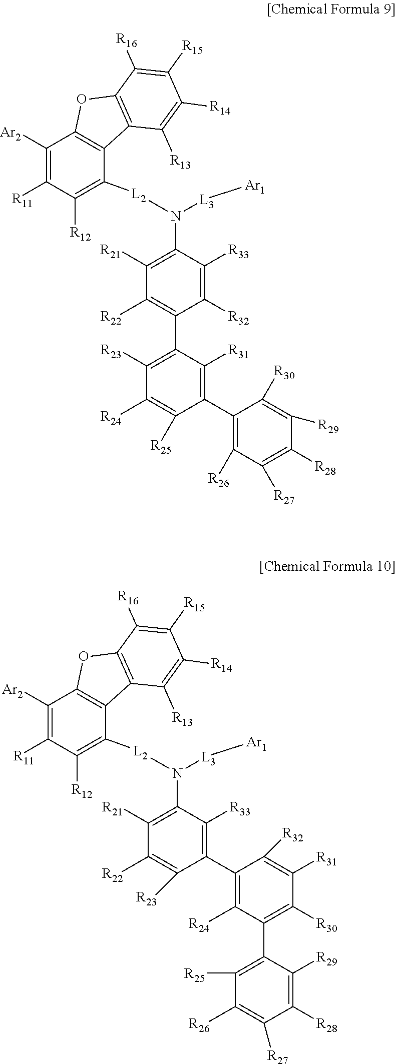 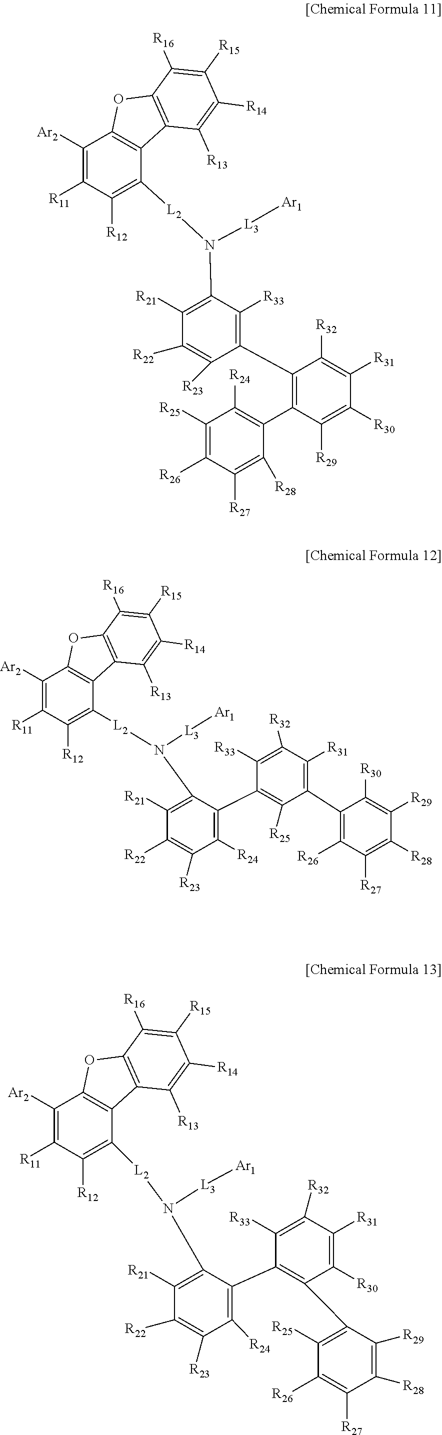
wherein in each of the Chemical Formulas 6 to 13,
each of L₂, L₃, R₁₁ to R₁₆, Ar₁ and Ar₂ is the same as defined in the Chemical Formula 1,
R₂₁ to R₃₃ are identical with or different from each other, wherein each of R₂₁ to R₃₃ independently represents one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6 to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms.

7. The organic compound of claim 1, wherein the Chemical Formula 1 is selected from following Chemical Formula 14 to Chemical Formula 45: 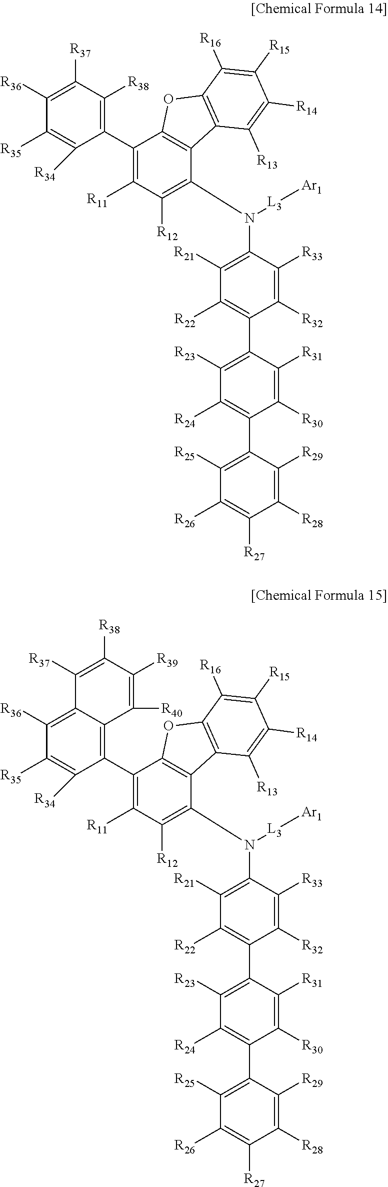 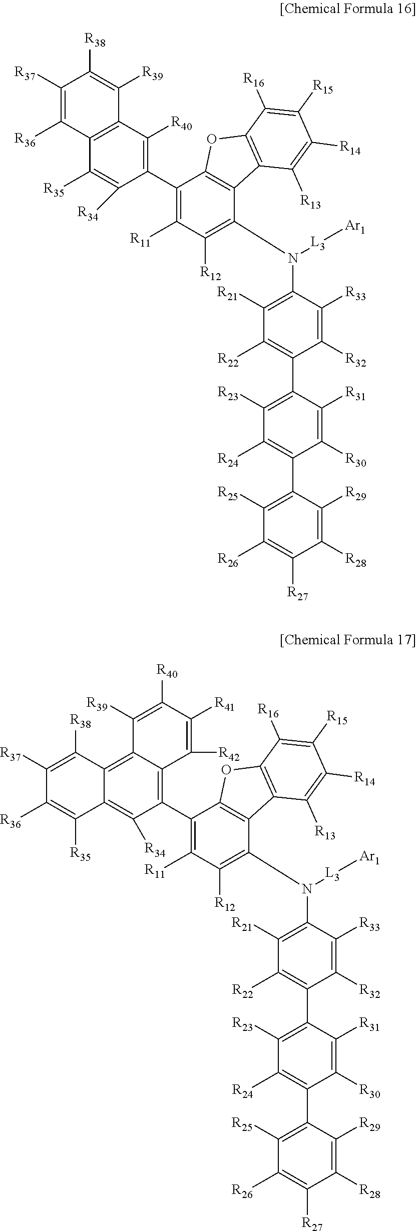 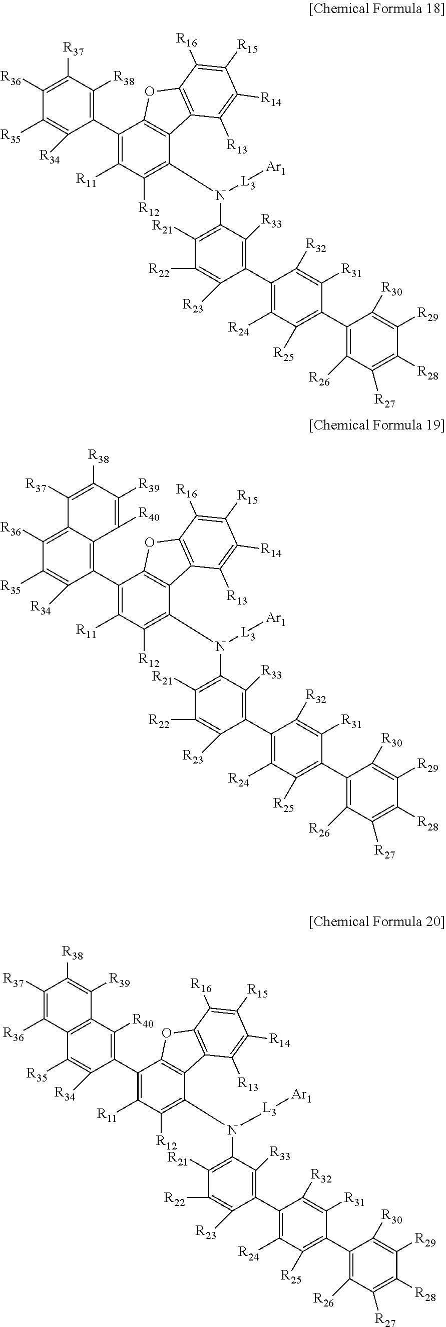 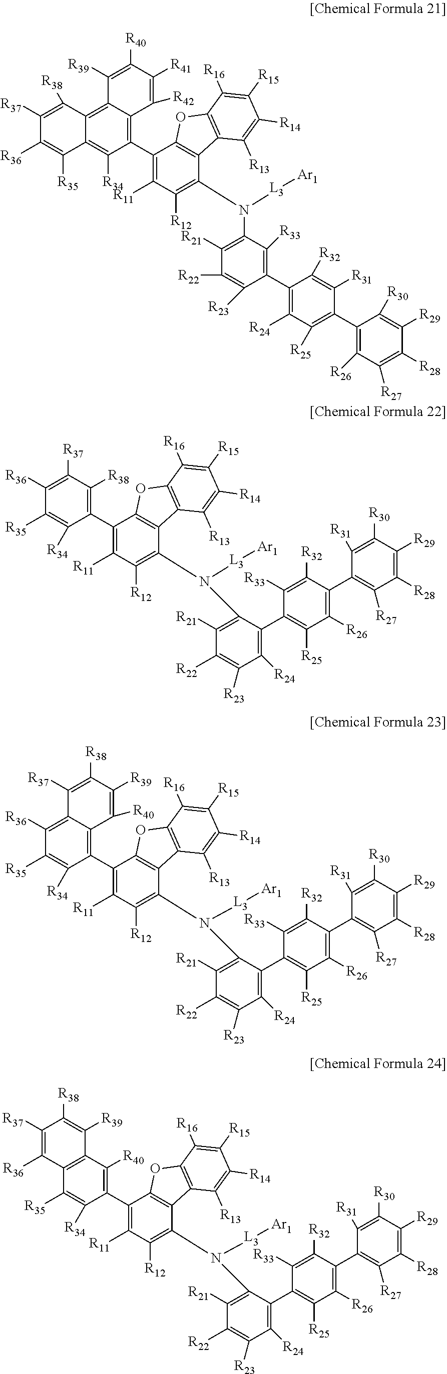 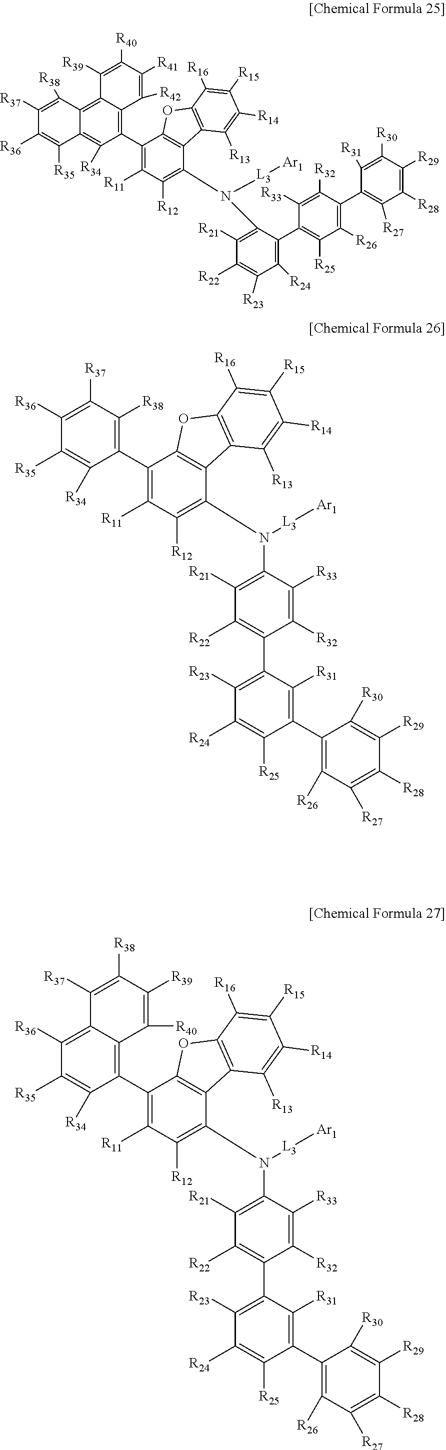 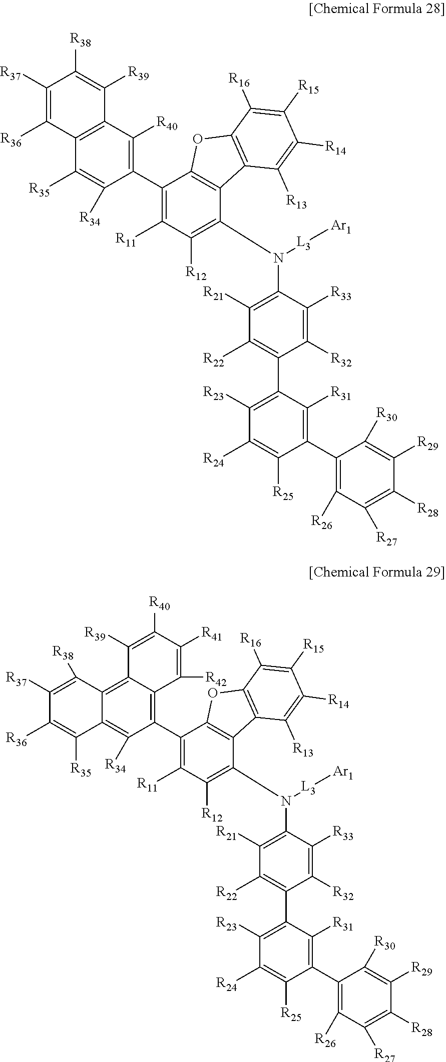 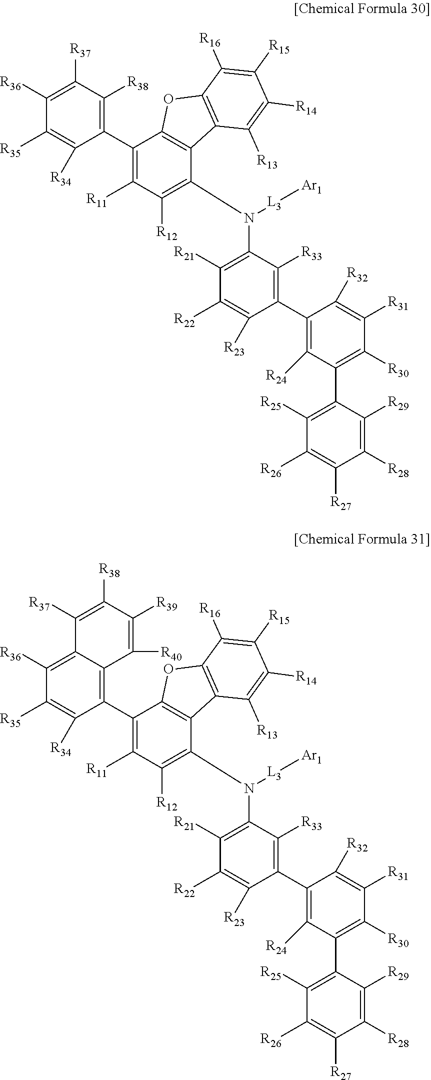 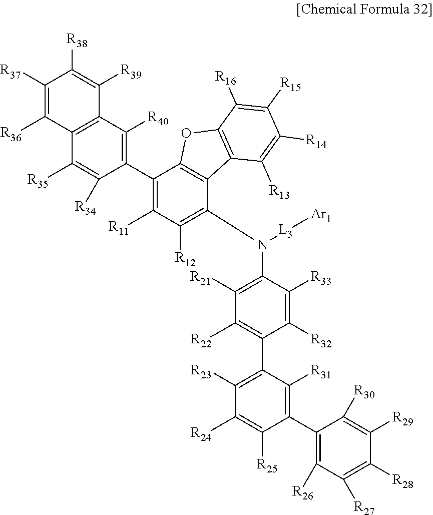 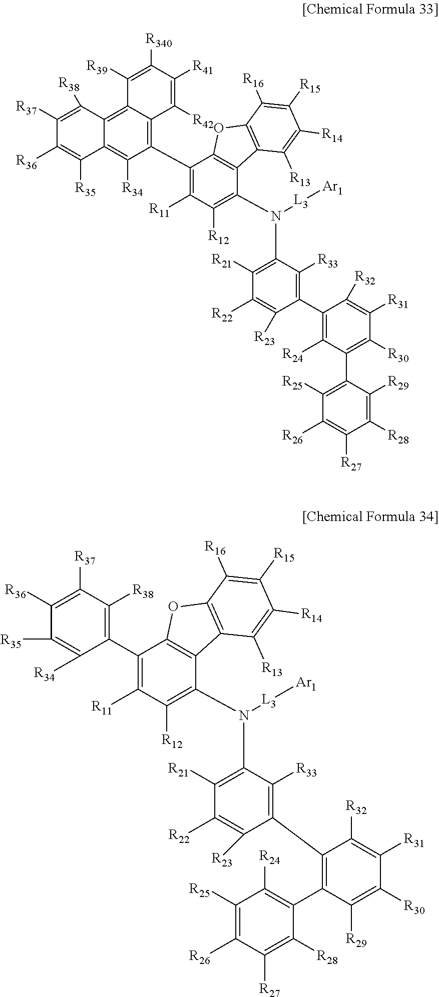 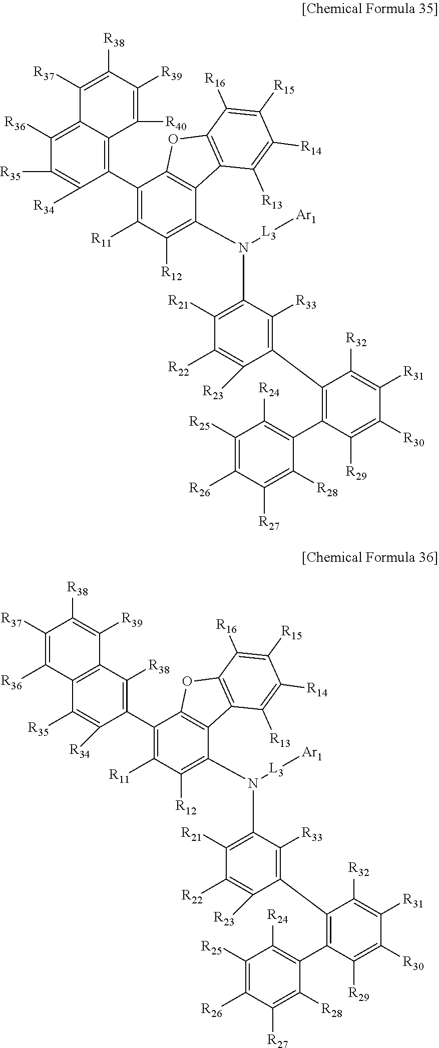 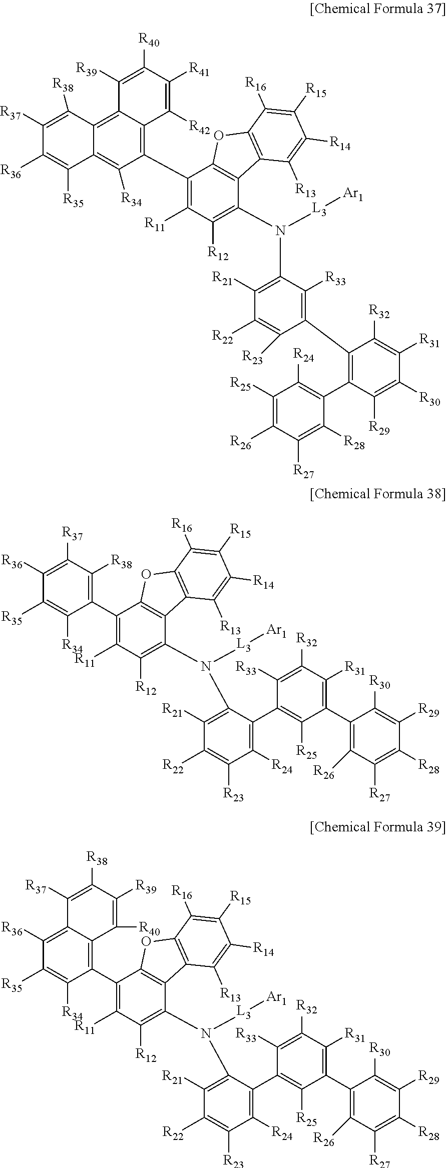 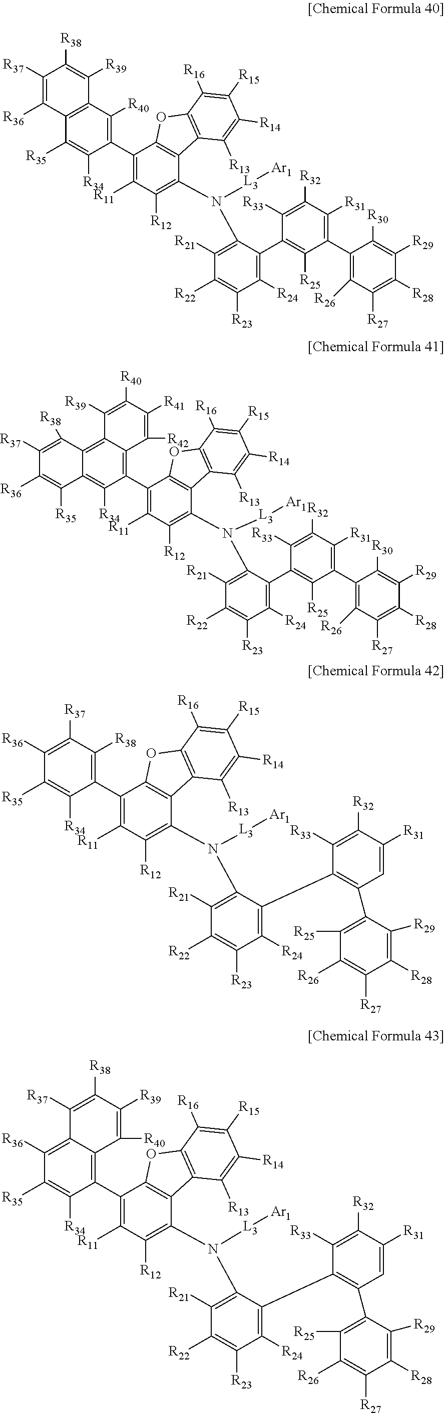 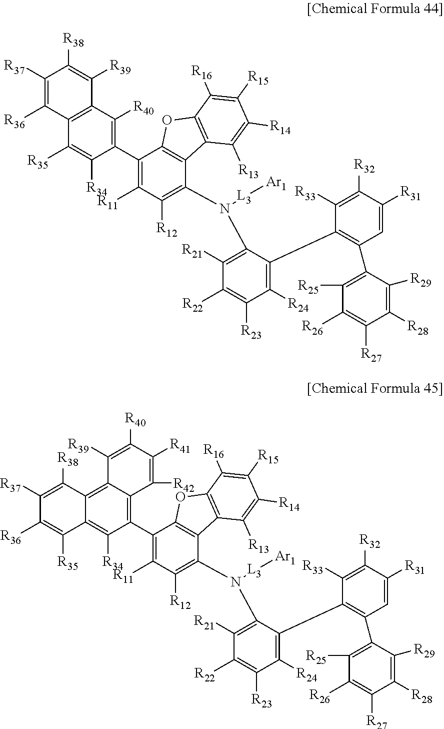
wherein in each of the Chemical Formulas 14 to 45,
each of L₃, R₁₁ to R₁₆ and Ar₁ is the same as defined in the Chemical Formula 1,
R₂₁ to R₄₂ are identical with or different from each other, wherein each of R₂₁ to R₄₂ independently represents one selected from a group consisting of hydrogen, deuterium, a cyano group, a trifluoromethyl group, a nitro group, a halogen group, a hydroxy group, a trimethylsilylethynyl group (TMS), a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted cycloalkenyl group having 3 to 20 carbon atoms, a substituted or unsubstituted alkynyl group having 2 to 30 carbon atoms, a substituted or unsubstituted cycloalkynyl group having 3 to 20 carbon atoms, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, a substituted or unsubstituted heteroaralkyl group having 6 to 60 carbon atoms, a substituted or unsubstituted amine group, a substituted or unsubstituted alkylamino group having 1 to 30 carbon atoms, a substituted or unsubstituted aralkylamino group having 7 to 30 carbon atoms, a substituted or unsubstituted arylamino group having 6 to 30 carbon atoms, a substituted or unsubstituted heteroarylamino group having 5 to 60 carbon atoms, a substituted or unsubstituted silyl group, a substituted or unsubstituted alkylsilyl group having 1 to 30 carbon atoms, a substituted or unsubstituted arylsilyl group having 6 to 30 carbon atoms, a substituted or unsubstituted alkoxy group having 1 to 30 carbon atoms, a substituted or unsubstituted aryloxy group having 6 to 30 carbon atoms, a substituted or unsubstituted alkylthio group having 1 to 30 carbon atoms, and a substituted or unsubstituted arylthio group having 6 to 30 carbon atoms.

8. An organic light-emitting diode comprising:
a positive electrode;
a negative electrode facing the positive electrode; and
at least one organic material layer disposed between the positive electrode and the negative electrode,
wherein at least one of the organic material layer contains the organic compound according to claim 1.

9. The organic light-emitting diode of claim 8, wherein the organic material layer containing the organic compound according to claim 1 is an hole transport auxiliary layer.

10. The organic light-emitting diode of claim 8, wherein the at least one organic material layer further includes at least one selected from a group consisting of a hole injection layer, a hole transport layer, a light-emitting layer, an electron transport layer, and an electron injection layer.
